(19) United States
(12) Patent Application Publication
(10) Pub. No.: US 2026/0335420 A1
(43) Pub. Date: Oct. 29, 2026

(54) LYME DISEASE VACCINES

(71) Applicant: ModernaTX, Inc., Cambridge, MA (US)

(72) Inventors: Obadiah Plante, Cambridge, MA (US); Sunny Himansu, Winchester, MA (US); Guillaume Stewart-Jones, Cambridge, MA (US); Gwo-Yu Chuang, Cambridge, MA (US); Meredith Finn, Cambridge, MA (US)

(73) Assignee: ModernaTX, Inc., Cambridge, MA (US)

(21) Appl. No.: 19/473,846

(22) PCT Filed: Apr. 10, 2024
(86) PCT No.: PCT/US2024/023822

§ 371 Date: Oct. 9, 2025

Related U.S. Application Data
(60) Provisional application No. 63/458,409, filed on Apr. 10, 2023.
(60) Provisional application No. 63/465,469, filed on May 10, 2023.
(60) Provisional application No. 63/513,969, filed on Jul. 17, 2023.
(60) Provisional application No. 63/593,154, filed on Oct. 25, 2023.

(51) Int. Cl. A61K 39/02; A61K 9/00; A61K 39/00; A61K 39/116; A61P 31/04; C12N 15/11

(52) U.S. Cl. CPC A61K 39/0225; A61K 9/0019; A61K 39/116; A61P 31/04; C12N 15/11; A61K 2039/53; A61K 2039/54; A61K 2039/545; A61K 2039/55555; A61K 2039/575; A61K 2039/58; A61K 2039/70

(57) ABSTRACT

This disclosure provides methods and compositions related to messenger ribonucleic acid (mRNA) vaccines for Lyme disease and related methods of prevention. The mRNA vaccines comprise an mRNA comprising an open reading frame(s) encoding for a protein comprising a Borrelia outer surface protein A (OspA) extracellular domain. In some aspects of the disclosure, the mRNA vaccines comprise mRNA polynucleotides collectively encoding for at least seven different Borrelia outer surface protein A (OspA) extracellular domains.

### RELATED APPLICATIONS

This application claims the benefit under 35 U.S.C. § 119(e) to U.S. Provisional Application No. 63/458,409, entitled “LYME DISEASE VACCINES,” filed on Apr. 10, 2023, and U.S. Provisional Application No. 63/465,469, entitled “LYME DISEASE VACCINES,” filed on May 10, 2023, and U.S. Provisional Application No. 63/513,969, entitled “LYME DISEASE VACCINES,” filed on Jul. 17, 2023, and U.S. Provisional Application No. 63/593,154, entitled “LYME DISEASE VACCINES,” filed on Oct. 25, 2023; the entire contents of each of which are herein incorporated by reference.

### REFERENCE TO AN ELECTRONIC SEQUENCE LISTING

The contents of the electronic sequence listing (M137870198WO00-SEQ-CRP.xml; Size: 89,256 bytes; and Date of Creation: Apr. 10, 2024) is herein incorporated by reference in its entirety.

### BACKGROUND

Lyme disease in the United States is primarily caused by the bacterium Borrelia burgdorferi, which is transmitted to humans through the bite of infected black-legged ticks. In Europe and Asia, Lyme disease is primarily caused by Borrelia afzelii, Borrelia garinii, and/or Borrelia bavariensis. Symptoms of Lyme disease include fever, headache, body aches fatigue, and a skin rash called erythema migrans. Left untreated, the infection can develop into a chronic illness that spreads to joints, the heart, and the nervous system, leading to long-term and serious health problems, such as Bell's palsy and liver inflammation. There were two Lyme disease vaccines available in the United States, LYMERIX® and IMULYME®, but both were withdrawn from the market years ago due to low demand and concerns about possible side effects. Currently, there are no Lyme disease vaccines approved for use in humans in the United States, yet almost 40,000 cases are reported annually, and the Centers for Disease Control and Prevention estimates that the real number of infections may be 10 times greater.

### SUMMARY

The present disclosure relates to mRNA vaccines for the prevention of Lyme disease caused by Borrelia infection. Some aspects provide a messenger ribonucleic acid (mRNA) vaccine, comprising: an mRNA polynucleotide comprising an open reading frame encoding a protein comprising a Borrelia outer surface protein A (OspA) extracellular domain and optionally a heterologous amino terminal transmembrane domain; and a lipid nanoparticle. Other aspects provide an mRNA vaccine comprises an mRNA polynucleotide comprising: an open reading frame encoding a nonlipidated Borrelia OspA protein; and a lipid nanoparticle. In some embodiments, the mRNA vaccine comprises multiple mRNA polynucleotides each comprising an open reading frame encoding a different nonlipidated Borrelia OspA protein. In another embodiment, the mRNA vaccine comprises seven different open reading frames encoding seven different nonlipidated Borrelia OspA proteins.

In some embodiments, the Borrelia OspA protein(s) comprises a heterologous amino terminal transmembrane domain. In some embodiments, the Borrelia OspA protein(s) comprises a heterologous carboxyl terminal transmembrane domain.

In some embodiments, the heterologous amino terminal transmembrane domain is from an influenza virus neuraminidase protein.

In some embodiments, the Borrelia OspA protein(s) comprises one or more modified N glycosylation sites, relative to a corresponding naturally occurring Borrelia OspA protein.

In some embodiments, the Borrelia OspA protein comprises a Borrelia burgdorferi OspA serotype 1 (S1, also referred to herein as SR1) extracellular domain.

In some embodiments, the Borrelia OspA S1 protein comprises a human leukocyte function-associated antigen (hLFA-1) epitope that is modified or removed relative to a corresponding naturally occurring Borrelia OspA S1 protein.

In some embodiments, the hLFA-1 epitope of the Borrelia OspA S1 protein comprises the following mutations Y165F, V166I, T170R, and L171F, relative to a naturally occurring Borrelia OspA S1 protein comprising the amino acid sequence of SEQ ID NO: 24.

In some embodiments, mRNA vaccine further comprises one or more polynucleotides mRNA polynucleotide comprising an open reading frame encoding a nonlipidated Borrelia OspA protein extracellular domain selected from the group consisting of: a Borrelia afzelii OspA serotype 2 (S2, also referred to herein as SR2) extracellular domain, a Borrelia garinii OspA serotype 3 (S3, also referred to herein as SR3) extracellular domain, a Borrelia bavariensis OspA serotype 4 (S4, also referred to herein as SR4) extracellular domain, a Borrelia garinii OspA serotype 5 (S5, also referred to herein as SR5) extracellular domain, a Borrelia garinii OspA serotype 6 (S6, also referred to herein as SR6) extracellular domain, and a Borrelia garinii OspA serotype 7 (S7, also referred to herein as SR7) extracellular domain, optionally wherein the nonlipidated Borrelia OspA protein encoded by the additional mRNA polynucleotide comprises a heterologous amino terminal transmembrane domain and/or N-linked glycan site mutations.

In some embodiments, the mRNA vaccine further comprises two, three, four, five or six additional mRNAs, each comprising an open reading frame encoding a nonlipidated Borrelia OspA protein comprising a different Borrelia OspA extracellular domain selected from a Borrelia afzelii OspA S2 extracellular domain, a Borrelia garinii OspA S3 extracellular domain, a Borrelia bavariensis OspA S4 extracellular domain, a Borrelia garinii OspA S5 extracellular domain, a Borrelia garinii S6 protein, and a Borrelia garinii OspA S7 extracellular domain, optionally wherein each of the nonlipidated Borrelia OspA proteins encoded by the additional mRNAs comprises a heterologous amino terminal transmembrane domain and/or N-linked glycan site mutations.

In some embodiments, the mRNA vaccine further comprises a second mRNA polynucleotide comprising an open reading frame encoding a nonlipidated Borrelia OspA protein comprising a Borrelia afzelii OspA S2 extracellular domain, optionally wherein the nonlipidated Borrelia OspA protein encoded by the second mRNA polynucleotide comprises a heterologous amino terminal transmembrane domain and/or N-linked glycan site mutations.

In some embodiments, the mRNA vaccine further comprises a third mRNA polynucleotide comprising an open reading frame encoding a nonlipidated Borrelia OspA protein comprising a Borrelia garinii OspA S3 extracellular domain, optionally wherein the nonlipidated Borrelia OspA protein encoded by the third mRNA polynucleotide comprises a heterologous amino terminal transmembrane domain and/or N-linked glycan site mutations.

In some embodiments, the mRNA vaccine further comprises a fourth mRNA polynucleotide comprising an open reading frame encoding a nonlipidated Borrelia OspA protein comprising a Borrelia bavariensis OspA S4 extracellular domain, optionally wherein the nonlipidated Borrelia OspA protein encoded by the fourth mRNA polynucleotide comprises a heterologous amino terminal transmembrane domain and/or N-linked glycan site mutations.

In some embodiments, the mRNA vaccine further comprises a fifth mRNA polynucleotide comprising an open reading frame encoding a nonlipidated Borrelia OspA protein comprising a Borrelia garinii OspA S5 extracellular domain, optionally wherein the nonlipidated Borrelia OspA protein encoded by the fifth mRNA polynucleotide comprises a heterologous amino terminal transmembrane domain and/or N-linked glycan site mutations.

In some embodiments, the mRNA vaccine further comprises a six mRNA polynucleotide comprising an open reading frame encoding a nonlipidated Borrelia OspA protein comprising a Borrelia garinii OspA S6 extracellular domain, optionally wherein the nonlipidated Borrelia OspA protein encoded by the sixth mRNA polynucleotide comprises a heterologous amino terminal transmembrane domain and/or N-linked glycan site mutations.

In some embodiments, the nonlipidated Borrelia OspA protein comprising a Borrelia garinii OspA S6 extracellular domain comprises one or more amino acid modification(s), relative to a corresponding naturally occurring Borrelia garinii OspA S6 protein, to stabilize expression in mammalian cells.

In some embodiments, the one or more amino acid modification(s) is a mutation corresponding to a S88L mutation or an A200V mutation of the naturally occurring Borrelia garinii OspA S6 protein.

In some embodiments, the one or more amino acid modification(s) is a mutation corresponding to a S88L mutation and an A200V mutation of the naturally occurring Borrelia garinii OspA S6 protein.

In some embodiments, the mRNA vaccine further comprises a seventh mRNA polynucleotide comprising an open reading frame encoding a nonlipidated Borrelia OspA protein comprising a Borrelia garinii OspA S7 extracellular domain, optionally wherein the nonlipidated Borrelia OspA protein encoded by the seventh mRNA polynucleotide comprises a heterologous amino terminal transmembrane domain and/or N-linked glycan site mutations.

Some aspects relate to an mRNA vaccine, comprising: (a) an mRNA polynucleotide comprising an open reading frame encoding an outer surface protein A (OspA) protein comprising an extracellular domain of a first serotype of Borrelia; (b) an mRNA polynucleotide comprising an open reading frame encoding an OspA protein comprising an extracellular domain of a second serotype of Borrelia, wherein the second serotype of Borrelia is of a species that is different from that of the first serotype of Borrelia; and a lipid nanoparticle.

Other aspects relate to an mRNA vaccine, comprising: at least two, three, four, five, six, or seven mRNAs, each comprising an open reading frame encoding a Borrelia outer surface protein A (OspA) protein comprising an extracellular domain, wherein the extracellular domain of each of the Borrelia OspA proteins is of a different Borrelia serotype; and a lipid nanoparticle.

Yet other aspects relate to an mRNA vaccine, comprising at least four mRNAs, optionally seven mRNAs, each encoding a Borrelia OspA protein comprising an extracellular domain of one of four different serotypes of Borrelia, optionally selected from Borrelia burgdorferi, Borrelia afzelii, Borrelia garinii, and Borrelia bavariensis; and a lipid nanoparticle.

In some embodiments, each of the Borrelia OspA proteins comprises a heterologous amino terminal transmembrane domain, optionally an influenza virus neuraminidase transmembrane domain.

Some aspects relate to an mRNA vaccine, comprising: an mRNA polynucleotide comprising an open reading frame encoding a protein comprising an extracellular domain from a Borrelia garinii OspA serotype 6 protein, wherein the extracellular domain comprises one or more amino acid modification(s), optionally selected from a S88L mutation and an A200V mutation, relative to a corresponding naturally occurring Borrelia garinii OspA S6 extracellular domain, to stabilize expression in mammalian cells; and a lipid nanoparticle.

In some embodiments, the protein further comprises a heterologous amino terminal transmembrane domain and/or one or more N-linked glycan site mutation(s).

In some embodiments, the one or more mRNA(s) of the vaccine comprises a chemical modification.

In some embodiments, 100% of the uracil nucleotides of the one or more mRNA(s) comprise a chemical modification. In some embodiments, the chemical modification is 1-methylpseudouracil.

In some embodiments, the lipid nanoparticle comprises an ionizable lipid, a neutral lipid, a sterol, and a PEG-modified lipid. In some embodiments, the lipid nanoparticle comprises 40-50 mol % ionizable lipid, 5-15 mol % neutral lipid, 30-50 mol % sterol, and 0.5-3 mol % PEG-modified lipid. In some embodiments, the ionizable lipid comprises a structure of Compound (I):

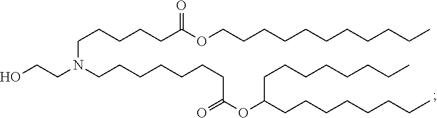 [SMILES: CCCCCCCCCCCOC(=O)CCCCCN(CCO)CCCCCCCC(=O)OC(CCCCCCCC)CCCCCCCC]

the neutral lipid is distearoylphosphatidylcholine (DSPC);
the sterol is cholesterol; and/or
the PEG-modified lipid is 1,2 dimyristoyl-sn-glycerol, methoxypolyethyleneglycol (PEG-DMG).

Some aspects relate to a method of preventing Lyme disease in a subject in need thereof, the method comprising administering to the subject one or more doses of the vaccine of any one of the preceding claims in an effective amount to produce an immune response to a Borrelia bacterial infection.

In some embodiments, the vaccine is administered intramuscularly.

In some embodiments, the method comprises administering a single dose of the vaccine to the subject.

In some embodiments, the method comprises administering more than one dose of the vaccine to the subject.

In some embodiments, an Borrelia burgdorferi OspA S1 protein described herein comprises an amino acid sequence having at least 80%, at least 85%, at least 90%, at least 95%, or 100% identity to an amino acid sequence consisting of amino acid residues 40-287 of SEQ ID NO: 1. In some embodiments, the Borrelia burgdorferi OspA S1 protein comprises an amino acid sequence having at least 80%, at least 85%, at least 90%, at least 95%, or 100% identity to the amino acid sequence of SEQ ID NO: 1. In some embodiments, the amino acid sequence of the Borrelia burgdorferi OspA S1 protein (e.g., an amino acid sequence having at least 80%, at least 85%, at least 90%, at least 95%, or 100% identity to an amino acid sequence consisting of amino acid residues 40-287 of SEQ ID NO: 1 and/or at least at least 80%, at least 85%, at least 90%, at least 95%, or 100% identity to the amino acid sequence of SEQ ID NO: 1) comprises the conserved OspA amino acids depicted with an asterisk (*) in FIG. 16. In some embodiments, the amino acid sequence of the Borrelia burgdorferi OspA S1 protein (e.g., an amino acid sequence having at least 80%, at least 85%, at least 90%, at least 95%, or 100% identity to an amino acid sequence consisting of amino acid residues 40-287 of SEQ ID NO: 1 and/or at least at least 80%, at least 85%, at least 90%, at least 95%, or 100% identity to the amino acid sequence of SEQ ID NO: 1) comprises:

the amino acid sequence
(SEQ ID NO: 67)
EKNSXSVDLPGXMXVLVSKEKXKDGKYXLXATVXKXELKGTSDKXNGXGX
LEGXKXXKSKXKXTIXXDLXXTXXEXFKEDGKTLVSXKVXXKDKXSXXEX
FNXKGXXSXKXXXRXXGTXLEYTXXXXDGXGKAKEVLKXXXLEG,
wherein X is any amino acid;
and/or
the amino acid sequence
(SEQ ID NO: 68)
LXVXXGTVXLSKXIXXSGEXXXXLXDXXXXXAXKKXXXWXSXTSTLTIXX
NSXKXXXXVFTKXXTITVQXYXXXGXXLEGXXXEIXXLXXXKXALK,
wherein X is any amino acid.

In some embodiments, an Borrelia afzelii OspA S2 protein described herein comprises an amino acid sequence having at least 80%, at least 85%, at least 90%, at least 95%, or 100% identity to an amino acid sequence consisting of amino acid residues 40-287 of SEQ ID NO: 2.

In some embodiments, the Borrelia afzelii OspA S2 protein comprises an amino acid sequence having at least 80%, at least 85%, at least 90%, at least 95%, or 100% identity to the amino acid sequence of SEQ ID NO: 2. In some embodiments, the amino acid sequence of the Borrelia afzelii OspA S2 protein (e.g., an amino acid sequence having at least 80%, at least 85%, at least 90%, at least 95%, or 100% identity to an amino acid sequence consisting of amino acid residues 40-287 of SEQ ID NO: 2 and/or at least at least 80%, at least 85%, at least 90%, at least 95%, or 100% identity to the amino acid sequence of SEQ ID NO: 2) comprises the conserved OspA amino acids depicted with an asterisk (*) in FIG. 16. In some embodiments, the amino acid sequence of the Borrelia afzelii OspA S2 protein (e.g., an amino acid sequence having at least 80%, at least 85%, at least 90%, at least 95%, or 100% identity to an amino acid sequence consisting of amino acid residues 40-287 of SEQ ID NO: 2 and/or at least at least 80%, at least 85%, at least 90%, at least 95%, or 100% identity to the amino acid sequence of SEQ ID NO: 2) comprises:

the amino acid sequence
(SEQ ID NO: 67)
EKNSXSVDLPGXMXVLVSKEKXKDGKYXLXATVXKXELKGTSDKXNGXGX
LEGXKXXKSKXKXTIXXDLXXTXXEXFKEDGKTLVSXKVXXKDKXSXXEX
FNXKGXXSXKXXXRXXGTXLEYTXXXXDGXGKAKEVLKXXXLEG,
wherein X is any amino acid;
and/or
the amino acid sequence
(SEQ ID NO: 68)
LXVXXGTVXLSKXIXXSGEXXXXLXDXXXXXAXKKXXXWXSXTSTLTIXX
NSXKXXXXVFTKXXTITVQXYXXXGXXLEGXXXEIXXLXXXKXALK,
wherein X is any amino acid.

In some embodiments, an Borrelia garinii OspA S3 protein described herein comprises an amino acid sequence having at least 80%, at least 85%, at least 90%, at least 95%, or 100% identity to an amino acid sequence consisting of amino acid residues 40-288 of SEQ ID NO: 3. In some embodiments, the Borrelia garinii OspA S3 protein comprises an amino acid sequence having at least 80%, at least 85%, at least 90%, at least 95%, or 100% identity to the amino acid sequence of SEQ ID NO: 3. In some embodiments, the amino acid sequence of the Borrelia garinii OspA S3 protein (e.g., an amino acid sequence having at least 80%, at least 85%, at least 90%, at least 95%, or 100% identity to an amino acid sequence consisting of amino acid residues 40-288 of SEQ ID NO: 3 and/or at least at least 80%, at least 85%, at least 90%, at least 95%, or 100% identity to the amino acid sequence of SEQ ID NO: 3) comprises the conserved OspA amino acids depicted with an asterisk (*) in FIG. 16. In some embodiments, the amino acid sequence of the Borrelia garinii OspA S3 protein (e.g., an amino acid sequence having at least 80%, at least 85%, at least 90%, at least 95%, or 100% identity to an amino acid sequence consisting of amino acid residues 40-288 of SEQ ID NO: 3 and/or at least at least 80%, at least 85%, at least 90%, at least 95%, or 100% identity to the amino acid sequence of SEQ ID NO: 3)

the amino acid sequence
(SEQ ID NO: 67)
EKNSXSVDLPGXMXVLVSKEKXKDGKYXLXATVXKXELKGTSDKXNGXGX
LEGXKXXKSKXKXTIXXDLXXTXXEXFKEDGKTLVSXKVXXKDKXSXXEX
FNXKGXXSXKXXXRXXGTXLEYTXXXXDGXGKAKEVLKXXXLEG,
wherein X is any amino acid;
and/or
the amino acid sequence
(SEQ ID NO: 68)
LXVXXGTVXLSKXIXXSGEXXXXLXDXXXXXAXKKXXXWXSXTSTLTIXX
NSXKXXXXVFTKXXTITVQXYXXXGXXLEGXXXEIXXLXXXKXALK,
wherein X is any amino acid.

In some embodiments, an Borrelia bavariensis OspA S4 protein described herein comprises an amino acid sequence having at least 80%, at least 85%, at least 90%, at least 95%, or 100% identity to an amino acid sequence consisting of amino acid residues 40-287 of SEQ ID NO: 4. In some embodiments, the Borrelia bavariensis OspA S4 protein comprises an amino acid sequence having at least 80%, at least 85%, at least 90%, at least 95%, or 100% identity to the amino acid sequence of SEQ ID NO: 4. In some embodiments, the amino acid sequence of the Borrelia bavariensis OspA S4 protein (e.g., an amino acid sequence having at least 80%, at least 85%, at least 90%, at least 95%, or 100% identity to an amino acid sequence consisting of amino acid residues 40-287 of SEQ ID NO: 4 and/or at least at least 80%, at least 85%, at least 90%, at least 95%, or 100% identity to the amino acid sequence of SEQ ID NO: 4) comprises the conserved OspA amino acids depicted with an asterisk (*) in FIG. 16. In some embodiments, the amino acid sequence of the Borrelia bavariensis OspA S4 protein (e.g., an amino acid sequence having at least 80%, at least 85%, at least 90%, at least 95%, or 100% identity to an amino acid sequence consisting of amino acid residues 40-287 of SEQ ID NO: 4 and/or at least at least 80%, at least 85%, at least 90%, at least 95%, or 100% identity to the amino acid sequence of SEQ ID NO: 4) comprises:

the amino acid sequence
(SEQ ID NO: 67)
EKNSXSVDLPGXMXVLVSKEKXKDGKYXLXATVXKXELKGTSDKXNGXGX
LEGXKXXKSKXKXTIXXDLXXTXXEXFKEDGKTLVSXKVXXKDKXSXXEX
FNXKGXXSXKXXXRXXGTXLEYTXXXXDGXGKAKEVLKXXXLEG,
wherein X is any amino acid;
and/or
the amino acid sequence
(SEQ ID NO: 68)
LXVXXGTVXLSKXIXXSGEXXXXLXDXXXXXAXKKXXXWXSXTSTLTIXX
NSXKXXXXVFTKXXTITVQXYXXXGXXLEGXXXEIXXLXXXKXALK,
wherein X is any amino acid.

In some embodiments, an Borrelia garinii OspA S5 protein described herein comprises an amino acid sequence having at least 80%, at least 85%, at least 90%, at least 95%, or 100% identity to an amino acid sequence consisting of amino acid residues 40-288 of SEQ ID NO: 5. In some embodiments, the Borrelia garinii OspA S5 protein comprises an amino acid sequence having at least 80%, at least 85%, at least 90%, at least 95%, or 100% identity to the amino acid sequence of SEQ ID NO: 5. In some embodiments, the amino acid sequence of the Borrelia garinii OspA S5 protein (e.g., an amino acid sequence having at least 80%, at least 85%, at least 90%, at least 95%, or 100% identity to an amino acid sequence consisting of amino acid residues 40-288 of SEQ ID NO: 5 and/or at least at least 80%, at least 85%, at least 90%, at least 95%, or 100% identity to the amino acid sequence of SEQ ID NO: 5) comprises the conserved OspA amino acids depicted with an asterisk (*) in FIG. 16. In some embodiments, the amino acid sequence of the Borrelia garinii OspA S5 protein (e.g., an amino acid sequence having at least 80%, at least 85%, at least 90%, at least 95%, or 100% identity to an amino acid sequence consisting of amino acid residues 40-288 of SEQ ID NO: 5 and/or at least at least 80%, at least 85%, at least 90%, at least 95%, or 100% identity to the amino acid sequence of SEQ ID NO: 5) comprises:

the amino acid sequence
(SEQ ID NO: 67)
EKNSXSVDLPGXMXVLVSKEKXKDGKYXLXATVXKXELKGTSDKXNGXGX
LEGXKXXKSKXKXTIXXDLXXTXXEXFKEDGKTLVSXKVXXKDKXSXXEX
FNXKGXXSXKXXXRXXGTXLEYTXXXXDGXGKAKEVLKXXXLEG,
wherein X is any amino acid;
and/or
the amino acid sequence
(SEQ ID NO: 68)
LXVXXGTVXLSKXIXXSGEXXXXLXDXXXXXAXKKXXXWXSXTSTLTIXX
NSXKXXXXVFTKXXTITVQXYXXXGXXLEGXXXEIXXLXXXKXALK,
wherein X is any amino acid.

In some embodiments, an Borrelia garinii OspA S6 protein described herein comprises an amino acid sequence having at least 80%, at least 85%, at least 90%, at least 95%, or 100% identity to an amino acid sequence consisting of amino acid residues 40-288 of SEQ ID NO: 6.

In some embodiments, the Borrelia garinii OspA S6 protein comprises an amino acid sequence having at least 80%, at least 85%, at least 90%, at least 95%, or 100% identity to the amino acid sequence of SEQ ID NO: 6. In some embodiments, the amino acid sequence of the Borrelia garinii OspA S6 protein (e.g., an amino acid sequence having at least 80%, at least 85%, at least 90%, at least 95%, or 100% identity to an amino acid sequence consisting of amino acid residues 40-288 of SEQ ID NO: 6 and/or at least at least 80%, at least 85%, at least 90%, at least 95%, or 100% identity to the amino acid sequence of SEQ ID NO: 6) comprises the conserved OspA amino acids depicted with an asterisk (*) in FIG. 16. In some embodiments, the amino acid sequence of the Borrelia garinii OspA S6 protein (e.g., an amino acid sequence having at least 80%, at least 85%, at least 90%, at least 95%, or 100% identity to an amino acid sequence consisting of amino acid residues 40-288 of SEQ ID NO: 6 and/or at least at least 80%, at least 85%, at least 90%, at least 95%, or 100% identity to the amino acid sequence of SEQ ID NO: 6) comprises:

the amino acid sequence
(SEQ ID NO: 67)
EKNSXSVDLPGXMXVLVSKEKXKDGKYXLXATVXKXELKGTSDKXNGXGX
LEGXKXXKSKXKXTIXXDLXXTXXEXFKEDGKTLVSXKVXXKDKXSXXEX
FNXKGXXSXKXXXRXXGTXLEYTXXXXDGXGKAKEVLKXXXLEG,
wherein X is any amino acid;
and/or
the amino acid sequence
(SEQ ID NO: 68)
LXVXXGTVXLSKXIXXSGEXXXXLXDXXXXXAXKKXXXWXSXTSTLTIXX
NSXKXXXXVFTKXXTITVQXYXXXGXXLEGXXXEIXXLXXXKXALK,
wherein X is any amino acid.

In some embodiments, an Borrelia garinii OspA S7 protein described herein comprises an amino acid sequence having at least 80%, at least 85%, at least 90%, at least 95%, or 100% identity to an amino acid sequence consisting of amino acid residues 40-288 of SEQ ID NO: 7. In some embodiments, the Borrelia garinii OspA S7 protein comprises an amino acid sequence having at least 80%, at least 85%, at least 90%, at least 95%, or 100% identity to the amino acid sequence of SEQ ID NO: 7. In some embodiments, the amino acid sequence of the Borrelia garinii OspA S7 protein (e.g., an amino acid sequence having at least 80%, at least 85%, at least 90%, at least 95%, or 100% identity to an amino acid sequence consisting of amino acid residues 40-288 of SEQ ID NO: 7 and/or at least at least 80%, at least 85%, at least 90%, at least 95%, or 100% identity to the amino acid sequence of SEQ ID NO: 7) comprises the conserved OspA amino acids depicted with an asterisk (*) in FIG. 16. In some embodiments, the amino acid sequence of the Borrelia garinii OspA S7 protein (e.g., an amino acid sequence having at least 80%, at least 85%, at least 90%, at least 95%, or 100% identity to an amino acid sequence consisting of amino acid residues 40-288 of SEQ ID NO: 7 and/or at least at least 80%, at least 85%, at least 90%, at least 95%, or 100% identity to the amino acid sequence of SEQ ID NO: 7)

the amino acid sequence
(SEQ ID NO: 67)
EKNSXSVDLPGXMXVLVSKEKXKDGKYXLXATVXKXELKGTSDKXNGXGX
LEGXKXXKSKXKXTIXXDLXXTXXEXFKEDGKTLVSXKVXXKDKXSXXEX
FNXKGXXSXKXXXRXXGTXLEYTXXXXDGXGKAKEVLKXXXLEG,
wherein X is any amino acid;
and/or
the amino acid sequence
(SEQ ID NO: 68)
LXVXXGTVXLSKXIXXSGEXXXXLXDXXXXXAXKKXXXWXSXTSTLTIXX
NSXKXXXXVFTKXXTITVQXYXXXGXXLEGXXXEIXXLXXXKXALK,
wherein X is any amino acid.

In some embodiments: (a) the Borrelia burgdorferi OspA S1 protein comprises an amino acid sequence having at least 90% identity to the amino acid sequence of SEQ ID NO: 1; (b) the Borrelia afzelii OspA S2 protein comprises an amino acid sequence having at least 90% identity to the amino acid sequence of SEQ ID NO: 2; (c) the Borrelia garinii OspA S3 protein comprises an amino acid sequence having at least 90% identity to the amino acid sequence of SEQ ID NO: 3; (d) the Borrelia bavariensis OspA S4 protein comprises an amino acid sequence having at least 90% identity to the amino acid sequence of SEQ ID NO: 4; (e) the Borrelia garinii OspA S5 protein comprises an amino acid sequence having at least 90% identity to the amino acid sequence of SEQ ID NO: 5; (f) the Borrelia garinii OspA S6 protein comprises an amino acid sequence having at least 90% identity to the amino acid sequence of SEQ ID NO: 6; and (g) the Borrelia garinii OspA S7 protein comprises an amino acid sequence having at least 90% identity to the amino acid sequence of SEQ ID NO: 7.

In some embodiments: (a) the Borrelia burgdorferi OspA S1 protein comprises an amino acid sequence having at least 95% identity to the amino acid sequence of SEQ ID NO: 1; (b) the Borrelia afzelii OspA S2 protein comprises an amino acid sequence having at least 95% identity to the amino acid sequence of SEQ ID NO: 2; (c) the Borrelia garinii OspA S3 protein comprises an amino acid sequence having at least 95% identity to the amino acid sequence of SEQ ID NO: 3; (d) the Borrelia bavariensis OspA S4 protein comprises an amino acid sequence having at least 95% identity to the amino acid sequence of SEQ ID NO: 4; (e) the Borrelia garinii OspA S5 protein comprises an amino acid sequence having at least 95% identity to the amino acid sequence of SEQ ID NO: 5; (f) the Borrelia garinii OspA S6 protein comprises an amino acid sequence having at least 95% identity to the amino acid sequence of SEQ ID NO: 6; and (g) the Borrelia garinii OspA S7 protein comprises an amino acid sequence having at least 95% identity to the amino acid sequence of SEQ ID NO: 7.

In some embodiments: (a) the Borrelia burgdorferi OspA S1 protein comprises an amino acid sequence having at least 98% identity to the amino acid sequence of SEQ ID NO: 1; (b) the Borrelia afzelii OspA S2 protein comprises an amino acid sequence having at least 98% identity to the amino acid sequence of SEQ ID NO: 2; (c) the Borrelia garinii OspA S3 protein comprises an amino acid sequence having at least 98% identity to the amino acid sequence of SEQ ID NO: 3; (d) the Borrelia bavariensis OspA S4 protein comprises an amino acid sequence having at least 98% identity to the amino acid sequence of SEQ ID NO: 4; (e) the Borrelia garinii OspA S5 protein comprises an amino acid sequence having at least 98% identity to the amino acid sequence of SEQ ID NO: 5; (f) the Borrelia garinii OspA S6 protein comprises an amino acid sequence having at least 98% identity to the amino acid sequence of SEQ ID NO: 6; and (g) the Borrelia garinii OspA S7 protein comprises an amino acid sequence having at least 98% identity to the amino acid sequence of SEQ ID NO: 7.

With respect to such related sequences (e.g., at least 80%, at least 85%, at least 90%, at least 95%, or at least 98% identity to the amino acid sequences of any one of SEQ ID NOs: 1-7) one of ordinary skill in the art would recognize based on FIGS. 16-17 what amino acid positions are amenable to alteration without affecting protein function.

In some embodiments: (a) the Borrelia burgdorferi OspA S1 protein comprises the amino acid sequence of SEQ ID NO: 1; (b) the Borrelia afzelii OspA S2 protein comprises the amino acid sequence of SEQ ID NO: 2; (c) the Borrelia garinii OspA S3 protein comprises the amino acid sequence of SEQ ID NO: 3; (d) the Borrelia bavariensis OspA S4 protein comprises the amino acid sequence of SEQ ID NO: 4; (e) the Borrelia garinii OspA S5 protein comprises the amino acid sequence of SEQ ID NO: 5; (f) the Borrelia garinii OspA S6 protein comprises the amino acid sequence of SEQ ID NO: 6; and (g) the Borrelia garinii OspA S7 protein comprises the amino acid sequence of SEQ ID NO: 7.

Some aspects of the present disclosure relate to a method of vaccinating a subject, the method comprising administering to the subject one or more doses of an mRNA vaccine of the present disclosure in an effective amount to produce an immune response to a Borrelia bacterial infection.

In some embodiments, the vaccine is administered intramuscularly.

In some embodiments, the method comprises administering a dose of 12.5-150 μg of the mRNA comprised in the mRNA vaccine.

In some embodiments, the method comprises administering a single dose of the vaccine to the subject.

In some embodiments, the method comprises administering a first dose of the vaccine, a second dose of the vaccine, and a third dose of the vaccine. In some embodiments, the second dose is administered two months following the first dose, the third dose is administered six months following the first dose, or a combination thereof. In some embodiments, the first dose, the second dose, and the third dose is 50 μg of the mRNA, and wherein the second dose is administered 2 months after the first dose, and the third dose is administered 4 months after the second dose. In some embodiments, the first dose, the second dose, and the third dose is 100 μg of the mRNA, and wherein the second dose is administered 2 months after the first dose, and the third dose is administered 4 months after the second dose. In some embodiments, the first dose, the second dose, and the third dose is 150 μg of the mRNA, and wherein the second dose is administered 2 months after the first dose, and the third dose is administered 4 months after the second dose.

In some embodiments, the lipid nanoparticle comprises a mixture of lipids that comprises 20-60 mol % ionizable lipid heptadecan-9-yl 8 ((2 hydroxyethyl)(6 oxo 6-(undecyloxy)hexyl)amino)octanoate (Compound 1); 5-25 mol % 1,2 distearoyl sn glycero-3 phosphocholine (DSPC); 25-55 mol % cholesterol; and 0.5-15 mol % 1-monomethoxypolyethyleneglycol-2,3-dimyristylglycerol with polyethylene glycol of average molecular weight 2000 (PEG2000 DMG).

In some embodiments, the lipid nanoparticle comprises: 47 mol % ionizable cationic lipid; 11.5 mol % neutral lipid; 38.5 mol % sterol; and 3.0 mol % PEG-modified lipid; 48 mol % ionizable cationic lipid; 11 mol % neutral lipid; 38.5 mol % sterol; and 2.5 mol % PEG-modified lipid; 49 mol % ionizable cationic lipid; 10.5 mol % neutral lipid; 38.5 mol % sterol; and 2.0 mol % PEG-modified lipid; 50 mol % ionizable cationic lipid; 10 mol % neutral lipid; 38.5 mol % sterol; and 1.5 mol % PEG-modified lipid; or 51 mol % ionizable cationic lipid; 9.5 mol % neutral lipid; 38.5 mol % sterol; and 1.0 mol % PEG-modified lipid. In some embodiments, the mRNA vaccine further comprises Tris buffer, sucrose, and sodium acetate, or any combination thereof.

Some aspects of the present disclosure relate to a method of inducing an immune response in a subject (e.g., a human subject) to a Borrelia bacteria, the method comprising, administering to the subject an mRNA vaccine described herein in an amount effective at inducing a population of neutralizing antibodies in the subject. In some embodiments, the population of neutralizing antibodies comprises antibodies that bind to OspA S1, antibodies that bind to OspA S2, antibodies that bind to OspA S3, antibodies that bind to OspA S4, antibodies that bind to OspA S5, antibodies that bind to OspA S6, antibodies that bind to OspA S7, or any combination thereof. In some embodiments, the population of neutralizing antibodies comprises: IgG antibodies.

Some aspects of the present disclosure relate to compositions comprising a polynucleotide comprising a sequence having at least 80%, 85%, 90%, 95%, or 98% identity to the nucleic acid sequence of any one of SEQ ID NOs: 8-14. In some embodiments, the polynucleotide comprises the nucleic acid sequence of any one of SEQ ID NOs: 8-14.

Some aspects of the present disclosure relate to mRNA vaccine compositions comprising a plurality of polynucleotides, wherein the plurality comprises two or more of: a first polynucleotide comprising a sequence having at least 80%, 85%, 90%, 95%, or 98% identity to the nucleic acid sequence of SEQ ID NO: 8; a second polynucleotide comprising a sequence having at least 80%, 85%, 90%, 95%, or 98% identity to the nucleic acid sequence of SEQ ID NO: 9; a third polynucleotide comprising a sequence having at least 80%, 85%, 90%, 95%, or 98% identity to the nucleic acid sequence of SEQ ID NO: 10; a fourth polynucleotide comprising a sequence having at least 80%, 85%, 90%, 95%, or 98% identity to the nucleic acid sequence of SEQ ID NO: 11; a fifth polynucleotide comprising a sequence having at least 80%, 85%, 90%, 95%, or 98% identity to the nucleic acid sequence of SEQ ID NO: 12; a sixth polynucleotide comprising a sequence having at least 80%, 85%, 90%, 95%, or 98% identity to the nucleic acid sequence of SEQ ID NO: 13; and a seventh polynucleotide comprising a sequence having at least 80%, 85%, 90%, 95%, or 98% identity to the nucleic acid sequence of SEQ ID NO: 14.

In some embodiments, the first polynucleotide comprises the nucleic acid sequence of SEQ ID NO: 8; the second polynucleotide comprises the nucleic acid sequence of SEQ ID NO: 9; the third polynucleotide comprises the nucleic acid sequence of SEQ ID NO: 10; the fourth polynucleotide comprises the nucleic acid sequence of SEQ ID NO: 11; the fifth polynucleotide comprises the nucleic acid sequence of SEQ ID NO: 12; the sixth polynucleotide comprises the nucleic acid sequence of SEQ ID NO: 13; and the seventh polynucleotide comprises the nucleic acid sequence of SEQ ID NO: 14.

### BRIEF DESCRIPTION OF THE DRAWINGS

FIG. 1 shows a graph of in vitro cellular expression levels of protein encoded by mRNA OspA S1 and S6 variants (OspA-S1-ND165-173-NtermTM and OspA-S6-S88L-A200V-NTerm-TM) compared to mock-transfected cells and non-transfected cells. Two concentrations (500 ng/μL and 125 ng/μL) were tested and measured for mean fluorescence intensity (MFI)×Frequency (Freq).

FIGS. 2A-2B show graphs of total OspA-specific IgG titers (FIG. 2A) and LA-2 Competition ELISAs (FIG. 2B). The groups tested were non-translating mRNA (NTFIX), and mRNA having an ORF encoding a wild-type (WT) (i.e., naturally occurring) full-length, soluble, OspA Serotype 1 (S1) (WT-Soluble), or an OspA S1 with the LFA epitope removed (Soluble), or an OspA S1 with the LFA epitope removed and an N-terminal transmembrane domain (Nterm-TM), or an OspA S1 with the LFA epitope removed and a C-terminal transmembrane domain (Cterm-TM). Samples were tested on days 21 (d21), 44 (d44), and 56 (d56) for total OspA-specific IgG titers and on d44 and d56 for LA-2 Competition ELISA. The total OspA-specific IgG titers were measured based on log 10 Lyme OspA-specific IgG titers (FIG. 2A) and the LA-2 Competition ELISA was measure by percent (%) signal reduction (1:10) (FIG. 2B).

FIGS. 3A-3B show graphs of the serum bactericidal activity of OspA S1 with the LFA epitope removed and an N-terminal transmembrane domain (OspA S1 Nterm-TM). The OspA S1 Nterm-TM construct was compared to non-translating mRNA (NTFIX) and LA-2, measured by the Survival Index (FIG. 3A). Additionally, the OspA S1 Nterm-TM (Pool 2) was compared to an OspA S1 with the LFA epitope removed and a C-terminal transmembrane domain (OspA S1 Cterm-TM; Pool 3) and NTFIX (Pool 1), measured by the Survival Index (FIG. 3B).

FIGS. 4A-4B show graphs of total OspA-specific IgG titers (FIG. 4A) and LA-2 Competition ELISAs (FIG. 4B). The groups tested were non-translating mRNA (NTFIX), and mRNA having an ORF encoding an OspA S1 with the LFA epitope removed (Soluble), or an OspA S1 with the LFA epitope removed and an N-terminal transmembrane domain (Nterm-TM), or an OspA S1 with the LFA epitope removed and a C-terminal transmembrane domain (Cterm-TM). Samples were tested on days 21 (d21), 44 (d44), 56 (d56) and Pre-Challenge and Post-Challenge for total OspA-specific IgG titers and Pre-Challenge and Post-Challenge for LA-2 Competition ELISA. The total OspA-specific IgG titers were measured based on log 10 Lyme OspA-specific IgG titers (FIG. 4A) and the LA-2 Competition ELISA was measure by percent (%) signal reduction (1:10) (FIG. 4B). Mice were challenged using a tick bite challenge by administering 5 ticks per mouse with Borrelia burgdorferi strain B31. An unknown bacterial load was administered, and the challenge readout was 2 weeks post tick feed with the presence of spirochetes in skin, heart and bladder observed for 3 weeks.

FIGS. 5A-5C show graphs of the total and functional antibody titers to recombinant antibodies. Shown are the total OspA-specific IgG titers (FIG. 5A) and functional equivalents, LA-2 (only bactericidal against S1) (FIG. 5B) and a positive control antibody (bactericidal against all 7 serotypes) (FIG. 5C). The groups tested were non-translating mRNA (NTFIX), and mRNA having an ORF encoding either an OspA S1 with the LFA epitope removed and an N-terminal transmembrane domain (OspA S1 Nterm-TM), or mRNA having an ORF(s) encoding OspA S2-S5, S7, S1 with the LFA epitope removed and an N-terminal transmembrane domain and S6 with amino acid mutations S88L and A200V (OspA S1-S7 NtermTM) or mRNA having an ORF(s) encoding OspA S1 with a C-terminal transmembrane domain and ferritin stabilization (OspA S1 Cterm ferritin stabilized), and a purified Recombitek® (i.e., lipidated OspA protein)+Alum. The total OspA-specific IgG titers were measured using mean fluorescence intensity (MFI) (FIG. 5A) and LA-2 competition ELISA and positive control antibodies were measured using signal reduction (1:20) (FIGS. 5B-5C).

FIG. 6 shows graphs of antibodies generated by administration of either OspA S1 with the LFA epitope removed and an N-terminal transmembrane domain (OspA S1 NtermTM; mRNA-1982), or mRNA having an ORF(s) encoding OspA S2-S5, S7, S1 with the LFA epitope removed and an N-terminal transmembrane domain and S6 with amino acid mutations S88L and A200V (OspA S1-S7 NtermTM; mRNA-1975). OspA S1 NtermTM was administered at either 0.5 μg or 1 μg and OspA S1-S7 NtermTM was administered at either 3.5 μg or 7 μg. The mRNA vaccines were compared to mice administered PBS or Recombitek® or S1-S7 protein. Results were measured based on the mean fluorescence intensity (MFI) of the total OspA-specific IgG titers (S1, S2, S3, S4, S5, S6, S7) on day 20 and 35.

FIGS. 7A-7B show the ability of antibodies generated in response to OspA S1 NtermTM and OspA S1-S7 NtermTM to bind to Borrelia bacteria using a Surface binding assay. Groups tested include OspA S1 with the LFA epitope removed and an N-terminal transmembrane domain (S1), or mRNA having an ORF(s) encoding OspA S2-S5, S7, S1 with the LFA epitope removed and an N-terminal transmembrane domain and S6 with amino acid mutations S88L and A200V (S1-S7). The mRNA vaccines were compared to mice administered PBS or Recombitek® or S1-S7 protein, LA-2 or S1 Hyper+. The results are measured by percent (%) positive for Alexa-647 fluorescence (FIG. 7A) or the geometric mean (MFI) (FIG. 7B).

FIGS. 8A-8B show the ability of antibodies generated in response to OspA S1 NtermTM and OspA S1-S7 NtermTM to agglutinate (FIG. 8A) and compromise the membranes of Borrelia bacteria (FIG. 8B). These were measured using an Agglutination assay (FIG. 8A) and a Membrane Integrity assay (FIG. 8B). Groups tested include OspA S1 with the LFA epitope removed and an N-terminal transmembrane domain (S1), or mRNA having an ORF(s) encoding OspA S2-S5, S7, S1 with the LFA epitope removed and an N-terminal transmembrane domain and S6 with amino acid mutations S88L and A200V (S1-S7). The mRNA vaccines were compared to mice administered PBS or Recombitek® or S1-S7 protein, LA-2 or S1 Hyper+. The results are measured by percent (%) agglutination (FIG. 8A) and percent (%) propidium iodide positive (FIG. 8B).

FIG. 9 shows the serum bactericidal activity of antibodies generated in response to OspA S1 NtermTM and OspA S1-S7 NtermTM to kill Borrelia bacteria. This was measured using a serum bactericidal assay (SBA). Groups tested include OspA S1 with the LFA epitope removed and an N-terminal transmembrane domain (S1 mRNA), or mRNA having an ORF(s) encoding OspA S2-S5, S7, S1 with the LFA epitope removed and an N-terminal transmembrane domain and S6 with amino acid mutations S88L and A200V (S1-S7 mRNA). S1 mRNA was administered at either 0.5 μg or 1 μg and S1-S7 mRNA was administered at either 3.5 μg or 7 μg. The mRNA vaccines were compared to untreated IPTG, positive control antibody (10 μg/ml), LA-2 (10 μg/ml), PBS, S1 Hyper Immune or Recombitek® (1 μg, 2 μg or 5 μg) or S1-S7 (7 μg or 14 μg) protein. Results were measured based on mScarlet fluorescence for serum dilutions of 1:400, 1:800, 1:1600 or 1:3200.

FIGS. 10A-10B show the ability of either 2 or 3 doses of an mRNA vaccine delivered at different dosage amounts to protect mice from Borrelia infection in a tick bite model pre-challenge (FIG. 10A) and post-challenge (FIG. 10B). Mice were received either 2 or 3 doses of OspA S1 with the LFA epitope removed and an N-terminal transmembrane domain (0.2 μg, 0.5 μg or 1 μg) and compared to Recombitek®+Alum and non-translating mRNA (NTFIX). Results were measured using mean fluorescence intensity (MFI).

FIG. 11 shows the ability of 2 doses of the indicated mRNA or protein vaccines to protect mice from Borrelia infection in a tick bite model challenge (see also Example 2).

FIG. 12 shows graphs of antibodies generated by administration of mRNA having an ORF(s) encoding OspA S1-S7 NtermTM at a variety of doses with either two or three administrations (see also Example 9).

FIG. 13 shows the ability of 2 or 3 doses of the indicated mRNA or protein vaccines to protect mice from Borrelia infection at a variety of doses in a tick bite model challenge (see also Example 9).

FIG. 14 shows OspA S1 IgG amounts in blood samples isolated at 42, 84, 126, 168, and 189 days following initial administration of the indicated mRNA or protein vaccines (see also Example 10).

FIGS. 15A-15D are graphs depicting the results of studies involving C3H/HeN mice administered mRNA having an ORF(s) encoding Lyme OspA antigens from Serotype 1, Serotypes 1-7 or Serotypes 2-7, as described in Table 13, to test the cross-protection of the vaccine (see also Example 11). Antigen specific IgG titers, represented by mean fluorescence intensity (MFI), from vaccinated mice directed towards OspA S1 are shown in FIG. 15A. The percent (%) signal reduction measured by LA-2 or positive control antibody competition ELISA assays are shown in FIGS. 15B, and 15C, respectively. The percent (%) infected mice after tick bite challenge with Borrelia burgdorferi S1 is shown in FIG. 15D.

FIG. 16 shows a multiple sequence alignment of the OspA proteins of SEQ ID NOs: 1-7 and 24-30. The multiple sequence alignment was performed using Clustal Omega.

FIG. 17 shows a percent identity matrix comparing the amino acid sequences of the OspA proteins of SEQ ID NOs: 24-30 (which include OspA S1-S7 proteins) compared using multiple sequence alignment performed with Clustal Omega. The following additional comparisons were performed between OspA S1 proteins, between OspA S2 proteins, between OspA S3 proteins, between OspA S4 proteins, between OspA S5 proteins, between OspA S6 proteins, and between OspA S7 proteins described herein: Borrelia burgdorferi OspA S1 (SEQ ID NO: 24) is 89.59% identical to OspA S1 (SEQ ID NO: 1); Borrelia afzelii OspA S2 (SEQ ID NO: 25) is 90.71% identical to OspA S2 (SEQ ID NO: 2); Borrelia garinii OspA S3 (SEQ ID NO: 26) is 90.37% identical to OspA S3 (SEQ ID NO: 3); Borrelia bavariensis OspA S4 (SEQ ID NO: 27) is 90.71% identical to OspA S4 (SEQ ID NO: 4); Borrelia garinii OspA S5 (SEQ ID NO: 28) is 91.85% identical to OspA S5 (SEQ ID NO: 5); Borrelia garinii OspA S6 (SEQ ID NO: 29) is 91.11% identical to OspA S6 (SEQ ID NO: 6); Borrelia garinii OspA S7 (SEQ ID NO: 30) is 91.48% identical to OspA S7 (SEQ ID NO: 7).

### DETAILED DESCRIPTION

Lyme disease is an infectious disease caused by various species of Borrelia bacteria which are transmitted to humans through the bite of infected black-legged ticks. The disease is most commonly found in the northeastern, mid-Atlantic, and north-central regions of the United States, as well as in some parts of Europe and Asia. Lyme disease can cause a range of symptoms, including fever, fatigue, headache, and a characteristic “bull's-eye” rash. If left untreated, Lyme disease can lead to more serious complications. Vaccines have been developed to prevent Lyme disease, although currently there are no vaccines approved for use in the United States. In the past, two vaccines were available in the U.S., but they were withdrawn from the market primarily due to concerns about side effects, such as autoantibody production. The development of effective Lyme disease vaccines remains an important area of research, as tick-borne diseases continue to be a major public health concern in many parts of the world.

The development of a safe and effective vaccine against Lyme disease has been met with several challenges, including a limited understanding of the disease, complexity of the bacterium that causes the disease, low prevalence of the disease, and limited animal models. Furthermore, relative to viral antigens, bacterial antigens are more complex and diverse. Borrelia burgdorferi (B. burgdorferi), a pathogenic spirochete responsible for Lyme disease, for example, has the ability to change its surface proteins, making it difficult for the immune system to recognize and respond to the bacterium. This antigenic variability also makes it challenging to develop a vaccine that will be effective against more than one strain of the bacterium. As described herein, while Borrelia burgdorferi is the predominant strain that causes Lyme disease in the U.S., Borrelia afzelii, Borrelia garinii, and Borrelia bavariensis are the predominant strains to cause Lyme disease outside of the U.S. Furthermore, bacterial lipidation is thought to be essential for the proper function of Borrelia proteins (see, e.g., Zuckert W R Biochim Biophys Acta. 2014 August; 1843(8):1509-16), but is challenging to reproduce in mammalian cells. Lastly, Borrelia bacterial proteins are associated with high reactogenicity, which refers to their ability to cause adverse reactions or side effects in the body, that can lead to inflammation, fever, or other adverse reactions.

Surprisingly, the Borrelia proteins encoded by the mRNA vaccines provided herein elicit low reactogenicity and high immunogenicity, in some embodiments in response to multiple strains/serotypes of Borrelia, despite the absence of bacterial lipidation thought to be essential for proper functioning of the bacterial proteins. This is in part because these bacterial proteins are designed such that, when expressed in vivo by mammalian cells, they are still capable of folding into protein antigens having conformationally correct extracellular domains that preserve important epitopes critical for these properties. Moreover, the administration of these Borrelia proteins to a subject in the form of an mRNA vaccine stimulates the production of antibodies that exhibit high bactericidal and agglutination activity, properties that are not shared by identical Borrelia proteins when administered in the form of a protein vaccine.

Thus, in some embodiments, the mRNA vaccines encode Borrelia OspA proteins that have conformationally correct extracellular domains. A conformationally correct protein domain is a protein domain that has the correct three-dimensional structure (i.e., conformation) that is necessary for its proper function.

In some embodiments, the mRNA vaccines are capable of eliciting the production of bactericidal antibodies. Bactericidal activity simply refers to the ability of the vaccine, or more precisely the antibodies produced by the body in response to the vaccine, to kill the bacteria.

In some embodiments, the mRNA vaccines are capable of eliciting the production of antibodies that exhibit high agglutination activity. Agglutination activity refers to the ability of those antibodies to cause agglutination (the clumping together) of the bacterial cells expressing the specific epitopes to which the antibodies bind.

### Borrelia Proteins

Borrelia is a genus of bacteria that includes several species, some of which are known to cause Lyme disease in humans. The most common species associated with Lyme disease is Borrelia burgdorferi, which is primarily transmitted to humans through the bite of infected blacklegged ticks. Borrelia bacteria are spiral-shaped, Gram-negative bacteria that are capable of surviving in a variety of environments, including inside the bodies of animals and insects. The bacteria have a complex life cycle that involves both ticks and mammals, and they are capable of adapting to different hosts and environments.

Borrelia bacteria are known for their ability to evade the immune system and cause persistent infections. The bacteria are able to change their surface proteins, making it difficult for the immune system to recognize and respond to the infection. This ability to evade the immune system is thought to be one of the reasons why Lyme disease can become a chronic condition in some individuals. Despite methods by the bacteria to evade the host immune response, both the innate and adaptive immune systems respond to Borrelia infection. Most manifestations and symptoms of Lyme disease are caused by inflammation generated by the immune response because Borrelia does not produce toxins or extracellular matrix-degrading proteases. The host immune response can reduce bacterial infection and decrease symptoms within several weeks to months, even without antibiotic intervention. Nonetheless, persistent bacteria can survive for years in untreated patients resulting in chronic illness.

Borrelia infection is characterized by several pathogenic processes. Such processes include adherence of bacteria to host target cells, local multiplication, and migration to distant sites. A number of proteins produced by Borrelia are important in mediating these pathogenic processes, including Outer Surface Protein A (OspA) and Outer Surface Protein C (OspC), making them attractive candidate vaccine antigens. OspA is an abundant surface protein of Borrelia and is expressed primarily in the tick vector when the bacteria are present in the midgut. OspA is important for the initial attachment of the bacteria to the midgut of the tick and plays a critical role in the colonization of the tick vector. After the tick attaches to the mammalian host, the bacterium is stimulated in the midgut of the tick to undergo transformation from a state of colonization to infection. Once the bacteria are transmitted to a mammalian host during a tick bite, the expression of OspA is downregulated and OspC is upregulated. Thus, B. burgdorferi selectively produces OspA in ticks and OspC in mammals, after the bacteria has been transmitted to a mammalian host. OspC is thought to be critical for the establishment of the bacteria in the mammalian host and plays an important role in the early stages of the disease. OspC is involved in a number of processes that are important for the bacteria's survival in the mammalian host, including evasion of the host immune system and adhesion to host cells.

Borrelia OspA protein is a 31 kilodalton lipoprotein having two globular domains that are connected via a unique single-layer beta-sheet. The C terminus of the protein contains a receptor binding domain and is variable among different OspA serotypes, while the N terminus attaches to the bacterial cell membrane via post-translational palmitoylation on the signal sequence. The OspA protein also contains human lymphocyte function associated antigen-1 (hLFA-1) epitope regions localized within OspA amino acid residues 165-173 (OspA₁₆₅₋₁₇₃). In some embodiments, the hLFA-1 epitopes are modified or removed. In some embodiments the OspA₁₆₅₋₁₇₃ epitope is removed. In some embodiments, the Borrelia protein encoded by an mRNA of the present disclosure is a Borrelia OspA protein. In some embodiments, the Borrelia protein is an OspA protein variant, relative to a naturally occurring Borrelia OspA protein. The terms “naturally occurring” and “wild type” are used interchangeably herein. A naturally occurring Borrelia OspA protein is an Borrelia OspA protein (e.g., one of Borrelia serotypes 1-7) that occurs in nature, i.e., which is a naturally occurring isolate. As is known in the art, a naturally occurring protein is not genetically engineered. A naturally occurring protein is not genetically (or otherwise) modified to substitute, remove, or add any amino acids.

At least seven naturally occurring Borrelia OspA serotypes have been identified with eight monoclonal antibodies against different epitopes of the Borrelia OspA protein. As determined by 16S rRNA sequence analysis, these serotypes correlated well with recently delineated genospecies: serotype 1 (S1) corresponds to B. burgdorferi, serotype 2 (S2) corresponds to Borrelia afzelii, serotype 4 corresponds to Borrelia bavariensis, and serotypes 3 (S3) and 5-7 (S5-S7) correspond to Borrelia garinii. The mRNA vaccines provided herein may encode any one or more Borrelia OspA proteins selected from Borrelia burgdorferi OspA serotype 1 (S1); Borrelia afzelii OspA serotype 2 (S2); Borrelia garinii OspA serotype 3 (S3); Borrelia bavariensis OspA serotype 4 (S4); Borrelia garinii OspA serotype 5 (S5); Borrelia garinii OspA serotype 6 (S6); and Borrelia garinii OspA serotype 7 (S7).

In some embodiments, a vaccine of the present disclosure comprises an mRNA polynucleotide comprising an open reading frame encoding Borrelia burgdorferi OspA S1. In some embodiments, a vaccine of the present disclosure comprises an mRNA polynucleotide comprising an open reading frame encoding Borrelia afzelii OspA S2. In some embodiments, a vaccine of the present disclosure comprises an mRNA polynucleotide comprising an open reading frame encoding Borrelia garinii OspA S3. In some embodiments, a vaccine of the present disclosure comprises an mRNA polynucleotide comprising an open reading frame encoding Borrelia bavariensis OspA S4. In some embodiments, a vaccine of the present disclosure comprises an mRNA polynucleotide comprising an open reading frame encoding Borrelia garinii OspA S5. In some embodiments, a vaccine of the present disclosure comprises an mRNA polynucleotide comprising an open reading frame encoding Borrelia garinii OspA S6. In some embodiments, a vaccine of the present disclosure comprises an mRNA polynucleotide comprising an open reading frame encoding Borrelia garinii OspA S7.

In some embodiments, a vaccine of the present disclosure comprises an mRNA polynucleotide comprising an open reading frame encoding Borrelia burgdorferi OspA S1 (e.g., SEQ ID NO: 1), an mRNA polynucleotide comprising an open reading frame encoding Borrelia afzelii OspA S2 (e.g., SEQ ID NO: 2), an mRNA polynucleotide comprising an open reading frame encoding Borrelia garinii OspA S3 (e.g., SEQ ID NO: 3), an mRNA polynucleotide comprising an open reading frame encoding Borrelia bavariensis OspA S4 (e.g., SEQ ID NO: 4), an mRNA polynucleotide comprising an open reading frame encoding Borrelia garinii OspA S5 (e.g., SEQ ID NO: 5), an mRNA polynucleotide comprising an open reading frame encoding Borrelia garinii OspA S6 (e.g., SEQ ID NO: 6), and an mRNA polynucleotide comprising an open reading frame encoding Borrelia garinii OspA S7 (e.g., SEQ ID NO: 7).

In some embodiments, a vaccine of the present disclosure comprises an mRNA polynucleotide comprising an open reading frame encoding Borrelia afzelii OspA S2 (e.g., SEQ ID NO: 2), an mRNA polynucleotide comprising an open reading frame encoding Borrelia garinii OspA S3 (e.g., SEQ ID NO: 3), an mRNA polynucleotide comprising an open reading frame encoding Borrelia bavariensis OspA S4 (e.g., SEQ ID NO: 4), an mRNA polynucleotide comprising an open reading frame encoding Borrelia garinii OspA S5 (e.g., SEQ ID NO: 5), an mRNA polynucleotide comprising an open reading frame encoding Borrelia garinii OspA S6 (e.g., SEQ ID NO: 6), and an mRNA polynucleotide comprising an open reading frame encoding Borrelia garinii OspA S7 (e.g., SEQ ID NO: 7).

### Transmembrane Domains

In some embodiments, the bacterial proteins encoded by the mRNA vaccines comprises a heterologous transmembrane domain. A transmembrane domain is a region of a protein that spans the lipid bilayer of a biological membrane, such as a cell membrane. Transmembrane domains are composed of hydrophobic amino acids that are able to interact with the hydrophobic core of the membrane, anchoring the protein to the membrane and allowing it to interact with other proteins or molecules on either side of the membrane. A transmembrane domain is “heterologous” to a protein if the protein does not naturally occur with the transmembrane domain. For example, a viral transmembrane domain is heterologous to a bacterial protein or a protein comprising a bacterial domain, such as a bacterial extracellular domain. In some embodiments, a Borrelia OspA protein comprises a Borrelia OspA extracellular domain and a viral transmembrane domain. In some embodiments, the viral transmembrane domain is from an influenza virus protein, for example, an influenza virus neurominidase transmembrane domain. Other viral transmembrane domains may also be used to anchor bacterial proteins to a host cell membrane, including without limitation, the transmembrane domain from any of the following: human immunodeficiency virus (HIV) envelope glycoprotein (Env), hepatitis C virus (HCV) envelope glycoproteins E1 and E2, herpes simplex virus (HSV) glycoprotein D (gD), and other influenza virus proteins, such as hemagglutinin and M2 protein.

In bacterial cells, the bacterial proteins are naturally anchored to the cell surface via lipidation at the N terminus of the protein. The bacterial antigens encoded by the mRNA of the disclosure, in some embodiments, have been modified to include a heterologous amino terminal (N terminal) transmembrane domain, which anchors the antigen to the surface of the mammalian cell. In some embodiments, the bacterial antigens encoded by the mRNA of the disclosure have been modified to include a heterologous carboxyl terminal (C terminal) transmembrane domain. When presented at the cell surface, the bacterial antigens are more conformationally accurate, thereby capable of eliciting a more effective immune response. Surprisingly, the data herein demonstrates that the location of the heterologous transmembrane domain impacts the strength of the antibody response. Higher neutralizing antibody titers were observed following administration of mRNA vaccines encoding Borrelia OspA proteins comprising an N-terminal heterologous transmembrane domain.

### N-Linked Glycan Site Mutations

In some embodiments, the bacterial proteins encoded by the mRNA vaccines comprises one or more N-linked glycan site mutations. N-linked glycan site mutations refer to alterations in the amino acid sequence of a protein that affect the attachment of carbohydrate molecules, or glycans, at specific sites. N-linked glycans are attached to proteins at asparagine residues that are located in a specific amino acid sequence context (NxS/T/C, where x can be any amino acid except proline). These glycans can affect the structure, stability, and function of proteins and play important roles in many biological processes. Bacterial systems rarely glycosylate proteins; however, glycosylation does occur somewhat frequently in mammalian cells. To prevent glycosylation, the mRNA described herein, in some embodiments, encodes bacterial proteins wherein residues prone to N-linked glycosylation (e.g., asparagine) have been removed, modified, or substituted in order to prevent glycosylation.

### Modifications to Increase Stability/Expression

In some embodiments, the bacterial proteins encoded by the mRNA vaccines comprises one or more stabilizing mutation. Protein stabilizing mutations refer to alterations in the amino acid sequence of a protein that improve its stability, or its ability to maintain its folded structure and resist denaturation or aggregation. These mutations can enhance protein stability by strengthening intramolecular interactions, improving the packing of the protein core, or reducing the flexibility of the protein structure. As demonstrated herein, the naturally occurring form of the Borrelia OspA S6 protein, for example, was not detected following expression in vitro. Two mutations (S88L and A200V) were made to enhance protein stability by providing better hydrophobic packing of the protein. Thus, in some embodiments, the Borrelia OspA proteins of the disclosure may be modified to improve hydrophobic packing of the protein, thereby stabilizing the protein and increasing expression of the protein, relative to the corresponding naturally occurring protein.

Non-limiting examples of mRNA sequences encoding Borrelia OspA proteins and their corresponding amino acid sequences are provided in Table 1.

TABLE 1
Vaccine Sequences and Naturally Occurring Protein Sequences

| | | |
|---|---|---|
| | | SEQ ID |
| Name | Sequence | NO: |

OspA-S1-ND165-173-Nterm-TM

| SEQ ID NO: 15 consists of from 5′ end to 3′ end: 5′ UTR SEQ ID NO: 22, mRNA ORF SEQ ID NO: | | 15 |
|---|---|---|
| 8, and 3′ UTR SEQ ID NO: 23. | | |
| Chemical | 1-methylpseudouridine | |
| Modification | | |
| Cap | 7mG(5′)ppp(5′)NlmpNp | |
| 5′ UTR | GAGGAAAUCGCAAAAUUUGCUCUUCGCGUUAGAUUUCUUUUAGUUUUCUCGCAAC | 22 |
| | UAGCAAGCUUUUUGUUCUCGCC | |
| mRNA ORF | AUGAACCCCAACCAGAAGAUCAUCACCAUCGGCAGCGUGAGCCUGACAAUCAGCA | 8 |
| | CCAUCUGCUUCUUCAUGCAGAUCGCCAUCCUGAUCACCACCGUCACCCUGCACUU | |
| | CAAGCAGGAGAAGAACAGCGUGAGCGUGGAUCUGCCCGGCGAGAUGAAGGUGCUG | |
| | GUCAGCAAAGAGAAGAACAAGGACGGUAAGUACGACCUGAUCGCCACCGUGGACA | |
| | AGCUGGAGCUGAAGGGCACCAGCGACAAGAACAACGGAGCUGGCGUGCUGGAGGG | |
| | CGUGAAGGCCGACAAGAGCAAGGUGAAGCUGACCAUCAGCGACGACCUGGGCCAG | |
| | ACAACCCUGGAGGUGUUCAAGGAGGACGGCAAGACCCUGGUGAGCAAGAAGGUGA | |
| | CCAGCAAGGACAAGAGCAGCACCGAGGAGAAGUUCAACGAGAAGGGCGAGGUGAG | |
| | CGAGAAGAUCAUCACAAGGGCUGACGGCACCAGACUGGAGUACACCGGCAUCAAG | |
| | AGCGACGGCAGUGGCAAGGCCAAGGAGGUGCUGAAGGGCUUCAUCCUGGAGGGCC | |
| | GGUUUACCGCCGAGAAGACCACCCUGGUGGUGAAGGAGGGCACCGUGACCCUGAG | |
| | CAAGGACAUCAGCAAGAGCGGCGAGGUGUCUGUGGAGCUGCAGGACACCGACAGC | |
| | AGCGCCGCCACAAAGAAGACCGCCGCCUGGAAUAGCGGCACCAGCACCCUGACCA | |
| | UCACCGUGAACAGCAAGAAGACCAAGGACCUGGUGUUCACCAAGGAGAACACCAU | |
| | CACUGUGCAGCAGUACGACAGCAACGGCGUGAAACUGGAGGGCAGCGCCGUGGAG | |
| | AUCACCAAGCUGGACGAGAUCAAGAACGCCCUGAAG | |
| 3′ UTR | UAAAGCUCCCCGGGGGCCUCGGUGGCCUAGCUUCUUGCCCCUUGGGCCUCCCCCC | 23 |
| | AGCCCCUCCUCCCCUUCCUGCACCCGUACCCCCGUGGUCUUUGAAUAAAGUCUGA | |
| | GUGGGCGGC | |
| PolyA | 100 nucleotides | |
| Corresponding | MNPNQKIITIGSVSLTISTICFFMQIAILITTVTLHFKQEKNSVSVDLPGEMKVL | 1 |
| Protein | VSKEKNKDGKYDLIATVDKLELKGTSDKNNGAGVLEGVKADKSKVKLTISDDLGQ | |
| | TTLEVFKEDGKTLVSKKVTSKDKSSTEEKFNEKGEVSEKIITRADGTRLEYTGIK | |
| | SDGSGKAKEVLKGFILEGRFTAEKTTLVVKEGTVTLSKDISKSGEVSVELQDTDS | |
| | SAATKKTAAWNSGTSTLTITVNSKKTKDLVFTKENTITVQQYDSNGVKLEGSAVE | |
| | ITKLDEIKNALK | |

OspA-S1-ND165-173-Nterm-TM (v2)

| SEQ ID NO: 40 consists of from 5′ end to 3′ end: 5′ UTR SEQ ID NO: 50, mRNA ORF SEQ ID NO: | | 40 |
|---|---|---|
| 8, and 3′ UTR SEQ ID NO: 53. | | |
| Chemical | 1-methylpseudouridine | |
| Modification | | |
| Cap | 5′7MeGpppG2′Ome | |
| 5′ UTR | AGGAAAUCGCAAAAUUUGCUCUUCGCGUUAGAUUUCUUUUAGUUUUCUCGCAACU | 50 |
| | AGCAAGCUUUUUGUUCUCGCC | |
| mRNA ORF | AUGAACCCCAACCAGAAGAUCAUCACCAUCGGCAGCGUGAGCCUGACAAUCAGCA | 8 |
| | CCAUCUGCUUCUUCAUGCAGAUCGCCAUCCUGAUCACCACCGUCACCCUGCACUU | |
| | CAAGCAGGAGAAGAACAGCGUGAGCGUGGAUCUGCCCGGCGAGAUGAAGGUGCUG | |
| | GUCAGCAAAGAGAAGAACAAGGACGGUAAGUACGACCUGAUCGCCACCGUGGACA | |
| | AGCUGGAGCUGAAGGGCACCAGCGACAAGAACAACGGAGCUGGCGUGCUGGAGGG | |
| | CGUGAAGGCCGACAAGAGCAAGGUGAAGCUGACCAUCAGCGACGACCUGGGCCAG | |
| | ACAACCCUGGAGGUGUUCAAGGAGGACGGCAAGACCCUGGUGAGCAAGAAGGUGA | |
| | CCAGCAAGGACAAGAGCAGCACCGAGGAGAAGUUCAACGAGAAGGGCGAGGUGAG | |
| | CGAGAAGAUCAUCACAAGGGCUGACGGCACCAGACUGGAGUACACCGGCAUCAAG | |
| | AGCGACGGCAGUGGCAAGGCCAAGGAGGUGCUGAAGGGCUUCAUCCUGGAGGGCC | |
| | GGUUUACCGCCGAGAAGACCACCCUGGUGGUGAAGGAGGGCACCGUGACCCUGAG | |
| | CAAGGACAUCAGCAAGAGCGGCGAGGUGUCUGUGGAGCUGCAGGACACCGACAGC | |
| | AGCGCCGCCACAAAGAAGACCGCCGCCUGGAAUAGCGGCACCAGCACCCUGACCA | |
| | UCACCGUGAACAGCAAGAAGACCAAGGACCUGGUGUUCACCAAGGAGAACACCAU | |
| | CACUGUGCAGCAGUACGACAGCAACGGCGUGAAACUGGAGGGCAGCGCCGUGGAG | |
| | AUCACCAAGCUGGACGAGAUCAAGAACGCCCUGAAG | |
| 3′ UTR | UAAAGCUCCCCGGGGGCCUCGGUGGCCUAGCUUCUUGCCCCUUGGGCCUCCCCCC | 53 |
| | AGCCCCUCCUCCCCUUCCUGCAGGUUAAGGAAGUGGUCUUUGAAUAAAGUCUGAG | |
| | UGGGCGGC | |
| PolyA | 100 nucleotides | |
| Corresponding | MNPNQKIITIGSVSLTISTICFFMQIAILITTVTLHFKQEKNSVSVDLPGEMKVL | 1 |
| Protein | VSKEKNKDGKYDLIATVDKLELKGTSDKNNGAGVLEGVKADKSKVKLTISDDLGQ | |
| | TTLEVFKEDGKTLVSKKVTSKDKSSTEEKFNEKGEVSEKIITRADGTRLEYTGIK | |
| | SDGSGKAKEVLKGFILEGRFTAEKTTLVVKEGTVTLSKDISKSGEVSVELQDTDS | |
| | SAATKKTAAWNSGTSTLTITVNSKKTKDLVFTKENTITVQQYDSNGVKLEGSAVE | |
| | ITKLDEIKNALK | |

OspA-S2-Nterm-TM

| SEQ ID NO: 16 consists of from 5′ end to 3′ end: 5′ UTR SEQ ID NO: 22, mRNA ORF SEQ ID NO: | | 16 |
|---|---|---|
| 9, and 3′ UTR SEQ ID NO: 23. | | |
| Chemical | 1-methylpseudouridine | |
| Modification | | |
| Cap | 7mG(5′)ppp(5′)NlmpNp | |
| 5′ UTR | GAGGAAAUCGCAAAAUUUGCUCUUCGCGUUAGAUUUCUUUUAGUUUUCUCGCAAC | 22 |
| | UAGCAAGCUUUUUGUUCUCGCC | |
| mRNA ORF | AUGAACCCCAACCAGAAGAUCAUCACCAUCGGCAGCGUGUCGUUGACCAUCAGCA | 9 |
| | CCAUCUGCUUCUUCAUGCAGAUCGCCAUCCUGAUCACCACCGUGACCCUGCACUU | |
| | CAAGCAGGAGAAGAACAGCGCCAGCGUGGACCUGCCCGGAGAGAUGAAGGUGCUG | |
| | GUGAGUAAGGAGAAGGACAAAGACGGCAAGUACAGCCUGAAGGCCACCGUGGAUA | |
| | AGAUCGAGCUGAAGGGCACCAGCGACAAGGACAACGGAGCCGGCGUGCUGGAGGG | |
| | AACCAAGGACGACAAGAGCAAGGCAAAGCUGACCAUCGCCGACGACCUGAGUAAG | |
| | ACCACCUUCGAGCUGUUCAAGGAGGACGGCAAGACCCUGGUGAGCCGGAAGGUGA | |
| | GCAGCAAGGAUAAGACCAGCACCGACGAGAUGUUCAACGAGAAGGGCGAACUGAG | |
| | CGCCAAGACCAUGACCAGAGAGGACGGCACCAAGCUGGAGUACACCGAGAUGAAG | |
| | AGCGACGGCACCGGCAAGGCCAAGGAGGUGCUGAAGGGCUUCACCCUGGAAGGCA | |
| | AGGUGGCCAACGACAAGGUGACCCUGGAGGUGAAGGAGGGCACCGUGACCCUGAG | |
| | CAAGGAGAUCGCUAAGAGCGGCGAGGUGACCGUGGCCCUGCAGGACACCGACACC | |
| | ACCCAGGCCACCAAGAAGACCGGCGCCUGGGACAGCAAGACCAGCACCCUGACCA | |
| | UCUCCGUGAACAGCAAGAAGACCACCCAGCUGGUGUUCACAAAGCAGGACACCAU | |
| | CACCGUGCAGAAGUACGACAGCGCCGGCACCAACCUGGAGGGCACCGCCGUGGAG | |
| | AUCAAGACACUGGACGAGCUGAAGAACGCCCUGAAG | |
| 3′ UTR | UAAAGCUCCCCGGGGGCCUCGGUGGCCUAGCUUCUUGCCCCUUGGGCCUCCCCCC | 23 |
| | AGCCCCUCCUCCCCUUCCUGCACCCGUACCCCCGUGGUCUUUGAAUAAAGUCUGA | |
| | GUGGGCGGC | |
| PolyA | 100 nucleotides | |
| Corresponding | MNPNQKIITIGSVSLTISTICFFMQIAILITTVTLHFKQEKNSASVDLPGEMKVL | 2 |
| Protein | VSKEKDKDGKYSLKATVDKIELKGTSDKDNGAGVLEGTKDDKSKAKLTIADDLSK | |
| | TTFELFKEDGKTLVSRKVSSKDKTSTDEMFNEKGELSAKTMTREDGTKLEYTEMK | |
| | SDGTGKAKEVLKGFTLEGKVANDKVTLEVKEGTVTLSKEIAKSGEVTVALQDTDT | |
| | TQATKKTGAWDSKTSTLTISVNSKKTTQLVFTKQDTITVQKYDSAGTNLEGTAVE | |
| | IKTLDELKNALK | |

OspA-S2-Nterm-TM (v2)

| SEQ ID NO: 55 consists of from 5′ end to 3′ end: 5′ UTR SEQ ID NO: 50, mRNA ORF SEQ ID NO: | | 55 |
|---|---|---|
| 9, and 3′ UTR SEQ ID NO: 56. | | |
| Chemical | 1-methylpseudouridine | |
| Modification | | |
| Cap | 5′7MeGpppG2′Ome | |
| 5′ UTR | AGGAAAUCGCAAAAUUUGCUCUUCGCGUUAGAUUUCUUUUAGUUUUCUCGCAACU | 50 |
| | AGCAAGCUUUUUGUUCUCGCC | |
| mRNA ORF | AUGAACCCCAACCAGAAGAUCAUCACCAUCGGCAGCGUGUCGUUGACCAUCAGCA | 9 |
| | CCAUCUGCUUCUUCAUGCAGAUCGCCAUCCUGAUCACCACCGUGACCCUGCACUU | |
| | CAAGCAGGAGAAGAACAGCGCCAGCGUGGACCUGCCCGGAGAGAUGAAGGUGCUG | |
| | GUGAGUAAGGAGAAGGACAAAGACGGCAAGUACAGCCUGAAGGCCACCGUGGAUA | |
| | AGAUCGAGCUGAAGGGCACCAGCGACAAGGACAACGGAGCCGGCGUGCUGGAGGG | |
| | AACCAAGGACGACAAGAGCAAGGCAAAGCUGACCAUCGCCGACGACCUGAGUAAG | |
| | ACCACCUUCGAGCUGUUCAAGGAGGACGGCAAGACCCUGGUGAGCCGGAAGGUGA | |
| | GCAGCAAGGAUAAGACCAGCACCGACGAGAUGUUCAACGAGAAGGGCGAACUGAG | |
| | CGCCAAGACCAUGACCAGAGAGGACGGCACCAAGCUGGAGUACACCGAGAUGAAG | |
| | AGCGACGGCACCGGCAAGGCCAAGGAGGUGCUGAAGGGCUUCACCCUGGAAGGCA | |
| | AGGUGGCCAACGACAAGGUGACCCUGGAGGUGAAGGAGGGCACCGUGACCCUGAG | |
| | CAAGGAGAUCGCUAAGAGCGGCGAGGUGACCGUGGCCCUGCAGGACACCGACACC | |
| | ACCCAGGCCACCAAGAAGACCGGCGCCUGGGACAGCAAGACCAGCACCCUGACCA | |
| | UCUCCGUGAACAGCAAGAAGACCACCCAGCUGGUGUUCACAAAGCAGGACACCAU | |
| | CACCGUGCAGAAGUACGACAGCGCCGGCACCAACCUGGAGGGCACCGCCGUGGAG | |
| | AUCAAGACACUGGACGAGCUGAAGAACGCCCUGAAG | |
| 3′ UTR | UAAAGCUCCCCGGGGGCCUCGGUGGCCUAGCUUCUUGCCCCUUGGGCCUCCCCCC | 56 |
| | AGCCCCUCCUCCCCUUCCUGCAGGUGACCUUAGAGUGGUCUUUGAAUAAAGUCUG | |
| | AGUGGGCGGC | |
| PolyA | 100 nucleotides | |
| Corresponding | MNPNQKIITIGSVSLTISTICFFMQIAILITTVTLHFKQEKNSASVDLPGEMKVL | 2 |
| Protein | VSKEKDKDGKYSLKATVDKIELKGTSDKDNGAGVLEGTKDDKSKAKLTIADDLSK | |
| | TTFELFKEDGKTLVSRKVSSKDKTSTDEMFNEKGELSAKTMTREDGTKLEYTEMK | |
| | SDGTGKAKEVLKGFTLEGKVANDKVTLEVKEGTVTLSKEIAKSGEVTVALQDTDT | |
| | TQATKKTGAWDSKTSTLTISVNSKKTTQLVFTKQDTITVQKYDSAGTNLEGTAVE | |
| | IKTLDELKNALK | |

OspA-S3-P233T-Nterm-TM

| SEQ ID NO: 17 consists of from 5′ end to 3′ end: 5′ UTR SEQ ID NO: 22, mRNA ORF SEQ ID NO: | | 17 |
|---|---|---|
| 10, and 3′ UTR SEQ ID NO: 23. | | |
| Chemical | 1-methylpseudouridine | |
| Modification | | |
| Cap | 7mG(5′)ppp(5′)NlmpNp | |
| 5′ UTR | GAGGAAAUCGCAAAAUUUGCUCUUCGCGUUAGAUUUCUUUUAGUUUUCUCGCAAC | 22 |
| | UAGCAAGCUUUUUGUUCUCGCC | |
| mRNA ORF | AUGAACCCCAACCAGAAGAUCAUCACCAUCGGCAGCGUGUCGUUGACCAUCAGCA | 10 |
| | CCAUCUGCUUCUUCAUGCAGAUCGCCAUCCUGAUCACCACCGUGACCCUGCACUU | |
| | CAAGCAGGAGAAGAACAGCGUGAGCGUGGACCUGCCCGGAGGCAUGAAGGUGCUG | |
| | GUGAGUAAGGAGAAGGACAAAGACGGCAAGUACAGCCUGAUGGCCACCGUGGAGA | |
| | AGCUGGAGCUGAAGGGCACCAGCGACAAGUCCAACGGAGCCGGCGUGCUGGAGGG | |
| | AGAGAAGGCCGACAAGAGCAAGGCAAAGCUGACCAUCAGCCAGGACCUGAGUCAG | |
| | ACCACCUUCGAGAUCUUCAAGGAGGACGGCAAGACCCUGGUGAGCCGGAAGGUGA | |
| | ACAGCAAGGAUAAGAGCAGCACCGAGGAGAAGUUCAACGACAAGGGCAAGCUGAG | |
| | CGAGAAGGUGGUGACCAGAGCAGACGGCACCCGGCUGGAGUACACCGAGAUCAAG | |
| | AACGACGGCAGCGGCAAGGCCAAGGAGGUGCUGAAGGGCUUCGCCCUGGAAGGCA | |
| | CCCUGACCGACGGCGGCGAGACCAAGCUGACCGUGACCGAGGGCACCGUGACCCU | |
| | GAGCAAGGACAUCAGCAAGAGCGGCGAGAUCACCGUGGCCCUGCAGGACACCGAG | |
| | ACCACCCCAGCCGACAAGAAGACCGGCGAGUGGAAGAGCGACACCAGCACCCUGA | |
| | CCAUCUCCAAGAACAGCCAGAAGACCAAGCAGCUGGUGUUCACAAAGGAGAACAC | |
| | CAUCACCGUGCAGAACUACAACCGGGCCGGCAACGCACUGGAGGGCAGCCCCGCA | |
| | GAGAUCAAGGACCUGGCCGAGCUGAAGGCCGCCCUGAAG | |
| 3′ UTR | UAAAGCUCCCCGGGGGCCUCGGUGGCCUAGCUUCUUGCCCCUUGGGCCUCCCCCC | 23 |
| | AGCCCCUCCUCCCCUUCCUGCACCCGUACCCCCGUGGUCUUUGAAUAAAGUCUGA | |
| | GUGGGCGGC | |
| PolyA | 100 nucleotides | |
| Corresponding | MNPNQKIITIGSVSLTISTICFFMQIAILITTVTLHFKQEKNSVSVDLPGGMKVL | 3 |
| Protein | VSKEKDKDGKYSLMATVEKLELKGTSDKSNGAGVLEGEKADKSKAKLTISQDLSQ | |
| | TTFEIFKEDGKTLVSRKVNSKDKSSTEEKFNDKGKLSEKVVTRADGTRLEYTEIK | |
| | NDGSGKAKEVLKGFALEGTLTDGGETKLTVTEGTVTLSKDISKSGEITVALQDTE | |
| | TTPADKKTGEWKSDTSTLTISKNSQKTKQLVFTKENTITVQNYNRAGNALEGSPA | |
| | EIKDLAELKAALK | |

OspA-S3-P233T-Nterm-TM (v2)

| SEQ ID NO: 57 consists of from 5′ end to 3′ end: 5′ UTR SEQ ID NO: 50, mRNA ORF SEQ ID NO: | | 57 |
|---|---|---|
| 10, and 3′ UTR SEQ ID NO: 58. | | |
| Chemical | 1-methylpseudouridine | |
| Modification | | |
| Cap | 5′7MeGpppG2′Ome | |
| 5′ UTR | AGGAAAUCGCAAAAUUUGCUCUUCGCGUUAGAUUUCUUUUAGUUUUCUCGCAACU | 50 |
| | AGCAAGCUUUUUGUUCUCGCC | |
| mRNA ORF | AUGAACCCCAACCAGAAGAUCAUCACCAUCGGCAGCGUGUCGUUGACCAUCAGCA | 10 |
| | CCAUCUGCUUCUUCAUGCAGAUCGCCAUCCUGAUCACCACCGUGACCCUGCACUU | |
| | CAAGCAGGAGAAGAACAGCGUGAGCGUGGACCUGCCCGGAGGCAUGAAGGUGCUG | |
| | GUGAGUAAGGAGAAGGACAAAGACGGCAAGUACAGCCUGAUGGCCACCGUGGAGA | |
| | AGCUGGAGCUGAAGGGCACCAGCGACAAGUCCAACGGAGCCGGCGUGCUGGAGGG | |
| | AGAGAAGGCCGACAAGAGCAAGGCAAAGCUGACCAUCAGCCAGGACCUGAGUCAG | |
| | ACCACCUUCGAGAUCUUCAAGGAGGACGGCAAGACCCUGGUGAGCCGGAAGGUGA | |
| | ACAGCAAGGAUAAGAGCAGCACCGAGGAGAAGUUCAACGACAAGGGCAAGCUGAG | |
| | CGAGAAGGUGGUGACCAGAGCAGACGGCACCCGGCUGGAGUACACCGAGAUCAAG | |
| | AACGACGGCAGCGGCAAGGCCAAGGAGGUGCUGAAGGGCUUCGCCCUGGAAGGCA | |
| | CCCUGACCGACGGCGGCGAGACCAAGCUGACCGUGACCGAGGGCACCGUGACCCU | |
| | GAGCAAGGACAUCAGCAAGAGCGGCGAGAUCACCGUGGCCCUGCAGGACACCGAG | |
| | ACCACCCCAGCCGACAAGAAGACCGGCGAGUGGAAGAGCGACACCAGCACCCUGA | |
| | CCAUCUCCAAGAACAGCCAGAAGACCAAGCAGCUGGUGUUCACAAAGGAGAACAC | |
| | CAUCACCGUGCAGAACUACAACCGGGCCGGCAACGCACUGGAGGGCAGCCCCGCA | |
| | GAGAUCAAGGACCUGGCCGAGCUGAAGGCCGCCCUGAAG | |
| 3′ UTR | UAAAGCUCCCCGGGGGCCUCGGUGGCCUAGCUUCUUGCCCCUUGGGCCUCCCCCC | 58 |
| | AGCCCCUCCUCCCCUUCCUGCAGUCCACAUAUCGAAGUGGUCUUUGAAUAAAGUC | |
| | UGAGUGGGCGGC | |
| PolyA | 100 nucleotides | |
| Corresponding | MNPNQKIITIGSVSLTISTICFFMQIAILITTVTLHFKQEKNSVSVDLPGGMKVL | 3 |
| Protein | VSKEKDKDGKYSLMATVEKLELKGTSDKSNGAGVLEGEKADKSKAKLTISQDLSQ | |
| | TTFEIFKEDGKTLVSRKVNSKDKSSTEEKFNDKGKLSEKVVTRADGTRLEYTEIK | |
| | NDGSGKAKEVLKGFALEGTLTDGGETKLTVTEGTVTLSKDISKSGEITVALQDTE | |
| | TTPADKKTGEWKSDTSTLTISKNSQKTKQLVFTKENTITVQNYNRAGNALEGSPA | |
| | EIKDLAELKAALK | |

OspA-S4-Nterm-TM

| SEQ ID NO: 18 consists of from 5′ end to 3′ end: 5′ UTR SEQ ID NO: 22, mRNA ORF SEQ ID NO: | | 18 |
|---|---|---|
| 11, and 3′ UTR SEQ ID NO: 23. | | |
| Chemical | 1-methylpseudouridine | |
| Modification | | |
| Cap | 7mG(5′)ppp(5′)NlmpNp | |
| 5′ UTR | GAGGAAAUCGCAAAAUUUGCUCUUCGCGUUAGAUUUCUUUUAGUUUUCUCGCAAC | 22 |
| | UAGCAAGCUUUUUGUUCUCGCC | |
| mRNA ORF | AUGAACCCCAACCAGAAGAUCAUCACCAUCGGCAGCGUGUCGUUGACCAUCAGCA | 11 |
| | CCAUCUGCUUCUUCAUGCAGAUCGCCAUCCUGAUCACCACCGUGACCCUGCACUU | |
| | CAAGCAGGAGAAGAACAGCGUGAGCGUGGACCUGCCCGGAGAGAUGAAGGUGCUG | |
| | GUGAGUAAGGAGAAGGACAAAGACGGCAAGUACAGCCUGAUGGCCACCGUGGAUA | |
| | AGCUGGAGCUGAAGGGCACCAGCGACAAGUCCAACGGAGCCGGCACUCUGGAGGG | |
| | AGAGAAGAGCGACAAGAGCAAGGCAAAGCUGACCAUCAGCGAGGACCUGAGUAAG | |
| | ACCACCUUCGAGAUCUUCAAGGAGGACGGCAAGACCCUGGUGAGCAAGAAGGUGA | |
| | ACAGCAAGGAUAAGAGCAGCAUCGAGGAGAAGUUCAACGCCAAGGGCGAACUGAG | |
| | CGAGAAGACCAUCCUGAGAGCAGACGGCACCCGGCUGGAGUACACCGAGAUCAAG | |
| | AGCGACGGCACCGGCAAGGCCAAGGAGGUGCUGAAGGACUUCGCCCUGGAAGGCA | |
| | CCCUGGCCGCCGACAAGACCACCCUGAAGGUGACCGAGGGCACCGUGGUGCUGAG | |
| | CAAGCACAUCCCCAACAGCGGCGAGAUCACCGUGGAGCUGCAGGACAGCGACAGC | |
| | ACCCAGGCCACCAAGAAGACCGGCAAGUGGGACAGCCAGACCAGCACCCUGACCA | |
| | UCUCCGUGAACAGCAAGAAGACCAAGAACAUCGUGUUCACAAAGGAGGACACCAU | |
| | CACCGUGCAGAAGUACGACAGCGCCGGCACCAACCUGGAGGGCAACGCCGUGGAG | |
| | AUCAAGACACUGGACGAGCUGAAGAACGCCCUGAAG | |
| 3′ UTR | UAAAGCUCCCCGGGGGCCUCGGUGGCCUAGCUUCUUGCCCCUUGGGCCUCCCCCC | 23 |
| | AGCCCCUCCUCCCCUUCCUGCACCCGUACCCCCGUGGUCUUUGAAUAAAGUCUGA | |
| | GUGGGCGGC | |
| PolyA | 100 nucleotides | |
| Corresponding | MNPNQKIITIGSVSLTISTICFFMQIAILITTVTLHFKQEKNSVSVDLPGEMKVL | 4 |
| Protein | VSKEKDKDGKYSLMATVDKLELKGISDKSNGAGTLEGEKSDKSKAKLTISEDLSK | |
| | TTFEIFKEDGKTLVSKKVNSKDKSSIEEKFNAKGELSEKTILRADGTRLEYTEIK | |
| | SDGTGKAKEVLKDFALEGTLAADKTTLKVTEGTVVLSKHIPNSGEITVELQDSDS | |
| | TQATKKTGKWDSQTSTLTISVNSKKTKNIVFTKEDTITVQKYDSAGTNLEGNAVE | |
| | IKTLDELKNALK | |

OspA-S4-Nterm-TM (v2)

| SEQ ID NO: 59 consists of from 5′ end to 3′ end: 5′ UTR SEQ ID NO: 50, mRNA ORF SEQ ID NO: | | 59 |
|---|---|---|
| 11, and 3′ UTR SEQ ID NO: 60. | | |
| Chemical | 1-methylpseudouridine | |
| Modification | | |
| Cap | 5′7MeGpppG2′Ome | |
| 5′ UTR | AGGAAAUCGCAAAAUUUGCUCUUCGCGUUAGAUUUCUUUUAGUUUUCUCGCAACU | 50 |
| | AGCAAGCUUUUUGUUCUCGCC | |
| mRNA ORF | AUGAACCCCAACCAGAAGAUCAUCACCAUCGGCAGCGUGUCGUUGACCAUCAGCA | 11 |
| | CCAUCUGCUUCUUCAUGCAGAUCGCCAUCCUGAUCACCACCGUGACCCUGCACUU | |
| | CAAGCAGGAGAAGAACAGCGUGAGCGUGGACCUGCCCGGAGAGAUGAAGGUGCUG | |
| | GUGAGUAAGGAGAAGGACAAAGACGGCAAGUACAGCCUGAUGGCCACCGUGGAUA | |
| | AGCUGGAGCUGAAGGGCACCAGCGACAAGUCCAACGGAGCCGGCACUCUGGAGGG | |
| | AGAGAAGAGCGACAAGAGCAAGGCAAAGCUGACCAUCAGCGAGGACCUGAGUAAG | |
| | ACCACCUUCGAGAUCUUCAAGGAGGACGGCAAGACCCUGGUGAGCAAGAAGGUGA | |
| | ACAGCAAGGAUAAGAGCAGCAUCGAGGAGAAGUUCAACGCCAAGGGCGAACUGAG | |
| | CGAGAAGACCAUCCUGAGAGCAGACGGCACCCGGCUGGAGUACACCGAGAUCAAG | |
| | AGCGACGGCACCGGCAAGGCCAAGGAGGUGCUGAAGGACUUCGCCCUGGAAGGCA | |
| | CCCUGGCCGCCGACAAGACCACCCUGAAGGUGACCGAGGGCACCGUGGUGCUGAG | |
| | CAAGCACAUCCCCAACAGCGGCGAGAUCACCGUGGAGCUGCAGGACAGCGACAGC | |
| | ACCCAGGCCACCAAGAAGACCGGCAAGUGGGACAGCCAGACCAGCACCCUGACCA | |
| | UCUCCGUGAACAGCAAGAAGACCAAGAACAUCGUGUUCACAAAGGAGGACACCAU | |
| | CACCGUGCAGAAGUACGACAGCGCCGGCACCAACCUGGAGGGCAACGCCGUGGAG | |
| | AUCAAGACACUGGACGAGCUGAAGAACGCCCUGAAG | |
| 3′ UTR | UAAAGCUCCCCGGGGGCCUCGGUGGCCUAGCUUCUUGCCCCUUGGGCCUCCCCCC | 60 |
| | AGCCCCUCCUCCCCUUCCUGCAGAUAACUGAAGGAAGAGUGGUCUUUGAAUAAAG | |
| | UCUGAGUGGGCGGC | |
| PolyA | 100 nucleotides | |
| Corresponding | MNPNQKIITIGSVSLTISTICFFMQIAILITTVTLHFKQEKNSVSVDLPGEMKVL | 4 |
| Protein | VSKEKDKDGKYSLMATVDKLELKGTSDKSNGAGTLEGEKSDKSKAKLTISEDLSK | |
| | TTFEIFKEDGKTLVSKKVNSKDKSSIEEKFNAKGELSEKTILRADGTRLEYTEIK | |
| | SDGTGKAKEVLKDFALEGTLAADKTTLKVTEGTVVLSKHIPNSGEITVELQDSDS | |
| | TQATKKTGKWDSQTSTLTISVNSKKTKNIVFTKEDTITVQKYDSAGTNLEGNAVE | |
| | IKTLDELKNALK | |

OspA-S5-Nterm-TM

| SEQ ID NO: 19 consists of from 5′ end to 3′ end: 5′ UTR SEQ ID NO: 22, mRNA ORF SEQ ID NO: | | 19 |
|---|---|---|
| 12, and 3′ UTR SEQ ID NO: 23. | | |
| Chemical | 1-methylpseudouridine | |
| Modification | | |
| Cap | 7mG(5′)ppp(5′)NLmpNp | |
| 5′ UTR | GAGGAAAUCGCAAAAUUUGCUCUUCGCGUUAGAUUUCUUUUAGUUUUCUCGCAAC | 22 |
| | UAGCAAGCUUUUUGUUCUCGCC | |
| mRNA ORF | AUGAACCCCAACCAGAAGAUCAUCACCAUCGGCAGCGUGUCGUUGACCAUCAGCA | 12 |
| | CCAUCUGCUUCUUCAUGCAGAUCGCCAUCCUGAUCACCACCGUGACCCUGCACUU | |
| | CAAGCAGGAGAAGAACAGCGUGAGCGUGGACCUGCCCGGAGGCAUGAAGGUGCUG | |
| | GUGAGUAAGGAGAAGGACAAAGACGGCAAGUACAGCCUGAUGGCCACCGUGGAGA | |
| | AGCUGGAGCUGAAGGGCACCAGCGACAAGAACAACGGAGCCGGCACUCUGGAGGG | |
| | AGAGAAGACAGACAAGAGCAAGGUGAAGCUGACCAUCGCCGAGGACCUGAGUAAG | |
| | ACCACCUUCGAGAUCUUCAAGGAGGACGGCAAGACCCUGGUGAGCAAGAAGGUGA | |
| | CACUGAAGGAUAAGAGCAGCACCGAGGAGAAGUUCAACGAGAAGGGCGAAAUCAG | |
| | CGAGAAGACCAUCGUGAGAGCAGACGGCACCCGGCUGGAGUACACCGACAUCAAG | |
| | AGCGACGGCAGCGGCAAGGCCAAGGAGGUGCUGAAGGACUUCACCCUGGAAGGCA | |
| | CCCUGGCCGCCGACGGCAAGACCACCCUGAAGGUGACCGAGGGCACCGUGGUGCU | |
| | GAGCAAGAACAUCCUGAAGAGCGGCGAGAUCACCGUGGCCCUGGACGACAGCGAC | |
| | ACCACCCAGGCCACCAAGAAGACCGGCAAGUGGGACAGCAAGACCAGCACCCUGA | |
| | CCAUCUCCGUGAACAGCCAGAAGACCAAGAACCUGGUGUUCACAAAGGAGGACAC | |
| | CAUCACCGUGCAGAAGUACGACAGCGCCGGCACCAACCUGGAGGGCAAGGCCGUG | |
| | GAGAUCACCACACUGGAGAAACUGAAGGACGCCCUGAAG | |
| 3′ UTR | UAAAGCUCCCCGGGGGCCUCGGUGGCCUAGCUUCUUGCCCCUUGGGCCUCCCCCC | 23 |
| | AGCCCCUCCUCCCCUUCCUGCACCCGUACCCCCGUGGUCUUUGAAUAAAGUCUGA | |
| | GUGGGCGGC | |
| PolyA | 100 nucleotides | |
| Corresponding | MNPNQKIITIGSVSLTISTICFFMQIAILITTVTLHFKQEKNSVSVDLPGGMKVL | 5 |
| Protein | VSKEKDKDGKYSLMATVEKLELKGTSDKNNGAGTLEGEKTDKSKVKLTIAEDLSK | |
| | TTFEIFKEDGKTLVSKKVTLKDKSSTEEKFNEKGEISEKTIVRADGTRLEYTDIK | |
| | SDGSGKAKEVLKDFTLEGTLAADGKTTLKVTEGTVVLSKNILKSGEITVALDDSD | |
| | TTQATKKTGKWDSKTSTLTISVNSQKTKNLVFTKEDTITVQKYDSAGTNLEGKAV | |
| | EITTLEKLKDALK | |

OspA-S5-Nterm-TM (v2)

| SEQ ID NO: 61 consists of from 5′ end to 3′ end: 5′ UTR SEQ ID NO: 50, mRNA ORF SEQ ID NO: | | 61 |
|---|---|---|
| 12, and 3′ UTR SEQ ID NO: 62. | | |
| Chemical | 1-methylpseudouridine | |
| Modification | | |
| Cap | 5′7MeGpppG2′Ome | |
| 5′ UTR | AGGAAAUCGCAAAAUUUGCUCUUCGCGUUAGAUUUCUUUUAGUUUUCUCGCAACU | 50 |
| | AGCAAGCUUUUUGUUCUCGCC | |
| mRNA ORF | AUGAACCCCAACCAGAAGAUCAUCACCAUCGGCAGCGUGUCGUUGACCAUCAGCA | 12 |
| | CCAUCUGCUUCUUCAUGCAGAUCGCCAUCCUGAUCACCACCGUGACCCUGCACUU | |
| | CAAGCAGGAGAAGAACAGCGUGAGCGUGGACCUGCCCGGAGGCAUGAAGGUGCUG | |
| | GUGAGUAAGGAGAAGGACAAAGACGGCAAGUACAGCCUGAUGGCCACCGUGGAGA | |
| | AGCUGGAGCUGAAGGGCACCAGCGACAAGAACAACGGAGCCGGCACUCUGGAGGG | |
| | AGAGAAGACAGACAAGAGCAAGGUGAAGCUGACCAUCGCCGAGGACCUGAGUAAG | |
| | ACCACCUUCGAGAUCUUCAAGGAGGACGGCAAGACCCUGGUGAGCAAGAAGGUGA | |
| | CACUGAAGGAUAAGAGCAGCACCGAGGAGAAGUUCAACGAGAAGGGCGAAAUCAG | |
| | CGAGAAGACCAUCGUGAGAGCAGACGGCACCCGGCUGGAGUACACCGACAUCAAG | |
| | AGCGACGGCAGCGGCAAGGCCAAGGAGGUGCUGAAGGACUUCACCCUGGAAGGCA | |
| | CCCUGGCCGCCGACGGCAAGACCACCCUGAAGGUGACCGAGGGCACCGUGGUGCU | |
| | GAGCAAGAACAUCCUGAAGAGCGGCGAGAUCACCGUGGCCCUGGACGACAGCGAC | |
| | ACCACCCAGGCCACCAAGAAGACCGGCAAGUGGGACAGCAAGACCAGCACCCUGA | |
| | CCAUCUCCGUGAACAGCCAGAAGACCAAGAACCUGGUGUUCACAAAGGAGGACAC | |
| | CAUCACCGUGCAGAAGUACGACAGCGCCGGCACCAACCUGGAGGGCAAGGCCGUG | |
| | GAGAUCACCACACUGGAGAAACUGAAGGACGCCCUGAAG | |
| 3′ UTR | UAAAGCUCCCCGGGGGCCUCGGUGGCCUAGCUUCUUGCCCCUUGGGCCUCCCCCC | 62 |
| | AGCCCCUCCUCCCCUUCCUGCAGGAGAGUGAGUGUAGUGAGGUGGUCUUUGAAUA | |
| | AAGUCUGAGUGGGCGGC | |
| PolyA | 100 nucleotides | |
| Corresponding | MNPNQKIITIGSVSLTISTICFFMQIAILITTVTLHFKQEKNSVSVDLPGGMKVL | 5 |
| Protein | VSKEKDKDGKYSLMATVEKLELKGTSDKNNGAGTLEGEKTDKSKVKLTIAEDLSK | |
| | TTFEIFKEDGKTLVSKKVTLKDKSSTEEKFNEKGEISEKTIVRADGTRLEYTDIK | |
| | SDGSGKAKEVLKDFTLEGTLAADGKTTLKVTEGTVVLSKNILKSGEITVALDDSD | |
| | TTQATKKTGKWDSKTSTLTISVNSQKTKNLVFTKEDTITVQKYDSAGTNLEGKAV | |
| | EITTLEKLKDALK | |

OspA-S6-S88L-A200V-Nterm-TM

| SEQ ID NO: 20 consists of from 5′ end to 3′ end: 5′ UTR SEQ ID NO: 22, mRNA ORF SEQ ID NO: | | 20 |
|---|---|---|
| 13, and 3′ UTR SEQ ID NO: 23. | | |
| Chemical | 1-methylpseudouridine | |
| Modification | | |
| Cap | 7mG(5′)ppp(5′)NlmpNp | |
| 5′ UTR | GAGGAAAUCGCAAAAUUUGCUCUUCGCGUUAGAUUUCUUUUAGUUUUCUCGCAAC | 22 |
| | UAGCAAGCUUUUUGUUCUCGCC | |
| mRNA ORF | AUGAACCCCAACCAGAAGAUCAUCACCAUCGGCAGCGUGUCGUUGACCAUCAGCA | 13 |
| | CCAUCUGCUUCUUCAUGCAGAUCGCCAUCCUGAUCACCACCGUGACCCUGCACUU | |
| | CAAGCAGGAGAAGAACAGCGUGAGCGUGGACCUGCCCGGAGGCAUGACCGUGCUG | |
| | GUGAGUAAGGAGAAGGACAAAGACGGCAAGUACAGCCUGGAGGCCACCGUGGAUA | |
| | AGCUGGAGCUGAAGGGCACCAGCGACAAGAACAACGGAGCCGGCACUCUGGAGGG | |
| | AGAGAAGACAGACAAGAGCAAGGUGAAGCUGACCAUCGCCGACGACCUGAGUCAG | |
| | ACCAAGUUCGAGAUCUUCAAGGAGGACGGCAAGACCCUGGUGAGCAAGAAGGUGA | |
| | CACUGAAGGAUAAGAGCAGCACCGAGGAGAAGUUCAACGGCAAGGGCGAAACCAG | |
| | CGAGAAGACCAUCGUGAGAGCAGACGGCACCCGGCUGGAGUACACCGACAUCAAG | |
| | AGCGACGGCAGCGGCAAGGCCAAGGAGGUGCUGAAGGACUUCACCCUGGAAGGCA | |
| | CCCUGGCCGCCGACGGCAAGACCACCCUGAAGGUGACCGAGGGCACCGUGGUGCU | |
| | GAGCAAGAACAUCCUGAAGAGCGGCGAGAUCACCGUGGCCCUGGACGACAGCGAC | |
| | ACCACCCGGGCCACCAAGAAGACCGGCAAGUGGGACAGCAAGACCAGCACCCUGA | |
| | CCAUCUCCGUGAACAGCCAGAAGACCAAGAACCUGGUGUUCACAAAGGAGGACAC | |
| | CAUCACCGUGCAGCGGUACGACAGCGCCGGCACCAACCUGGAGGGCAAGGCCGUG | |
| | GAGAUCACCACACUGAAAGAGCUGAAGAACGCCCUGAAG | |
| 3′ UTR | UAAAGCUCCCCGGGGGCCUCGGUGGCCUAGCUUCUUGCCCCUUGGGCCUCCCCCC | 23 |
| | AGCCCCUCCUCCCCUUCCUGCACCCGUACCCCCGUGGUCUUUGAAUAAAGUCUGA | |
| | GUGGGCGGC | |
| PolyA | 100 nucleotides | |
| Corresponding | MNPNQKIITIGSVSLTISTICFFMQIAILITTVTLHFKQEKNSVSVDLPGGMTVL | 6 |
| Protein | VSKEKDKDGKYSLEATVDKLELKGTSDKNNGAGTLEGEKTDKSKVKLTIADDLSQ | |
| | TKFEIFKEDGKTLVSKKVTLKDKSSTEEKFNGKGETSEKTIVRADGTRLEYTDIK | |
| | SDGSGKAKEVLKDFTLEGTLAADGKTTLKVTEGTVVLSKNILKSGEITVALDDSD | |
| | TTRATKKTGKWDSKTSTLTISVNSQKTKNLVFTKEDTITVQRYDSAGTNLEGKAV | |
| | EITTLKELKNALK | |

OspA-S6-S88L-A200V-Nterm-TM (v2)

| SEQ ID NO: 63 consists of from 5′ end to 3′ end: 5′ UTR SEQ ID NO: 50, mRNA ORF SEQ ID NO: | | 53 |
|---|---|---|
| 13, and 3′ UTR SEQ ID NO: 64. | | |
| Chemical | 1-methylpseudouridine | |
| Modification | | |
| Cap | 5′7MeGpppG2′Ome | |
| 5′ UTR | AGGAAAUCGCAAAAUUUGCUCUUCGCGUUAGAUUUCUUUUAGUUUUCUCGCAACU | 50 |
| | AGCAAGCUUUUUGUUCUCGCC | |
| mRNA ORF | AUGAACCCCAACCAGAAGAUCAUCACCAUCGGCAGCGUGUCGUUGACCAUCAGCA | 13 |
| | CCAUCUGCUUCUUCAUGCAGAUCGCCAUCCUGAUCACCACCGUGACCCUGCACUU | |
| | CAAGCAGGAGAAGAACAGCGUGAGCGUGGACCUGCCCGGAGGCAUGACCGUGCUG | |
| | GUGAGUAAGGAGAAGGACAAAGACGGCAAGUACAGCCUGGAGGCCACCGUGGAUA | |
| | AGCUGGAGCUGAAGGGCACCAGCGACAAGAACAACGGAGCCGGCACUCUGGAGGG | |
| | AGAGAAGACAGACAAGAGCAAGGUGAAGCUGACCAUCGCCGACGACCUGAGUCAG | |
| | ACCAAGUUCGAGAUCUUCAAGGAGGACGGCAAGACCCUGGUGAGCAAGAAGGUGA | |
| | CACUGAAGGAUAAGAGCAGCACCGAGGAGAAGUUCAACGGCAAGGGCGAAACCAG | |
| | CGAGAAGACCAUCGUGAGAGCAGACGGCACCCGGCUGGAGUACACCGACAUCAAG | |
| | AGCGACGGCAGCGGCAAGGCCAAGGAGGUGCUGAAGGACUUCACCCUGGAAGGCA | |
| | CCCUGGCCGCCGACGGCAAGACCACCCUGAAGGUGACCGAGGGCACCGUGGUGCU | |
| | GAGCAAGAACAUCCUGAAGAGCGGCGAGAUCACCGUGGCCCUGGACGACAGCGAC | |
| | ACCACCCGGGCCACCAAGAAGACCGGCAAGUGGGACAGCAAGACCAGCACCCUGA | |
| | CCAUCUCCGUGAACAGCCAGAAGACCAAGAACCUGGUGUUCACAAAGGAGGACAC | |
| | CAUCACCGUGCAGCGGUACGACAGCGCCGGCACCAACCUGGAGGGCAAGGCCGUG | |
| | GAGAUCACCACACUGAAAGAGCUGAAGAACGCCCUGAAG | |
| 3′ UTR | UAAAGCUCCCCGGGGGCCUCGGUGGCCUAGCUUCUUGCCCCUUGGGCCUCCCCCC | 64 |
| | AGCCCCUCCUCCCCUUCCUGCAGGAGAGUGAGUGUAGUGAGUAAGUGGUCUUUGA | |
| | AUAAAGUCUGAGUGGGCGGC | |
| PolyA | 100 nucleotides | |
| Corresponding | MNPNQKIITIGSVSLTISTICFFMQIAILITTVTLHFKQEKNSVSVDLPGGMTVL | 6 |
| Protein | VSKEKDKDGKYSLEATVDKLELKGTSDKNNGAGTLEGEKTDKSKVKLTIADDLSQ | |
| | TKFEIFKEDGKTLVSKKVTLKDKSSTEEKFNGKGETSEKTIVRADGTRLEYTDIK | |
| | SDGSGKAKEVLKDFTLEGTLAADGKTTLKVTEGTVVLSKNILKSGEITVALDDSD | |
| | TTRATKKTGKWDSKTSTLTISVNSQKTKNLVFTKEDTITVQRYDSAGTNLEGKAV | |
| | EITTLKELKNALK | |

OspA-S7-Nterm-TM

| SEQ ID NO: 21 consists of from 5′ end to 3′ end: 5′ UTR SEQ ID NO: 22, mRNA ORF SEQ ID NO: | | 21 |
|---|---|---|
| 14, and 3′ UTR SEQ ID NO: 23. | | |
| Chemical | 1-methylpseudouridine | |
| Modification | | |
| Cap | 7mG(5′)ppp(5′)NImpNp | |
| 5′ UTR | GAGGAAAUCGCAAAAUUUGCUCUUCGCGUUAGAUUUCUUUUAGUUUUCUCGCAAC | 22 |
| | UAGCAAGCUUUUUGUUCUCGCC | |
| mRNA ORF | AUGAACCCCAACCAGAAGAUCAUCACCAUCGGCAGCGUGUCGUUGACCAUCAGCA | 14 |
| | CCAUCUGCUUCUUCAUGCAGAUCGCCAUCCUGAUCACCACCGUGACCCUGCACUU | |
| | CAAGCAGGAGAAGAACAGCGUGAGCGUGGACCUGCCCGGAGAGAUGAAGGUGCUG | |
| | GUGAGUAAGGAGAAGGACAAAGACGGCAAGUACAGCCUGGAGGCCACCGUGGAUA | |
| | AGCUGGAGCUGAAGGGCACCAGCGACAAGAACAACGGAGCCGGCGUGCUGGAGGG | |
| | AGUGAAGGCCGCCAAGAGCAAGGCAAAGCUGACCAUCGCCGACGACCUGAGUCAG | |
| | ACCAAGUUCGAGAUCUUCAAGGAGGACGGCAAGACCCUGGUGAGCAAGAAGGUGA | |
| | CACUGAAGGAUAAGAGCAGCACCGAGGAGAAGUUCAACGACAAGGGCAAGCUGAG | |
| | CGAGAAGGUGGUGACCAGAGCAGACGGCACCCGGCUGGAGUACACCGAGAUCCAG | |
| | AACGACGGCAGCGGCAAGGCCAAGGAGGUGCUGAAGAGCCUGACCCUGGAAGGCA | |
| | CCCUGACCGCCGACGGCGAGACCAAGCUGACCGUGGAGGCCGGCACCGUGACCCU | |
| | GAGCAAGGACAUCAGCGAGAGCGGCGAGAUCACCGUGGAGCUGAAAGACACCGAG | |
| | ACCACCCCAGCCGACAAGAAGAGCGGCACCUGGGACAGCAAGACCAGCACCCUGA | |
| | CCAUCUCCAAGAACAGCCAGAAGACCAAGCAGCUGGUGUUCACAAAGGAGAACAC | |
| | CAUCACCGUGCAGAAGUACAACACCGCCGGCACCAAGCUGGAGGGCAGCCCCGCA | |
| | GAGAUCAAGGACCUGGAGGCCCUGAAGGCCGCCCUGAAG | |
| 3′ UTR | UAAAGCUCCCCGGGGGCCUCGGUGGCCUAGCUUCUUGCCCCUUGGGCCUCCCCCC | 23 |
| | AGCCCCUCCUCCCCUUCCUGCACCCGUACCCCCGUGGUCUUUGAAUAAAGUCUGA | |
| | GUGGGCGGC | |
| PolyA | 100 nucleotides | |
| Corresponding | MNPNQKIITIGSVSLTISTICFFMQIAILITTVTLHFKQEKNSVSVDLPGEMKVL | 7 |
| Protein | VSKEKDKDGKYSLEATVDKLELKGTSDKNNGAGVLEGVKAAKSKAKLTIADDLSQ | |
| | TKFEIFKEDGKTLVSKKVTLKDKSSTEEKFNDKGKLSEKVVTRADGTRLEYTEIQ | |
| | NDGSGKAKEVLKSLTLEGTLTADGETKLTVEAGTVTLSKDISESGEITVELKDTE | |
| | TTPADKKSGTWDSKTSTLTISKNSQKTKQLVFTKENTITVQKYNTAGTKLEGSPA | |
| | EIKDLEALKAALK | |

OspA-S7-Nterm-TM (v2)

| SEQ ID NO: 65 consists of from 5′ end to 3′ end: 5′ UTR SEQ ID NO: 50, mRNA ORF SEQ ID NO: | | 65 |
|---|---|---|
| 14, and 3′ UTR SEQ ID NO: 66. | | |
| Chemical | 1-methylpseudouridine | |
| Modification | | |
| Cap | 5′7MeGpppG2′Ome | |
| 5′ UTR | AGGAAAUCGCAAAAUUUGCUCUUCGCGUUAGAUUUCUUUUAGUUUUCUCGCAACU | 50 |
| | AGCAAGCUUUUUGUUCUCGCC | |
| mRNA ORF | AUGAACCCCAACCAGAAGAUCAUCACCAUCGGCAGCGUGUCGUUGACCAUCAGCA | 14 |
| | CCAUCUGCUUCUUCAUGCAGAUCGCCAUCCUGAUCACCACCGUGACCCUGCACUU | |
| | CAAGCAGGAGAAGAACAGCGUGAGCGUGGACCUGCCCGGAGAGAUGAAGGUGCUG | |
| | GUGAGUAAGGAGAAGGACAAAGACGGCAAGUACAGCCUGGAGGCCACCGUGGAUA | |
| | AGCUGGAGCUGAAGGGCACCAGCGACAAGAACAACGGAGCCGGCGUGCUGGAGGG | |
| | AGUGAAGGCCGCCAAGAGCAAGGCAAAGCUGACCAUCGCCGACGACCUGAGUCAG | |
| | ACCAAGUUCGAGAUCUUCAAGGAGGACGGCAAGACCCUGGUGAGCAAGAAGGUGA | |
| | CACUGAAGGAUAAGAGCAGCACCGAGGAGAAGUUCAACGACAAGGGCAAGCUGAG | |
| | CGAGAAGGUGGUGACCAGAGCAGACGGCACCCGGCUGGAGUACACCGAGAUCCAG | |
| | AACGACGGCAGCGGCAAGGCCAAGGAGGUGCUGAAGAGCCUGACCCUGGAAGGCA | |
| | CCCUGACCGCCGACGGCGAGACCAAGCUGACCGUGGAGGCCGGCACCGUGACCCU | |
| | GAGCAAGGACAUCAGCGAGAGCGGCGAGAUCACCGUGGAGCUGAAAGACACCGAG | |
| | ACCACCCCAGCCGACAAGAAGAGCGGCACCUGGGACAGCAAGACCAGCACCCUGA | |
| | CCAUCUCCAAGAACAGCCAGAAGACCAAGCAGCUGGUGUUCACAAAGGAGAACAC | |
| | CAUCACCGUGCAGAAGUACAACACCGCCGGCACCAAGCUGGAGGGCAGCCCCGCA | |
| | GAGAUCAAGGACCUGGAGGCCCUGAAGGCCGCCCUGAAG | |
| 3′ UTR | UAAAGCUCCCCGGGGGCCUCGGUGGCCUAGCUUCUUGCCCCUUGGGCCUCCCCCC | 66 |
| | AGCCCCUCCUCCCCUUCCUGCAGUUGAGGCACGCGUCGGCAGGCGCAGUGGUCUU | |
| | UGAAUAAAGUCUGAGUGGGCGGC | |
| PolyA | 100 nucleotides | |
| Corresponding | MNPNQKIITIGSVSLTISTICFFMQIAILITTVTLHFKQEKNSVSVDLPGEMKVL | 7 |
| Protein | VSKEKDKDGKYSLEATVDKLELKGTSDKNNGAGVLEGVKAAKSKAKLTIADDLSQ | |
| | TKFEIFKEDGKTLVSKKVTLKDKSSTEEKFNDKGKLSEKVVTRADGTRLEYTEIQ | |
| | NDGSGKAKEVLKSLTLEGTLTADGETKLTVEAGTVTLSKDISESGEITVELKDTE | |
| | TTPADKKSGTWDSKTSTLTISKNSQKTKQLVFTKENTITVQKYNTAGTKLEGSPA | |
| | EIKDLEALKAALK | |

TABLE 2
provides examples of naturally occurring Borrelia OspA proteins.
Table 2: Naturally Occurring Borrelia Protein Sequence

| Borrelia | | | |
|---|---|---|---|
| Strain and | | | SEQ |
| OspA | NCBI accession | | ID |
| Serotype | ID | Naturally Occurring Protein Sequence | NO: |
| Borrelia | WP_010890378.1 | MKKYLLGIGLILALIACKQNVSSLDEKNSVSVDLPGEMKVLVSK | 24 |
| burgdorferi | | EKNKDGKYDLIATVDKLELKGTSDKNNGSGVLEGVKADKSKVKL | |
| (serotype 1) | | TISDDLGQTTLEVFKEDGKTLVSKKVTSKDKSSTEEKFNEKGEV | |
| | | SEKIITRADGTRLEYTGIKSDGSGKAKEVLKGYVLEGTLTAEKT | |
| | | TLVVKEGTVTLSKNISKSGEVSVELNDTDSSAATKKTAAWNSGT | |
| | | STLTITVNSKKTKDLVFTKENTITVQQYDSNGTKLEGSAVEITK | |
| | | LDEIKNALK | |
| Borrelia | WP_011703777.1 | MKKYLLGIGLILALIACKQNVSSLDEKNSASVDLPGEMKVLVSK | 25 |
| afzelii | | EKDKDGKYSLKATVDKIELKGTSDKDNGSGVLEGTKDDKSKAKL | |
| (serotype 2) | | TIADDLSKTTFELFKEDGKTLVSRKVSSKDKTSTDEMFNEKGEL | |
| | | SAKTMTRENGTKLEYTEMKSDGTGKAKEVLKNFTLEGKVANDKV | |
| | | TLEVKEGTVTLSKEIAKSGEVTVALNDTNTTQATKKTGAWDSKT | |
| | | STLTISVNSKKTTQLVFTKQDTITVQKYDSAGTNLEGTAVEIKT | |
| | | LDELKNALK | |
| Borrelia | CAA56549.1 | MKKYLLGIGLILALIACKQNVSSLDEKNSVSVDLPGGMKVLVSK | 26 |
| garinii | | EKDKDGKYSLMATVEKLELKGTSDKSNGSGVLEGEKADKSKAKL | |
| (serotype 3) | | TISQDLNQTTFEIFKEDGKTLVSRKVNSKDKSSTEEKFNDKGKL | |
| | | SEKVVTRANGTRLEYTEIKNDGSGKAKEVLKGFALEGTLTDGGE | |
| | | TKLTVTEGTVTLSKNISKSGEITVALNDTETTPADKKTGEWKSD | |
| | | TSTLTISKNSQKPKQLVFTKENTITVQNYNRAGNALEGSPAEIK | |
| | | DLAELKAALK | |
| Borrelia | WP_011187157.1 | MKKYLLGIGLILALIACKQNVSSLDEKNSVSVDLPGEMKVLVSK | 27 |
| bavariensis | | EKDKDGKYSLMATVDKLELKGTSDKSNGSGTLEGEKSDKSKAKL | |
| (serotype 4) | | TISEDLSKTTFEIFKEDGKTLVSKKVNSKDKSSIEEKFNAKGEL | |
| | | SEKTILRANGTRLEYTEIKSDGTGKAKEVLKDFALEGTLAADKT | |
| | | TLKVTEGTVVLSKHIPNSGEITVELNDSNSTQATKKIGKWDSNT | |
| | | STLTISVNSKKTKNIVFTKEDTITVQKYDSAGTNLEGNAVEIKT | |
| | | LDELKNALK | |
| Borrelia | CAA59727.1 | MKKYLLGIGLILALIACKQNVSSLDEKNSVSVDLPGGMKVLVSK | 28 |
| garinii | | EKDKDGKYSLMATVEKLELKGTSDKNNGSGTLEGEKTDKSKVKL | |
| (serotype 5) | | TIAEDLSKTTFEIFKEDGKTLVSKKVTLKDKSSTEEKFNEKGEI | |
| | | SEKTIVRANGTRLEYTDIKSDGSGKAKEVLKDFTLEGTLAADGK | |
| | | TTLKVTEGTVVLSKNILKSGEITVALDDSDTTQATKKTGKWDSK | |
| | | TSTLTISVNSQKTKNLVFTKEDTITVQKYDSAGTNLEGKAVEIT | |
| | | TLEKLKDALK | |
| Borrelia | CAA45010.1 | MKKYLLGIGLILALIACKQNVSSLDEKNSVSVDLPGGMTVLVSK | 29 |
| garinii | | EKDKDGKYSLEATVDKLELKGTSDKNNGSGTLEGEKTDKSKVKS | |
| (serotype 6) | | TIADDLSQTKFEIFKEDGKTLVSKKVTLKDKSSTEEKFNGKGET | |
| | | SEKTIVRANGTRLEYTDIKSDGSGKAKEVLKDFTLEGTLAADGK | |
| | | TTLKVTEGTVVLSKNILKSGEITAALDDSDTTRATKKTGKWDSK | |
| | | TSTLTISVNSQKTKNLVFTKEDTITVQRYDSAGTNLEGKAVEIT | |
| | | TLKELKNALK | |
| Borrelia | CAA56547.1 | MKKYLLGIGLILALIACKQNVSSLDEKNSVSVDLPGEMKVLVSK | 30 |
| garinii | | EKDKDGKYSLEATVDKLELKGTSDKNNGSGVLEGVKAAKSKAKL | |
| (serotype 7) | | TIADDLSQTKFEIFKEDGKTLVSKKVTLKDKSSTEEKFNDKGKL | |
| | | SEKVVTRANGTRLEYTEIQNDGSGKAKEVLKSLTLEGTLTADGE | |
| | | TKLTVEAGTVTLSKNISESGEITVELKDTETTPADKKSGTWDSK | |
| | | TSTLTISKNSQKTKQLVFTKENTITVQKYNTAGTKLEGSPAEIK | |
| | | DLEALKAALK | |

In some embodiments, a vaccine of the present disclosure comprises an mRNA polynucleotide comprising an open reading frame encoding a Borrelia OspA S1 protein, wherein the OspA S1 protein comprises an amino acid sequence having at least 80%, at least 85%, at least 90%, at least 95%, at least 98%, or at least 99% identity to the amino acid sequence of SEQ ID NO: 1. In some embodiments, a vaccine of the present disclosure comprises an mRNA polynucleotide comprising an open reading frame encoding a Borrelia OspA 82 protein, wherein the OspA 82 protein comprises an amino acid sequence having at least 80%, at least 85%, at least 90%, at least 95%, at least 98%, or at least 99% identity to the amino acid sequence of SEQ ID NO: 2. In some embodiments, a vaccine of the present disclosure comprises an mRNA polynucleotide comprising an open reading frame encoding a Borrelia OspA 83 protein, wherein the OspA S3 protein comprises an amino acid sequence having at least 80%, at least 85%, at least 90%, at least 95%, at least 98%, or at least 99% identity to the amino acid sequence of SEQ ID NO: 3. In some embodiments, a vaccine of the present disclosure comprises an mRNA polynucleotide comprising an open reading frame encoding a Borrelia OspA S4 protein, wherein the OspA 84 protein comprises an amino acid sequence having at least 80%, at least 85%, at least 90%, at least 95%, at least 98%, or at least 99% identity to the amino acid sequence of SEQ ID NO: 4. In some embodiments, a vaccine of the present disclosure comprises an mRNA polynucleotide comprising an open reading frame encoding a Borrelia OspA 85 protein, wherein the OspA S5 protein comprises an amino acid sequence having at least 80%, at least 85%, at least 90%, at least 95%, at least 98%, or at least 99% identity to the amino acid sequence of SEQ ID NO: 5. In some embodiments, a vaccine of the present disclosure comprises an mRNA polynucleotide comprising an open reading frame encoding a Borrelia OspA S6 protein, wherein the OspA S6 protein comprises an amino acid sequence having at least 80%, at least 85%, at least 90%, at least 95%, at least 98%, or at least 99% identity to the amino acid sequence of SEQ ID NO: 6. In some embodiments, a vaccine of the present disclosure comprises an mRNA polynucleotide comprising an open reading frame encoding a Borrelia OspA S7 protein, wherein the OspA S7 protein comprises an amino acid sequence having at least 80%, at least 85%, at least 90%, at least 95%, at least 98%, or at least 99% identity to the amino acid sequence of SEQ ID NO: 7.

In some embodiments, a vaccine of the present disclosure comprises an mRNA polynucleotide comprising an open reading frame encoding a Borrelia OspA S1 protein, wherein the OspA S1 protein comprises the amino acid sequence of SEQ ID NO: 1. In some embodiments, a vaccine of the present disclosure comprises an mRNA polynucleotide comprising an open reading frame encoding a Borrelia OspA S2 protein, wherein the OspA S2 protein comprises the amino acid sequence of SEQ ID NO: 2. In some embodiments, a vaccine of the present disclosure comprises an mRNA polynucleotide comprising an open reading frame encoding a Borrelia OspA S3 protein, wherein the OspA S3 protein comprises the amino acid sequence of SEQ ID NO: 3. In some embodiments, a vaccine of the present disclosure comprises an mRNA polynucleotide comprising an open reading frame encoding a Borrelia OspA S4 protein, wherein the OspA S4 protein comprises the amino acid sequence of SEQ ID NO: 4. In some embodiments, a vaccine of the present disclosure comprises an mRNA polynucleotide comprising an open reading frame encoding a Borrelia OspA S5 protein, wherein the OspA S5 protein comprises the amino acid sequence of SEQ ID NO: 5. In some embodiments, a vaccine of the present disclosure comprises an mRNA polynucleotide comprising an open reading frame encoding a Borrelia OspA S6 protein, wherein the OspA S6 protein comprises the amino acid sequence of SEQ ID NO: 6. In some embodiments, a vaccine of the present disclosure comprises an mRNA polynucleotide comprising an open reading frame encoding a Borrelia OspA S7 protein, wherein the OspA S7 protein comprises the amino acid sequence of SEQ ID NO: 7.

The mRNA polynucleotides of the present disclosure encode at least one Borrelia protein of interest, intended to raise an immune response which is protective against subsequent Borrelia infection in mammalian subjects. Thus, when administered to a subject, the mRNA vaccines lead to expression of Borrelia proteins of the present disclosure which are antigenic, i.e., they provoke a protective immune response. Antigenicity can be measured by the ability to generate cellular and/or humoral immune responses. Delivery of the mRNA polynucleotides of the present disclosure is achieved by formulating the mRNA in appropriate carriers or delivery vehicles (e.g., lipid nanoparticles) such that upon administration to cells, tissues or subjects, the mRNA is taken up by cells which, in turn, express the protein(s) encoded by the mRNA.

It should be understood that the term “protein” encompasses peptides (and polypeptides shorter than a full-length protein), and the term “antigen” encompasses antigenic fragments.

The vaccines of the present disclosure provide a unique advantage over traditional protein-based vaccination approaches in which protein antigens are purified or produced in vitro, e.g., recombinant protein production technologies. The vaccines of the present disclosure comprise mRNA encoding the desired Borrelia antigen(s), which when introduced into the body, i.e., administered to a mammalian subject (for example a human) in vivo, cause the cells of the body to express the desired antigen(s). In order to facilitate delivery of the mRNA to the cells of the body, the mRNA is formulated (e.g., encapsulated) in a lipid nanoparticle. Upon delivery and uptake by cells of the body, the mRNA is translated in the cytosol and the antigens are generated by the host cell machinery. The antigens are expressed and presented by the host cells and elicit humoral and/or cellular immune responses. Neutralizing antibodies are directed against the expressed antigens, and hence the antigens are considered relevant target antigens for vaccine development.

Many proteins have a quaternary or three-dimensional structure, which includes more than one polypeptide or several polypeptide chains that associate into an oligomeric molecule. As used herein the term “subunit” refers to a single protein molecule, for example, a polypeptide or polypeptide chain resulting from processing of a nascent protein molecule, which subunit assembles (or “coassembles”) with other protein molecules (e.g., subunits or chains) to form a protein complex. Proteins can have a relatively small number of subunits and therefore be described as “oligomeric” or can consist of a large number of subunits and therefore be described as “multimeric”. The subunits of an oligomeric or multimeric protein may be identical, homologous or totally dissimilar and dedicated to disparate tasks.

Proteins or protein subunits can further comprise domains. As used herein, the term “domain” refers to a distinct functional and/or structural unit within a protein. Typically, a “domain” is responsible for a particular function or interaction, contributing to the overall role of a protein. Domains can exist in a variety of biological contexts. Similar domains (i.e., domains sharing structural, functional and/or sequence homology) can exist within a single protein or can exist within distinct proteins having similar or different functions. A protein domain is often a conserved part of a given protein tertiary structure or sequence that can function and exist independently of the rest of the protein or subunit thereof. An extracellular protein domain is a part of a protein molecule that is located outside of a cell. It is typically exposed to the extracellular environment and can interact with other proteins, molecules, or the cell surface. A transmembrane domain is a structural component of a protein that spans the lipid bilayer of a cell membrane. Transmembrane domains typically include one or more alpha helices or beta strands that cross the hydrophobic lipid bilayer of the cell membrane.

As used herein, the term “antigen” is distinct from the term “epitope,” which is a substructure of an antigen. An epitope of a part of an antigen to which an antibody attaches. An epitope may be a peptide, for example, a 7-10 amino acid peptide, or a carbohydrate structure. The art describes protein antigens that are delivered to subjects or immune cells in isolated form, e.g., isolated proteins, however, the design, testing, validation, and production of protein antigens can be costly and time-consuming, especially when producing proteins at large scale. By contrast, mRNA technology is amenable to rapid design and testing of mRNA encoding a variety of antigens. Moreover, rapid production of mRNA coupled with formulation in appropriate delivery vehicles (e.g., lipid nanoparticles), can proceed quickly and can rapidly produce mRNA vaccines at large scale. Potential benefit also arises from the fact that antigens encoded by the mRNAs of the present disclosure are expressed by the cells of the subject, e.g., are expressed by the human body, and thus the subject, e.g., the human body, serves as the “factory” to produce the antigens which, in turn, elicits the desired immune response.

The vaccines, as provided herein, may include an mRNA polynucleotide or multiple mRNA polynucleotides encoding two or more antigens of the same or different Borrelia strains. For example, multiple OspA antigens, e.g., S1, S2, S3, S4, S5, S6, and S7 can be included in the subject vaccines. Also provided herein are combination vaccines that include mRNA encoding one or more Borrelia antigens and one or more antigen(s) of a different organism. Thus, the vaccines of the present disclosure may be combination vaccines that target one or more antigens of the same strain/species, or one or more antigens of different strains/species, e.g., antigens that induce immunity to organisms that are found in the same geographic areas where the risk of Borrelia infection is high or organisms to which an individual is likely to be exposed to when exposed to Borrelia.

### Percent Identity

In some embodiments, the compositions of the present disclosure include mRNA that encodes a Borrelia protein variant. Protein variants are proteins (including full length proteins and peptides) that differ in their amino acid sequence relative to a naturally occurring or reference amino acid sequence. A protein variant may possess one or more substitutions, deletions, and/or insertions at certain positions within its amino acid sequence, as compared to a naturally occurring or reference amino acid sequence. Ordinarily, protein variants have at least 50% identity to a naturally occurring or reference sequence. In some embodiments, a protein variant has at least 60%, at least 70%, at least 75%, at least 80%, at least 85%, at least 90%, or at least 95% identity to a naturally occurring or reference sequence.

Global sequence alignment and local sequence alignment are two common methods used to compare and analyze sequences of DNA, RNA, or protein. Global sequence alignment compares the entire length of two sequences and finds the best possible alignment of the entire length of the sequences. It is useful, for example, when the two sequences being compared are similar in length and share significant homology. Local sequence alignment, on the other hand, identifies regions of similarity between sequences, allowing for gaps and mismatches in the alignment. This method is useful for identifying short regions of homology within larger sequences, and can be used to identify functional domains, protein families, and binding sites. Local alignment can be computationally more efficient than global alignment, and can be applied to sequences of different lengths.

Unless stated otherwise herein, “percent (%) identity” between two mRNA polynucleotides or between two proteins refers to percent (%) identity determined using a global sequence alignment, comparing the length of entire sequences (e.g., entire mRNA polynucleotide, entire open reading frame of an mRNA, or entire protein encoded by an mRNA, as described herein).

A protein variant encoded by an mRNA of the disclosure may contain amino acid changes that confer any of a number of desirable properties, for example, that enhance its immunogenicity, enhance its expression, and/or improve its stability or PK/PD properties in a subject. Protein variants can be made using routine mutagenesis techniques and assayed as appropriate to determine whether they possess the desired property. Assays to determine expression levels and immunogenicity of proteins, including protein variants, are well known in the art. Similarly, PK/PD properties of a protein variant can be measured using art recognized techniques, for example, by determining expression of the protein variant in a vaccinated subject over time and/or by looking at the durability of an induced immune response. The stability of a protein variant encoded by an mRNA may be measured by assaying thermal stability or stability upon urea denaturation or may be measured using in silico prediction, for example. Methods for such experiments and in silico determinations are known in the art. Other methods for determining protein variant expression levels, immunogenicity and/or PK/PD properties of a protein variant may be used.

In some embodiments, an mRNA polynucleotide comprises an open reading frame that comprises a nucleotide sequence of any one of the sequences provided herein or comprises a nucleotide sequence that has at least 50%, at least 60%, at least 70%, at least 75%, at least 80%, at least 85%, at least 90%, or at least 95% identity to a nucleotide sequence of any one of the sequences provided herein. See, e.g., SEQ ID NOs: 8-14.

In some embodiments, an mRNA polynucleotide comprises an open reading frame that comprises a nucleotide sequence that has at least 70% identity to the nucleotide sequence of SEQ ID NO: 8. In some embodiments, an mRNA polynucleotide comprises an open reading frame that comprises a nucleotide sequence that has at least 70% identity to the nucleotide sequence of SEQ ID NO: 9. In some embodiments, an mRNA polynucleotide comprises an open reading frame that comprises a nucleotide sequence that has at least 70% identity to the nucleotide sequence of SEQ ID NO: 10. In some embodiments, an mRNA polynucleotide comprises an open reading frame that comprises a nucleotide sequence that has at least 70% identity to the nucleotide sequence of SEQ ID NO: 11. In some embodiments, an mRNA polynucleotide comprises an open reading frame that comprises a nucleotide sequence that has at least 70% identity to the nucleotide sequence of SEQ ID NO: 12. In some embodiments, an mRNA polynucleotide comprises an open reading frame that comprises a nucleotide sequence that has at least 70% identity to the nucleotide sequence of SEQ ID NO: 13. In some embodiments, an mRNA polynucleotide comprises an open reading frame that comprises a nucleotide sequence that has at least 70% identity to the nucleotide sequence of SEQ ID NO: 14. In some embodiments, an mRNA polynucleotide comprises an open reading frame that comprises a nucleotide sequence that has at least 75% identity to the nucleotide sequence of SEQ ID NO: 8. In some embodiments, an mRNA polynucleotide comprises an open reading frame that comprises a nucleotide sequence that has at least 75% identity to the nucleotide sequence of SEQ ID NO: 9. In some embodiments, an mRNA polynucleotide comprises an open reading frame that comprises a nucleotide sequence that has at least 75% identity to the nucleotide sequence of SEQ ID NO: 10. In some embodiments, an mRNA polynucleotide comprises an open reading frame that comprises a nucleotide sequence that has at least 75% identity to the nucleotide sequence of SEQ ID NO: 11. In some embodiments, an mRNA polynucleotide comprises an open reading frame that comprises a nucleotide sequence that has at least 75% identity to the nucleotide sequence of SEQ ID NO: 12. In some embodiments, an mRNA polynucleotide comprises an open reading frame that comprises a nucleotide sequence that has at least 75% identity to the nucleotide sequence of SEQ ID NO: 13. In some embodiments, an mRNA polynucleotide comprises an open reading frame that comprises a nucleotide sequence that has at least 75% identity to the nucleotide sequence of SEQ ID NO: 14.

In some embodiments, an mRNA polynucleotide comprises an open reading frame that comprises a nucleotide sequence that has at least 80% identity to the nucleotide sequence of SEQ ID NO: 8. In some embodiments, an mRNA polynucleotide comprises an open reading frame that comprises a nucleotide sequence that has at least 80% identity to the nucleotide sequence of SEQ ID NO: 9. In some embodiments, an mRNA polynucleotide comprises an open reading frame that comprises a nucleotide sequence that has at least 80% identity to the nucleotide sequence of SEQ ID NO: 10. In some embodiments, an mRNA polynucleotide comprises an open reading frame that comprises a nucleotide sequence that has at least 80% identity to the nucleotide sequence of SEQ ID NO: 11. In some embodiments, an mRNA polynucleotide comprises an open reading frame that comprises a nucleotide sequence that has at least 80% identity to the nucleotide sequence of SEQ ID NO: 12. In some embodiments, an mRNA polynucleotide comprises an open reading frame that comprises a nucleotide sequence that has at least 80% identity to the nucleotide sequence of SEQ ID NO: 13. In some embodiments, an mRNA polynucleotide comprises an open reading frame that comprises a nucleotide sequence that has at least 80% identity to the nucleotide sequence of SEQ ID NO: 14. In some embodiments, an mRNA polynucleotide comprises an open reading frame that comprises a nucleotide sequence that has at least 85% identity to the nucleotide sequence of SEQ ID NO: 8. In some embodiments, an mRNA polynucleotide comprises an open reading frame that comprises a nucleotide sequence that has at least 85% identity to the nucleotide sequence of SEQ ID NO: 9. In some embodiments, an mRNA polynucleotide comprises an open reading frame that comprises a nucleotide sequence that has at least 85% identity to the nucleotide sequence of SEQ ID NO: 10. In some embodiments, an mRNA polynucleotide comprises an open reading frame that comprises a nucleotide sequence that has at least 85% identity to the nucleotide sequence of SEQ ID NO: 11. In some embodiments, an mRNA polynucleotide comprises an open reading frame that comprises a nucleotide sequence that has at least 85% identity to the nucleotide sequence of SEQ ID NO: 12. In some embodiments, an mRNA polynucleotide comprises an open reading frame that comprises a nucleotide sequence that has at least 85% identity to the nucleotide sequence of SEQ ID NO: 13. In some embodiments, an mRNA polynucleotide comprises an open reading frame that comprises a nucleotide sequence that has at least 85% identity to the nucleotide sequence of SEQ ID NO: 14. In some embodiments, an mRNA polynucleotide comprises an open reading frame that comprises a nucleotide sequence that has at least 90% identity to the nucleotide sequence of SEQ ID NO: 8. In some embodiments, an mRNA polynucleotide comprises an open reading frame that comprises a nucleotide sequence that has at least 90% identity to the nucleotide sequence of SEQ ID NO: 9. In some embodiments, an mRNA polynucleotide comprises an open reading frame that comprises a nucleotide sequence that has at least 90% identity to the nucleotide sequence of SEQ ID NO: 10. In some embodiments, an mRNA polynucleotide comprises an open reading frame that comprises a nucleotide sequence that has at least 90% identity to the nucleotide sequence of SEQ ID NO: 11. In some embodiments, an mRNA polynucleotide comprises an open reading frame that comprises a nucleotide sequence that has at least 90% identity to the nucleotide sequence of SEQ ID NO: 12. In some embodiments, an mRNA polynucleotide comprises an open reading frame that comprises a nucleotide sequence that has at least 90% identity to the nucleotide sequence of SEQ ID NO: 13. In some embodiments, an mRNA polynucleotide comprises an open reading frame that comprises a nucleotide sequence that has at least 90% identity to the nucleotide sequence of SEQ ID NO: 14. In some embodiments, an mRNA polynucleotide comprises an open reading frame that comprises a nucleotide sequence that has at least 95% identity to the nucleotide sequence of SEQ ID NO: 8. In some embodiments, an mRNA polynucleotide comprises an open reading frame that comprises a nucleotide sequence that has at least 95% identity to the nucleotide sequence of SEQ ID NO: 9. In some embodiments, an mRNA polynucleotide comprises an open reading frame that comprises a nucleotide sequence that has at least 95% identity to the nucleotide sequence of SEQ ID NO: 10. In some embodiments, an mRNA polynucleotide comprises an open reading frame that comprises a nucleotide sequence that has at least 95% identity to the nucleotide sequence of SEQ ID NO: 11. In some embodiments, an mRNA polynucleotide comprises an open reading frame that comprises a nucleotide sequence that has at least 95% identity to the nucleotide sequence of SEQ ID NO: 12. In some embodiments, an mRNA polynucleotide comprises an open reading frame that comprises a nucleotide sequence that has at least 95% identity to the nucleotide sequence of SEQ ID NO: 13. In some embodiments, an mRNA polynucleotide comprises an open reading frame that comprises a nucleotide sequence that has at least 95% identity to the nucleotide sequence of SEQ ID NO: 14. In some embodiments, an mRNA polynucleotide comprises an open reading frame that comprises the nucleotide sequence of SEQ ID NO: 8. In some embodiments, an mRNA polynucleotide comprises an open reading frame that comprises the nucleotide sequence of SEQ ID NO: 9. In some embodiments, an mRNA polynucleotide comprises an open reading frame that comprises the nucleotide sequence of SEQ ID NO: 10. In some embodiments, an mRNA polynucleotide comprises an open reading frame that comprises the nucleotide sequence of SEQ ID NO: 11. In some embodiments, an mRNA polynucleotide comprises an open reading frame that comprises the nucleotide sequence of SEQ ID NO: 12. In some embodiments, an mRNA polynucleotide comprises an open reading frame that comprises the nucleotide sequence of SEQ ID NO: 13. In some embodiments, an mRNA polynucleotide comprises an open reading frame that comprises the nucleotide sequence of SEQ ID NO: 14.

In some embodiments, an mRNA polynucleotide comprises an open reading frame that comprises a nucleotide sequence that has at least 70% identity to the nucleotide sequence of SEQ ID NO: 15. In some embodiments, an mRNA polynucleotide comprises an open reading frame that comprises a nucleotide sequence that has at least 70% identity to the nucleotide sequence of SEQ ID NO: 16. In some embodiments, an mRNA polynucleotide comprises an open reading frame that comprises a nucleotide sequence that has at least 70% identity to the nucleotide sequence of SEQ ID NO: 17. In some embodiments, an mRNA polynucleotide comprises an open reading frame that comprises a nucleotide sequence that has at least 70% identity to the nucleotide sequence of SEQ ID NO: 18. In some embodiments, an mRNA polynucleotide comprises an open reading frame that comprises a nucleotide sequence that has at least 70% identity to the nucleotide sequence of SEQ ID NO: 19. In some embodiments, an mRNA polynucleotide comprises an open reading frame that comprises a nucleotide sequence that has at least 70% identity to the nucleotide sequence of SEQ ID NO: 20. In some embodiments, an mRNA polynucleotide comprises an open reading frame that comprises a nucleotide sequence that has at least 70% identity to the nucleotide sequence of SEQ ID NO: 21. In some embodiments, an mRNA polynucleotide comprises an open reading frame that comprises a nucleotide sequence that has at least 75% identity to the nucleotide sequence of SEQ ID NO: 15. In some embodiments, an mRNA polynucleotide comprises an open reading frame that comprises a nucleotide sequence that has at least 75% identity to the nucleotide sequence of SEQ ID NO: 16. In some embodiments, an mRNA polynucleotide comprises an open reading frame that comprises a nucleotide sequence that has at least 75% identity to the nucleotide sequence of SEQ ID NO: 17. In some embodiments, an mRNA polynucleotide comprises an open reading frame that comprises a nucleotide sequence that has at least 75% identity to the nucleotide sequence of SEQ ID NO: 18. In some embodiments, an mRNA polynucleotide comprises an open reading frame that comprises a nucleotide sequence that has at least 75% identity to the nucleotide sequence of SEQ ID NO: 19. In some embodiments, an mRNA polynucleotide comprises an open reading frame that comprises a nucleotide sequence that has at least 75% identity to the nucleotide sequence of SEQ ID NO: 20. In some embodiments, an mRNA polynucleotide comprises an open reading frame that comprises a nucleotide sequence that has at least 75% identity to the nucleotide sequence of SEQ ID NO: 21. In some embodiments, an mRNA polynucleotide comprises an open reading frame that comprises a nucleotide sequence that has at least 80% identity to the nucleotide sequence of SEQ ID NO: 15. In some embodiments, an mRNA polynucleotide comprises an open reading frame that comprises a nucleotide sequence that has at least 80% identity to the nucleotide sequence of SEQ ID NO: 16. In some embodiments, an mRNA polynucleotide comprises an open reading frame that comprises a nucleotide sequence that has at least 80% identity to the nucleotide sequence of SEQ ID NO: 17. In some embodiments, an mRNA polynucleotide comprises an open reading frame that comprises a nucleotide sequence that has at least 80% identity to the nucleotide sequence of SEQ ID NO: 18. In some embodiments, an mRNA polynucleotide comprises an open reading frame that comprises a nucleotide sequence that has at least 80% identity to the nucleotide sequence of SEQ ID NO: 19. In some embodiments, an mRNA polynucleotide comprises an open reading frame that comprises a nucleotide sequence that has at least 80% identity to the nucleotide sequence of SEQ ID NO: 20. In some embodiments, an mRNA polynucleotide comprises an open reading frame that comprises a nucleotide sequence that has at least 80% identity to the nucleotide sequence of SEQ ID NO: 21. In some embodiments, an mRNA polynucleotide comprises an open reading frame that comprises a nucleotide sequence that has at least 85% identity to the nucleotide sequence of SEQ ID NO: 15. In some embodiments, an mRNA polynucleotide comprises an open reading frame that comprises a nucleotide sequence that has at least 85% identity to the nucleotide sequence of SEQ ID NO: 16. In some embodiments, an mRNA polynucleotide comprises an open reading frame that comprises a nucleotide sequence that has at least 85% identity to the nucleotide sequence of SEQ ID NO: 17. In some embodiments, an mRNA polynucleotide comprises an open reading frame that comprises a nucleotide sequence that has at least 85% identity to the nucleotide sequence of SEQ ID NO: 18. In some embodiments, an mRNA polynucleotide comprises an open reading frame that comprises a nucleotide sequence that has at least 85% identity to the nucleotide sequence of SEQ ID NO: 19. In some embodiments, an mRNA polynucleotide comprises an open reading frame that comprises a nucleotide sequence that has at least 85% identity to the nucleotide sequence of SEQ ID NO: 20. In some embodiments, an mRNA polynucleotide comprises an open reading frame that comprises a nucleotide sequence that has at least 85% identity to the nucleotide sequence of SEQ ID NO: 21. In some embodiments, an mRNA polynucleotide comprises an open reading frame that comprises a nucleotide sequence that has at least 90% identity to the nucleotide sequence of SEQ ID NO: 15. In some embodiments, an mRNA polynucleotide comprises an open reading frame that comprises a nucleotide sequence that has at least 90% identity to the nucleotide sequence of SEQ ID NO: 16. In some embodiments, an mRNA polynucleotide comprises an open reading frame that comprises a nucleotide sequence that has at least 90% identity to the nucleotide sequence of SEQ ID NO: 17. In some embodiments, an mRNA polynucleotide comprises an open reading frame that comprises a nucleotide sequence that has at least 90% identity to the nucleotide sequence of SEQ ID NO: 18. In some embodiments, an mRNA polynucleotide comprises an open reading frame that comprises a nucleotide sequence that has at least 90% identity to the nucleotide sequence of SEQ ID NO: 19. In some embodiments, an mRNA polynucleotide comprises an open reading frame that comprises a nucleotide sequence that has at least 90% identity to the nucleotide sequence of SEQ ID NO: 20. In some embodiments, an mRNA polynucleotide comprises an open reading frame that comprises a nucleotide sequence that has at least 90% identity to the nucleotide sequence of SEQ ID NO: 21. In some embodiments, an mRNA polynucleotide comprises an open reading frame that comprises a nucleotide sequence that has at least 95% identity to the nucleotide sequence of SEQ ID NO: 15. In some embodiments, an mRNA polynucleotide comprises an open reading frame that comprises a nucleotide sequence that has at least 95% identity to the nucleotide sequence of SEQ ID NO: 16. In some embodiments, an mRNA polynucleotide comprises an open reading frame that comprises a nucleotide sequence that has at least 95% identity to the nucleotide sequence of SEQ ID NO: 17. In some embodiments, an mRNA polynucleotide comprises an open reading frame that comprises a nucleotide sequence that has at least 95% identity to the nucleotide sequence of SEQ ID NO: 18. In some embodiments, an mRNA polynucleotide comprises an open reading frame that comprises a nucleotide sequence that has at least 95% identity to the nucleotide sequence of SEQ ID NO: 19. In some embodiments, an mRNA polynucleotide comprises an open reading frame that comprises a nucleotide sequence that has at least 95% identity to the nucleotide sequence of SEQ ID NO: 20. In some embodiments, an mRNA polynucleotide comprises an open reading frame that comprises a nucleotide sequence that has at least 95% identity to the nucleotide sequence of SEQ ID NO: 21. In some embodiments, an mRNA polynucleotide comprises an open reading frame that comprises a nucleotide sequence that has 100% identity to the nucleotide sequence of SEQ ID NO: 15 (i.e., comprises the nucleotide sequence of SEQ ID NO: 15). In some embodiments, an mRNA polynucleotide comprises an open reading frame that comprises the nucleotide sequence of SEQ ID NO: 16. In some embodiments, an mRNA polynucleotide comprises an open reading frame that comprises the nucleotide sequence of SEQ ID NO: 17. In some embodiments, an mRNA polynucleotide comprises an open reading frame that comprises the nucleotide sequence of SEQ ID NO: 18. In some embodiments, an mRNA polynucleotide comprises an open reading frame that comprises the nucleotide sequence of SEQ ID NO: 19. In some embodiments, an mRNA polynucleotide comprises an open reading frame that comprises a the nucleotide sequence of SEQ ID NO: 20. In some embodiments, an mRNA polynucleotide comprises an open reading frame that comprises the nucleotide sequence of SEQ ID NO: 21.

In some embodiments, an mRNA polynucleotide comprises an open reading frame that encodes a protein comprising an amino acid sequence of any one of the sequences provided herein or comprises an amino acid sequence that has at least 50%, at least 60%, at least 70%, at least 75%, at least 80%, at least 85%, at least 90%, or at least 95% identity to the amino acid sequence of any one of the sequences provided herein. See, e.g., SEQ ID NOs: 1-7. In some embodiments, an mRNA polynucleotide comprises an open reading frame that encodes a protein comprising an amino acid sequence that has at least 80% identity to the amino acid sequence of SEQ ID NO: 1. In some embodiments, an mRNA polynucleotide comprises an open reading frame that encodes a protein comprising an amino acid sequence that has at least 80% identity to the amino acid sequence of SEQ ID NO: 2. In some embodiments, an mRNA polynucleotide comprises an open reading frame that encodes a protein comprising an amino acid sequence that has at least 80% identity to the amino acid sequence of SEQ ID NO: 3. In some embodiments, an mRNA polynucleotide comprises an open reading frame that encodes a protein comprising an amino acid sequence that has at least 80% identity to the amino acid sequence of SEQ ID NO: 4. In some embodiments, an mRNA polynucleotide comprises an open reading frame that encodes a protein comprising an amino acid sequence that has at least 80% identity to the amino acid sequence of SEQ ID NO: 5. In some embodiments, an mRNA polynucleotide comprises an open reading frame that encodes a protein comprising an amino acid sequence that has at least 80% identity to the amino acid sequence of SEQ ID NO: 6. In some embodiments, an mRNA polynucleotide comprises an open reading frame that encodes a protein comprising an amino acid sequence that has at least 80% identity to the amino acid sequence of SEQ ID NO: 7. In some embodiments, an mRNA polynucleotide comprises an open reading frame that encodes a protein comprising an amino acid sequence that has at least 85% identity to the amino acid sequence of SEQ ID NO: 1. In some embodiments, an mRNA polynucleotide comprises an open reading frame that encodes a protein comprising an amino acid sequence that has at least 85% identity to the amino acid sequence of SEQ ID NO: 2. In some embodiments, an mRNA polynucleotide comprises an open reading frame that encodes a protein comprising an amino acid sequence that has at least 85% identity to the amino acid sequence of SEQ ID NO: 3. In some embodiments, an mRNA polynucleotide comprises an open reading frame that encodes a protein comprising an amino acid sequence that has at least 85% identity to the amino acid sequence of SEQ ID NO: 4. In some embodiments, an mRNA polynucleotide comprises an open reading frame that encodes a protein comprising an amino acid sequence that has at least 85% identity to the amino acid sequence of SEQ ID NO: 5. In some embodiments, an mRNA polynucleotide comprises an open reading frame that encodes a protein comprising an amino acid sequence that has at least 85% identity to the amino acid sequence of SEQ ID NO: 6. In some embodiments, an mRNA polynucleotide comprises an open reading frame that encodes a protein comprising an amino acid sequence that has at least 85% identity to the amino acid sequence of SEQ ID NO: 7. In some embodiments, an mRNA polynucleotide comprises an open reading frame that encodes a protein comprising an amino acid sequence that has at least 90% identity to the amino acid sequence of SEQ ID NO: 1. In some embodiments, an mRNA polynucleotide comprises an open reading frame that encodes a protein comprising an amino acid sequence that has at least 90% identity to the amino acid sequence of SEQ ID NO: 2. In some embodiments, an mRNA polynucleotide comprises an open reading frame that encodes a protein comprising an amino acid sequence that has at least 90% identity to the amino acid sequence of SEQ ID NO: 3. In some embodiments, an mRNA polynucleotide comprises an open reading frame that encodes a protein comprising an amino acid sequence that has at least 90% identity to the amino acid sequence of SEQ ID NO: 4. In some embodiments, an mRNA polynucleotide comprises an open reading frame that encodes a protein comprising an amino acid sequence that has at least 90% identity to the amino acid sequence of SEQ ID NO: 5. In some embodiments, an mRNA polynucleotide comprises an open reading frame that encodes a protein comprising an amino acid sequence that has at least 90% identity to the amino acid sequence of SEQ ID NO: 6. In some embodiments, an mRNA polynucleotide comprises an open reading frame that encodes a protein comprising an amino acid sequence that has at least 90% identity to the amino acid sequence of SEQ ID NO: 7. In some embodiments, an mRNA polynucleotide comprises an open reading frame that encodes a protein comprising an amino acid sequence that has at least 95% identity to the amino acid sequence of SEQ ID NO: 1. In some embodiments, an mRNA polynucleotide comprises an open reading frame that encodes a protein comprising an amino acid sequence that has at least 95% identity to the amino acid sequence of SEQ ID NO: 2. In some embodiments, an mRNA polynucleotide comprises an open reading frame that encodes a protein comprising an amino acid sequence that has at least 95% identity to the amino acid sequence of SEQ ID NO: 3. In some embodiments, an mRNA polynucleotide comprises an open reading frame that encodes a protein comprising an amino acid sequence that has at least 95% identity to the amino acid sequence of SEQ ID NO: 4. In some embodiments, an mRNA polynucleotide comprises an open reading frame that encodes a protein comprising an amino acid sequence that has at least 95% identity to the amino acid sequence of SEQ ID NO: 5. In some embodiments, an mRNA polynucleotide comprises an open reading frame that encodes a protein comprising an amino acid sequence that has at least 95% identity to the amino acid sequence of SEQ ID NO: 6. In some embodiments, an mRNA polynucleotide comprises an open reading frame that encodes a protein comprising an amino acid sequence that has at least 95% identity to the amino acid sequence of SEQ ID NO: 7. In some embodiments, an mRNA polynucleotide comprises an open reading frame that encodes a protein comprising an amino acid sequence that has 95% identity to the amino acid sequence of SEQ ID NO: 1. In some embodiments, an mRNA polynucleotide comprises an open reading frame that encodes a protein comprising an amino acid sequence that has 95% identity to the amino acid sequence of SEQ ID NO: 2. In some embodiments, an mRNA polynucleotide comprises an open reading frame that encodes a protein comprising an amino acid sequence that has 95% identity to the amino acid sequence of SEQ ID NO: 3. In some embodiments, an mRNA polynucleotide comprises an open reading frame that encodes a protein comprising an amino acid sequence that has 95% identity to the amino acid sequence of SEQ ID NO: 4. In some embodiments, an mRNA polynucleotide comprises an open reading frame that encodes a protein comprising an amino acid sequence that has 95% identity to the amino acid sequence of SEQ ID NO: 5. In some embodiments, an mRNA polynucleotide comprises an open reading frame that encodes a protein comprising an amino acid sequence that has 95% identity to the amino acid sequence of SEQ ID NO: 6. In some embodiments, an mRNA polynucleotide comprises an open reading frame that encodes a protein comprising an amino acid sequence that has 95% identity to the amino acid sequence of SEQ ID NO: 7. In some embodiments, an mRNA polynucleotide comprises an open reading frame that encodes a protein comprising the amino acid sequence of SEQ ID NO: 1. In some embodiments, an mRNA polynucleotide comprises an open reading frame that encodes a protein comprising the amino acid sequence of SEQ ID NO: 2. In some embodiments, an mRNA polynucleotide comprises an open reading frame that encodes a protein comprising the amino acid sequence of SEQ ID NO: 3. In some embodiments, an mRNA polynucleotide comprises an open reading frame that encodes a protein comprising the amino acid sequence of SEQ ID NO: 4. In some embodiments, an mRNA polynucleotide comprises an open reading frame that encodes a protein comprising the amino acid sequence of SEQ ID NO: 5. In some embodiments, an mRNA polynucleotide comprises an open reading frame that encodes a protein comprising the amino acid sequence of SEQ ID NO: 6. In some embodiments, an mRNA polynucleotide comprises an open reading frame that encodes a protein comprising the amino acid sequence of SEQ ID NO: 7.

“Identity” refers to a relationship between two or among three or more sequences (e.g., amino acid sequences or nucleotide sequences) as determined by comparing the sequences to each other. Identity also refers to the degree of sequence relatedness between or among sequences as determined by the number of matches between or among strings of amino acids (polypeptides) or strings of nucleotides (polynucleotides). Identity is a measure of the percent of identical matches between the smaller of two or more sequences with gap alignments (if any) addressed by a particular mathematical model or computer program (e.g., “algorithms”). Identity of related polypeptides and polynucleotides can be readily calculated by known methods. “Percent (%) identity” as it applies to polypeptide or polynucleotide sequences is defined as the percentage of residues (amino acid or nucleic acid residues) in the candidate (first) polypeptide or polynucleotide sequence that are identical with the residues in a second polypeptide or polynucleotide sequence after aligning the sequences and introducing gaps, if necessary, to achieve the maximum percent identity. As indicated above, unless stated otherwise herein, “percent (%) identity” between two mRNA polynucleotides or between two proteins refers to percent (%) identity determined using a global sequence alignment, comparing the length of entire sequences (e.g., entire mRNA polynucleotide, entire open reading frame of an mRNA, or entire protein encoded by an mRNA, as described herein).

Methods and computer programs for the alignment are well known in the art. It is understood that identity depends on a calculation of percent identity but may differ in value due to gaps and penalties introduced in the calculation. Generally, variants of a particular polynucleotide or polypeptide have at least 90%, 91%, 92%, 93%, 94%, 95%, 96%, 97%, 98%, 99% but less than 100% sequence identity to that particular naturally occurring or reference sequence as determined by sequence alignment programs and parameters described herein and known to those skilled in the art. Such tools for alignment include but are not limited to those of the BLAST suite (Altschul, S. F., et al. Nucleic Acids Res. 1997; 25:3389-3402); and those based on the Smith-Waterman algorithm (Smith, T. F. & Waterman, M. S. J. Mol. Biol. 1981; 147:195-197). A general global alignment technique based on dynamic programming is the Needleman-Wunsch algorithm (Needleman, S. B. & Wunsch, C. D. J. Mol. Biol. 1920; 48:443-453). A Fast Optimal Global Sequence Alignment Algorithm (FOGSAA) also has been developed that purportedly produces global alignment of nucleotide and protein sequences faster than other optimal global alignment methods, including the Needleman-Wunsch algorithm. Other non-limiting examples of global alignment tools include Needle (from EMBOSS), Clustal Omega, MUSCLE (Multiple Sequence Comparison by Log-Expectation), MAFFT (Multiple Alignment using Fast Fourier Transform), and T-Coffee (Tree-based Consistency Objective Function for Alignment Evaluation).

Polynucleotides and polypeptides containing substitutions, insertions and/or deletions (e.g., indels), and covalent modifications with respect to naturally occurring or reference sequence, for example, the polypeptide (e.g., protein) sequences disclosed herein, are included within the scope of this disclosure. For example, sequence tags or amino acids, such as one or more lysine(s), can be added to polypeptide sequences (e.g., at the N-terminal and/or C-terminal end). Sequence tags can be used for peptide detection, purification and/or localization. Lysines can be used to increase peptide solubility or to allow for biotinylation. Alternatively, amino acid residues located at the N-terminal and/or C-terminal regions of the amino acid sequence of a protein may optionally be deleted providing for truncated sequences. Certain amino acids (e.g., C-terminal or N-terminal amino acids) may be deleted depending on the use of the sequence, as for example, expression of the sequence as part of a larger sequence that is soluble or linked to a solid support. In some embodiments, sequences for (or encoding) signal sequences, termination sequences, transmembrane domains, linkers, multimerization domains (e.g., foldon regions) and the like are substituted with alternative sequences that achieve the same or a similar function. In some embodiments, cavities in the core of proteins can be filled to improve stability, e.g., by introducing larger amino acids. In other embodiments, buried hydrogen bond networks are replaced with hydrophobic resides to improve stability. In yet other embodiments, glycosylation sites are removed and replaced with appropriate residues. Such sequences are readily identifiable to one of skill in the art. It should also be understood that some of the sequences provided herein contain sequence tags or terminal peptide sequences (e.g., at the N-terminal or C-terminal ends) that may be deleted, for example, prior to use in the preparation of an mRNA vaccine.

As recognized by those skilled in the art, protein fragments, functional protein domains, and homologous proteins are also considered to be within the scope of Borrelia proteins provided herein. For example, provided herein is any protein fragment of (meaning a polypeptide sequence at least one amino acid residue shorter than but otherwise identical to) a naturally occurring or reference sequence, provided that the fragment is immunogenic and confers a protective immune response to LD. In addition to protein variants that are identical to the naturally occurring or reference protein but are truncated, in some embodiments, a protein includes 2, 3, 4, 5, 6, 7, 8, 9, 10, or more mutations (e.g., substitutions, insertions and/or deletions), as shown in any of the sequences provided or referenced herein. Protein variants can range in length from about 4, 6, or 8 amino acids to full length proteins.

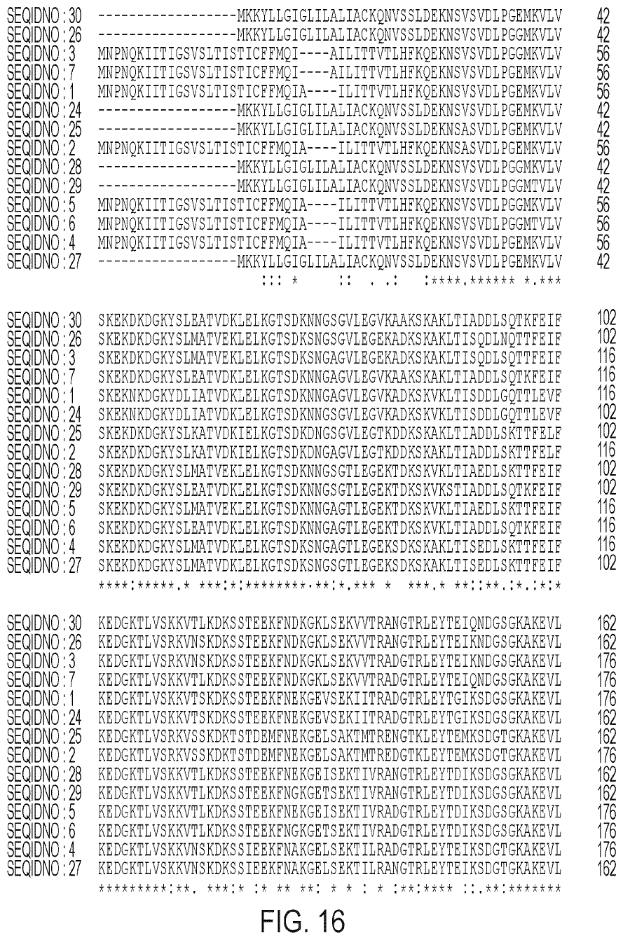

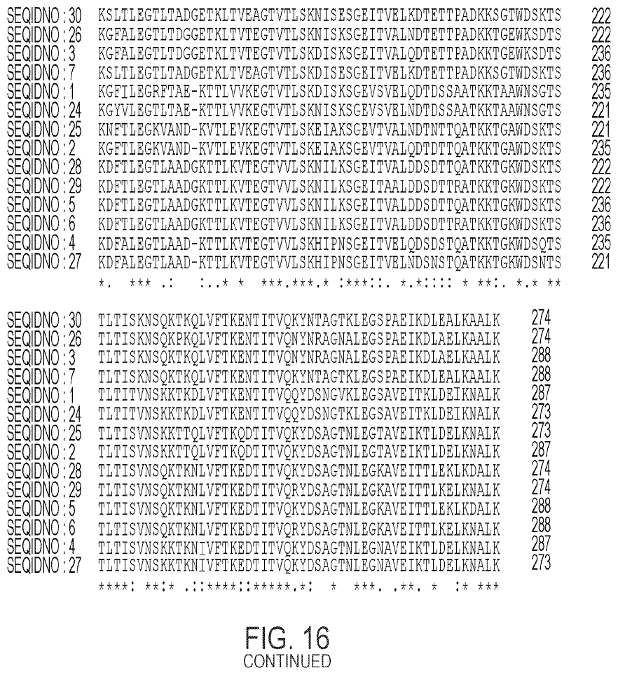

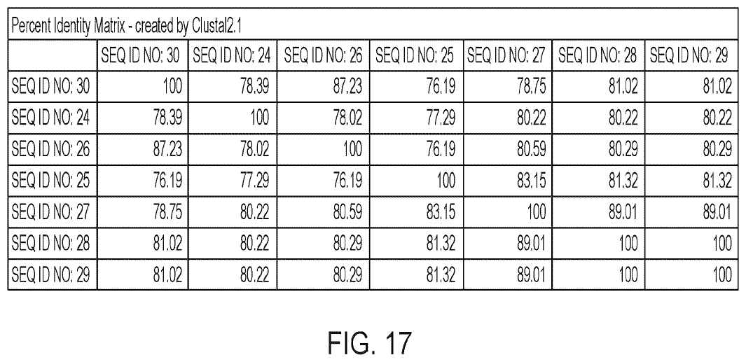

Non-limiting examples of Borrelia protein variants are provided in Table 1. Exemplary sequence comparisons for OspA proteins (full length sequence comparisons) are provided in FIG. 17 (and its corresponding description). A full-length sequence alignment of exemplary OspA proteins is provided in FIG. 16. One having ordinary skill in the art would be able to identify from these comparisons which amino acids are amenable to alteration (e.g., those that are not conserved).

### Signal Peptides

In some embodiments, an mRNA has an open reading frame that encodes a signal peptide fused to the Borrelia protein. Signal peptides, comprising the N-terminal 15-60 amino acids of proteins, are typically needed for the translocation across the membrane on the secretory pathway and, thus, universally control the entry of most proteins both in eukaryotes and prokaryotes to the secretory pathway. In eukaryotes, the signal peptide of a nascent precursor protein (pre-protein) directs the ribosome to the rough endoplasmic reticulum (ER) membrane and initiates the transport of the growing peptide chain across it for processing. ER processing produces mature proteins, wherein the signal peptide is cleaved from precursor proteins, typically by an ER-resident signal peptidase of the host cell, or they remain uncleaved and function as a membrane anchor. A signal peptide may also facilitate the targeting of the protein to the cell membrane.

A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-amino acids.

Signal peptides from heterologous genes (which regulate expression of genes other than Borrelia proteins in nature) are known in the art and can be tested for desired properties and then incorporated into a nucleic acid of the disclosure.

### Fusion Proteins

In some embodiments, an mRNA polynucleotide comprises an ORF that encodes a fusion protein. Thus, an encoded protein may include two or more proteins (e.g., protein and/or protein fragment) joined together with or without a linker. Fusion proteins, in some embodiments, retain the functional property of each independent (nonfusion) protein.

In some embodiments, a fusion protein comprises 2, 3, 4, 5, 6, or 7 of the following Borrelia proteins: OspA serotype 1 (S1); OspA serotype 2 (S2); OspA serotype 3 (S3); OspA serotype 4 (S4); OspA serotype 5 (S5); OspA serotype 6 (S6); and OspA serotype 7 (S7).

### Linkers and Cleavable Peptides

In some embodiments, an mRNA that encodes a fusion protein further encodes a linker located between at least one or each domain of the fusion protein. The linker may be, for example, a cleavable linker or protease-sensitive linker. In some embodiments, the linker is selected from the group consisting of F2A linker, P2A linker, T2A linker, E2A linker, and combinations thereof (see, e.g., WO 2017/127750). This family of self-cleaving peptide linkers, referred to as 2A peptides, has been described in the art (see, e.g., Kim, J. H. et al. PLoS ONE 2011; 6:e18556). In some embodiments, the linker is an F2A linker.

In some embodiments, the linker is a GS linker. GS linkers are polypeptide linkers that include glycine and serine amino acids repeats. They comprise flexible and hydrophilic residues and can be used to perform fusion of protein subunits without interfering in the folding and function of the protein domains, and without formation of secondary structures. In some embodiments, an mRNA polynucleotide comprises an ORF that encodes a fusion protein that comprises a GS linker that is 3 to 20 amino acids long. For example, the GS linker may have a length of (or have a length of at least) 3, 4, 5, 6, 7, 8, 9, 10, 11, 12, 13, 14, 15, 16, 17, 18, 19, or amino acids. In some embodiments, a GS linker is (or is at least) 15 amino acids long (e.g., GGSGGSGGSGGSGGG (SEQ ID NO: 31)). In some embodiments, a GS linker is (or is at least) 8 amino acids long (e.g., GGGSGGGS (SEQ ID NO: 32)). In some embodiments, a GS linker is (or is at least) 7 amino acids long (e.g., GGGSGGG (SEQ ID NO: 33)). In some embodiments, a GS linker is (or is at least) 4 amino acid long (e.g., GGGS (SEQ ID NO: 34)). In some embodiments, the GS linker comprises (GGGS)n (SEQ ID NO: 35), where n is any integer from 1-5. In some embodiments, a GS linker is (or is at least) 4 amino acid long (e.g., GSGG (SEQ ID NO: 36)). In some embodiments, the GS linker comprises (GSGG)n (SEQ ID NO: 37), where n is any integer from 1-5. In some embodiments, a linker is a glycine linker, for example having a length of (or a length of at least) 3 amino acids (e.g., GGG). In some embodiments, a protein encoded by an mRNA includes two or more linkers, which may be the same or different from each other.

The skilled artisan will appreciate that other art-recognized linkers may be suitable for use in the constructs of the disclosure (e.g., encoded by the nucleic acids of the disclosure). The skilled artisan will likewise appreciate that other polycistronic constructs (mRNA encoding more than one protein separately within the same molecule) may be suitable for use as provided herein.

### Stabilization Domains

Protein stabilization domains are protein sequences or structures that can enhance the stability of a protein to various environmental stresses, such as temperature, pH, and proteolysis. Non-limiting examples of protein stabilization domains for use to stabilize a Borrelia protein expressed by an mRNA include: lumazine synthase, ferritin, and thioredoxin. In some embodiments, a Borrelia OspA protein, e.g., an OspA S1 protein is fused to a lumazine synthase. Lumazine synthase is protein from bacteria and plants that can stabilize fusion partners by forming homodimers or oligomers, which can enhance the solubility and stability of the target protein. In some embodiments, a Borrelia OspA protein, e.g., an OspA S1 protein is fused to ferritin. Ferritin is a protein found in animals, plants, and bacteria that can form a cage-like structure that can store and sequester iron ions, protecting the cell from oxidative damage. Fusion of a target protein with ferritin can improve its stability and solubility. In some embodiments, a Borrelia OspA protein, e.g., an OspA S1 protein is fused to thioredoxin. Thioredoxin is small protein found in bacteria and eukaryotes that can act as a reducing agent and stabilize proteins by forming disulfide bonds.

### Nucleic Acids Encoding Lyme Disease Proteins

Nucleic acids comprise a polymer of nucleotides (nucleotide monomers). Thus, nucleic acids are also referred to as polynucleotides. Nucleic acids may be or may include, for example, deoxyribonucleic acid (DNA), ribonucleic acid (RNA), threose nucleic acid (TNA), glycol nucleic acid (GNA), peptide nucleic acid (PNA), locked nucleic acid (LNA, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acid (ENA), cyclohexenyl nucleic acid (CeNA) and/or chimeras and/or combinations thereof.

mRNA polynucleotides of the present disclosure comprises an open reading frame (ORF) encoding a Borrelia protein. In some embodiments, the mRNA polynucleotides further comprises a 5′ untranslated region (UTR), 3′ UTR, a poly(A) tail and/or a 5′ cap analog.

Some aspects of the present disclosure relate to polynucleotide comprising a sequence having at least 80%, 85%, 90%, 95%, or 98% identity to the nucleic acid sequence of any one of SEQ ID NOs: 8-14. In some embodiments, the polynucleotide comprises a sequence having at least 80%, 85%, 90%, 95%, or 98% identity to the nucleic acid sequence of SEQ ID NO: 8. In some embodiments, the polynucleotide comprises a sequence having at least 80%, 85%, 90%, 95%, or 98% identity to the nucleic acid sequence of SEQ ID NO: 9. In some embodiments, the polynucleotide comprises a sequence having at least 80%, 85%, 90%, 95%, or 98% identity to the nucleic acid sequence of SEQ ID NO: 10. In some embodiments, the polynucleotide comprises a sequence having at least 80%, 85%, 90%, 95%, or 98% identity to the nucleic acid sequence of SEQ ID NO: 11. In some embodiments, the polynucleotide comprises a sequence having at least 80%, 85%, 90%, 95%, or 98% identity to the nucleic acid sequence of SEQ ID NO: 12. In some embodiments, the polynucleotide comprises a sequence having at least 80%, 85%, 90%, 95%, or 98% identity to the nucleic acid sequence of SEQ ID NO: 13. In some embodiments, the polynucleotide comprises a sequence having at least 80%, 85%, 90%, 95%, or 98% identity to the nucleic acid sequence of SEQ ID NO: 14.

In some embodiments, the polynucleotide comprises the nucleic acid sequence of any one of SEQ ID NOs: 8-14. In some embodiments, the polynucleotide comprises the nucleic acid sequence of SEQ ID NO: 8. In some embodiments, the polynucleotide comprises the nucleic acid sequence of SEQ ID NO: 9. In some embodiments, the polynucleotide comprises the nucleic acid sequence of SEQ ID NO: 10. In some embodiments, the polynucleotide comprises the nucleic acid sequence of SEQ ID NO: 11. In some embodiments, the polynucleotide comprises the nucleic acid sequence of SEQ ID NO: 12. In some embodiments, the polynucleotide comprises the nucleic acid sequence of SEQ ID NO: 13. In some embodiments, the polynucleotide comprises the nucleic acid sequence of SEQ ID NO: 14.

Some aspects of the present disclosure relates to a compositions comprising a plurality of polynucleotides, wherein the plurality comprises two or more of, three or more of, four or more of, five or more of, six or more of, or all seven of: a first polynucleotide comprising a sequence having at least 80%, 85%, 90%, 95%, or 98% identity to the nucleic acid sequence of SEQ ID NO: 8; a second polynucleotide comprising a sequence having at least 80%, 85%, 90%, 95%, or 98% identity to the nucleic acid sequence of SEQ ID NO: 9; a third polynucleotide comprising a sequence having at least 80%, 85%, 90%, 95%, or 98% identity to the nucleic acid sequence of SEQ ID NO: 10; a fourth polynucleotide comprising a sequence having at least 80%, 85%, 90%, 95%, or 98% identity to the nucleic acid sequence of SEQ ID NO: 11; a fifth polynucleotide comprising a sequence having at least 80%, 85%, 90%, 95%, or 98% identity to the nucleic acid sequence of SEQ ID NO: 12; a sixth polynucleotide comprising a sequence having at least 80%, 85%, 90%, 95%, or 98% identity to the nucleic acid sequence of SEQ ID NO: 13; and a seventh polynucleotide comprising a sequence having at least 80%, 85%, 90%, 95%, or 98% identity to the nucleic acid sequence of SEQ ID NO: 14.

In some embodiments, the plurality comprises two or more of, three or more of, four or more of, five or more of, six or more of, or all seven of: a first polynucleotide comprising a sequence having at least 80% identity to the nucleic acid sequence of SEQ ID NO: 8; a second polynucleotide comprising a sequence having at least 80% identity to the nucleic acid sequence of SEQ ID NO: 9; a third polynucleotide comprising a sequence having at least 80% identity to the nucleic acid sequence of SEQ ID NO: 10; a fourth polynucleotide comprising a sequence having at least 80% identity to the nucleic acid sequence of SEQ ID NO: 11; a fifth polynucleotide comprising a sequence having at least 80% identity to the nucleic acid sequence of SEQ ID NO: 12; a sixth polynucleotide comprising a sequence having at least 80% identity to the nucleic acid sequence of SEQ ID NO: 13; and a seventh polynucleotide comprising a sequence having at least 80% identity to the nucleic acid sequence of SEQ ID NO: 14.

In some embodiments, the plurality comprises two or more of, three or more of, four or more of, five or more of, six or more of, or all seven of: a first polynucleotide comprising a sequence having at least 85% identity to the nucleic acid sequence of SEQ ID NO: 8; a second polynucleotide comprising a sequence having at least 85% identity to the nucleic acid sequence of SEQ ID NO: 9; a third polynucleotide comprising a sequence having at least 85% identity to the nucleic acid sequence of SEQ ID NO: 10; a fourth polynucleotide comprising a sequence having at least 85% identity to the nucleic acid sequence of SEQ ID NO: 11; a fifth polynucleotide comprising a sequence having at least 85% identity to the nucleic acid sequence of SEQ ID NO: 12; a sixth polynucleotide comprising a sequence having at least 85% identity to the nucleic acid sequence of SEQ ID NO: 13; and a seventh polynucleotide comprising a sequence having at least 85% identity to the nucleic acid sequence of SEQ ID NO: 14.

In some embodiments, the plurality comprises two or more of, three or more of, four or more of, five or more of, six or more of, or all seven of: a first polynucleotide comprising a sequence having at least 90% identity to the nucleic acid sequence of SEQ ID NO: 8; a second polynucleotide comprising a sequence having at least 90% identity to the nucleic acid sequence of SEQ ID NO: 9; a third polynucleotide comprising a sequence having at least 90% identity to the nucleic acid sequence of SEQ ID NO: 10; a fourth polynucleotide comprising a sequence having at least 90% identity to the nucleic acid sequence of SEQ ID NO: 11; a fifth polynucleotide comprising a sequence having at least 90% identity to the nucleic acid sequence of SEQ ID NO: 12; a sixth polynucleotide comprising a sequence having at least 90% identity to the nucleic acid sequence of SEQ ID NO: 13; and a seventh polynucleotide comprising a sequence having at least 90% identity to the nucleic acid sequence of SEQ ID NO: 14.

In some embodiments, the plurality comprises two or more of, three or more of, four or more of, five or more of, six or more of, or all seven of: a first polynucleotide comprising a sequence having at least 95% identity to the nucleic acid sequence of SEQ ID NO: 8; a second polynucleotide comprising a sequence having at least 95% identity to the nucleic acid sequence of SEQ ID NO: 9; a third polynucleotide comprising a sequence having at least 95% identity to the nucleic acid sequence of SEQ ID NO: 10; a fourth polynucleotide comprising a sequence having at least 95% identity to the nucleic acid sequence of SEQ ID NO: 11; a fifth polynucleotide comprising a sequence having at least 95% identity to the nucleic acid sequence of SEQ ID NO: 12; a sixth polynucleotide comprising a sequence having at least 95% identity to the nucleic acid sequence of SEQ ID NO: 13; and a seventh polynucleotide comprising a sequence having at least 95% identity to the nucleic acid sequence of SEQ ID NO: 14.

In some embodiments, the plurality comprises two or more of, three or more of, four or more of, five or more of, six or more of, or all seven of: a first polynucleotide comprising a sequence having at least 98% identity to the nucleic acid sequence of SEQ ID NO: 8; a second polynucleotide comprising a sequence having at least 98% identity to the nucleic acid sequence of SEQ ID NO: 9; a third polynucleotide comprising a sequence having at least 98% identity to the nucleic acid sequence of SEQ ID NO: 10; a fourth polynucleotide comprising a sequence having at least 98% identity to the nucleic acid sequence of SEQ ID NO: 11; a fifth polynucleotide comprising a sequence having at least 98% identity to the nucleic acid sequence of SEQ ID NO: 12; a sixth polynucleotide comprising a sequence having at least 98% identity to the nucleic acid sequence of SEQ ID NO: 13; and a seventh polynucleotide comprising a sequence having at least 98% identity to the nucleic acid sequence of SEQ ID NO: 14.

In some embodiments, the first polynucleotide comprises the nucleic acid sequence of SEQ ID NO: 8; the second polynucleotide comprises the nucleic acid sequence of SEQ ID NO: 9; the third polynucleotide comprises the nucleic acid sequence of SEQ ID NO: 10; the fourth polynucleotide comprises the nucleic acid sequence of SEQ ID NO: 11; the fifth polynucleotide comprises the nucleic acid sequence of SEQ ID NO: 12; the sixth polynucleotide comprises the nucleic acid sequence of SEQ ID NO: 13; and the seventh polynucleotide comprises the nucleic acid sequence of SEQ ID NO: 14.

In some embodiments, the second polynucleotide comprises the nucleic acid sequence of SEQ ID NO: 9; the third polynucleotide comprises the nucleic acid sequence of SEQ ID NO: 10; the fourth polynucleotide comprises the nucleic acid sequence of SEQ ID NO: 11; the fifth polynucleotide comprises the nucleic acid sequence of SEQ ID NO: 12; the sixth polynucleotide comprises the nucleic acid sequence of SEQ ID NO: 13; and the seventh polynucleotide comprises the nucleic acid sequence of SEQ ID NO: 14.

### Messenger RNA

Messenger RNA (mRNA) is RNA that encodes a (at least one) protein (a naturally-occurring, non-naturally-occurring, or modified polymer of amino acids) and can be translated to produce the encoded protein in vitro, in vivo, in situ, or ex vivo. It is understood that mRNA is not self-amplifying RNA (saRNA) (see, e.g., Bloom K et al. Gene Therapy 2021; 28: 117-129 for a comparison of mRNA and saRNA). saRNAs include alphavirus replicase sequences that encode an RNA-dependent RNA polymerase. mRNA does not include alphavirus replicase sequences.

The skilled artisan will appreciate that, except where otherwise noted, nucleic acid sequences set forth in the instant application may recite “T” s in a representative DNA sequence but where the sequence represents mRNA, the “T” s would be substituted for “U” s. Thus, any of the DNAs disclosed and identified by a particular sequence identification number herein also disclose the corresponding mRNA sequence complementary to the DNA, where each “T” of the DNA sequence is substituted with “U.”

Naturally-occurring eukaryotic mRNA molecules can contain stabilizing elements, including, but not limited to, UTRs at their 5′-end (5′ UTR) and/or at their 3′-end (3′ UTR), in addition to other structural features, such as a 5′-cap structure or a 3′-poly(A) tail. Both the 5′ UTR and the 3′ UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing.

Exemplary sequences of mRNA that encode Borrelia proteins of the present disclosure are provided in Table 1. In some embodiments, the mRNA polynucleotide comprises an ORF that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or identical to a sequence selected from SEQ ID NOs: 8-14 and 15-21. In some embodiments, the mRNA polynucleotide comprises a nucleotide sequence that is 80%, 81%, 82%, 83%, 84%, 85%, 86%, 87%, 88%, 89%, 90%, 91%, 92%, 93%, 94%, 95%, 96%, 97%, 98%, 99% or identical to a sequence selected from SEQ ID NOs: 8-14, 15-21, 40, 55, 57, 59, 61, 63, and 65.

In some embodiments, the mRNA polynucleotide comprises a sequence having at least 80%, 85%, 90%, 95%, or 98% identity to the nucleic acid sequence of SEQ ID NO: 15. In some embodiments, the mRNA polynucleotide comprises a sequence having at least 80%, 85%, 90%, 95%, or 98% identity to the nucleic acid sequence of SEQ ID NO: 40. In some embodiments, the mRNA polynucleotide comprises a sequence having at least 80%, 85%, 90%, 95%, or 98% identity to the nucleic acid sequence of SEQ ID NO: 16. In some embodiments, the mRNA polynucleotide comprises a sequence having at least 80%, 85%, 90%, 95%, or 98% identity to the nucleic acid sequence of SEQ ID NO: 55. In some embodiments, the mRNA polynucleotide comprises a sequence having at least 80%, 85%, 90%, 95%, or 98% identity to the nucleic acid sequence of SEQ ID NO: 17. In some embodiments, the mRNA polynucleotide comprises a sequence having at least 80%, 85%, 90%, 95%, or 98% identity to the nucleic acid sequence of SEQ ID NO: 57. In some embodiments, the mRNA polynucleotide comprises a sequence having at least 80%, 85%, 90%, 95%, or 98% identity to the nucleic acid sequence of SEQ ID NO: 18. In some embodiments, the mRNA polynucleotide comprises a sequence having at least 80%, 85%, 90%, 95%, or 98% identity to the nucleic acid sequence of SEQ ID NO: 59. In some embodiments, the mRNA polynucleotide comprises a sequence having at least 80%, 85%, 90%, 95%, or 98% identity to the nucleic acid sequence of SEQ ID NO: 19. In some embodiments, the mRNA polynucleotide comprises a sequence having at least 80%, 85%, 90%, 95%, or 98% identity to the nucleic acid sequence of SEQ ID NO: 61. In some embodiments, the mRNA polynucleotide comprises a sequence having at least 80%, 85%, 90%, 95%, or 98% identity to the nucleic acid sequence of SEQ ID NO: 20. In some embodiments, the mRNA polynucleotide comprises a sequence having at least 80%, 85%, 90%, 95%, or 98% identity to the nucleic acid sequence of SEQ ID NO: 63. In some embodiments, the mRNA polynucleotide comprises a sequence having at least 80%, 85%, 90%, 95%, or 98% identity to the nucleic acid sequence of SEQ ID NO: 21. In some embodiments, the mRNA polynucleotide comprises a sequence having at least 80%, 85%, 90%, 95%, or 98% identity to the nucleic acid sequence of SEQ ID NO: 65.

In some embodiments, the mRNA polynucleotide comprises the nucleic acid sequence of Table 1. In some embodiments, the mRNA polynucleotide comprises the nucleic acid sequence of SEQ ID NO: 15. In some embodiments, the mRNA polynucleotide comprises the nucleic acid sequence of SEQ ID NO: 40. In some embodiments, the mRNA polynucleotide comprises the nucleic acid sequence of SEQ ID NO: 16. In some embodiments, the mRNA polynucleotide comprises the nucleic acid sequence of SEQ ID NO: 55. In some embodiments, the mRNA polynucleotide comprises the nucleic acid sequence of SEQ ID NO: 17. In some embodiments, the mRNA polynucleotide comprises the nucleic acid sequence of SEQ ID NO: 57. In some embodiments, the mRNA polynucleotide comprises the nucleic acid sequence of SEQ ID NO: 18. In some embodiments, the mRNA polynucleotide comprises the nucleic acid sequence of SEQ ID NO: 59. In some embodiments, the mRNA polynucleotide comprises the nucleic acid sequence of SEQ ID NO: 19. In some embodiments, the mRNA polynucleotide comprises the nucleic acid sequence of SEQ ID NO: 61. In some embodiments, the mRNA polynucleotide comprises the nucleic acid sequence of SEQ ID NO: 20. In some embodiments, the mRNA polynucleotide comprises the nucleic acid sequence of SEQ ID NO: 63. In some embodiments, the mRNA polynucleotide comprises the nucleic acid sequence of SEQ ID NO: 21. In some embodiments, the mRNA polynucleotide comprises the nucleic acid sequence of SEQ ID NO: 65.

### Untranslated Regions (UTRs)

The mRNAs of the present disclosure may comprise one or more regions or parts which act or function as an untranslated region. A “5′ untranslated region” (UTR) refers to a region of an mRNA that is directly upstream (i.e., 5′) from the start codon (i.e., the first codon of an mRNA transcript translated by a ribosome) that does not encode a polypeptide. A “3′ untranslated region” (UTR) refers to a region of an mRNA that is directly downstream (i.e., 3′) from the stop codon (i.e., the codon of an mRNA transcript that signals a termination of translation) that does not encode a polypeptide. When RNA transcripts are being generated, the 5′ UTR may comprise a promoter sequence. Such promoter sequences are known in the art. It should be understood that such promoter sequences will not be present in a vaccine of the disclosure.

Where mRNAs are designed to encode a (at least one) bacterial protein, the mRNA may comprise a 5′ UTR and/or 3′ UTR. UTRs of an mRNA are transcribed but not translated. In mRNA, the 5′ UTR starts at the transcription start site and continues to the start codon but does not include the start codon; the 3′ UTR starts immediately following the stop codon and continues until the transcriptional termination signal. There is a growing body of evidence about the regulatory roles played by the UTRs in terms of stability of the nucleic acid molecule and translation. The regulatory features of a UTR can be incorporated into the polynucleotides of the present disclosure to, among other things, enhance the stability of the molecule. The specific features can also be incorporated to ensure controlled down-regulation of the transcript in case they are misdirected to undesired organs sites. A variety of 5′ UTR and 3′ UTR sequences are known.

It should also be understood that the mRNA of the present disclosure may include any 5′ UTR and/or any 3′ UTR. Exemplary UTR sequences include SEQ ID NOs: 22, 23, 38, 39, 41-50, 53, 56, 58, 60, 62, 64, and 66; however, other UTR sequences may be used or exchanged for any of the UTR sequences described herein.

In some embodiments, a 5′ UTR of the present disclosure comprises a sequence selected from:

(SEQ ID NO: 38)
GGGAAAUAAGAGAGAAAAGAAGAGUAAGAAGAAAUAUAAGAGCCACC,
(SEQ ID NO: 39)
GGGAAAUAAGAGAGAAAAGAAGAGUAAGAAGAAAUAUAAGACCCCGGCGC
CGCCACC,
(SEQ ID NO: 22)
GAGGAAAUCGCAAAAUUUGCUCUUCGCGUUAGAUUUCUUUUAGUUUUCUC
GCAACUAGCAAGCUUUUUGUUCUCGCC,
(SEQ ID NO: 41)
GGAAAUCGCAAAAUUUGCUCUUCGCGUUAGAUUUCUUUUAGUUUUCUCGC
AACUAGCAAGCUUUUUGUUCUCGCC,
(SEQ ID NO: 50)
AGGAAAUCGCAAAAUUUGCUCUUCGCGUUAGAUUUCUUUUAGUUUUCUCG
CAACUAGCAAGCUUUUUGUUCUCGCC,

and a sequence having at least 80%, 85%, 90%, 95%, or 98% identity to the nucleic acid sequence of any one of SEQ ID NOs: 38, 39, 22, 41, and 50.

In some embodiments, a 3′ UTR of the present disclosure comprises a sequence selected from

(SEQ ID NO: 42)
UGAUAAUAGGCUGGAGCCUCGGUGGCCAUGCUUCUUGCCCCUUGGGCCUCCCCCC
AGCCCCUCCUCCCCUUCCUGCACCCGUACCCCCGUGGUCUUUGAAUAAAGUCUGA
GUGGGCGGC,
(SEQ ID NO: 43)
UGAUAAUAGGCUGGAGCCUCGGUGGCCUAGCUUCUUGCCCCUUGGGCCUCCCCCC
AGCCCCUCCUCCCCUUCCUGCACCCGUACCCCCGUGGUCUUUGAAUAAAGUCUGA
GUGGGCGGC,
(SEQ ID NO: 44)
UAAAGCUCCCCGGGGGCCUCGGUGGCCUAGCUUCUUGCCCCUUGGGCCUCCCCCC
AGCCCCUCCUCCCCUUCCUGCAGGAGAUUGAGUGUAGUGACUAGUGGUCUUUGA
AUAAAGUCUGAGUGGGCGGC,
(SEQ ID NO: 45)
UAAAGCUCCCCGGGGGCCUCGGUGGCCUAGCUUCUUGCCCCUUGGGCCUCCCCCC
AGCCCCUCCUCCCCUUCCUGCAGGAUUGAGACUACGGGUGGUCUUUGAAUAAAG
UCUGAGUGGGCGGC,
(SEQ ID NO: 46)
UAAAGCUCCCCGGGGGCCUCGGUGGCCUAGCUUCUUGCCCCUUGGGCCUCCCCCC
AGCCCCUCCUCCCCUUCCUGCAGCAUAGACACUACGUGGUCUUUGAAUAAAGUCU
GAGUGGGGGC,
(SEQ ID NO: 47)
UAAAGCUCCCCGGGGGCCUCGGUGGCCUAGCUUCUUGCCCCUUGGGCCUCCCCCC
AGCCCCUCCUCCCCUUCCUGCAGGAGAUUGAGUGUAGUGGUGGUCUUUGAAUAA
AGUCUGAGUGGGCGGC,
(SEQ ID NO: 48)
UAAAGCUCCCCGGGGGCCUCGGUGGCCUAGCUUCUUGCCCCUUGGGCCUCCCCCC
AGCCCCUCCUCCCCUUCCUGCAGGAGAUUGAGUGUAGUGACGUGGUCUUUGAAU
AAAGUCUGAGUGGGCGGC,
(SEQ ID NO: 49)
UAAAGCUCCCCGGGGGCCUCGGUGGCCUAGCUUCUUGCCCCUUGGGCCUCCCCCC
AGCCCCUCCUCCCCUUCCUGCAGUGGUCUUUGAAUAAAGUCUGAGUGGGCGGC,
(SEQ ID NO: 23)
UAAAGCUCCCCGGGGGCCUCGGUGGCCUAGCUUCUUGCCCCUUGGGCCUCCCCCC
AGCCCCUCCUCCCCUUCCUGCACCCGUACCCCCGUGGUCUUUGAAUAAAGUCUGA
GUGGGCGGC,
(SEQ ID NO: 53)
UAAAGCUCCCCGGGGGCCUCGGUGGCCUAGCUUCUUGCCCCUUGGGCCUCCCCCC
AGCCCCUCCUCCCCUUCCUGCAGGUUAAGGAAGUGGUCUUUGAAUAAAGUCUGA
GUGGGCGGC,
(SEQ ID NO: 56)
UAAAGCUCCCCGGGGGCCUCGGUGGCCUAGCUUCUUGCCCCUUGGGCCUCCCCCC
AGCCCCUCCUCCCCUUCCUGCAGGUGACCUUAGAGUGGUCUUUGAAUAAAGUCUG
AGUGGGCGGC,
(SEQ ID NO: 58)
UAAAGCUCCCCGGGGGCCUCGGUGGCCUAGCUUCUUGCCCCUUGGGCCUCCCCCC
AGCCCCUCCUCCCCUUCCUGCAGUCCACAUAUCGAAGUGGUCUUUGAAUAAAGUC
UGAGUGGGCGGC,
(SEQ ID NO: 60)
UAAAGCUCCCCGGGGGCCUCGGUGGCCUAGCUUCUUGCCCCUUGGGCCUCCCCCC
AGCCCCUCCUCCCCUUCCUGCAGAUAACUGAAGGAAGAGUGGUCUUUGAAUAAA
GUCUGAGUGGGCGGC,
(SEQ ID NO: 62)
UAAAGCUCCCCGGGGGCCUCGGUGGCCUAGCUUCUUGCCCCUUGGGCCUCCCCCC
AGCCCCUCCUCCCCUUCCUGCAGGAGAGUGAGUGUAGUGAGGUGGUCUUUGAAU
AAAGUCUGAGUGGGCGGC,
(SEQ ID NO: 64)
UAAAGCUCCCCGGGGGCCUCGGUGGCCUAGCUUCUUGCCCCUUGGGCCUCCCCCC
AGCCCCUCCUCCCCUUCCUGCAGGAGAGUGAGUGUAGUGAGUAAGUGGUCUUUG
AAUAAAGUCUGAGUGGGCGGC,
(SEQ ID NO: 66)
UAAAGCUCCCCGGGGGCCUCGGUGGCCUAGCUUCUUGCCCCUUGGGCCUCCCCCC
AGCCCCUCCUCCCCUUCCUGCAGUUGAGGCACGCGUCGGCAGGCGCAGUGGUCUU
UGAAUAAAGUCUGAGUGGGCGGC,

and a sequence having at least 80%, 85%, 90%, 95%, or 98% identity to the nucleic acid sequence of any one of SEQ ID NOs: 42-49, 23, 53, 56, 58, 60, 62, 64, and 66.

In some embodiments, a 3′ UTR comprises, in 5′-to-3′ order: (a) the nucleic acid sequence

UAAAGCUCCCCGGGGGCCUCGGUGGCCUAGCUUCUUGCCCCUUGGGCCUCCCCCC AGCCCCUCCUCCCCUUCCUGCAG (SEQ ID NO: 51), (b) an identification and ratio determination (IDR) sequence, and (c) the nucleic acid sequence UGGUCUUUGAAUAAAGUCUGAGUGGGCGGC (SEQ ID NO: 52). IDR sequences are described herein in the section entitled “Identification and Ratio Determination (IDR) Sequences.” UTRs may also be omitted from the mRNA provided herein.

A 5′ UTR does not encode a protein (is non-coding). Natural 5′ UTRs have features that play roles in translation initiation. They harbor signatures like Kozak sequences which are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′UTR also have been known to form secondary structures which are involved in elongation factor binding.

In some embodiments of the disclosure, a 5′ UTR is a heterologous UTR, i.e., is a UTR found in nature associated with a different ORF. In other embodiments, a 5′ UTR is a synthetic UTR, i.e., does not occur in nature. Synthetic UTRs include UTRs that have been mutated to improve their properties, e.g., which increase gene expression as well as those which are completely synthetic. Exemplary 5′ UTRs include Xenopus or human derived a-globin or b-globin (U.S. Pat. Nos. 8,278,063; 9,012,219), human cytochrome b-245 a polypeptide, and hydroxysteroid (17b) dehydrogenase, and Tobacco etch virus (U.S. Pat. Nos. 8,278,063, 9,012,219). CMV immediate-early 1 (IE1) gene (US2014/0206753, WO2013/185069), the sequence GGGAUCCUACC (SEQ ID NO: 54) (WO2014/144196) may also be used. In other embodiments, a 5′ UTR is a 5′ UTR of a TOP gene lacking the 5′ TOP motif (the oligopyrimidine tract) (e.g., WO2015/101414, WO2015/101415, WO2015/062738, WO2015/024667, WO2015/024667); 5′ UTR element derived from ribosomal protein Large 32 (L32) gene (WO/2015101414, WO2015101415, WO/2015/062738), 5′ UTR element derived from the 5′ UTR of an hydroxysteroid (17-β) dehydrogenase 4 gene (HSD17B4) (WO2015024667), or a 5′ UTR element derived from the 5′ UTR of ATP5A1 (WO2015/024667) can be used. In some embodiments, an internal ribosome entry site (IRES) is used instead of a 5′ UTR.

A 3′ UTR does not encode a protein (is non-coding). Natural or wild type 3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-α. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of mRNA of the disclosure. When engineering specific nucleic acids, one or more copies of an ARE can be introduced to make nucleic acids of the disclosure less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using nucleic acids of the disclosure and protein production can be assayed at various time points post-transfection. For example, cells can be transfected with different ARE-engineering molecules and by using an ELISA kit to the relevant protein and assaying protein produced at 6 hours, 12 hours, 1 day, 2 days, and 7 days post-transfection.

Those of ordinary skill in the art will understand that 5′ UTRs that are heterologous or synthetic may be used with any desired 3′ UTR sequence. For example, a heterologous or synthetic 5′ UTR may be used with a synthetic 3′ UTR or with a heterologous 3′ UTR.

Non-UTR sequences may also be used as regions or subregions within a nucleic acid. For example, introns or portions of introns sequences may be incorporated into regions of nucleic acid of the disclosure. Incorporation of intronic sequences may increase protein production as well as nucleic acid levels.

Combinations of features may be included in flanking regions and may be contained within other features. For example, the ORF may be flanked by a 5′ UTR which may contain a strong Kozak translational initiation signal and/or a 3′ UTR which may include an oligo(dT) sequence for templated addition of a poly-A tail. 5′ UTR may comprise a first polynucleotide fragment and a second polynucleotide fragment from the same and/or different genes such as the 5′ UTRs described in US2010/0293625 and WO2015/085318A2, each of which is herein incorporated by reference.

It should be understood that any UTR from any gene may be incorporated into the regions of a nucleic acid. Furthermore, multiple wild-type UTRs of any known gene may be utilized. It is also within the scope of the present disclosure to provide artificial UTRs which are not variants of wild type regions. These UTRs or portions thereof may be placed in the same orientation as in the transcript from which they were selected or may be altered in orientation or location. Hence a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ UTR or 5′ UTR may be altered relative to a wild-type/native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ UTR or 3′ UTR may be used. As used herein, a “double” UTR is one in which two copies of the same UTR are encoded either in series or substantially in series. For example, a double beta-globin 3′ UTR may be used as described in US2010/0129877, which is incorporated herein by reference.

It is also within the scope of the present disclosure to have patterned UTRs. As used herein “patterned UTRs” are those UTRs which reflect a repeating or alternating pattern, such as ABABAB or AABBAABBAABB or ABCABCABC or variants thereof repeated once, twice, or more than 3 times. In these patterns, each letter, A, B, or C represent a different UTR at the nucleotide level.

In some embodiments, flanking regions are selected from a family of transcripts whose proteins share a common function, structure, feature, or property. For example, polypeptides of interest may belong to a family of proteins which are expressed in a particular cell, tissue or at some time during development. The UTRs from any of these genes may be swapped for any other UTR of the same or different family of proteins to create a new polynucleotide. As used herein, a “family of proteins” is used in the broadest sense to refer to a group of two or more polypeptides of interest which share at least one function, structure, feature, localization, origin, or expression pattern.

The untranslated region may also include translation enhancer elements (TEE). As a non-limiting example, the TEE may include those described in US 2009/0226470, herein incorporated by reference, and those known in the art.

### Open Reading Frames

An open reading frame (ORF) is a continuous stretch of DNA or RNA beginning with a start codon (e.g., methionine (ATG or AUG)) and ending with a stop codon (e.g., TAA, TAG or TGA, or UAA, UAG or UGA). An ORF typically encodes a protein. It will be understood that the sequences disclosed herein may further comprise additional elements, e.g., 5′ and/or 3′ UTRs, but that those elements, unlike the ORF, need not necessarily be present in an mRNA of the present disclosure.

### 5′ End Capping

In some embodiments, an mRNA polynucleotide comprises a 5′ terminal cap. 5′-capping of polynucleotides may be completed concomitantly during an in vitro transcription reaction using, for example, the following chemical RNA cap analogs to generate the 5′-guanosine cap structure according to manufacturer protocols: 3′-O-Me-m7G(5′)ppp(5′) G [the ARCA cap];G(5′)ppp(5′)A; G(5′)ppp(5′)G; m7G(5′)ppp(5′)A; m7G(5′)ppp(5′)G (New England BioLabs, Ipswich, MA). 5′-capping of modified mRNA may be completed post-transcriptionally using, for example, a Vaccinia Virus Capping Enzyme to generate the “Cap 0” structure: m7G(5′)ppp(5′)G (New England BioLabs, Ipswich, MA). Cap 1 structure may be generated using both Vaccinia Virus Capping Enzyme and a 2′-0 methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-0 methyl-transferase. Enzymes may be derived from a recombinant source. Other cap analogs may be used.

### Polyadenylation Tailing

A “poly(A) tail” is a region of mRNA that is downstream, e.g., directly downstream (i.e., 3′), from the 3′ UTR that contains multiple, consecutive adenosine monophosphates. A poly(A) tail may contain 10 to 300 adenosine monophosphates. It can, in some instances, comprise up to about 400 adenine nucleotides. For example, a poly(A) tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a poly(A) tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, and/or export of the mRNA from the nucleus and translation. In some embodiments, the length of the 3′-poly(A) tail may be an essential element with respect to the stability of the individual mRNA. In some embodiments, a poly(A) tail has a length of about 50, about 100, about 150, about 200, about 250, about 300, about 350, or about 400 nucleotides. In some embodiments, a poly(A) tail has a length of 100 nucleotides.

### Additional Stabilizing Elements

mRNA provided herein, in some embodiments, includes an additional stabilizing element. Stabilizing elements may include, for example, a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, an mRNA includes an open reading frame (coding region), a histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g., Luciferase, GFP, EGFP, β-Galactosidase, EGFP), or a marker or selection protein (e.g., alpha-Globin, Galactokinase and Xanthine:guanine phosphoribosyl transferase (GPT)).

In some embodiments, an mRNA includes the combination of a poly(A) sequence or polyadenylation signal and at least one histone stem-loop, even though both represent alternative mechanisms in nature, they act synergistically to increase the protein expression beyond the level observed with either of the individual elements. The synergistic effect of the combination of poly(A) and a histone stem-loop does not depend on the order of the elements or the length of the poly(A) sequence.

In some embodiments, an mRNA does not include a histone downstream element (HDE). “Histone downstream element” (HDE) includes a purine-rich polynucleotide stretch of approximately 15 to 20 nucleotides 3′ of naturally-occurring stem-loops, representing the binding site for the U7 snRNA, which is involved in processing of histone pre-mRNA into mature histone mRNA. In some embodiments, the nucleic acid does not include an intron.

An mRNA may or may not contain an enhancer and/or promoter sequence, which may be modified or unmodified or which may be activated or inactivated. In some embodiments, the histone stem-loop is generally derived from histone genes and includes an intramolecular base pairing of two neighbored partially or entirely reverse complementary sequences separated by a spacer, consisting of a short sequence, which forms the loop of the structure. The unpaired loop region is typically unable to base pair with either of the stem loop elements. It occurs more often in RNA, as is a key component of many RNA secondary structures but may be present in single-stranded DNA as well. Stability of the stem-loop structure generally depends on the length, number of mismatches or bulges, and base composition of the paired region. In some embodiments, wobble base pairing (non-Watson-Crick base pairing) may result. In some embodiments, the at least one histone stem-loop sequence comprises a length of 15 to 45 nucleotides.

In some embodiments, an mRNA has one or more AU-rich sequences removed. These sequences, sometimes referred to as AURES are destabilizing sequences found in the 3′UTR. The AURES may be removed from the mRNA. Alternatively, the AURES may remain in the mRNA.

### Sequence Optimization

In some embodiments, an open reading frame encoding a protein of the disclosure is codon optimized. Codon optimization methods are known in the art. An open reading frame of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park CA) and/or proprietary methods. In some embodiments, the open reading frame sequence is optimized using optimization algorithms.

In some embodiments, a codon optimized sequence shares less than 95% sequence identity to a naturally-occurring or wild-type sequence open reading frame (e.g., a naturally-occurring or wild-type mRNA sequence encoding a Borrelia protein antigen). In some embodiments, a codon optimized sequence shares less than 90% sequence identity to a naturally-occurring or wild-type sequence (e.g., a naturally-occurring or wild-type mRNA sequence encoding a Borrelia protein). In some embodiments, a codon optimized sequence shares less than 85% sequence identity to a naturally-occurring or wild-type sequence (e.g., a naturally-occurring or wild-type mRNA sequence encoding a Borrelia protein). In some embodiments, a codon optimized sequence shares less than 80% sequence identity to a naturally-occurring or wild-type sequence (e.g., a naturally-occurring or wild-type mRNA sequence encoding a Borrelia protein). In some embodiments, a codon optimized sequence shares less than 75% sequence identity to a naturally-occurring or wild-type sequence (e.g., a naturally-occurring or wild-type mRNA sequence encoding a Borrelia protein).

In some embodiments, a codon optimized sequence shares between 65% and 85% (e.g., between about 67% and about 85% or between about 67% and about 80%) sequence identity to a naturally-occurring or wild-type sequence (e.g., a naturally-occurring or wild-type mRNA sequence encoding a Borrelia protein). In some embodiments, a codon optimized sequence shares between 65% and 75% or about 80% sequence identity to a naturally-occurring or wild-type sequence (e.g., a naturally-occurring or wild-type mRNA sequence encoding a Borrelia protein).

In some embodiments, a codon-optimized sequence encodes an antigen that is as immunogenic as, or more immunogenic than (e.g., at least 10%, at least 20%, at least 30%, at least 40%, at least 50%, at least 100%, or at least 200% more), than a Borrelia protein encoded by a non-codon-optimized sequence.

When transfected into mammalian host cells, the modified mRNAs have a stability of between 12-18 hours, or greater than 18 hours, e.g., 24, 36, 48, 60, 72, or greater than 72 hours and are capable of being expressed by the mammalian host cells.

In some embodiments, a codon optimized mRNA may be one in which the levels of G/C are enhanced. The G/C-content of nucleic acid molecules (e.g., mRNA) may influence the stability of the RNA. mRNA having an increased amount of guanine (G) and/or cytosine (C) residues may be functionally more stable than RNA containing a large amount of adenine (A) and thymine (T) or uracil (U) nucleotides. As an example, WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the mRNA.

### Chemically Unmodified Nucleotides

In some embodiments, an mRNA is not chemically modified and comprises the standard ribonucleotides consisting of adenosine, guanosine, cytosine and uridine. In some embodiments, nucleotides and nucleosides of the present disclosure comprise standard nucleoside residues such as those present in transcribed RNA (e.g., A, G, C, or U). In some embodiments, nucleotides and nucleosides of the present disclosure comprise standard deoxyribonucleosides such as those present in DNA (e.g., dA, dG, dC, or dT).

### Chemically Modified Nucleotides

The compositions of the present disclosure comprise, in some embodiments, an RNA having an open reading frame encoding a Borrelia protein, wherein the nucleic acid comprises nucleotides and/or nucleosides that can be standard (unmodified) or modified as is known in the art. In some embodiments, nucleotides and nucleosides of the present disclosure comprise modified nucleotides or nucleosides. Such modified nucleotides and nucleosides can be naturally-occurring modified nucleotides and nucleosides or non-naturally-occurring modified nucleotides and nucleosides. Such modifications can include those at the sugar, backbone, or nucleobase portion of the nucleotide and/or nucleoside as are recognized in the art.

In some embodiments, a naturally-occurring modified nucleotide or nucleotide of the disclosure is one as is generally known or recognized in the art. Non-limiting examples of such naturally-occurring modified nucleotides and nucleotides can be found, inter alia, in the widely recognized MODOMICS database.

In some embodiments, a non-naturally-occurring modified nucleotide or nucleoside of the disclosure is one as is generally known or recognized in the art. Non-limiting examples of such non-naturally-occurring modified nucleotides and nucleosides can be found, inter alia, in international publication numbers WO2013052523A1; WO2014093924A1; WO2015051173A2; WO2015051169A2; WO2015089511A2; or WO2017153936A1, each of which is herein incorporated by reference in its entirety.

Hence, nucleic acids of the disclosure (e.g., DNA and RNA, such as mRNA) can comprise standard nucleotides and nucleosides, naturally-occurring nucleotides and nucleosides, non-naturally-occurring nucleotides and nucleosides, or any combination thereof.

Nucleic acids of the disclosure (e.g., DNA and RNA, such as mRNA), in some embodiments, comprise various (more than one) different types of standard and/or modified nucleotides and nucleosides. In some embodiments, a particular region of a nucleic acid contains one, two or more (optionally different) types of standard and/or modified nucleotides and nucleosides.

In some embodiments, a modified mRNA introduced to a cell or organism, exhibits reduced degradation in the cell or organism, respectively, relative to an unmodified nucleic acid comprising standard nucleotides and nucleosides.

In some embodiments, a modified mRNA introduced into a cell or organism, may exhibit reduced immunogenicity in the cell or organism, respectively (e.g., a reduced innate response) relative to an unmodified nucleic acid comprising standard nucleotides and nucleosides.

Nucleic acids (e.g., RNA, such as mRNA), in some embodiments, comprise non-natural modified nucleotides that are introduced during synthesis or post-synthesis of the nucleic acids to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

The present disclosure provides for modified nucleosides and nucleotides of a nucleic acid (e.g., RNA, such as mRNA). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, modified nucleobases in nucleic acids (e.g., RNA, such as mRNA) comprise 1-methyl-pseudouridine (m1ψ), 1-ethyl-pseudouridine (e1ψ), 5-methoxy-uridine (mo5U), 5-methyl-cytidine (m5C), and/or pseudouridine (ψ). In some embodiments, modified nucleobases in nucleic acids (e.g., RNA, such as mRNA) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, an mRNA of the disclosure comprises 1-methyl-pseudouridine (m1ψ) substitutions at one or more or all uridine positions of the nucleic acid.

In some embodiments, an mRNA of the disclosure comprises 1-methyl-pseudouridine (m1ψ) substitutions at one or more or all uridine positions of the nucleic acid and 5-methyl cytidine substitutions at one or more or all cytidine positions of the nucleic acid.

In some embodiments, an mRNA of the disclosure comprises pseudouridine (ψ) substitutions at one or more or all uridine positions of the nucleic acid.

In some embodiments, an mRNA of the disclosure comprises pseudouridine (ψ) substitutions at one or more or all uridine positions of the nucleic acid and 5-methyl cytidine substitutions at one or more or all cytidine positions of the nucleic acid.

In some embodiments, an mRNA includes N1-methylpseudouridine. In some embodiments, at least 70%, at least 80%, at least 85%, at least 90%, at least 95%, at least 97%, at least 98%, or at least 99% of uracil nucleotides in an mRNA comprise N1-methylpseudouridine. In some embodiments, each uracil nucleotide of an mRNA transcript comprises N1-methylpseudouridine. In some embodiments, an mRNA includes 5-methylcytidine. In some embodiments, at least 70%, at least 80%, at least 85%, at least 90%, at least 95%, at least 97%, at least 98%, or at least 99% of cytosine nucleotides in an mRNA comprise 5-methylcytidine. In some embodiments, each cytosine nucleotide of an mRNA transcript comprises 5-methylcytidine. In some embodiments, an mRNA includes 5-methyluridine. In some embodiments, at least 70%, at least 80%, at least 85%, at least 90%, at least 95%, at least 97%, at least 98%, or at least 99% of uracil nucleotides in an mRNA comprise 5-methyluridine. In some embodiments, each uracil nucleotide of an mRNA transcript comprises 5-methyluridine. In some embodiments, an mRNA includes 5-methylcytidine and 5-methyluridine. In some embodiments, at least 70%, at least 80%, at least 85%, at least 90%, at least 95%, at least 97%, at least 98%, or at least 99% of uracil nucleotides in an mRNA comprise 5-methyluridine and at least 70%, at least 80%, at least 85%, at least 90%, at least 95%, at least 97%, at least 98%, or at least 99% of cytosine nucleotides in an mRNA comprise 5-methylcytidine. In some embodiments, each cytosine nucleotide of an mRNA transcript comprises 5-methylcytidine and each uracil nucleotide of an mRNA transcript comprises 5-methyluridine.

In some embodiments, an mRNA of the disclosure comprises uridine at one or more or all uridine positions of the nucleic acid.

In some embodiments, RNAs (e.g., mRNAs) are uniformly modified (e.g., fully modified, modified throughout the entire sequence) for a particular modification. For example, a nucleic acid can be uniformly modified with 1-methyl-pseudouridine, meaning that all uridine residues in the mRNA sequence are replaced with 1-methyl-pseudouridine. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the poly(A) tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The mRNA may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure or can be replaced by a plurality of compounds having different structures (e.g., 2, 3, 4 or more unique structures).

### Identification and Ratio Determination (IDR) Sequences

An Identification and Ratio Determination (IDR) sequence is a sequence of a biological molecule (e.g., nucleic acid or protein) that, when combined with the sequence of a target biological molecule, serves to identify the target biological molecule. Typically, an IDR sequence is a heterologous sequence that is incorporated within or appended to a sequence of a target biological molecule and can be used as a reference to identify the target molecule. Thus, in some embodiments, a nucleic acid (e.g., mRNA) comprises (i) a target sequence of interest (e.g., a coding sequence encoding an antigenic peptide or protein); and (ii) a unique IDR sequence.

An RNA species (e.g., RNA having a given coding sequence) may comprise an IDR sequence that differs from the IDR sequence of other RNA species (e.g., RNA(s) having different coding sequence(s)). Each IDR sequence thus identifies a particular RNA species, and so the abundance of IDR sequences may be measured to determine the abundance of each RNA species in a composition. Use of distinct IDR sequences to identify RNA species allows for analysis of multivalent RNA compositions (e.g., containing multiple RNA species) containing RNA species with similar coding sequences and/or lengths, which could otherwise be difficult to distinguish using PCR- or chromatography-based analysis of full-length RNAs.

Each RNA species in a multivalent RNA composition may comprise an IDR sequence that is not a sequence isomer of an IDR sequence of another RNA species in a multivalent RNA composition (e.g., the IDR sequence does not have the same number of adenosine nucleotides, the same number of cytosine nucleotides, the same number of guanine nucleotides, and the same number of uracil nucleotides, as another IDR sequence in the composition, even if those sequences have different sequences). Having identical nucleotide compositions causes sequence isomers to have the same mass, presenting a challenge to distinguishing sequence isomers using mass-based identification methods (e.g., mass spectrometry).

Each RNA species in a multivalent RNA composition may comprise an IDR sequence having a mass that differs from the mass of IDR sequences of each other RNA species in a multivalent RNA composition. For example, the mass of each IDR sequence may differ from the mass of other IDR sequences by at least 9 Da, at least 25 Da, at least 25 Da, or at least 50 Da. Use of IDR sequences with distinct masses allows RNA fragments comprising different IDR sequences to be distinguished using mass-based analysis methods (e.g., mass spectrometry), which do not require reverse transcription, amplification, or sequencing of RNAs.

Each RNA species in an RNA composition may comprises an IDR sequence with a different length. For example, each IDR sequence may have a length independently selected from 0 to 25 nucleotides. The length of a nucleic acid influences the rate at which the nucleic acid traverses a chromatography column, and so the use of IDR sequences of different lengths on different RNA species allows RNA fragments having different IDR sequences to be distinguished using chromatography-based methods (e.g., LC-UV).

IDR sequences may be chosen such that no IDR sequence comprises a start codon, ‘AUG’. Lack of a start codon in an IDR sequence prevents undesired translation of nucleotide sequences within and/or downstream from the IDR sequence.

IDR sequences may be chosen such that no IDR sequence comprises a recognition site for a restriction enzyme. In one example, no IDR sequence comprises a recognition site for XbaI, ‘UCUAG’. Lack of a recognition site for a restriction enzyme (e.g., XbaI recognition site ‘UCUAG’) allows the restriction enzyme to be used in generating and modifying a DNA template for in vitro transcription, without affecting the IDR sequence or sequence of the transcribed RNA.

### Nucleic Acid Production

### Chemical Synthesis

Solid-phase chemical synthesis. Nucleic acids the present disclosure may be manufactured in whole or in part using solid phase techniques. Solid-phase chemical synthesis of nucleic acids is an automated method wherein molecules are immobilized on a solid support and synthesized step by step in a reactant solution. Solid-phase synthesis is useful in site-specific introduction of chemical modifications in the nucleic acid sequences.

The synthesis of nucleic acids of the present disclosure by the sequential addition of monomer building blocks may be carried out in a liquid phase.

The synthetic methods discussed above each has its own advantages and limitations. Attempts have been conducted to combine these methods to overcome the limitations. Such combinations of methods are within the scope of the present disclosure. The use of solid-phase or liquid-phase chemical synthesis in combination with enzymatic ligation provides an efficient way to generate long chain nucleic acids that cannot be obtained by chemical synthesis alone.

### Ligation

Assembling nucleic acids by a ligase may also be used. DNA or RNA ligases promote intermolecular ligation of the 5′ and 3′ ends of polynucleotide chains through the formation of a phosphodiester bond. Nucleic acids such as chimeric polynucleotides and/or circular nucleic acids may be prepared by ligation of one or more regions or subregions. DNA fragments can be joined by a ligase catalyzed reaction to create recombinant DNA with different functions. Two oligodeoxynucleotides, one with a 5′ phosphoryl group and another with a free 3′ hydroxyl group, serve as substrates for a DNA ligase.

### Purification

Purification of the nucleic acids described herein may include, but is not limited to, nucleic acid clean-up, quality assurance and quality control. Clean-up may be performed by methods known in the arts such as, but not limited to, AGENCOURT® beads (Beckman Coulter Genomics, Danvers, MA), poly-T beads, LNATM oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment or purification method.

A quality assurance and/or quality control check may be conducted using methods such as, but not limited to, gel electrophoresis, UV absorbance, or analytical HPLC.

In some embodiments, the nucleic acids may be sequenced by methods including, but not limited to reverse-transcriptase-PCR.

### Quantification

In some embodiments, the nucleic acids of the present disclosure may be quantified in exosomes or when derived from one or more bodily fluid. Bodily fluids include peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, exosomes may be retrieved from an organ selected from the group consisting of lung, heart, pancreas, stomach, intestine, bladder, kidney, ovary, testis, skin, colon, breast, prostate, brain, esophagus, liver, and placenta.

Assays may be performed using construct specific probes, cytometry, qRT-PCR, real-time PCR, PCR, flow cytometry, electrophoresis, mass spectrometry, or combinations thereof while the exosomes may be isolated using immunohistochemical methods such as enzyme linked immunosorbent assay (ELISA) methods. Exosomes may also be isolated by size exclusion chromatography, density gradient centrifugation, differential centrifugation, nanomembrane ultrafiltration, immunoabsorbent capture, affinity purification, microfluidic separation, or combinations thereof.

These methods afford the investigator the ability to monitor, in real time, the level of nucleic acids remaining or delivered. This is possible because the nucleic acids of the present disclosure, in some embodiments, differ from the endogenous forms due to the structural or chemical modifications.

In some embodiments, the nucleic acid may be quantified using methods such as, but not limited to, ultraviolet visible spectroscopy (UV/Vis). A non-limiting example of a UV/Vis spectrometer is a NANODROP® spectrometer (ThermoFisher, Waltham, MA). The quantified nucleic acid may be analyzed in order to determine if the nucleic acid may be of proper size, check that no degradation of the nucleic acid has occurred. Degradation of the nucleic acid may be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

### In Vitro Transcription

cDNA encoding the polynucleotides described herein may be transcribed using an in vitro transcription (IVT) system. In vitro transcription of mRNA is known in the art and is described in International Publication WO 2014/152027, which is incorporated by reference herein in its entirety. In some embodiments, the RNA of the present disclosure is prepared in accordance with any one or more of the methods described in WO 2018/053209 or WO 2019/036682, each of which is incorporated by reference herein.

In some embodiments, the mRNA transcript is generated using a non-amplified, linearized DNA template in an in vitro transcription reaction to generate the RNA transcript. In some embodiments, the template DNA is isolated DNA. In some embodiments, the template DNA is cDNA. In some embodiments, the cDNA is formed by reverse transcription of an RNA, for example, but not limited to Borrelia mRNA. In some embodiments, cells, e.g., bacterial cells, e.g., E. coli, e.g., DH-1 cells are transfected with the plasmid DNA template. In some embodiments, the transfected cells are cultured to replicate the plasmid DNA which is then isolated and purified. In some embodiments, the DNA template includes an RNA polymerase promoter, e.g., a T7 promoter located 5′ to and operably linked to the gene of interest.

In some embodiments, an in vitro transcription template encodes a 5′ untranslated (UTR) region, contains an open reading frame, and encodes a 3′ UTR and a poly(A) tail. The particular nucleic acid sequence composition and length of an in vitro transcription template will depend on the mRNA encoded by the template.

In some embodiments, a nucleic acid (e.g., template DNA and/or RNA) includes 200 to 3,000 nucleotides. For example, a nucleic acid may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides.

An in vitro transcription system typically comprises a transcription buffer (e.g., with magnesium), nucleotide triphosphates (NTPs), an RNase inhibitor and a polymerase (e.g., T7 RNA polymerase). In some embodiments, one or more of the NTPs is a chemically modified NTP (e.g., with 1-methylpseudouridine or other chemical modifications described herein and/or known in the art).

In some embodiments, the NTPs comprise adenosine triphosphate (ATP), cytidine triphosphate (CTP), uridine triphosphate (UTP), and guanosine triphosphate (GTP), or an analog of each respective NTP. The ratio of NTPs may vary. In some embodiments, the ratio of GTP:ATP:CTP:UTP is 1:1:1:1. In some embodiments, the amount of the GTP or an analogue thereof is greater than an amount of the UTP or an analogue thereof. In some embodiments, the amount of the GTP is greater than the amount of the UTP. In some embodiments, the amount of ATP is greater than the amount of UTP, and the amount of CTP is greater than the amount of UTP. In some embodiments, the amount of the GTP or an analogue thereof is greater than an amount of the UTP or an analogue thereof. In some embodiments, an IVT system comprises an at least 2:1 ratio of GTP concentration to ATP concentration, an at least 2:1 ratio of GTP concentration to CTP concentration, and an at least 4:1 ratio of GTP concentration to UTP concentration. In some embodiments, an IVT system comprises a 2:1 ratio of GTP concentration to ATP concentration, a 2:1 ratio of GTP concentration to CTP concentration, and a 4:1 ratio of GTP concentration to UTP concentration. In some embodiments, an IVT system comprises guanosine diphosphate (GDP). In some embodiments, an IVT system comprises an at least 3:1 ratio of GTP plus GDP concentration to ATP concentration, an at least 6:1 ratio of GTP plus GDP concentration to CTP concentration, and an at least 6:1 ratio of GTP plus GDP concentration to UTP concentration.

The NTPs may be manufactured in house, may be selected from a supplier, or may be synthesized as described herein. The NTPs may be selected from, but are not limited to, those described herein including natural and unnatural (modified) NTPs.

Any number of RNA polymerases or variants may be used in the method of the present disclosure. The polymerase may be selected from, but is not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides. Some embodiments exclude the use of DNase.

An IVT system, in some embodiments, comprises magnesium buffer, dithiothreitol (DTT) spermidine, pyrophosphatase, and/or RNase inhibitor. In some embodiments, an IVT system omits an RNase inhibitor. An IVT system may be incubated at 25 degrees Celsius or at 37 degrees Celsius. Other temperatures may be used, depending in part on the polymerase (e.g., use of a variant polymerase).

In some embodiments, the RNA transcript is capped via enzymatic capping. In some embodiments, the RNA comprises 5′ terminal cap, for example, 7mG(5′)ppp(5′)NlmpNp.

### Lipid Compositions

In some embodiments, the nucleic acids of in (e.g., formulated as) a lipid composition, such as a composition comprising a lipid nanoparticle, a liposome, and/or a lipoplex. In some embodiments, nucleic acids of the present disclosure are in (e.g., formulated as) lipid nanoparticle (LNP) compositions. Lipid nanoparticles typically comprise amino lipid, non-cationic lipid, structural lipid, and PEG lipid components along with the nucleic acid cargo (e.g., RNA, such as mRNA) of interest. A lipid nanoparticles of the present disclosure can be generated using components, compositions, and methods as are generally known in the art, see for example PCT/US2016/052352; PCT/US2016/068300; PCT/US2017/037551; PCT/US2015/027400; PCT/US2016/047406; PCT/US2016/000129; PCT/US2016/014280; PCT/US2017/038426; PCT/US2014/027077; PCT/US2014/055394; PCT/US2016/052117; PCT/US2012/069610; PCT/US2017/027492; PCT/US2016/059575; PCT/US2016/069491; PCT/US2016/069493; and PCT/US2014/066242, all of which are incorporated by reference herein in their entirety.

In some embodiments, a lipid nanoparticle comprises at least one ionizable amino lipid, at least one non-cationic lipid, at least one sterol, and/or at least one polyethylene glycol (PEG)-modified lipid.

In some embodiments, a lipid nanoparticle comprises 20-60 mole percent (mol %) ionizable amino lipid, 5-25 mol % non-cationic lipid, 25-55 mol % structural lipid, and 0.5-15 mol % PEG-modified lipid.

In some embodiments, a lipid nanoparticle comprises 20-60 mol % ionizable amino lipid, 5-30 mol % non-cationic lipid, 10-55 mol % structural lipid, and 0.5-15 mol % PEG-modified lipid.

In some embodiments, a lipid nanoparticle comprises 40-50 mol % ionizable lipid, optionally 45-50 mol %, for example, 45-46 mol %, 46-47 mol %, 47-48 mol %, 48-49 mol %, or 49-50 mol % for example about 45 mol %, 45.5 mol %, 46 mol %, 46.5 mol %, 47 mol %, 47.5 mol %, 48 mol %, 48.5 mol %, 49 mol %, or 49.5 mol %.

In some embodiments, a lipid nanoparticle comprises 20-60 mol % ionizable amino lipid. For example, a lipid nanoparticle may comprise 20-50 mol %, 20-40 mol %, 20-30 mol %, 30-60 mol %, 30-50 mol %, 30-40 mol %, 40-60 mol %, 40-50 mol %, or 50-60 mol % ionizable amino lipid. In some embodiments, a lipid nanoparticle comprises 20 mol %, 30 mol %, 40 mol %, 50 mol %, or 60 mol % ionizable amino lipid. In some embodiments, a lipid nanoparticle comprises mol %, 36 mol %, 37 mol %, 38 mol %, 39 mol %, 40 mol %, 41 mol %, 42 mol %, 43 mol %, 44 mol %, 45 mol %, 46 mol %, 47 mol %, 48 mol %, 49 mol %, 50 mol %, 51 mol %, 52 mol %, 53 mol %, 54 mol %, or 55 mol % ionizable amino lipid.

In some embodiments, a lipid nanoparticle comprises 45-55 mol % ionizable amino lipid. For example, lipid nanoparticle may comprise 45, 46, 47, 48, 49, 50, 51, 52, 53, 54, or 55 mol % ionizable amino lipid.

### Ionizable Amino Lipids

In some embodiments, the ionizable amino lipid of the present disclosure is a compound of Formula (AI):

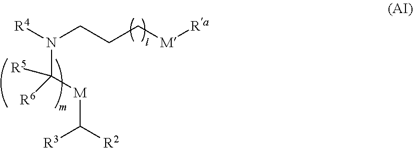

or its N-oxide, or a salt or isomer thereof,
wherein R′^{a} is R′^{branched}; wherein
R′^{branched} is:

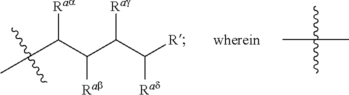

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH, wherein n is selected from the group consisting of 1, 2, 3, 4, and 5, and

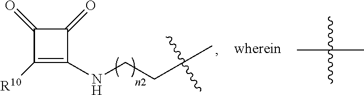

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments of the compounds of Formula (AI), R′^{a} is R′^{branched}; R′^{branched} is

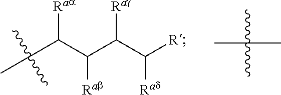

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 5; and m is 7.

In some embodiments of the compounds of Formula (AI), R′^{a} is R′^{branched}; R′^{branched} is

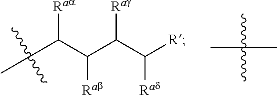

denotes a point of attachment; R^{aα}, R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 3; and m is 7.

In some embodiments of the compounds of Formula (AI), R′^{a} is R′^{branched}; R′^{branched} is

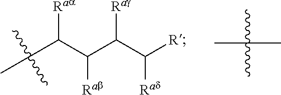

denotes a point of attachment; R^{aα} is C₂₋₁₂ alkyl; R^{aβ}, R^{aγ}, and R^{aδ} are each H; R² and R³ are each C₁₋₁₄ alkyl; R⁴ is

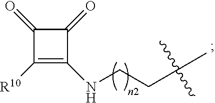

R¹⁰ NH(C₁₋₆ alkyl); n2 is 2; R⁵ is H; each R⁶ is H; M and M′ are each —C(O)O—; R′ is a C₁₋₁₂ alkyl; l is 5; and m is 7.

In some embodiments of the compounds of Formula (I), R′^{a} is R′^{branched}; R′^{branched} is

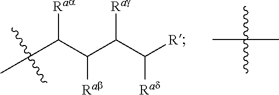

denotes a point of attachment; R^{aα}, R^{aβ}, and R^{aδ} are each H; R^{aγ} is C₂₋₁₂ alkyl; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 5; and m is 7.

In some embodiments, the compound of Formula (I) is selected from:

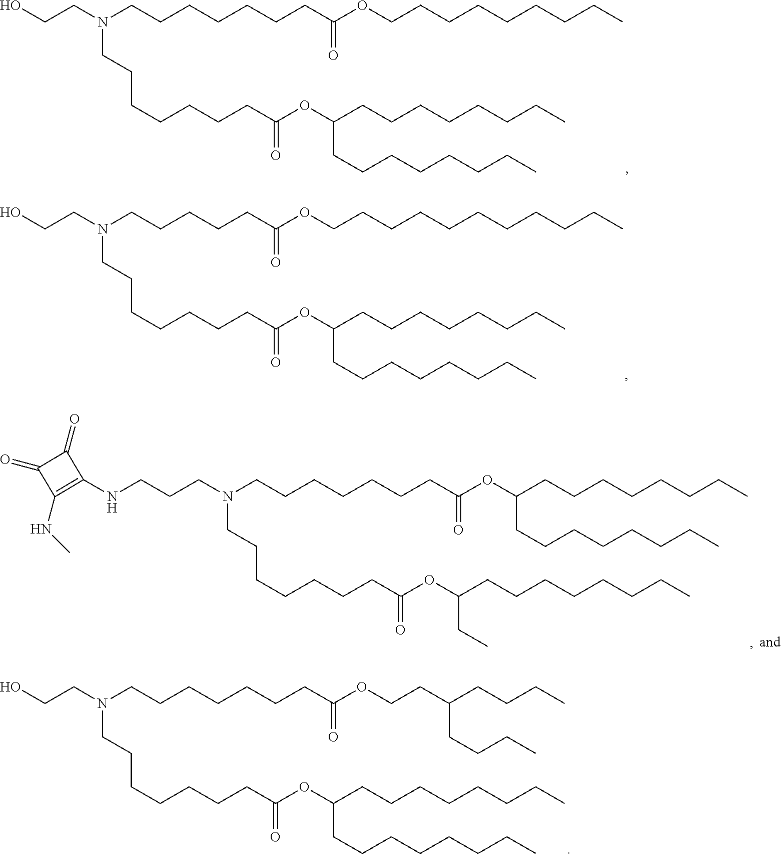

In some embodiments, the ionizable amino lipid is a compound of Formula (Ala):

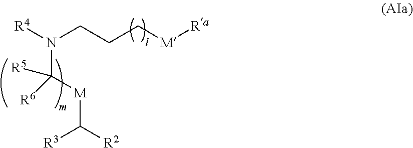

or its N-oxide, or a salt or isomer thereof,
wherein R′^{a} is R′^{branched}; wherein
R′^{branched} is:

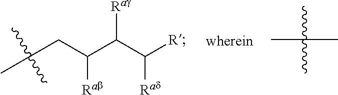

denotes a point of attachment;
wherein R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

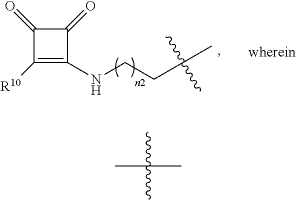

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, the ionizable amino lipid is a compound of Formula (AIb):

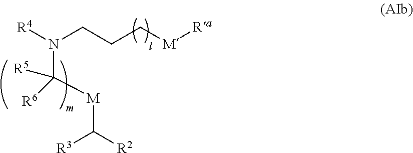

or its N-oxide, or a salt or isomer thereof,
wherein R′^{a} is R′^{branched}; wherein
R′^{branched} is:

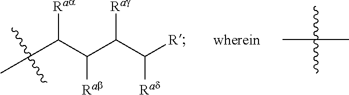

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is —(CH₂)ₙOH, wherein n is selected from the group consisting of 1, 2, 3, 4, and 5; each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments of Formula (AI) or (AIb), R′^{a} is R′^{branched}; R′^{branched} is

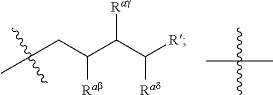

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 5; and m is 7.

In some embodiments of Formula (AI) or (AIb), R′^{a} is R′^{branched}; R′^{branched} is

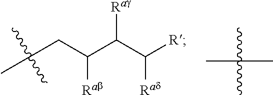

denotes a point of attachment; R^{aβ}, R^{aγ}, and R^{aδ} are each H; R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 3; and m is 7.

In some embodiments of Formula (AI) or (AIb), R′^{a} is R′^{branched}; R′^{branched} is

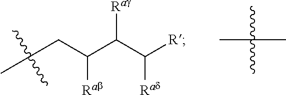

denotes a point of attachment; R^{aβ} and R^{aα} are each H; R^{aγ} is C₂₋₁₂ alkyl;
R² and R³ are each C₁₋₁₄ alkyl; R⁴ is —(CH₂)ₙOH; n is 2; each R⁵ is H; each R⁶ is H; M and M′ are each —C(O)O—; R′ is a C₁₋₁₂ alkyl; l is 5; and m is 7.

In some embodiments, the ionizable amino lipid is a compound of Formula (AIc):

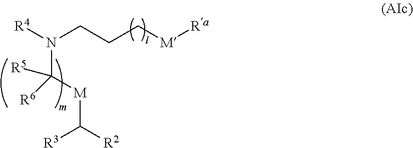

or its N-oxide, or a salt or isomer thereof,
wherein R′^{a} is R′^{branched}; wherein
   R′^{branched} is:

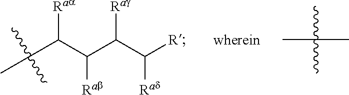

denotes a point of attachment;
wherein R^{aα}, R^{aβ}, R^{aγ}, and R^{aδ} are each independently selected from the group consisting of H, C₂₋₁₂ alkyl, and C₂₋₁₂ alkenyl;
R² and R³ are each independently selected from the group consisting of C₁₋₁₄ alkyl and C₂₋₁₄ alkenyl;
R⁴ is

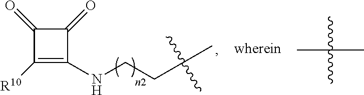

denotes a point of attachment; wherein R¹⁰ is N(R)₂; each R is independently selected from the group consisting of C₁₋₆ alkyl, C₂₋₃ alkenyl, and H; n2 is selected from the group consisting of 1, 2, 3, 4, 5, 6, 7, 8, 9, and 10;
each R⁵ is independently selected from the group consisting of C₁₋₃ alkyl, C₂₋₃ alkenyl, and H;
each R⁶ is independently selected from the group consisting of C₁₋₃ alkyl, C₂₋₃ alkenyl, and H;
M and M′ are each independently selected from the group consisting of —C(O)O— and —OC(O)—;
R′ is a C₁₋₁₂ alkyl or C₂₋₁₂ alkenyl;
l is selected from the group consisting of 1, 2, 3, 4, and 5; and
m is selected from the group consisting of 5, 6, 7, 8, 9, 10, 11, 12, and 13.

In some embodiments, R′^{a} is R′^{branched}; R′^{branched} is

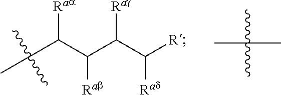

denotes a point of attachment; R^{aβ}, R^{aγ}, and R^{aδ} are each H; R^{aα} is C₂₋₁₂ alkyl; R² and R³ are each C₁₋₁₄ alkyl; R⁴ is

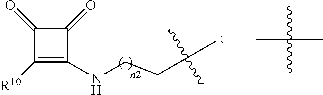

denotes a point of attachment; R¹⁰ is NH(C₁₋₆ alkyl); n2 is 2; each R⁵ is H; each R⁶ is H; M and M′ are each —C(O)O—; R′ is a C₁₋₁₂ alkyl; l is 5; and m is 7.

In some embodiments, the compound of Formula (AIc) is:

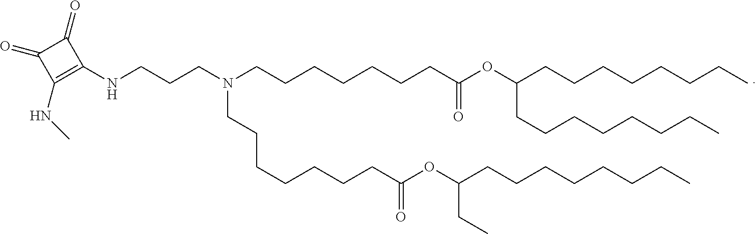 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

In some embodiments, the ionizable amino lipid is a compound of Formula (AII):

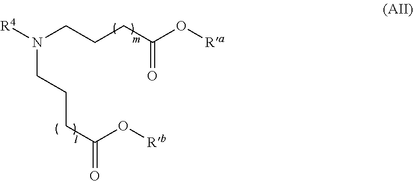

or its N-oxide, or a salt or isomer thereof,
wherein R′^{a} is R′^{branched} or R′^{cyclic}; wherein
R′^{branched} is:

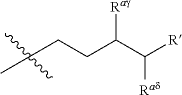

and R′^{cyclic} is:

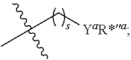

and
R′^{b} is:

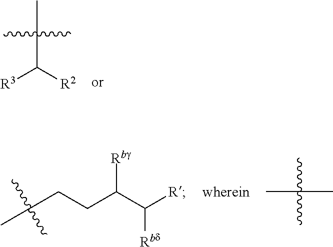

a point of attachment;
R^{aγ} and R^{aδ} are each independently selected from the group consisting of H, C₁₋₁₂ alkyl, and C₂₋₁₂ alkenyl, wherein at least one of R^{aγ} and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{b}r and R^{b}s is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

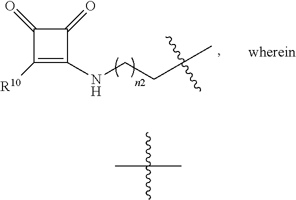

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
Y^{a} is a C₃₋₆ carbocycle;
R*″^{a} is selected from the group consisting of C₁₋₁₅ alkyl and C₂₋₁₅ alkenyl; and
s is 2 or 3;
m is selected from 1, 2, 3, 4, 5, 6, 7, 8, and 9;
l is selected from 1, 2, 3, 4, 5, 6, 7, 8, and 9.

In some embodiments, the ionizable amino lipid is a compound of Formula (AII-a):

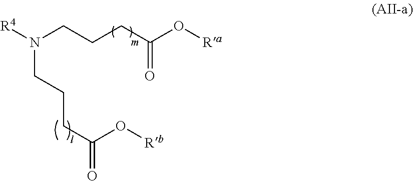

or its N-oxide, or a salt or isomer thereof,
wherein R′^{a} is R′^{branched} or R′^{cyclic}; wherein
R′^{branched} is:

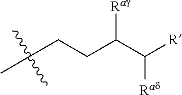

and R′^{b} is:

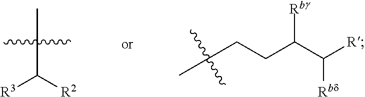

denotes a point of attachment;
R^{aγ} and R^{aδ} are each independently selected from the group consisting of H, C₁₋₁₂ alkyl, and C₂₋₁₂ alkenyl, wherein at least one of Ray and R^{aδ} is selected from the group consisting of C₁₋₁₂ alkyl and C₂₋₁₂ alkenyl;
R^{bγ} and R^{bδ} are each independently selected from the group consisting of H, C₁₋₁₂ alkyl, and C₂₋₁₂ alkenyl, wherein at least one of R^{bγ} and R^{bδ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

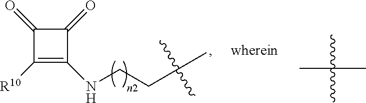

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid is a compound of Formula (AII-b):

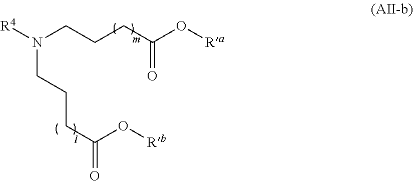

or its N-oxide, or a salt or isomer thereof,
wherein R′^{a} is R′^{branched} or R′^{cyclic}; wherein
R′_{branched} is:

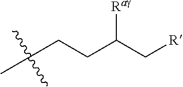

and R′^{b} is:

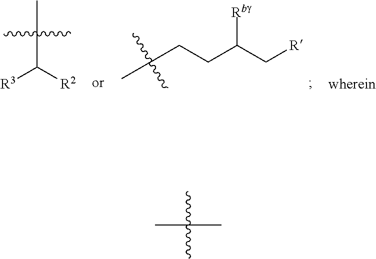

denotes a point of attachment;
R^{aγ} and R^{bγ} are each independently selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

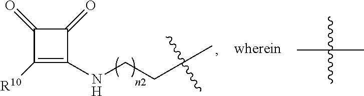

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid is a compound of Formula (AII-c):

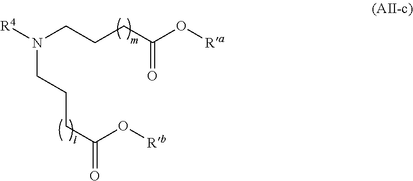

or its N-oxide, or a salt or isomer thereof, wherein R′^{a} is R′^{branched} or R′^{cyclic}; wherein
R′^{branched} is:

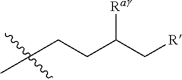

and R′^{b} is:

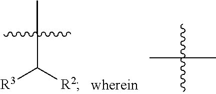

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is selected from the group consisting of —(CH₂)ₙOH wherein n is selected from the group consisting of 1, 2, 3, 4, and 5, and

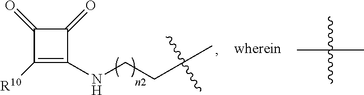

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid is a compound of Formula (AII-d):

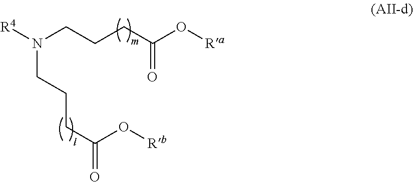

or its N-oxide, or a salt or isomer thereof,
wherein R′^{a} is R′^{branched} or R ^{cyclic}; wherein
R′^{branched} is:

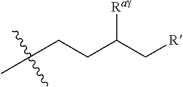

and R′^{b} is:

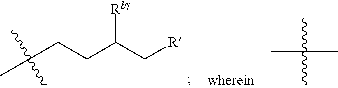

denotes a point of attachment;
wherein R^{aγ} and R^{bγ} are each independently selected from the group consisting of C₁₋₁₂ alkyl and C₂₋₁₂ alkenyl;
R⁴ is selected from the group consisting of —(CH₂)ₙOH wherein n is selected from the group consisting of 1, 2, 3, 4, and 5, and

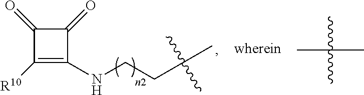

denotes a point of attachment; wherein R¹⁰ is N(R)₂; each R is independently selected from the group consisting of C₁₋₆ alkyl, C₂₋₃ alkenyl, and H; and n2 is selected from the group consisting of 1, 2, 3, 4, 5, 6, 7, 8, 9, and 10;
each R′ independently is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments, the ionizable amino lipid is a compound of Formula (AII-e):

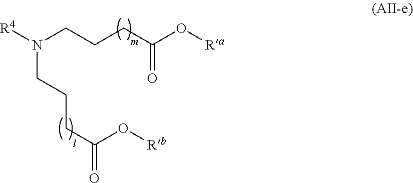

or its N-oxide, or a salt or isomer thereof,
wherein R′^{a} is R′^{branched} or R′^{cyclic}; wherein
R′^{branched} is:

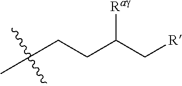

and R′^{b} is:

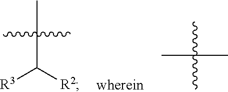

denotes a point of attachment;
wherein R^{aγ} is selected from the group consisting of C₁₋₁₂ alkyl and C₂₋₁₂ alkenyl;
R² and R³ are each independently selected from the group consisting of C₁₋₁₄ alkyl and C₂₋₁₄ alkenyl;
R⁴ is —(CH₂)ₙOH wherein n is selected from the group consisting of 1, 2, 3, 4, and 5;
R′ is a C₁₋₁₂ alkyl or C₂₋₁₂ alkenyl;
m is selected from 1, 2, 3, 4, 5, 6, 7, 8, and 9;
l is selected from 1, 2, 3, 4, 5, 6, 7, 8, and 9.

In some embodiments of the compound of Formula (AII), (AII-a), (AII-b), (AII-c), (AII-d), or (AII-e), m and l are each independently selected from 4, 5, and 6. In some embodiments of the compound of Formula (AII), (AII-a), (AII-b), (AII-c), (AII-d), or (AII-e), m and l are each 5.

In some embodiments of the compound of Formula (AII), (AII-a), (AII-b), (AII-c), (AII-d), or (AII-e), each R′ independently is a C₁₋₁₂ alkyl. In some embodiments of the compound of Formula (AII), (AII-a), (AII-b), (AII-c), (AII-d), or (AII-e), each R′ independently is a C₂₋₅ alkyl.

In some embodiments of the compound of Formula (AII), (AII-a), (AII-b), (AII-c), (AII-d), or (AII-e), R′^{b} is:

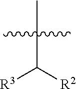

and R² and R³ are each independently a C₁₋₁₄ alkyl. In some embodiments of the compound of Formula (AII), (AII-a), (AII-b), (AII-c), (AII-d), or (AII-e), R′^{b} is:

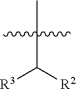

and R² and R³ are each independently a C₆₋₁₀ alkyl. In some embodiments of the compound of Formula (AII), (AII-a), (AII-b), (AII-c), (AII-d), or (AII-e), R′^{b} is:

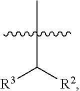

and R² and R³ are each a C₈ alkyl.

In some embodiments of the compound of Formula (AII), (AII-a), (AII-b), (AII-c), (AII-d), or (AII-e), R′^{branched} is:

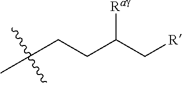

### and R′^{b} is:

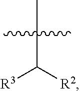

R^{aγ} is a C₁₋₁₂ alkyl and R² and R³ are each independently a C₆₋₁₀ alkyl. In some embodiments of the compound of Formula (AII), (AII-a), (AII-b), (AII-c), (AII-d), or (AII-e), R′^{branched} is:

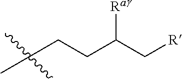

### and R′^{b} is:

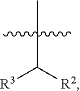

R^{aγ} is a C₂₋₆ alkyl and R² and R³ are each independently a C₆₋₁₀ alkyl. In some embodiments of the compound of Formula (AII), (AII-a), (AII-b), (AII-c), (AII-d), or (AII-e), R′^{branched} is:

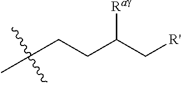

### and R′^{b} is:

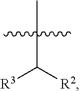

R^{aγ} is a C₂₋₆ alkyl, and R² and R³ are each a C₈ alkyl.

In some embodiments of the compound of Formula (AII), (AII-a), (AII-b), (AII-c), (AII-d), or (AII-e), R′^{branched} is:

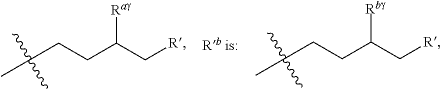

and R^{aγ} and R^{bγ} are each a C₁₋₁₂ alkyl. In some embodiments of the compound of Formula (AII), (AII-a), (AII-b), (AII-c), (AII-d), or (AII-e), R′^{branched} is:

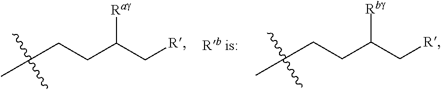

and R^{aγ} and R^{bγ} are each a C₂₋₆ alkyl.

In some embodiments of the compound of Formula (AII), (AII-a), (AII-b), (AII-c), (AII-d), or (AII-e), m and l are each independently selected from 4, 5, and 6 and each R′ independently is a C₁₋₁₂ alkyl. In some embodiments of the compound of Formula (AII), (AII-a), (AII-b), (AII-c), (AII-d), or (AII-e), m and l are each 5 and each R′ independently is a C₂₋₅ alkyl.

In some embodiments of the compound of (All), (AII-a), (AII-b), (AII-c), (AII-d), or (AII-e), R′^{branched} is:

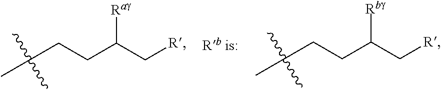

m and l are each independently selected from 4, 5, and 6, each R′ independently is a C₁₋₁₂ alkyl, and R^{aγ} and R^{bγ} are each a C₁₋₁₂ alkyl. In some embodiments of the compound of Formula (AII), (AII-a), (AII-b), (AII-c), (AII-d), or (AII-e), R′^{branched} is:

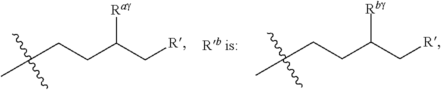

m and l are each 5, each R′ independently is a C₂₋₅ alkyl, and R^{aγ} and R^{bγ} are each a C₂₋₆ alkyl.

In some embodiments of the compound of Formula (AII), (AII-a), (AII-b), (AII-c), (AII-d), or (AII-e), R′^{branched} is:

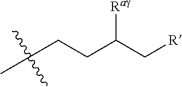

### and R′^{b} is:

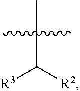

m and l are each independently selected from 4, 5, and 6, R′ is a C₁₋₁₂ alkyl, Ray is a C₁₋₁₂ alkyl and R² and R³ are each independently a C₆₋₁₀ alkyl.

In some embodiments of the compound of Formula (AII), (AII-a), (AII-b), (AII-c), (AII-d), or (AII-e), R′^{branched} is:

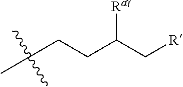

### and R′^{b} is:

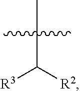

m and l are each 5, R′ is a C₂₋₅ alkyl, R^{aγ} is a C₂₋₆ alkyl, and R² and R³ are each a C₈ alkyl.

In some embodiments of the compound of (All), (AII-a), (AII-b), (AII-c), (AII-d), or (AII-e), R⁴ is

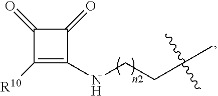

wherein R¹⁰ is NH(C₁₋₆ alkyl) and n2 is 2. In some embodiments of the compound of Formula (AII), (AII-a), (AII-b), (AII-c), (AII-d), or (AII-e), R⁴ is

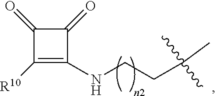

wherein R¹⁰ is NH(CH₃) and n2 is 2.

In some embodiments of the compound of Formula (AII), (AII-a), (AII-b), (AII-c), (AII-d), or (AII-e), R′^{branched} is:

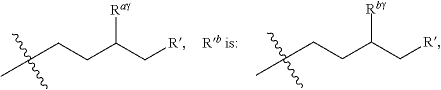

m and l are each independently selected from 4, 5, and 6, each R′ independently is a C₁₋₁₂ alkyl, R^{aγ} and R^{bγ} are each a C₁₋₁₂ alkyl, and R⁴ is

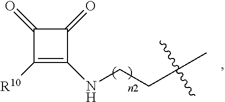

wherein R¹⁰ is NH(C₁₋₆ alkyl), and n2 is 2. In some embodiments of the compound of Formula (AII), (AII-a), (AII-b), (AII-c), (AII-d), or (AII-e), R′^{branched} is:

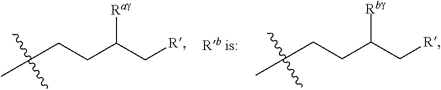

m and l are each 5, each R′ independently is a C₂₋₅ alkyl, R^{aγ} and R^{bγ} are each a C₂₋₆ alkyl, and R⁴ is

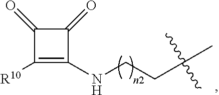

wherein R¹⁰ is NH(CH₃) and n2 is 2.

In some embodiments of the compound of Formula (AII), (AII-a), (AII-b), (AII-c), (AII-d), or (AII-e), R′^{branched} is:

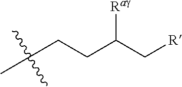

### and R′^{b} is:

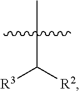

m and l are each independently selected from 4, 5, and 6, R′ is a C₁₋₁₂ alkyl, R² and R³ are each independently a C₆₋₁₀ alkyl, R^{aγ} is a C₁₋₁₂ alkyl, and R⁴ is

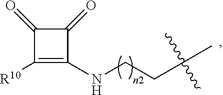

wherein R¹⁰ is NH(C₁₋₆ alkyl) and n2 is 2. In some embodiments of the compound of Formula (AII), (AII-a), (AII-b), (AII-c), (AII-d), or (AII-e), R′^{branched} is:

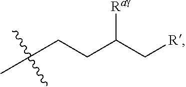

### and R′^{b} is:

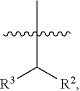

m and l are each 5, R′ is a C₂₋₅ alkyl, R^{aγ} is a C₂₋₆ alkyl, R² and R³ are each a C₈ alkyl, and R⁴ is

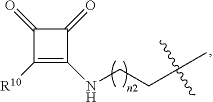

wherein R¹⁰ is NH(CH₃) and n2 is 2.

In some embodiments of the compound of Formula (AII), (AII-a), (AII-b), (AII-c), (AII-d), or (AII-e), R⁴ is —(CH₂)ₙOH and n is 2, 3, or 4. In some embodiments of the compound of Formula (AII), (AII-a), (AII-b), (AII-c), (AII-d), or (AII-e), R⁴ is —(CH₂)ₙOH and n is 2.

In some embodiments of the compound of Formula (AII), (AII-a), (AII-b), (AII-c), (AII-d), or (AII-e), R′^{branched} is:

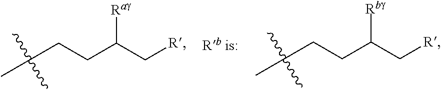

m and l are each independently selected from 4, 5, and 6, each R′ independently is a C₁₋₁₂ alkyl, R^{aγ} and R^{bγ} are each a C₁₋₁₂ alkyl, R⁴ is —(CH₂)ₙOH, and n is 2, 3, or 4. In some embodiments of the compound of Formula (AII), (AII-a), (AII-b), (AII-c), (AII-d), or (AII-e), R′^{branched} is:

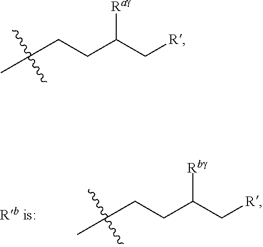

m and l are each 5, each R′ independently is a C₂₋₅ alkyl, R^{aγ} and R^{bγ} are each a C₂₋₆ alkyl, R⁴ is —(CH₂)ₙOH, and n is 2.

In some embodiments, the ionizable amino lipid is a compound of Formula (AII-f):

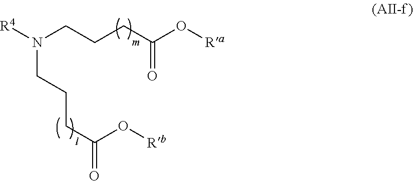

or its N-oxide, or a salt or isomer thereof,
wherein R′^{a} is R′^{branched} or R′^{cyclic}; wherein
R′^{branched} is:

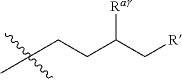

and R′^{b} is:

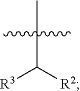

denotes a point of attachment;
R^{aγ} is a C₁₋₁₂ alkyl;
R² and R³ are each independently a C₁₋₁₄ alkyl;
R⁴ is —(CH₂)ₙOH wherein n is selected from the group consisting of 1, 2, 3, 4, and 5;
R′ is a C₁₋₁₂ alkyl;
m is selected from 4, 5, and 6; and
l is selected from 4, 5, and 6.

In some embodiments of the compound of Formula (AII-f), m and l are each 5, and n is 2, 3, or 4.

In some embodiments of the compound of Formula (AII-f) R′ is a C₂₋₅ alkyl, R^{aγ} is a C₂₋₆ alkyl, and R² and R³ are each a C₆₋₁₀ alkyl.

In some embodiments of the compound of Formula (AII-f), m and l are each 5, n is 2, 3, or 4, R′ is a C₂₋₅ alkyl, R^{aγ} is a C₂₋₆ alkyl, and R² and R³ are each a C₆₋₁₀ alkyl.

In some embodiments, the ionizable amino lipid is a compound of Formula (AII-g):

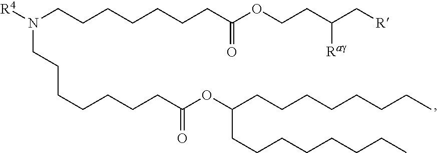

wherein
R^{aγ} is a C₂₋₆ alkyl;
R′ is a C₂₋₅ alkyl; and
R⁴ is selected from the group consisting of —(CH₂)ₙOH wherein n is selected from the group consisting of 3, 4, and 5, and

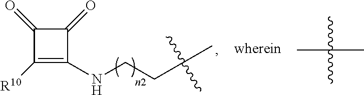

denotes a point of attachment, R¹⁰ is NH(C₁₋₆ alkyl), and n2 is selected from the group consisting of 1, 2, and 3.

In some embodiments, the ionizable amino lipid is a compound of Formula (AII-h):

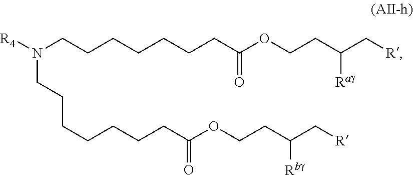

wherein
R^{aγ} and R^{bγ} are each independently a C₂₋₆ alkyl;
each R′ independently is a C₂₋₅ alkyl; and
R⁴ is selected from the group consisting of —(CH₂)ₙOH wherein n is selected from the group consisting of 3, 4, and 5, and

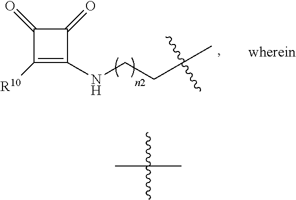

denotes a point of attachment, R¹⁰ is NH(C₁₋₆ alkyl), and n2 is selected from the group consisting of 1, 2, and 3.

In some embodiments of the compound of Formula (AII-g) or (AII-h), R⁴ is

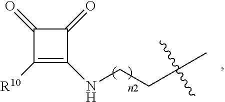

wherein R¹⁰ is NH(CH₃) and n2 is 2.

In some embodiments of the compound of Formula (AII-g) or (AII-h), R⁴ is —(CH₂)₂OH.

In some embodiments, the ionizable amino lipids of the present disclosure may be one or more of compounds of Formula (VI):

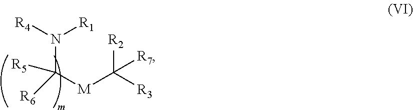

or their N-oxides, or salts or isomers thereof, wherein:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of hydrogen, a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —N(R)S(O)₂R⁸, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —OC(O)-M″-C(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;
R₇ is selected from the group consisting of C₁₋₁₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₅ alkyl and C₃₋₁₅ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and m is selected from 5, 6, 7, 8, 9, 10, 11, 12, and 13; and wherein when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, another subset of compounds of Formula (VI) includes those in which:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and a 5- to 14-membered heterocycloalkyl having one or more heteroatoms selected from N, O, and S which is substituted with one or more substituents selected from oxo (═O), OH, amino, mono- or di-alkylamino, and C₁₋₃ alkyl, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (VI) includes those in which:
R¹ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and —CQ(R)₂, then Q is either a 5- to 14-membered heteroaryl or 8- to 14-membered heterocycloalkyl;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H; R₈ is selected from the group consisting of C₃₋₆ carbocycle and heterocycle; R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₈ is alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (VI) includes those in which:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R⁸, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R⁸ is selected from the group consisting of C₃₋₆ carbocycle and heterocycle; R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (VI) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is —(CH₂)ₙQ or —(CH₂)ₙCHQR, where Q is —N(R)₂, and n is selected from 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H; each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (VI) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of —(CH₂)ₙQ, —(CH₂)·CHQR, —CHQR, and —CQ(R)₂, where Q is —N(R)₂, and n is selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H; each R is independently selected from the group consisting of C₁₋₃ alkyl, C₂₋₃ alkenyl, and H;
each R′ is independently selected from the group consisting of C₁₋₈ is alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In certain embodiments, a subset of compounds of Formula (VI) includes those of Formula (VI-A):

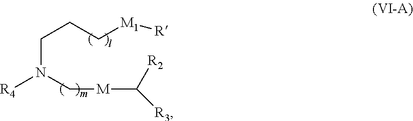

or its N-oxide, or a salt or isomer thereof, wherein 1 is selected from 1, 2, 3, 4, and 5; m is selected from 5, 6, 7, 8, and 9; M1 is a bond or M′; R₄ is hydrogen, unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R⁸, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (VI) includes those of Formula (VT-B):

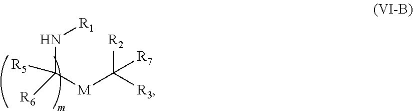

or its N-oxide, or a salt or isomer thereof in which all variables are as defined herein. For example, m is selected from 5, 6, 7, 8, and 9; R⁴ is hydrogen, unsubstituted C₁₋₃ alkyl, or —(CH₂)ₙQ, in which Q is H, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (VI) includes those of Formula (VII):

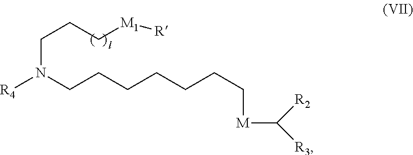

or its N-oxide, or a salt or isomer thereof, wherein 1 is selected from 1, 2, 3, 4, and 5; M₁ is a bond or M′; R₄ is hydrogen, unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, the compounds of Formula (VI) are of Formula (VIIa),

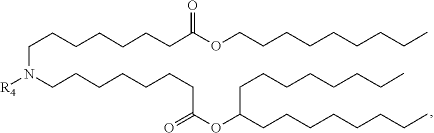

or their N-oxides, or salts or isomers thereof, wherein R₄ is as described herein.

In another embodiment, the compounds of Formula (VI) are of Formula (VIIb),

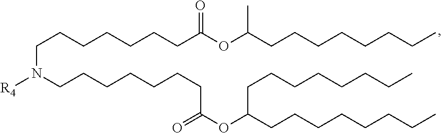

or their N-oxides, or salts or isomers thereof, wherein R₄ is as described herein.

In another embodiment, the compounds of Formula (VI) are of Formula (VIIc) or (VIIe):

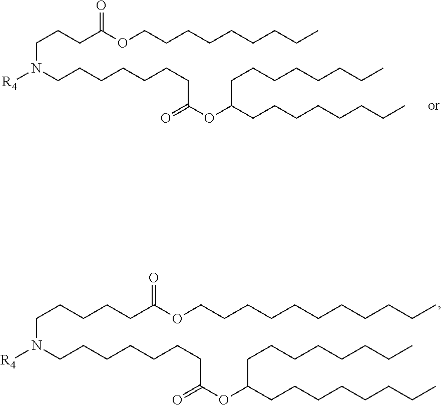

or their N-oxides, or salts or isomers thereof, wherein R₄ is as described herein.

In another embodiment, the compounds of Formula (VI) are of Formula (VIIf):

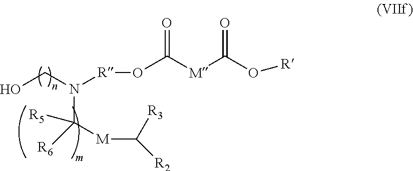

or their N-oxides, or salts or isomers thereof,
wherein M is —C(O)O— or —OC(O)—, M″ is C₁₋₆ alkyl or C₂₋₆ alkenyl, R₂ and R₃ are independently selected from the group consisting of C₅₋₁₄ alkyl and C₅₋₁₄ alkenyl, and n is selected from 2, 3, and 4.

In a further embodiment, the compounds of Formula (VI) are of Formula (VIId),

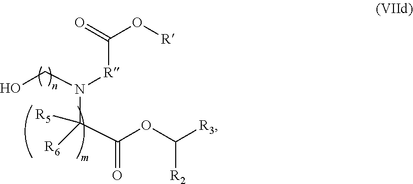

or their N-oxides, or salts or isomers thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In some embodiments, an ionizable amino lipid of the disclosure comprises a compound having structure:

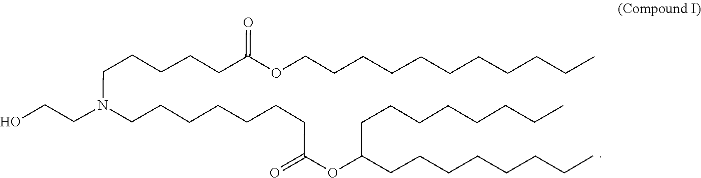 [SMILES: CCCCCCCCCCCOC(=O)CCCCCN(CCO)CCCCCCCC(=O)OC(CCCCCCCC)CCCCCCCC]

In some embodiments, an ionizable amino lipid of the disclosure comprises a compound having structure:

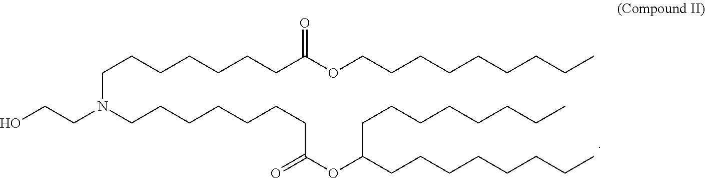 [SMILES: CCCCCCCCCOC(=O)CCCCCCCN(CCO)CCCCCCCC(=O)OC(CCCCCCCC)CCCCCCCC]

In a further embodiment, the compounds of Formula (VI) are of Formula (VIIg),

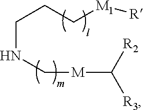

or their N-oxides, or salts or isomers thereof, wherein 1 is selected from 1, 2, 3, 4, and 5; m is selected from 5, 6, 7, 8, and 9; M₁ is a bond or M′; M and M′ are independently selected from —C(O)O—, —OC(O)—, —OC(O)-M″-C(O)O—, —C(O)N(R′)—, —P(O)(OR′)O—, —S—S—, an aryl group, and a heteroaryl group; and R₂ and R³ are independently selected from the group consisting of H, C₁₋₁₄ alkyl, and C₂₋₁₄ alkenyl. For example, M″ is C₁₋₆ alkyl (e.g., C₁₋₄ alkyl) or C₂₋₆ alkenyl (e.g. C₂₋₄ alkenyl). For example, R₂ and R₃ are independently selected from the group consisting of C₅₋₁₄ alkyl and C₅₋₁₄ alkenyl.

In some embodiments, the ionizable amino lipids are one or more of the compounds described in U.S. Application Nos. 62/220,091, 62/252,316, 62/253,433, 62/266,460, 62/333,557, 62/382,740, 62/393,940, 62/471,937, 62/471,949, 62/475,140, and 62/475,166, and PCT Application No. PCT/US2016/052352.

The central amine moiety of a lipid according to Formula (VI), (VI-A), (VI-B), (VII), (VIIa), (VIIb), (VIIc), (VIId), (VIIe), (VIIf), or (VIIg) may be protonated at a physiological pH. Thus, a lipid may have a positive or partial positive charge at physiological pH. Such amino lipids may be referred to as cationic lipids, ionizable lipids, cationic amino lipids, or ionizable amino lipids. Amino lipids may also be zwitterionic, i.e., neutral molecules having both a positive and a negative charge.

In some embodiments, the ionizable amino lipids of the present disclosure may be one or more of compounds of formula (VIII),

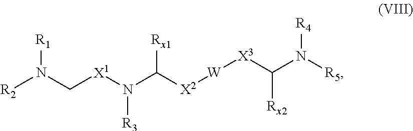

or salts or isomers thereof, wherein
W is

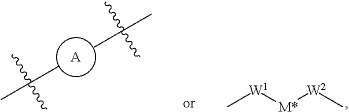

ring A is

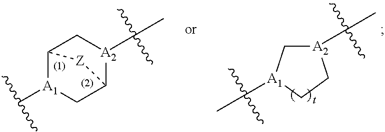

t is 1 or 2;
A₁ and A₂ are each independently selected from CH or N;
Z is CH₂ or absent wherein when Z is CH₂, the dashed lines (1) and (2) each represent a single bond; and when Z is absent, the dashed lines (1) and (2) are both absent;
R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;
R_{X1} and R_{X2} are each independently H or C₁₋₃ alkyl;
each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′) O—, —S(O)₂—, —C(O)S—, —SC(O)—, an aryl group, and a heteroaryl group;
M* is C₁-C₆ alkyl,
W¹ and W² are each independently selected from the group consisting of —O— and —N(R⁶)—;
each R₆ is independently selected from the group consisting of H and C₁₋₅ alkyl;
X¹, X², and X³ are independently selected from the group consisting of a bond, —CH₂—, —(CH₂)₂—, —CHR—, —CHY—, —C(O)—, —C(O)O—, —OC(O)—, —(CH₂)~-C(O)—, —C(O)—(CH₂)ₙ—, —(CH₂)ₙ—C(O)O—, —OC(O)—(CH₂)ₙ—, —(CH₂)ₙ—OC(O)—, —C(O)O—(CH₂)ₙ—, —CH(OH)—, —C(S)—, and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂₋₁₂ alkenyl, and H;
each R″ is independently selected from the group consisting of C₃₋₁₂ alkyl, C₃₋₁₂ alkenyl
and —R*MR′; and
n is an integer from 1-6;
wherein when ring A is

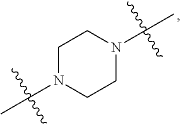 [SMILES: *N1CCN(*)CC1]

then
i) at least one of X¹, X², and X³ is not —CH₂—; and/or
ii) at least one of R₁, R₂, R₃, R₄, and R₅ is —R″MR′.

In some embodiments, the compound is of any of formulae (VIIIa1)-(VIIIa8):

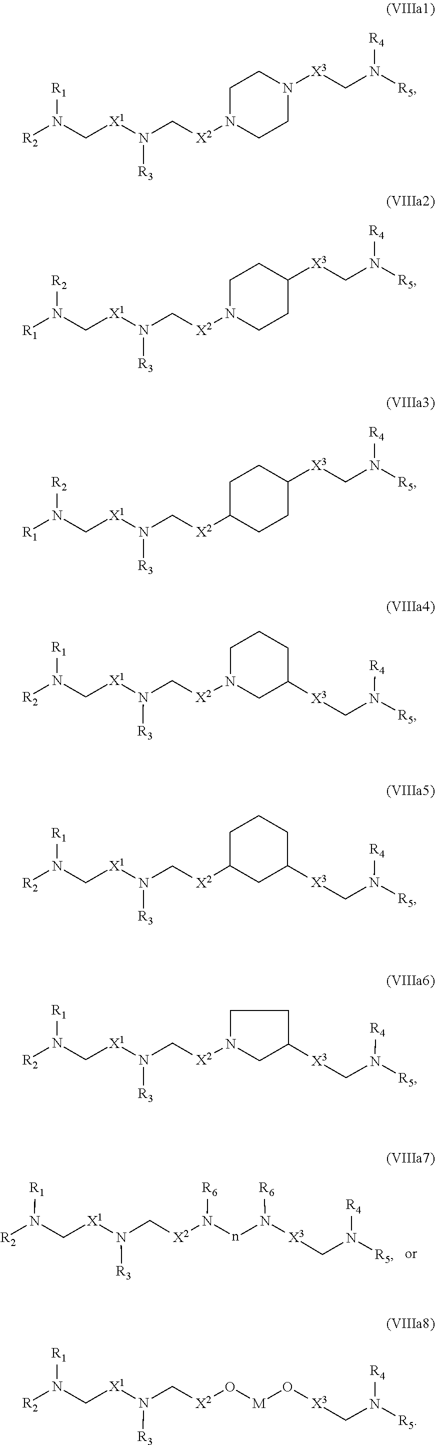

In some embodiments the ionizable amino lipid is

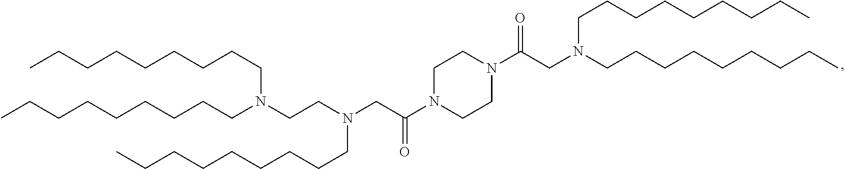 [SMILES: CCCCCCCCCN(CCCCCCCCC)CCN(CCCCCCCCC)CC(=O)N1CCN(C(=O)CN(CCCCCCCCC)CCCCCCCCC)CC1]

or a salt thereof.

The central amine moiety of a lipid according to Formula (VIII), (VIIIa1), (VIIIa2), (VIIIa3), (VIIIa4), (VIIIa5), (VIIIa6), (VIIIa7), or (VIIIa8) may be protonated at a physiological pH. Thus, a lipid may have a positive or partial positive charge at physiological pH.

In some embodiments, a lipid nanoparticle comprises a lipid having the structure:

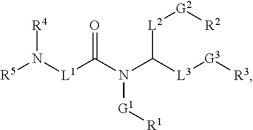

or a pharmaceutically acceptable salt, tautomer, or stereoisomer thereof, wherein:
R¹ is optionally substituted C₁-C₂₄ alkyl or optionally substituted C₂-C₂₄ alkenyl;
R² and R³ are each independently optionally substituted C₁-C₃₆ alkyl;
R⁴ and R⁵ are each independently optionally substituted C₁-C₆ alkyl, or R⁴ and R⁵ join, along with the N to which they are attached, to form a heterocyclyl or heteroaryl;
L¹, L², and L³ are each independently optionally substituted C₁-C₁₈ alkylene;
G¹ is a direct bond, —(CH₂)ₙO(C═O)—, —(CH₂)ₙ(C═O)O—, or —(C═O)—;
G² and G³ are each independently —(C═O)O— or —O(C═O)—; and n is an integer greater than 0.

In some embodiments, a lipid nanoparticle comprises a lipid having the structure:

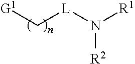

or a pharmaceutically acceptable salt, tautomer, or stereoisomer thereof, wherein:
G¹ is —N(R³)R⁴ or —OR⁵;
R¹ is optionally substituted branched, saturated or unsaturated C₁₂-C₃₆ alkyl;
R² is optionally substituted branched or unbranched, saturated or unsaturated C₁₂-C₃₆ alkyl when L is —C(═O)—; or R² is optionally substituted branched or unbranched, saturated or unsaturated C₄-C₃₆ alkyl when L is C₆-C₁₂ alkylene, C₆-C₁₂ alkenylene, or C₂-C₆ alkynylene;
R³ and R⁴ are each independently H, optionally substituted branched or unbranched, saturated or unsaturated C₁-C₆ alkyl; or R³ and R⁴ are each independently optionally substituted branched or unbranched, saturated or unsaturated C₁-C₆ alkyl when L is C₆-C₁₂ alkylene, C₆-C₁₂ alkenylene, or C₂-C₆ alkynylene; or R³ and R⁴, together with the nitrogen to which they are attached, join to form a heterocyclyl;
R⁵ is H or optionally substituted C₁-C₆ alkyl;
L is —C(═O)—, C₆-C₁₂ alkylene, C₆-C₁₂ alkenylene, or C₂-C₆ alkynylene; and n is an integer from 1 to 12.

In some embodiments, a lipid nanoparticle comprises a lipid having the structure:

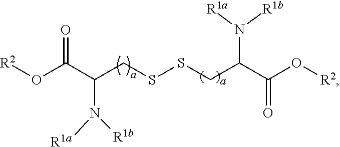

or a pharmaceutically acceptable salt thereof, wherein:
each R^{1a} is independently hydrogen, R^{1c}, or R^{1d};
each R^{1b} is independently R^{1c} or R^{1d};
each R^{1c} is independently —[CH₂]₂C(O)X¹R³;
each R^{1d} Is independently —C(O)R⁴;
each R² is independently —[C(R^{2a})₂]_{c}R^{2b};
each R^{2a} is independently hydrogen or C₁-C₆ alkyl;
R^{2b} is —N(L₁-B)₂; —(OCH₂CH₂)₆OH; or —(OCH₂CH₂)_{b}OCH₃;
each R³ and R⁴ is independently C₆-C₃₀ aliphatic;
each L₃ is independently C₁-C₁₀ alkylene;
each B is independently hydrogen or an ionizable nitrogen-containing group;
each X¹ is independently a covalent bond or O;
each a is independently an integer of 1-10;
each b is independently an integer of 1-10; and
each c is independently an integer of 1-10.

In some embodiments, a lipid nanoparticle comprises a lipid having the structure:

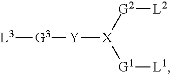

or a pharmaceutically acceptable salt, prodrug or stereoisomer thereof, wherein:
X is N, and Y is absent; or X is CR, and Y is NR;
L¹ is —O(C—O)R¹, —(C═O)OR¹, —C(═O)R¹, —OR¹, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c}, or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²
L³ is —O(C═O)R³ or —(C═O)OR³;
G¹ and G² are each independently C₂-C₁₂ alkylene or C₂-C₁₂ alkenylene;
G³ is C₁-C₂₄ alkylene, C₂-C₂₄ alkenylene, C₁-C₂₄ heteroalkylene or C₂-C₂₄ heteroalkenylene when X is CR, and Y is NR; and G³ is C₁-C₂₄ heteroalkylene or C₂-C₂₄ heteroalkenylene when X is N, and Y is absent;
R^{a}, R^{b}, R^{d} and R^{e} are each independently H or C₁-C₁₂ alkyl or C₁-C₁₂ alkenyl;
R^{c} and R^{f} are each independently C₁-C₁₂ alkyl or C₂-C₁₂ alkenyl;
each R is independently H or C₁-C₁₂ alkyl;
R¹, R² and R³ are each independently C₁-C₂₄ alkyl or C₂-C₂₄ alkenyl; and x is 0, 1 or 2, and
wherein each alkyl, alkenyl, alkylene, alkenylene, heteroalkylene and heteroalkenylene is independently substituted or unsubstituted unless otherwise specified.

In some embodiments, a lipid nanoparticle comprises a lipid having the structure:

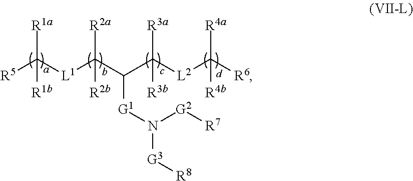

or a pharmaceutically acceptable salt, tautomer, prodrug or stereoisomer thereof, wherein:
L¹ and L² are each independently —O(C═O)—, —(C═O)O—, —C(═O)—, —O—, —S(O)x-, —S—S—, —C(═O)S—, —SC(═O)—, —NR^{a}C(═O)—, —C(═O)NR^{a}—, —NR^{a}C(═O)NR^{a}—, —OC(═O)NR^{a}—, —NR^{a}C(═O)O— or a direct bond;
G¹ is C₁-C₂ alkylene, —(C═O)—, —O(C═O)—, —SC(═O)—, —NR^{a}C(═O)— or a direct bond;
G² is —C(O)—, —(CO)O—, —C(═O)S—, —C(═O)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is H or C₁-C₂₀ alkyl;
R⁸ is OH, —N(R⁹)(C═O)R¹⁰, —(C═O)NR⁹R¹⁰, —NR⁹R¹⁰, —(C═O)OR″ or —O(C═O)R″, provided that G³ is C₄-C₆ alkylene when R′ is —NR⁹R¹⁰, R⁹ and R¹⁰ are each independently H or C₁-C₁₂ alkyl;
R″ is aralkyl;
a, b, c and d are each independently an integer from 1 to 24; and x is 0, 1 or 2,
wherein each alkyl, alkylene and aralkyl is optionally substituted.

In some embodiments, a lipid nanoparticle comprises a lipid having the structure:

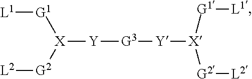

or a pharmaceutically acceptable salt, prodrug or stereoisomer thereof, wherein:
X and X′ are each independently N or CR;
Y and Y are each independently absent, —O(C═O)—, —(C═O)O— or NR, provided that:
   a) Y is absent when X is N;
   b) Y is absent when X is N;
   c) Y is —O(C═O)—, —(C═O)O— or NR when X is CR; and
   d) Y′ is —O(C═O)—, —(C═O)O— or NR when X′ is CR,
L¹ and L¹′ are each independently —O(C═O)R′, —(C═O)OR′, —C(═O)R′, —OR¹, —S(O)_{z}R′, —S—SR¹, —C(═O)SR′, —SC(═O)R′, —NR^{a}C(═O)R′, —C(═O)NR^{b}R^{c}, —NR^{a}C(═O)NR^{b}R^{c}, —OC(═O)NR^{b}R^{c} or —NR^{a}C(═O)OR′;
L² and L²′ are each independently —O(C═O)R², —(C═O)OR², —C(═O)R², —OR², —S(O)_{z}R², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}, —NR^{d}C(═O)OR² or a direct bond to R²
G¹. G¹′, G² and G²′ are each independently C₂-C₁₂ alkylene or C₂-C₁₂ alkenylene;
G is C₂-C₂₄ heteroalkylene or C₂-C₂₄ heteroalkenylene;
R^{a}, R^{b}, R^{d} and R^{e} are, at each occurrence, independently H, C₁-C₁₂ alkyl or C₂-C₁₂ alkenyl;
R^{c} and R^{f} are, at each occurrence, independently C₁-C₁₂ alkyl or C₂-C₁₂ alkenyl;
R is, at each occurrence, independently H or C₁-C₁₂ alkyl;
R¹ and R² are, at each occurrence, independently branched C₆-C₂₄ alkyl or branched C₆-C₂₄ alkenyl;
z is 0, 1 or 2, and wherein each alkyl, alkenyl, alkylene, alkenylene, heteroalkylene and heteroalkenylene is independently substituted or unsubstituted unless otherwise specified.

In some embodiments, a lipid nanoparticle comprises a lipid having the structure:

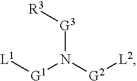

or a pharmaceutically acceptable salt, prodrug or stereoisomer thereof, wherein:
L¹ is —O(C═O)R¹, —(C═O)OR¹, —C(═O)R¹, —OR′, —S(O)ₓR¹, —S—SR¹, —C(═O)SR¹, —SC(═O)R′, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
G¹ and G² are each independently C₂-C₁₂ alkylene or C₂-C₁₂ alkenylene;
G³ is C₁-C₂₄ alkylene, C₂-C₂₄ alkenylene, C₃-C₈ cycloalkylene or C₃-C₈ cycloalkenylene;
R^{a}, R^{b}, R^{d} and R^{e} are each independently H or C₁-C₁₂ alkyl or C₁-C₁₂ alkenyl;
R^{c} and R^{f} are each independently C₁-C₁₂ alkyl or C₂-C₁₂ alkenyl;
R¹ and R² are each independently branched C₆-C₂₄ alkyl or branched C₆-C₂₄ alkenyl;
R³ is —N(R⁴)R⁵;
R⁴ is C₁-C₁₂ alkyl;
R⁵ is substituted C₁-C₁₂ alkyl; and
x is 0, 1 or 2, and
wherein each alkyl, alkenyl, alkylene, alkenylene, cycloalkylene, cycloalkenylene, aryl and aralkyl is independently substituted or unsubstituted unless otherwise specified.

In some embodiments, a lipid nanoparticle comprises a lipid having the structure:

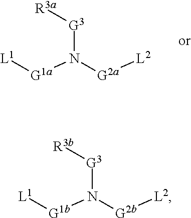

or a pharmaceutically acceptable salt, prodrug or stereoisomer thereof, wherein:
L¹ is —O(C═O)R¹, —(C═O)OR¹, —C(═O)R¹, —OR′, —S(O)ₓR¹, —S—SR¹, —C(═O)SR¹, —SC(═O)R¹, —NR^{a}C(═O)R¹, —C(═O)NR^{b}R^{c}, —NR^{a}C(═O)NR^{b}R^{c}, —OC(═O)NR^{b}R^{c} or —NR^{a}C(═O)OR¹;
L² is —O(C═O)R², —(C═O)OR², —C(═O)R², —OR², —S(O)ₓR², —S—SR², —C(═O)SR², —SC(═O)R², —NR^{d}C(═O)R², —C(═O)NR^{e}R^{f}, —NR^{d}C(═O)NR^{e}R^{f}, —OC(═O)NR^{e}R^{f}; —NR^{d}C(═O)OR² or a direct bond to R²;
G^{1a} and G^{2b} are each independently C₂-C₁₂ alkylene or C₂-C₁₂ alkenylene;
G^{1b} and G^{2b} are each independently C₁-C₁₂ alkylene or C₂-C₁₂ alkenylene;
G³ is C₁-C₂₄ alkylene, C₂-C₂₄ alkenylene, C₃-C₈ cycloalkylene or C₃-C₈ cycloalkenylene;
R^{a}, R^{b}, R^{d} and R^{e} are each independently H or C₁-C₁₂ alkyl or C₂-C₁₂ alkenyl;
R^{c} and R^{f} are each independently C₁-C₁₂ alkyl or C₂-C₁₂ alkenyl;
R¹ and R² are each independently branched C₆-C₂₄ alkyl or branched C₆-C₂₄ alkenyl;
R^{3a} is —C(═O)N(R^{4a})R^{5a} or —C(═O)OR⁶;
R^{3b} is —NR^{4b}C(═O)R^{5b};
R^{4a} is C₁-C₁₂ alkyl;
R^{4b} is H, C₁-C₁₂ alkyl or C₂-C₁₂ alkenyl;
R^{5a} is H, C₁-C₈alkyl or C₂-C₅ alkenyl;
R^{5b} is C₂-C₁₂ alkyl or C₂-C₁₂ alkenyl when R^{4b} is H; or R^{5b} is C₁-C₁₂ alkyl or C₂-C₁₂ alkenyl when R^{4b} is C₁-C₁₂ alkyl or C₂-C₁₂ alkenyl;
R⁶ is H, aryl or aralkyl; and
x is 0, 1 or 2, and
wherein each alkyl, alkenyl, alkylene, alkenylene, cycloalkylene, cycloalkenylene, aryl and aralkyl is independently substituted or unsubstituted.

In some embodiments, a lipid nanoparticle comprises a lipid having the structure:

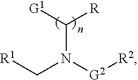

or a pharmaceutically acceptable salt, prodrug or stereoisomer thereof, wherein:
G¹ is —OH, —R³R⁴, —(C═O)R⁵ or —R³(C═O)R₈.
G² is —CH₂— or —(C═O)—;
R is, at each occurrence, independently H or OH;
R¹ and R² are each independently optionally substituted branched, saturated or unsaturated C₁₂-C₃₆ alkyl;
R³ and R⁴ are each independently H or optionally substituted straight or branched, saturated or unsaturated C₁-C₆ alkyl;
R⁵ is optionally substituted straight or branched, saturated or unsaturated C₁-C₆ alkyl; and
n is an integer from 2 to 6.

In some embodiments, a lipid nanoparticle comprises a lipid having the structure:

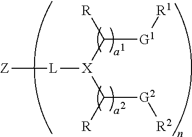

or a pharmaceutically acceptable salt, prodrug or stereoisomer thereof, wherein:
one of G¹ or G² is, at each occurrence, —O(C═O)—, —(C═O)O—, —C(═O)—, —O—, —S(O), —S—S—, —C(═O)S—, SC(═O)—, —N(R^{a})C(═O)—, —C(═O)N(R^{a})—, —N(R^{a})C(═O)N(R^{a})—, —OC(═O)N(R^{a})— or —N(R^{a})C(═O)O—, and the other of G¹ or G² is, at each occurrence, —O(C═O)—, —(C═O)O—, —C(═O)—, —O—, —S(O), —S—S—, —C(═O)S—, —SC(═O)—, —N(R^{a})C(═O)—, —C(═O)N(R^{a})—, —N(R^{a})C(═O)N(R^{a})—, —OC(═O)N(R^{a})— or —N(R^{a})C(═O)O— or a direct bond;
L is, at each occurrence, ~O(C═O)—, wherein ~ represents a covalent bond to X;
X is CR^{a};
Z is alkyl, cycloalkyl or a monovalent moiety comprising at least one polar functional group when n is 1; or Z is alkylene, cycloalkylene or a polyvalent moiety comprising at least one polar functional group when n is greater than 1;
R^{a} is, at each occurrence, independently H, C₁-C₁₂ alkyl, C₁-C₁₂ hydroxylalkyl, C₁-C₁₂ aminoalkyl, C₁-C₁₂ alkylaminylalkyl, C₁-C₁₂ alkoxyalkyl, C₁-C₁₂ alkoxycarbonyl, C₁-C₁₂ alkylcarbonyloxy, C₁-C₁₂ alkylcarbonyloxyalkyl or C₁-C₁₂ alkylcarbonyl;
R is, at each occurrence, independently either: (a) H or C₁-C₁₂ alkyl; or (b) R together with the carbon atom to which it is bound is taken together with an adjacent R and the carbon atom to which it is bound to form a carbon-carbon double bond;
R¹ and R² have, at each occurrence, the following structure, respectively:

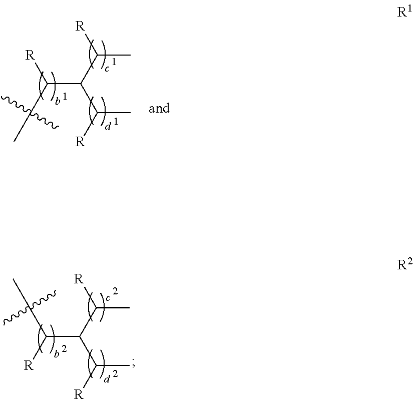

a¹ and a² are, at each occurrence, independently an integer from 3 to 12; b¹ and b² are, at each occurrence, independently 0 or 1; c¹ and c² are, at each occurrence, independently an integer from 5 to 10; d¹ and d² are, at each occurrence, independently an integer from 5 to 10; y is, at each occurrence, independently an integer from 0 to 2; and n is an integer from 1 to 6.
wherein each alkyl, alkylene, hydroxylalkyl, aminoalkyl, alkylaminylalkyl, alkoxyalkyl, alkoxycarbonyl, alkylcarbonyloxy, alkylcarbonyloxyalkyl and alkylcarbonyl is optionally substituted with one or more substituent.

In some embodiments, a lipid nanoparticle comprises a lipid having the structure:

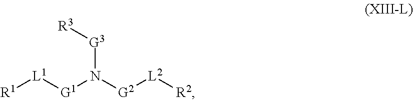

or a pharmaceutically acceptable salt, prodrug or stereoisomer thereof, wherein:
one of L¹ or L² is —O(C═O)—, —(C═O)O—, —C(═O)—, —O—, —S(O)ₓ—, —S—S—, —C(═O)S—, —SC(═O)—, —R^{a}C(═O)—, —C(═O)R^{a}—, R^{a}C(═O)R^{a}—, —OC(═O)R^{a}— or —R^{a}C(═O)O—, and the other of L¹ or L² is —O(C═O)—, —(C═O)O—, —C(═O)—, —O—, —S(O)ₓ—, —S—S—, —C(═O)S—, SC(═O)—, —R^{a}C(═O)—, —C(═O)R^{a}—, R^{a}C(═O)R^{a}—, —OC(═O)R^{a}— or —NR^{a}C(═O)O— or a direct bond;
G¹ and G² are each independently unsubstituted C₁-C₁₂ alkylene or C₁-C₁₂ alkenylene;
G³ is C₁-C₂₄ alkylene, C₁-C₂₄ alkenylene, C₃-C₈ cycloalkylene, C₃-C₈ cycloalkenylene;
R^{a} is H or C₁-C₁₂ alkyl;
R¹ and R² are each independently C₆-C₂₄ alkyl or C₆-C₂₄ alkenyl;
R³ is H, OR⁵, CN, —C(═O)OR⁴, —OC(═O)R⁴ or —R⁵C(═O)R⁴.
R⁴ is C₁-C₁₂ alkyl;
R⁵ is H or C₁-C₆ alkyl; and
x is 0, 1 or 2.

In some embodiments, a lipid nanoparticle comprises a lipid having the structure:

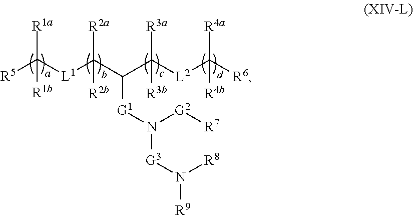

or a pharmaceutically acceptable salt, tautomer, prodrug or stereoisomer thereof, wherein:
L¹ and L² are each independently —O(C═O)—, —(C═O)O—, —C(═O)—, —O—, —S(O)ₓ—, —S—S—, —C(═O)S—, —SC(═O)—, —R^{a}C(═O)—, —C(═O)R^{a}—, —R^{a}C(═O)R^{a}—, —OC(═O)R^{a}—, —R^{a}C(═O)O— or a direct bond;
G¹ is C₁-C₂ alkylene, —(C═O)—, —O(C═O)—, —SC(═O)—, —R^{a}C(═O)— or a direct bond;
G² is —C(═O)—, —(C═O)O—, —C(═O)S—, —C(═O)NR^{a}— or a direct bond;
G³ is C₁-C₆ alkylene;
R^{a} is H or C₁-C₁₂ alkyl;
R^{1a} and R^{1b} are, at each occurrence, independently either: (a) H or C₁-C₁₂ alkyl; or (b)
R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond;
R^{4a} and R^{4b} are, at each occurrence, independently either: (a) H or C₁-C₁₂ alkyl; or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently H or methyl;
R⁷ is C₄-C₂₀ alkyl;
R⁸ and R⁹ are each independently C₁-C₁₂ alkyl; or R⁸ and R⁹, together with the nitrogen atom to which they are attached, form a 5, 6 or 7-membered heterocyclic ring;
a, b, c and d are each independently an integer from 1 to 24; and x is 0, 1 or 2.

In some embodiments, a lipid nanoparticle comprises a lipid having the structure:

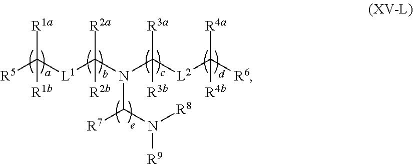

or a pharmaceutically acceptable salt, tautomer, prodrug or stereoisomer thereof, wherein:
L¹ and L² are each independently —O(C═O)—, —(C═O)O— or a carbon-carbon double bond;
R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently methyl or cycloalkyl;
R⁷ is, at each occurrence, independently H or C₁-C₁₂ alkyl; R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom;
a and d are each independently an integer from 0 to 24; b and c are each independently an integer from 1 to 24; and e is 1 or 2, provided that:
at least one of R¹, R^{2a}, R^{3a} or R^{4a} is C₁-C₁₂ alkyl, or at least one of L¹ or L² is —O(C═O)— or —(C═O)O—; and
R^{1a} and R^{1b} are not isopropyl when a is 6 or n-butyl when a is 8.

In some embodiments, a lipid nanoparticle comprises a lipid having the structure:

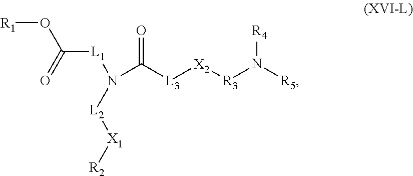

or a pharmaceutically acceptable salt thereof, wherein
R₁ and R₂ are the same or different, each a linear or branched alkyl with 1-9 carbons, or as alkenyl or alkynyl with 2 to 11 carbon atoms,
L₁ and L₂ are the same or different, each a linear alkyl having 5 to 18 carbon atoms, or form a heterocycle with N,
X₁ is a bond, or is —CG-G- whereby L2-CO—O—R₂ is formed,
X₂ is S or O,
L₃ is a bond or a lower alkyl, or form a heterocycle with N,
R₃ is a lower alkyl, and
R₄ and R₅ are the same or different, each a lower alkyl.

In some embodiments, a lipid nanoparticle comprises an ionizable lipid having the structure:

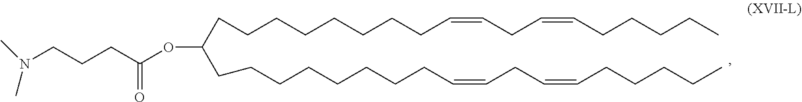 [SMILES: CCCCC/C=C\C/C=C\CCCCCCCCC(CCCCCCCC/C=C\C/C=C\CCCCC)OC(=O)CCCN(C)C]

or a pharmaceutically acceptable salt thereof.

In some embodiments, a lipid nanoparticle comprises a lipid having the structure:

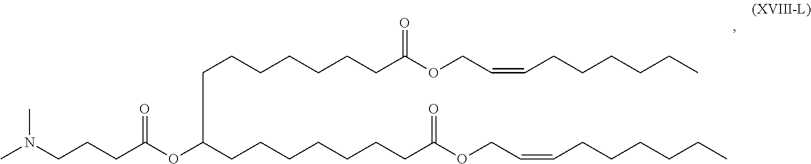 [SMILES: CCCCCC/C=C\COC(=O)CCCCCCCC(CCCCCCCC(=O)OC/C=C\CCCCCC)OC(=O)CCCN(C)C]

(XVIII-L), or a pharmaceutically acceptable salt thereof.

In some embodiments, a lipid nanoparticle comprises a lipid having the structure:

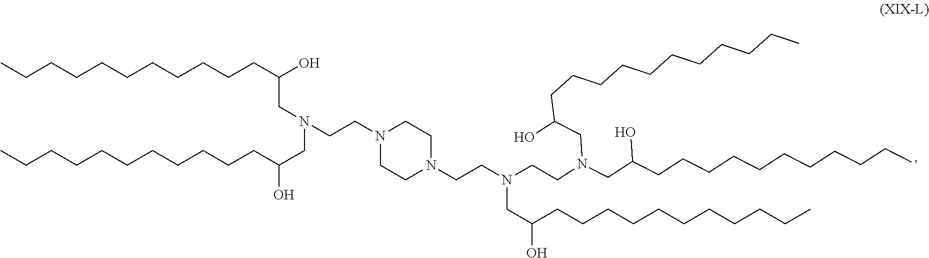 [SMILES: CCCCCCCCCCCC(O)CN(CCN1CCN(CCN(CC(O)CCCCCCCCCCC)CC(O)CCCCCCCCCCC)CC1)CCN(CC(O)CCCCCCCCCCC)CC(O)CCCCCCCCCCC]

or a pharmaceutically acceptable salt thereof.

In some embodiments, a lipid nanoparticle comprises a lipid having the structure:

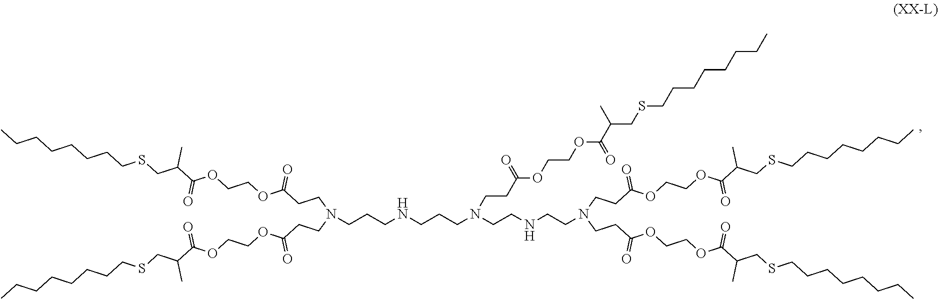 [SMILES: CCCCCCCCSCC(C)C(=O)OCCOC(=O)CCN(CCCNCCCN(CCC(=O)OCCOC(=O)C(C)CSCCCCCCCC)CCC(=O)OCCOC(=O)C(C)CSCCCCCCCC)CCNCCN(CCC(=O)OCCOC(=O)C(C)CSCCCCCCCC)CCC(=O)OCCOC(=O)C(C)CSCCCCCCCC]

or a pharmaceutically acceptable salt thereof.

In some embodiments, a lipid nanoparticle comprises a lipid having the structure:

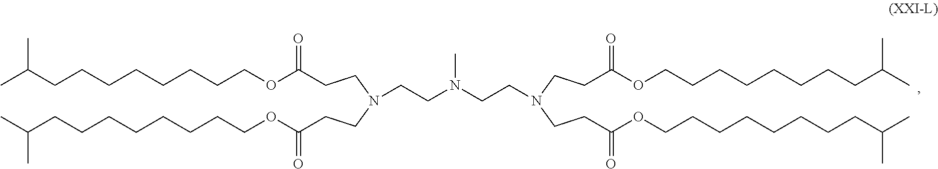 [SMILES: CC(C)CCCCCCCCOC(=O)CCN(CCC(=O)OCCCCCCCCC(C)C)CCN(C)CCN(CCC(=O)OCCCCCCCCC(C)C)CCC(=O)OCCCCCCCCC(C)C]

or a pharmaceutically acceptable salt thereof.

In some embodiments, a lipid nanoparticle comprises a lipid having the structure:

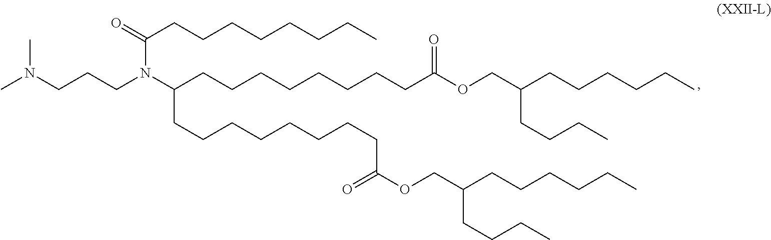 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

or a pharmaceutically acceptable salt thereof.

In some embodiments, a lipid nanoparticle comprises a lipid having the structure:

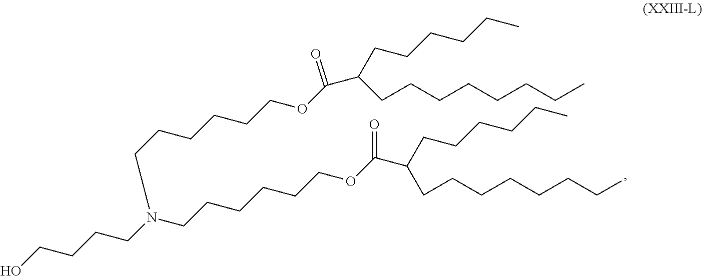

or a pharmaceutically acceptable salt thereof.

In some embodiments, a lipid nanoparticle comprises a lipid having the structure:

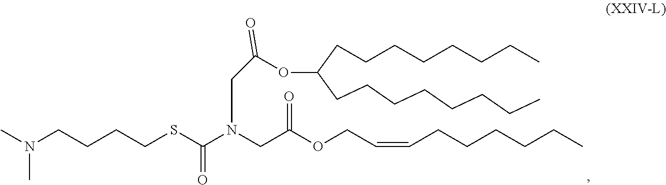 [SMILES: CCCCCC/C=C\COC(=O)CN(CC(=O)OC(CCCCCCCC)CCCCCCCC)C(=O)SCCCCN(C)C]

or a pharmaceutically acceptable salt thereof.

In some embodiments, a lipid nanoparticle comprises a lipid having the structure:

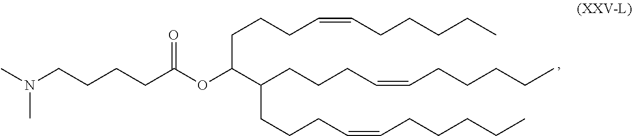 [SMILES: CCCCC/C=C\CCCC(CCC/C=C\CCCCC)C(CCC/C=C\CCCCC)OC(=O)CCCCN(C)C]

or a pharmaceutically acceptable salt thereof.

In some embodiments, a lipid nanoparticle comprises a lipid having the structure:

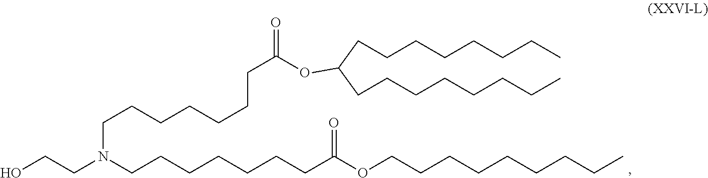 [SMILES: CCCCCCCCCOC(=O)CCCCCCCN(CCO)CCCCCCCC(=O)OC(CCCCCCCC)CCCCCCCC]

or a pharmaceutically acceptable salt thereof.

In some embodiments, a lipid nanoparticle comprises a lipid having the structure:

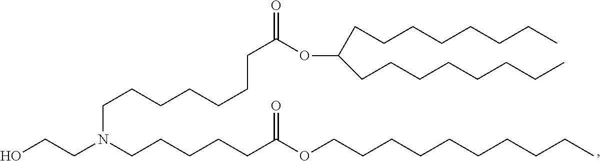 [SMILES: CCCCCCCCCCOC(=O)CCCCCN(CCO)CCCCCCCC(=O)OC(CCCCCCCC)CCCCCCCC]

or a pharmaceutically acceptable salt thereof.

In some embodiments, the ionizable lipid is a compound of Formula (IL*)

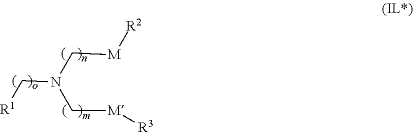

or a salt thereof, wherein:
R¹ is —OH, —NR^{N}—C₄₋₁₀ cycloalkenyl optionally substituted with one or more oxo or —N(R^{N}′R^{N}″);
R^{N} is H or C₁₋₆ alkyl;
R^{N}′ is H or C₁₋₆ alkyl;
R^{N}″ is H or C₁₋₆ alkyl;
o is 1, 2, 3, or 4;
n is 4, 5, 6, 7, or 8;
m is 4, 5, 6, 7, or 8;
M is —C(═O)—O—* or —O—C(═O)—*, wherein * indicates attachment to R²
M′ is —C(═O)—O—*or —O—C(═O)—*, wherein * indicates attachment to R³;
R² is

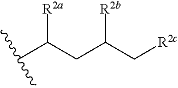

or —(C₁₋₆ alkylene)-(C₃₋₈ cycloalkyl)-C₁₋₆ alkyl;
R^{2a} is —H or C₁₋₁₀ alkyl;
R^{2b} is —H or C₁₋₁₀ alkyl;
R^{2c} is C₁₋₈ alkyl or C₂₋₈ alkenyl;
R³ is

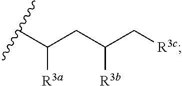

R^{3a} is H or C₁₋₁₀ alkyl;
R^{3b} is H or C₁₋₈ alkyl; and
R^{3c} is C₁₋₁₀ alkyl or C₂₋₈ alkenyl.

In some embodiments, the ionizable lipid is of Formula (IL**—I):

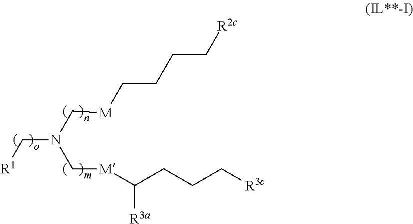

or a salt thereof, wherein:
R¹ is —OH;
o is 2, 3, or 4;
n is 4, 5, 6, 7, or 8;
M is —C(═O)—O—*, wherein * indicates attachment to R²
m is 6, 7, or 8;
M′ is —C(═O)—O—*, wherein * indicates attachment to R³;
R^{2c} is C₄₋₈ alkyl;
R^{3a} is C₇₋₁₀ alkyl; and
R^{3c} is C₃₋₅ alkyl.

In some embodiments, the ionizable lipid is of Formula (IL**—III):

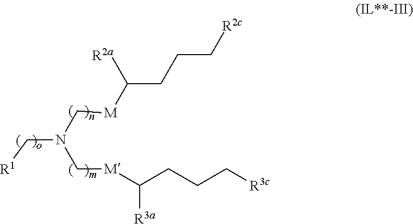

or a salt thereof, wherein:
R¹ is NR^{N}—C₄₋₁₀ cycloalkenyl optionally substituted with one or more oxo or —N(R^{N}′R^{N}″);
R^{N} is H;
R^{N}′ is C₁₋₂ alkyl;
R^{N}″ is H;
o is 2, 3, or 4;
n is 6, 7, or 8;
M is —C(═O)—O—*, wherein * indicates attachment to R²
m is 6, 7, or 8;
M′ is —C(═O)—O—*, wherein * indicates attachment to R³;
R^{2a} is C₇₋₁₀ alkyl;
R^{2c} is C₄₋₆ alkyl;
R^{3a} is C₁₋₃ alkyl; and
R^{3c} is C₄₋₆ alkyl.

In some embodiments, the ionizable lipid is of Formula (IL**—IV):

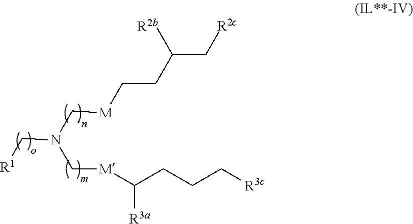

or a salt thereof, wherein:
R¹ is OH;
o is 2, 3, or 4;
n is 6, 7, or 8;
M is —C(═O)—O—*, wherein * indicates attachment to R²
m is 6, 7, or 8;
M′ is —C(═O)—O—*, wherein * indicates attachment to R³;
R^{2b} is C₃₋₅ alkyl;
R^{2c} is C₂₋₄ alkyl;
R^{3a} is C₇₋₁₀ alkyl; and
R^{3c} is C₄₋₆ alkyl.

In some embodiments, the ionizable lipid is of Formula (IL*—I):

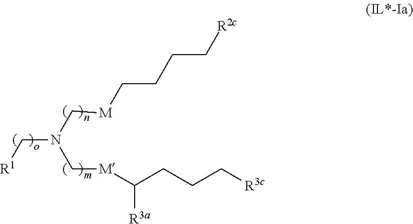

or a salt thereof, wherein:
R¹, o, m, n, M, M′, R^{2c}, and R^{3c} are as defined for variable IL*; and
R^{3a} is C₁₋₈ alkyl.

In some embodiments, ionizable lipid is of Formula (IL*-Ia):

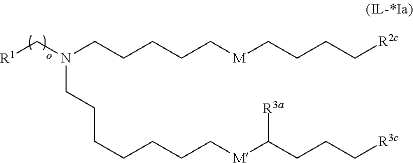

or a salt thereof, wherein:
R¹, o, m, n, M, M′, R^{2c}, and R^{3c} are as defined for Formula IL*; and
R^{3a} is C₁₋₈ alkyl.

In some embodiments, the ionizable lipid is of Formula (IL*-Ia′):

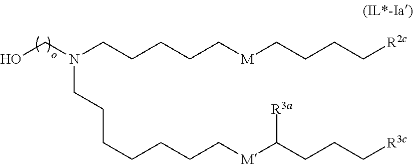

or a salt thereof, wherein:
o, M, M′, R^{2c} and R^{3c} are as defined for variable IL*; and
R^{3a} is C₁₋₈ alkyl.

In some embodiments, the ionizable lipid is of Formula (IL*-IIa):

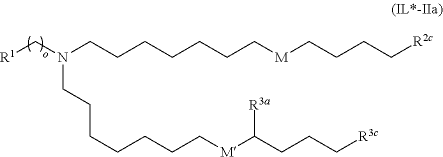

or a salt thereof, wherein:
R¹, o, m, n, M, M′, R^{2c}, and R^{3c} are as defined for Formula IL*; and
R^{3a} is C₁₋₈ alkyl.

In some embodiments, the ionizable lipid is of Formula (IL*-II′):

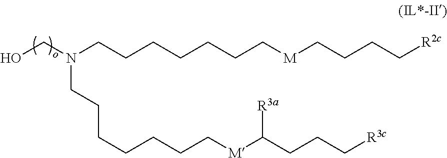

or a salt thereof, wherein:
o, M, M′, R^{2c} and R^{3c} are as defined for variable IL*; and
R^{3a} is C₁₋₈ alkyl.

In some embodiments, the ionizable lipid is of Formula (IL*—III):

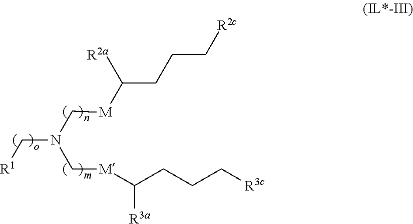

or a salt thereof, wherein:
R¹, o, m, n, M, M′, R^{2c}, and R^{3c} are as defined for variable IL*;
R^{2a} is a C₁₋₈ alkyl; and
R^{3a} is C₁₋₈ alkyl.

In some embodiments, the ionizable lipid is of Formula (IL*-IIIa):

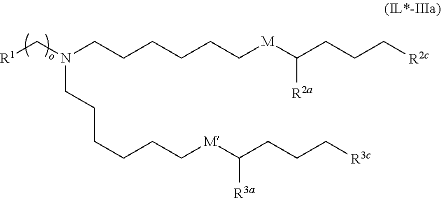

or a salt thereof, wherein:
R¹, o, m, n, M, M′, R^{2c}, and R^{3c} are as defined for variable IL*;
R^{2b} is a C₁₋₈ alkyl; and
R^{3a} is C₁₋₈ alkyl.

In some embodiments, the ionizable lipid is of Formula (IL*-IIIa):

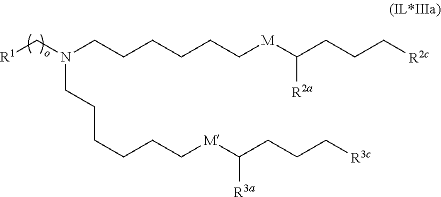

or a salt thereof, wherein:
R¹, o, M, M′, R^{2c}, and R^{3c} are as defined for variable IL*;
R₂ₐ is a C₁₋₈ alkyl; and
R^{3a} is C₁₋₈ alkyl.

In some embodiments, the ionizable lipid is of Formula (IL*-IIIa′):

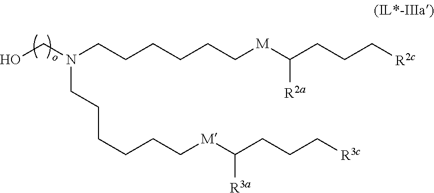

or a salt thereof, wherein:
R¹, o, M, M′, R^{2c}, and R^{3c} are as defined for variable IL*;
R^{2a} is a C₁₋₈ alkyl; and
R^{3a} is C₁₋₈ alkyl.

In some embodiments, the ionizable lipid is of Formula (IL*-IIIb):

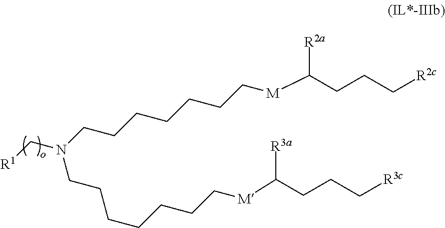

or a salt thereof, wherein:
R¹, o, M, M′, R^{2c}, and R^{3c} are as defined for variable IL*;
R^{2a} is a C₁₋₈ alkyl; and
R^{3a} is C₁₋₈ alkyl.

In some embodiments, the ionizable lipid is of Formula (IL*-IIIb′):

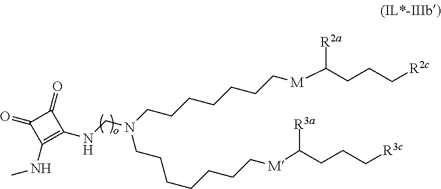

or a salt thereof, wherein:
R¹, o, M, M′, R^{2c}, and R^{3c} are as defined for variable IL*;
R^{2a} is a C₁₋₈ alkyl; and
R^{3a} is C₁₋₈ alkyl.

In some embodiments, the ionizable lipid is of Formula (IL*—IV):

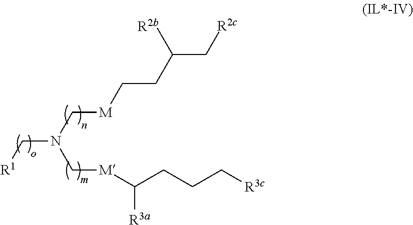

or a salt thereof, wherein:
R¹, o, m, n, M, M′, R^{2c}, and R^{3c} are as defined for variable IL*;
R^{2b} is a C₁₋₈ alkyl; and
R^{3a} is C₁₋₈ alkyl.

In some embodiments, the ionizable lipid is of Formula (IL*-IVa):

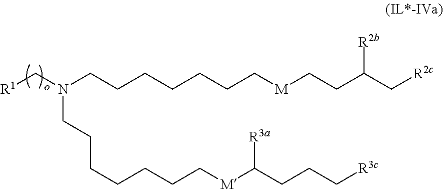

or a salt thereof, wherein:
R¹, o, m, n, M, M′, R^{2c}, and R^{3c} are as defined for variable IL*;
R^{2b} is a C₁₋₈ alkyl; and
R^{3a} is C₁₋₈ alkyl.

In some embodiments, the ionizable lipid is of Formula (IL*-Iva′):

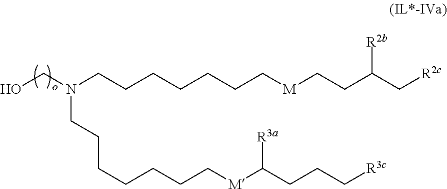

or a salt thereof, wherein:
o, M, M′, R^{2c}, and R^{3c} are as defined for variable IL*;
R^{2a} is a C₁₋₈ alkyl; and
R^{3a} is C₁₋₈ alkyl.
   Variables o, R′, R^{N}, R^{N}′, R^{N}″ of Ionizable Lipid

In some embodiments of the ionizable lipid, o is 1.

In some embodiments of the ionizable lipid, o is 2.

In some embodiments of the ionizable lipid, o is 3.

In some embodiments of the ionizable lipid, o is 4.

In some embodiments of the ionizable lipid, R¹ is —OH.

In some embodiments of the ionizable lipid, R^{N} is H.

In some embodiments of the ionizable lipid, R^{N} is methyl.

In some embodiments of the ionizable lipid, R^{N} is ethyl.

In some embodiments of the ionizable lipid, R¹ is —NR^{N}-cyclobutenyl, wherein the cyclobutenyl is optionally substituted with one or more oxo or —N(R^{N}′R^{N}″).

In some embodiments of the ionizable lipid, R^{N}′ is H.

In some embodiments of the ionizable lipid, R^{N}′ is methyl.

In some embodiments of the ionizable lipid, R^{N}′ is ethyl.

In some embodiments of the ionizable lipid, R^{N}″ is H.

In some embodiments of the ionizable lipid, R^{N}″ is methyl.

In some embodiments of the ionizable lipid, R^{N}″ is ethyl.

In some embodiments of the ionizable lipid, R^{N}′ is H and R^{N}″ is methyl.

In some embodiments of the ionizable lipid, R¹ is

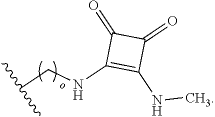

In some embodiments of the ionizable lipid, R¹ is

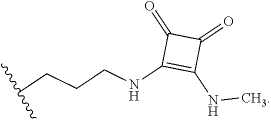 [SMILES: *CCCNc1c(NC)c(=O)c1=O]

### Variables m and n of the Ionizable Lipid

In some embodiments of the ionizable lipid, m is 4.

In some embodiments of the ionizable lipid, m is 5.

In some embodiments of the ionizable lipid, m is 6.

In some embodiments of the ionizable lipid, m is 7.

In some embodiments of the ionizable lipid, m is 8.

In some embodiments of the ionizable lipid, m is 4.

In some embodiments of the ionizable lipid, n is 5.

In some embodiments of the ionizable lipid, n is 6.

In some embodiments of the ionizable lipid, n is 7.

In some embodiments of the ionizable lipid, n is 8.

In some embodiments of the ionizable lipid, n is 5 and m is 7.

In some embodiments of the ionizable lipid, n is 7 and m is 7.

In some embodiments of the ionizable lipid, m is 6 and n is 6.

### Variables M and M′

In some embodiments of the ionizable lipid, M is —O—C(═O)—*, wherein * indicates attachment to R².

In some embodiments of the ionizable lipid, M is —C(═O)—O—* wherein * indicates attachment to R².

In some embodiments of the ionizable lipid, M′ is —O—C(═O)—*, wherein * indicates attachment to R³.

In some embodiments of the ionizable lipid, M′ is —C(═O)—O—* wherein * indicates attachment to R³.

In some embodiments of the ionizable lipid, M is —O—C(═O)—*, wherein * indicates attachment to R², and M′ is —C(═O)—O—* wherein * indicates attachment to R³.

Variables R², R^{2a}, R^{2b}, R^{2c}

In some embodiments of the ionizable lipid, R² is

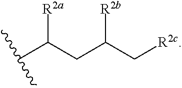

In some embodiments of the ionizable lipid, R^{2a} is hydrogen.

In some embodiments of the ionizable lipid, R^{2a} is methyl.

In some embodiments of the ionizable lipid, R^{2a} is ethyl.

In some embodiments of the ionizable lipid, R^{2a} is propyl.

In some embodiments of the ionizable lipid, R^{2a} is butyl.

In some embodiments of the ionizable lipid, R^{2a} is pentyl.

In some embodiments of the ionizable lipid, R^{2a} is hexyl.

In some embodiments of the ionizable lipid, R^{2a} is heptyl.

In some embodiments of the ionizable lipid, R^{2a} is octyl.

In some embodiments of the ionizable lipid, R^{2b} is hydrogen.

In some embodiments of the ionizable lipid, R^{2b} is methyl.

In some embodiments of the ionizable lipid, R^{2b} is ethyl.

In some embodiments of the ionizable lipid, R^{2b} is propyl.

In some embodiments of the ionizable lipid, R^{2b} is butyl.

In some embodiments of the ionizable lipid, R^{2b} is pentyl.

In some embodiments of the ionizable lipid, R^{2b} is hexyl.

In some embodiments of the ionizable lipid, R^{2b} is heptyl.

In some embodiments of the ionizable lipid, R^{2b} is octyl.

In some embodiments of the ionizable lipid, R^{2b} is hydrogen and R² is hydrogen.

In some embodiments of the ionizable lipid, R^{2a} is hexyl and R^{2b} is hydrogen.

In some embodiments of the ionizable lipid, R^{2a} is octyl and R^{2b} is hydrogen.

In some embodiments of the ionizable lipid, R^{2a} is hydrogen and R^{2b} is butyl.

In some embodiments of the ionizable lipid, R^{2c} is methyl.

In some embodiments of the ionizable lipid, R^{2c} is ethyl.

In some embodiments of the ionizable lipid, R^{2c} is propyl.

In some embodiments of the ionizable lipid, R^{2c} is butyl.

In some embodiments of the ionizable lipid, R^{2c} is pentyl.

In some embodiments of the ionizable lipid, R^{2c} is hexyl.

In some embodiments of the ionizable lipid, R^{2c} is heptyl.

In some embodiments of the ionizable lipid, R^{2c} is octyl.

In some embodiments of the ionizable lipid, R² is —(C₁₋₆ alkylene)-(C₃₋₈ cycloalkyl)-C₁₋₆ alkyl.

In some embodiments of the ionizable lipid, R² is —(C₁₋₆ alkylene)-(cyclohexyl)-C₁₋₆ alkyl.

In some embodiments of the ionizable lipid, R² is —(C₁₋₆ alkylene)-(cyclopentyl)-C₁₋₆ alkyl.

Variables R³, R^{3a}, R^{3b}, and R^{3c}

In some embodiments of the ionizable lipid, R³ is

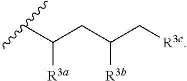

In some embodiments of the ionizable lipid, R^{3a} is hydrogen.

In some embodiments of the ionizable lipid, R^{3a} is methyl.

In some embodiments of the ionizable lipid, R^{3a} is ethyl.

In some embodiments of the ionizable lipid, R^{3a} is propyl.

In some embodiments of the ionizable lipid, R^{3a} is butyl.

In some embodiments of the ionizable lipid, R^{3a} is pentyl.

In some embodiments of the ionizable lipid, R^{3a} is hexyl.

In some embodiments of the ionizable lipid, R^{3a} is heptyl.

In some embodiments of the ionizable lipid, R^{3a} is octyl.

In some embodiments of the ionizable lipid, R^{3b} is hydrogen.

In some embodiments of the ionizable lipid, R^{3b} is methyl.

In some embodiments of the ionizable lipid, R^{3b} is ethyl.

In some embodiments of the ionizable lipid, R^{3b} is propyl.

In some embodiments of the ionizable lipid, R^{3b} is butyl.

In some embodiments of the ionizable lipid, R^{3b} is pentyl.

In some embodiments of the ionizable lipid, R^{3b} is hexyl.

In some embodiments of the ionizable lipid, R^{3b} is heptyl.

In some embodiments of the ionizable lipid, R^{3b} is octyl.

In some embodiments of the ionizable lipid, R^{3a} is octyl and R^{3b} is hydrogen.

In some embodiments of the ionizable lipid, R^{3a} is ethyl and R^{3b} is hydrogen.

In some embodiments of the ionizable lipid, R^{3a} is hexyl and R^{3b} is hydrogen.

In some embodiments of the ionizable lipid, R^{3c} is methyl.

In some embodiments of the ionizable lipid, R^{3c} is ethyl.

In some embodiments of the ionizable lipid, R^{3c} is propyl.

In some embodiments of the ionizable lipid, R^{3c} is butyl.

In some embodiments of the ionizable lipid, R^{3c} is pentyl.

In some embodiments of the ionizable lipid, R^{3c} is hexyl.

In some embodiments of the ionizable lipid, R^{3c} is heptyl.

In some embodiments of the ionizable lipid, R^{3c} is octyl.

It is understood that, for an ionizable lipid, variables o, R¹, R^{N}, R^{N}′, R^{N}′, m, n, M, M′, R², R^{2a}, R^{2b}, R^{2c}, R³, R^{3a}, R^{3b}, and R^{3c} can each be, where applicable, selected from the groups described herein, and any group described herein for any of variables o, R¹, R^{N}, R^{N}′, R^{N}′, m, n, M, M′, R², R^{2a}, R^{2b}, R^{2c}, R³, R^{3a}, R^{3b}, and R^{3c} can be combined, where applicable, with any group described herein for one or more of the remainder of variables o, R′, R^{N}, R^{N}′, R^{N}′, m, n, M, M′, R², R^{2a}, R^{2b}, R^{2c}, R³, R^{3a}, R^{3b}, and R^{3c}.

In some embodiments, the ionizable lipid is a compound selected from:

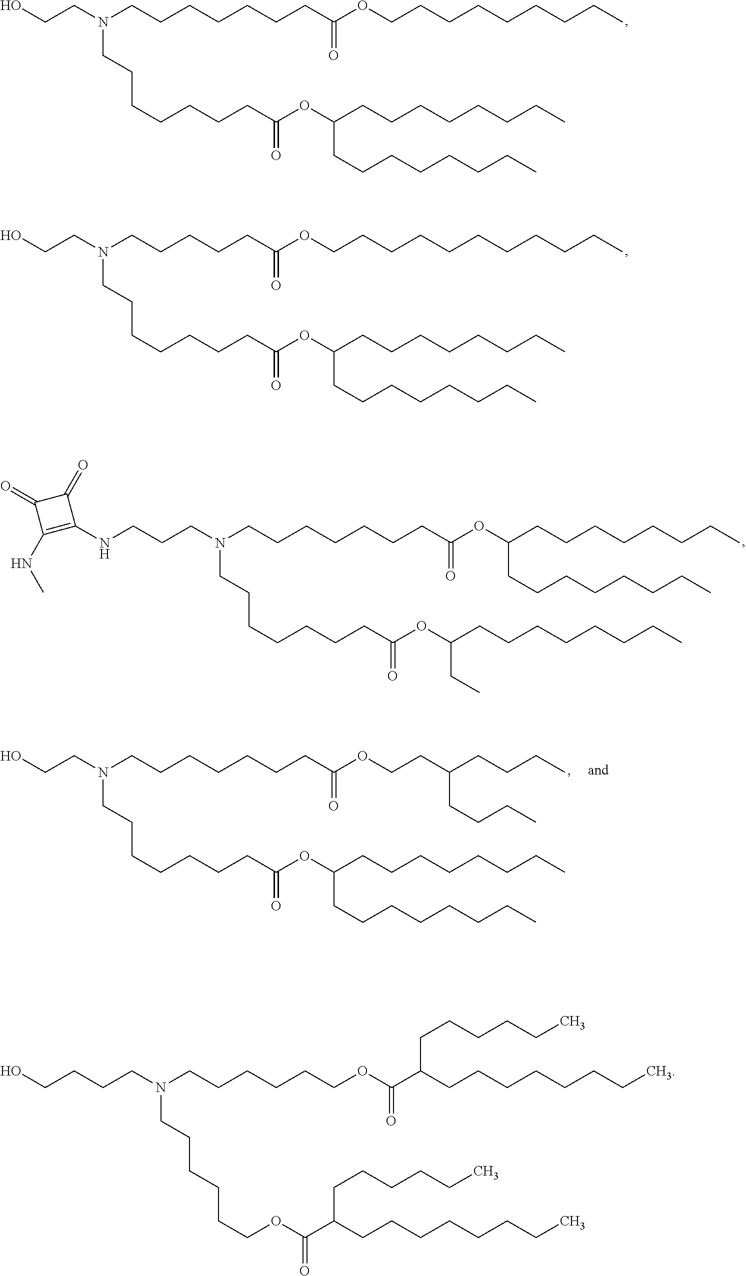 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC ; CCCCCCCCC(CC)OC(=O)CCCCCCCN(CCCCCCCC(=O)OC(CCCCCCCC)CCCCCCCC)CCCNc1c(NC)c(=O)c1=O ; CCCCCCCCC(CCCCCCCC)OC(=O)CCCCCCCN(CCO)CCCCCCCC(=O)OCCC(CCCC)CCCC ; CCCCCCCCC(CCCCCC)C(=O)OCCCCCCN(CCCCO)CCCCCCOC(=O)C(CCCCCC)CCCCCCCC]

In some embodiments, the ionizable lipid is

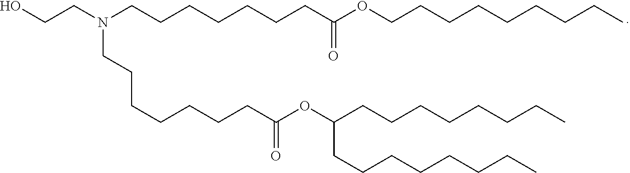 [SMILES: CCCCCCCCCOC(=O)CCCCCCCN(CCO)CCCCCCCC(=O)OC(CCCCCCCC)CCCCCCCC]

In some embodiments, the ionizable lipid is

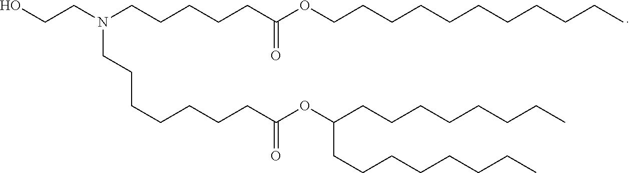 [SMILES: CCCCCCCCCCCOC(=O)CCCCCN(CCO)CCCCCCCC(=O)OC(CCCCCCCC)CCCCCCCC]

In some embodiments, the ionizable lipid is

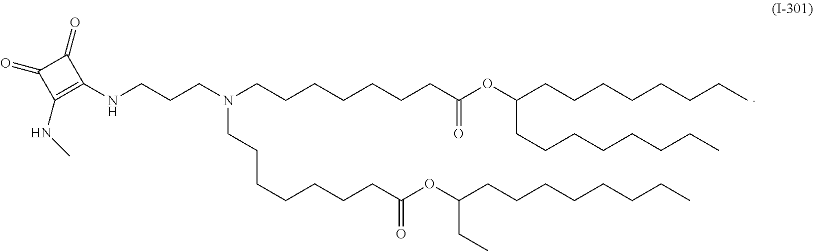 [SMILES: CCCCCCCCC(CC)OC(=O)CCCCCCCN(CCCCCCCC(=O)OC(CCCCCCCC)CCCCCCCC)CCCNc1c(NC)c(=O)c1=O]

In some embodiments, the ionizable lipid is

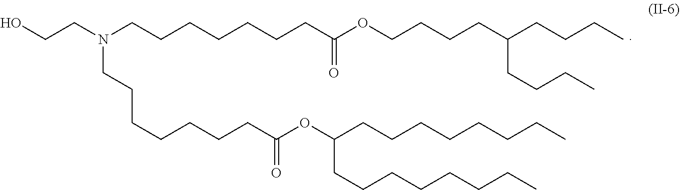 [SMILES: CCCCCCCCC(CCCCCCCC)OC(=O)CCCCCCCN(CCO)CCCCCCCC(=O)OCCCCC(CCCC)CCCC]

Without wishing to be bound by theory, it is understood that an ionizable lipid may have a positive or partial positive charge at physiological pH. Such lipids may be referred to as cationic or ionizable (amino)lipids. Lipids may also be zwitterionic, i.e., neutral molecules having both a positive and a negative charge.

mRNA-Lipid Adduct

It has been determined that certain ionizable lipids are susceptible to the formation of lipid-polynucleotide adducts. In particular, ionizable lipids that comprise a tertiary amine group may decompose into one or both of a secondary amine and a reactive aldehyde species capable of interacting with polynucleotides (such as mRNA) to form an ionizable lipid-polynucleotide adduct impurity that can be detected by reverse phase ion pair chromatography (RP—IP HPLC). For example, oxidation of the tertiary amine may lead to N-oxide formation that can undergo acid/base-catalyzed hydrolysis at the amine to generate aldehydes and secondary amines which may form adducts with mRNA. Thus, in some aspects, the ionizable lipid-polynucleotide adduct impurity is an aldehyde-mRNA adduct impurity.

It also has been determined that such adducts may disrupt mRNA translation and impact the activity of lipid nanoparticle (LNP) formulated mRNA products. Thus, it can be advantageous to prepare and use LNP compositions with a reduced content of ionizable lipid-polynucleotide adduct impurity, such as wherein less than about 20%, less than about 10%, less than about 5%, or less than about 1%, of the mRNA is in the form of ionizable lipid-polynucleotide adduct impurity, as may be measured by RP—IP HPLC. Thus, in accordance with some aspects, an LNP composition is provided wherein less than about 10%, less than about 5%, or less than about 1%, of the mRNA is in the form of ionizable lipid-polynucleotide adduct impurity, including less than 10%, less than 5%, or less than 1%, as may be measured by RP—IP HPLC.

In some aspects, an amount of lipid aldehydes in the composition is less than about 50 ppm, including less than 50 ppm. Additionally or alternatively, in some aspects an amount of N-oxide compounds in the composition is less than about 50 ppm, including less than 50 ppm. Additionally or alternatively, in some aspects an amount of transition metals, such as Fe, in the composition is less than about 50 ppm, including less than 50 ppm. Additionally or alternatively, in some aspects an amount of alkyl halide compounds in the composition is less than about 50 ppm, including less than 50 ppm. Additionally or alternatively, in some aspects an amount of anhydride compounds in the composition is less than about 50 ppm, including less than 50 ppm. Additionally or alternatively, in some aspects an amount of ketone compounds in the composition is less than about 50 ppm, including less than 50 ppm. Additionally or alternatively, in some aspects an amount of conjugated diene compounds in the composition is less than about 50 ppm, including less than 50 ppm.

In some aspects, the composition is stable against the formation of ionizable lipid-polynucleotide adduct impurity. In some aspects, an amount of ionizable lipid-polynucleotide adduct impurity in the composition increases at an average rate of less than about 2% per day when stored at a temperature of about 25° C. or below, including at an average rate of less than 2% per day. In some aspects, an amount of ionizable lipid-polynucleotide adduct impurity in the composition increases at an average rate of less than about 0.5% per day when stored at a temperature of about 5° C. or below, including at an average rate of less than 0.5% per day. In some aspects, an amount of ionizable lipid-polynucleotide adduct impurity in the composition increases at an average rate of less than about 0.5% per day when stored at a refrigerated temperature, optionally wherein the refrigerated temperature is about 5° C.

Lipid vehicle (e.g., LNP) compositions with a reduced content of ionizable lipid-polynucleotide adduct impurity can be prepared by methods that inhibit formation of one or both of N-oxides and aldehydes. Such methods may comprise treating a composition comprising an ionizable lipid comprising a tertiary amine group to inhibit formation of one or both of N-oxides and aldehydes, such as by treating the composition with a reducing agent; treating the composition with a chelating agent; adjusting the pH of the composition; adjusting the temperature of the composition; and adjusting the buffer in the composition. Such methods may comprise, prior to combining the ionizable lipid with a polynucleotide, one or more of treating the ionizable lipid with a scavenging agent; treating the ionizable lipid with a reductive treatment agent; treating the ionizable lipid with a reducing agent; treating the ionizable lipid with a chelating agent; treating the polynucleotide with a reducing agent; and treating the polynucleotide with a chelating agent.

In accordance with any of the foregoing, the scavenging agent, reductive treatment agent, and/or reducing agent may be an agent that reacts with aldehyde, ketone, anhydride and/or diene compounds. A scavenging agent may comprise one or more selected from (O-(2,3,4,5,6-Pentafluorobenzyl)hydroxylamine hydrochloride) (PFBHA), methoxyamine (e.g., methoxyamine hydrochloride), benzyloxyamine (e.g., benzyloxyamine hydrochloride), ethoxyamine (e.g., ethoxyamine hydrochloride), 4-[2-(aminooxy)ethyl]morpholine dihydrochloride, butoxyamine (e.g., tert-butoxyamine hydrochloride), 4-Dimethylaminopyridine (DMAP), 1,4-diazabicyclo[2.2.2]octane (DABCO), Triethylamine (TEA), Piperidine 4-carboxylate (BPPC), and combinations thereof. A reductive treatment agent may comprise a boron compound (e.g., sodium borohydride and/or bis(pinacolato)diboron). A reductive treatment agent may comprise a boron compound, such as one or both of sodium borohydride and bis(pinacolato)diboron). A chelating agent may comprise immobilized iminodiacetic acid. A reducing agent may comprise an immobilized reducing agent, such as immobilized diphenylphosphine on silica (Si-DPP), immobilized thiol on agarose (Ag-Thiol), immobilized cysteine on silica (Si-Cysteine), immobilized thiol on silica (Si-Thiol), or a combination thereof. A reducing agent may comprise a free reducing agent, such as potassium metabisulfite, sodium thioglycolate, tris(2-carboxyethyl)phosphine (TCEP), sodium thiosulfate, N-acetyl cysteine, glutathione, dithiothreitol (DTT), cystamine, dithioerythritol (DTE), dichlorodiphenyltrichloroethane (DDT), homocysteine, lipoic acid, or a combination thereof.

In accordance with any of the foregoing, the pH may be, or adjusted to be, a pH of from about 7 to about 9 (for example, about 7, about 7.5, about 8, about 8.5, or about 9). In accordance with any of the foregoing, a buffer may be selected from sodium phosphate, sodium citrate, sodium succinate, histidine, histidine-HCl, sodium malate, sodium carbonate, and TRIS (tris(hydroxymethyl)aminomethane). In accordance with any of the foregoing, a buffer may be TRIS and may be, or adjusted to be, from about 20 mM to about 150 mM TRIS.

In accordance with any of the foregoing, the temperature of the composition may be, or adjusted to be, 25° C. or less.

The composition may also comprise a free reducing agent or antioxidant.

### Non-Cationic Lipids

In certain embodiments, a lipid nanoparticle described herein comprise one or more non-cationic lipids. Non-cationic lipids may be phospholipids.

In some embodiments, a lipid nanoparticle comprises 5-25 mol % non-cationic lipid. For example, a lipid nanoparticle may comprise 5-20 mol %, 5-15 mol %, 5-10 mol %, 10-25 mol %, 10-20 mol %, 10-25 mol %, 15-25 mol %, 15-20 mol %, or 20-25 mol % non-cationic lipid. In some embodiments, a lipid nanoparticle comprises 5 mol %, 10 mol %, 15 mol %, 20 mol %, or 25 mol % non-cationic lipid.

In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

In some embodiments, a lipid nanoparticle comprises 5-15 mol %, 5-10 mol %, or 10-15 mol % DSPC. For example, a lipid nanoparticle may comprise 5, 6, 7, 8, 9, 10, 11, 12, 13, 14, or 15 mol % DSPC.

In certain embodiments, the lipid composition of a lipid nanoparticle composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., an mRNA) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidylglycerols, and phosphatidic acids. Phospholipids also include phosphosphingolipid, such as sphingomyelin.

In some embodiments, a phospholipid of the present disclosure comprises 1,2-distearoyl-sn-glycero-3-phosphocholine (DSPC), 1,2-Distearoyl-sn-glycero-3-phosphoethanolamine (DSPE), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, or mixtures thereof.

In certain embodiments, a phospholipid useful or potentially useful in the present disclosure is an analog or variant of DSPC. In certain embodiments, a phospholipid useful or potentially useful in the present disclosure is a compound of Formula (IX):

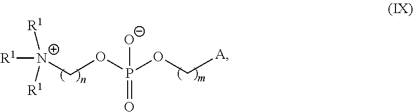

or a salt thereof, wherein:
each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;
A is of the formula:

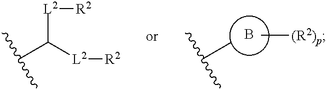

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), —NRNC(O)O, or NRNC(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2;
provided that the compound is not of the formula:

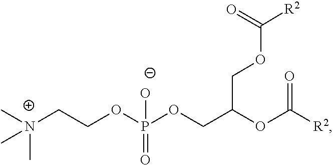

wherein each instance of R² is independently unsubstituted alkyl, unsubstituted alkenyl, or unsubstituted alkynyl.

In some embodiments, the phospholipids may be one or more of the phospholipids described in PCT Application No. PCT/US2018/037922.

In some embodiments, a lipid nanoparticle comprises 5-25 mol % non-cationic lipid relative to the other lipid components. For example, a lipid nanoparticle may comprise 5-30 mol %, 5-15 mol %, 5-10 mol %, 10-25 mol %, 10-20 mol %, 10-25 mol %, 15-25 mol %, 15-20 mol %, 20-25 mol %, or 25-30 mol % non-cationic lipid. In some embodiments, a lipid nanoparticle comprises a 5 mol %, 10 mol %, 15 mol %, 20 mol %, 25 mol %, or 30 mol % non-cationic lipid.

In some embodiments, a lipid nanoparticle comprises 5-25 mol % phospholipid relative to the other lipid components. For example, the lipid nanoparticle may comprise 5-30 mol %, 5-mol %, 5-10 mol %, 10-25 mol %, 10-20 mol %, 10-25 mol %, 15-25 mol %, 15-20 mol %, 20-25 mol %, or 25-30 mol % phospholipid. In some embodiments, the lipid nanoparticle 5 mol %, 10 mol %, 15 mol %, 20 mol %, 25 mol %, or 30 mol % phospholipid lipid.

### Structural Lipids

The lipid composition of a pharmaceutical composition disclosed herein can comprise one or more structural lipids. As used herein, the term “structural lipid” includes sterols and also to lipids containing sterol moieties.

Incorporation of structural lipids in a lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in U.S. application Ser. No. 16/493,814.

In some embodiments, a lipid nanoparticle comprises 25-55 mol % structural lipid relative to the other lipid components. For example, a lipid nanoparticle may comprise 10-55 mol %, 25-50 mol %, 25-45 mol %, 25-40 mol %, 25-35 mol %, 25-30 mol %, 30-55 mol %, 30-50 mol %, 30-45 mol %, 30-40 mol %, 30-35 mol %, 35-55 mol %, 35-50 mol %, 35-45 mol %, 35-40 mol %, 40-55 mol %, 40-50 mol %, 40-45 mol %, 45-55 mol %, 45-50 mol %, or 50-55 mol % structural lipid. In some embodiments, a lipid nanoparticle comprises 10 mol %, 15 mol %, 20 mol %, 25 mol %, 30 mol %, 35 mol %, 40 mol %, 45 mol %, 50 mol %, or 55 mol % structural lipid.

In some embodiments, a lipid nanoparticle comprises 30-45 mol % sterol, optionally 35-mol %, for example, 30-31 mol %, 31-32 mol %, 32-33 mol %, 33-34 mol %, 34-35 mol %, 35-36 mol %, 36-37 mol %, 37-38 mol %, 38-39 mol %, or 39-40 mol %. In some embodiments, a lipid nanoparticle comprises 25-55 mol % sterol. For example, a lipid nanoparticle may comprise 25-50 mol %, 25-45 mol %, 25-40 mol %, 25-35 mol %, 25-30 mol %, 30-55 mol %, 30-50 mol %, 30-45 mol %, 30-40 mol %, 30-35 mol %, 35-55 mol %, 35-50 mol %, 35-45 mol %, 35-40 mol %, 40-55 mol %, 40-50 mol %, 40-45 mol %, 45-55 mol %, 45-50 mol %, or 50-55 mol % sterol. In some embodiments, a lipid nanoparticle comprises 25 mol %, 30 mol %, 35 mol %, 40 mol %, 45 mol %, 50 mol %, or 55 mol % sterol.

In some embodiments, a lipid nanoparticle comprises 35-40 mol % cholesterol. For example, a lipid nanoparticle may comprise 35, 35.5, 36, 36.5, 37, 37.5, 38, 38.5, 39, 39.5, or 40 mol % cholesterol.

### Polyethylene Glycol (PEG)-Lipids

The lipid composition of a pharmaceutical composition disclosed herein can comprise one or more polyethylene glycol (PEG) lipids.

As used herein, the term “PEG-lipid” or “PEG-modified lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines, and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)](PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In some embodiments, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG, and/or PEG-DPG.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In some embodiments, the PEG-lipid is PEG₂ₖ-DMG.

In some embodiments, a lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

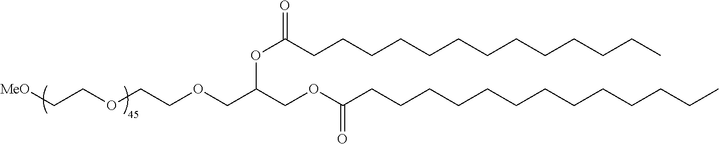

In some embodiments, PEG lipids useful in the present disclosure can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present disclosure.

In certain embodiments, a PEG lipid useful in the present disclosure is a compound of Formula (X):

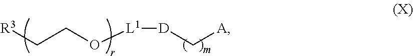

or salts thereof, wherein:
R³ is —OR^{O};
R^{O} is hydrogen, optionally substituted alkyl, or an oxygen protecting group;
r is an integer between 1 and 100, inclusive;
L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O), C(O)O, OC(O), OC(O)O, OC(O)N(R^{N}), NR^{N}C(O)O, or NR^{N}C(O)N(R^{N});
D is a moiety obtained by click chemistry or a moiety cleavable under physiological conditions;
m is 0, 1, 2, 3, 4, 5, 6, 7, 8, 9, or 10;
A is of the formula:

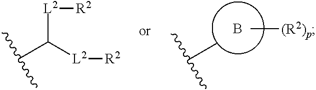

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), —NRNC(O)O, or NRNC(O)N(R^{N});
each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), —OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, —OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound of Formula (X) is a PEG-OH lipid (i.e., R³ is —OR^{O}, and R^{O} is hydrogen). In certain embodiments, the compound of Formula (X) is of Formula (X—OH):

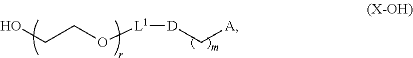

or a salt thereof.

In certain embodiments, a PEG lipid useful in the present disclosure is a PEGylated fatty acid. In certain embodiments, a PEG lipid useful in the present disclosure is a compound of Formula (XI). Provided herein are compounds of Formula (XI):

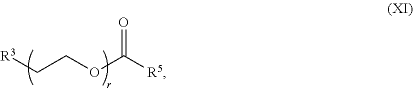

or a salts thereof, wherein:
R³ is —OR^{O};
R^{O} is hydrogen, optionally substituted alkyl or an oxygen protecting group;
r is an integer between 1 and 100, inclusive;
R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), —C(O)N(R^{N}), NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), —C(S)N(R^{N}), NR^{N}C(S), NR^{N}C(S)N(R^{N}), S(O), OS(O), S(OO, OS(O)O, OS(O)₂, S(O)₂O, —OS(O)₂O, N(R^{N})S(O), S(O)N(R^{N}), N(R^{N})S(O)N(R^{N}), OS(O)N(R^{N}), N(R^{N})S(O)O, S(O)₂, —N(R^{N})S(O)₂, S(O)₂N(R^{N}), N(R^{N})S(O)₂N(R^{N}), OS(O)₂N(R^{N}), or N(R^{N})S(O)₂O; and
each instance of R^{N} is independently hydrogen, optionally substituted alkyl, or a nitrogen protecting group.

In certain embodiments, the compound of Formula (XI) is of Formula (XI—OH):

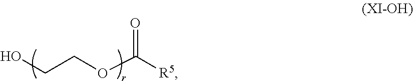

or a salt thereof. In some embodiments, r is 40-50.

In yet other embodiments the compound of Formula (XI) is:

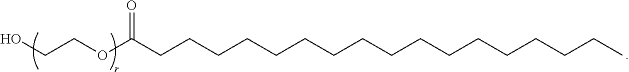

or a salt thereof.

In some embodiments, the compound of Formula (XI) is

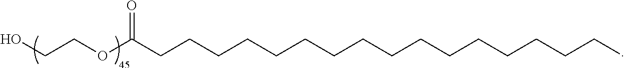

In some embodiments, the lipid composition of the pharmaceutical compositions disclosed herein does not comprise a PEG-lipid.

In some embodiments, the PEG-lipids may be one or more of the PEG lipids described in U.S. Application No. U.S. Ser. No. 15/674,872.

In some embodiments, a lipid nanoparticle comprises 0.5-15 mol % PEG lipid relative to the other lipid components. For example, a lipid nanoparticle may comprise 0.5-10 mol %, 0.5-5 mol %, 1-15 mol %, 1-10 mol %, 1-5 mol %, 2-15 mol %, 2-10 mol %, 2-5 mol %, 5-15 mol %, 5-10 mol %, or 10-15 mol % PEG lipid. In some embodiments, a lipid nanoparticle comprises 0.5 mol %, 1 mol %, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, or 15 mol % PEG-lipid.

In some embodiments, a lipid nanoparticle comprises 1-5% PEG-modified lipid, optionally 1-3 mol %, for example, 1.5 to 2.5 mol %, 1-2 mol %, 2-3 mol %, 3-4 mol %, or 4-5 mol %. In some embodiments, a lipid nanoparticle comprises 0.5-15 mol % PEG-modified lipid. For example, a lipid nanoparticle may comprise 0.5-10 mol %, 0.5-5 mol %, 1-15 mol %, 1-10 mol %, 1-5 mol %, 2-15 mol %, 2-10 mol %, 2-5 mol %, 5-15 mol %, 5-10 mol %, or 10-15 mol %. In some embodiments, a lipid nanoparticle comprises 0.5 mol %, 1 mol %, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, or 15 mol % PEG-modified lipid.

Some embodiments comprise adding PEG to a composition comprising an LNP encapsulating a nucleic acid (e.g., which already includes PEG in the amounts listed above). Without being bound by theory, it is believed that spiking an LNP composition with additional PEG can provide benefits during lyophilization. Thus, some embodiments, comprise adding additional PEG as compared to an amount used for a non-lyophilized LNP composition. In embodiments comprise adding about 0.5mo % or more PEG to an LNP composition, such as about 1 mol %, about 1.5 mol %, about 2 mol %, about 2.5 mol %, about 3 mol %, about 3.5 mol %, about 4 mol %, about 5 mol %, or more after formation of an LNP composition (e.g., which already contains PEG in amount listed elsewhere herein).

In some embodiments, a lipid nanoparticle comprises 20-60 mol % ionizable amino lipid, 5-25 mol % non-cationic lipid, 25-55 mol % sterol, and 0.5-15 mol % PEG-modified lipid.

In some embodiments, an LNP of the disclosure comprises an ionizable amino lipid of Compound 1, wherein the non-cationic lipid is DSPC, the structural lipid that is cholesterol, and the PEG lipid is DMG-PEG.

In some embodiments, an LNP of the present disclosure comprises an ionizable amino lipid of any of Formula VI, VII or VIIII, a phospholipid comprising DSPC, a structural lipid, and a PEG lipid comprising PEG-DMG.

In some embodiments, an LNP of the present disclosure comprises an ionizable amino lipid of any of Formula VI, VII or VIII, a phospholipid comprising DSPC, a structural lipid, and a PEG lipid comprising a compound having Formula XI.

In some embodiments, an LNP of the present disclosure comprises an ionizable amino lipid of Formula VI, VII or VIII, a phospholipid comprising a compound having Formula VIII, a structural lipid, and the PEG lipid comprising a compound having Formula X or XI.

In some embodiments, an LNP of the present disclosure comprises an ionizable amino lipid of Formula VI, VII or VIII, a phospholipid comprising a compound having Formula IX, a structural lipid, and the PEG lipid comprising a compound having Formula X or XI.

In some embodiments, an LNP of the present disclosure comprises an ionizable amino lipid of Formula VI, VII or VIII, a phospholipid having Formula IX, a structural lipid, and a PEG lipid comprising a compound having Formula XI.

In some embodiments, a lipid nanoparticle comprises 49 mol % ionizable amino lipid, 10 mol % DSPC, 38.5 mol % cholesterol, and 2.5 mol % DMG-PEG.

In some embodiments, a lipid nanoparticle comprises 49 mol % ionizable amino lipid, 11 mol % DSPC, 38.5 mol % cholesterol, and 1.5 mol % DMG-PEG.

In some embodiments, a lipid nanoparticle comprises 48 mol % ionizable amino lipid, 11 mol % DSPC, 38.5 mol % cholesterol, and 2.5 mol % DMG-PEG.

In some embodiments, an LNP of the present disclosure comprises an N:P ratio of from about 2:1 to about 30:1.

In some embodiments, an LNP of the present disclosure comprises an N:P ratio of about 6:1.

In some embodiments, an LNP of the present disclosure comprises an N:P ratio of about 3:1, 4:1, or 5:1.

In some embodiments, an LNP of the present disclosure comprises a wt/wt ratio of the ionizable amino lipid component to the RNA of from about 10:1 to about 100:1.

In some embodiments, an LNP of the present disclosure comprises a wt/wt ratio of the ionizable amino lipid component to the RNA of about 20:1.

In some embodiments, an LNP of the present disclosure comprises a wt/wt ratio of the ionizable amino lipid component to the RNA of about 10:1.

Some embodiments comprise a composition having one or more LNPs having a diameter of about 150 nm or less, such as about 140 nm, 130 nm, 120 nm, 110 nm, 100 nm, 90 nm, 80 nm, 70 nm, 60 nm, 50 nm, 40 nm, 30 nm, or 20 nm or less. Some embodiments comprise a composition having a mean LNP diameter of about 150 nm or less, such as about 140 nm, 130 nm, 120 nm, 110 nm, 100 nm, 90 nm, 80 nm, 70 nm, 60 nm, 50 nm, 40 nm, 30 nm, or 20 nm or less. In some embodiments, the composition has a mean LNP diameter from about 30 nm to about 150 nm, or a mean diameter from about 60 nm to about 120 nm.

An LNP may comprise or one or more types of lipids, including but not limited to amino lipids (e.g., ionizable amino lipids), neutral lipids, non-cationic lipids, charged lipids, PEG-modified lipids, phospholipids, structural lipids and sterols. In some embodiments, an LNP may further comprise one or more cargo molecules, including but not limited to nucleic acids (e.g., mRNA, plasmid DNA, DNA or RNA oligonucleotides, siRNA, shRNA, snRNA, snoRNA, lncRNA, etc.), small molecules, proteins and peptides.

In some embodiments, the composition comprises a liposome. A liposome is a lipid particle comprising lipids arranged into one or more concentric lipid bilayers around a central region. The central region of a liposome may comprise an aqueous solution, suspension, or other aqueous composition.

In some embodiments, a lipid nanoparticle may comprise two or more components (e.g., amino lipid and nucleic acid, PEG-lipid, phospholipid, structural lipid). For instance, a lipid nanoparticle may comprise an amino lipid and a nucleic acid. Compositions comprising lipid nanoparticles, such as those described herein, may be used for a wide variety of applications, including the stealth delivery of mRNA with minimal adverse innate immune response.

Effective in vivo delivery of nucleic acids represents a continuing medical challenge. Exogenous nucleic acids (i.e., originating from outside of a cell or organism) are readily degraded in the body, e.g., by the immune system. Accordingly, effective delivery of nucleic acids to cells often requires the use of a particulate carrier (e.g., lipid nanoparticles). The particulate carrier should be formulated to have minimal particle aggregation, be relatively stable prior to intracellular delivery, effectively deliver nucleic acids intracellularly, and illicit no or minimal immune response. To achieve minimal particle aggregation and pre-delivery stability, many conventional particulate carriers have relied on the presence and/or concentration of certain components (e.g., PEG-lipid). However, it has been discovered that certain components may decrease the stability of encapsulated nucleic acids (e.g., mRNA molecules). The reduced stability may limit the broad applicability of the particulate carriers. As such, there remains a need for methods by which to improve the stability of nucleic acid (e.g., mRNA) encapsulated within lipid nanoparticles.

In some embodiments, a lipid nanoparticles comprise one or more of ionizable molecules, polynucleotides, and optional components, such as structural lipids, sterols, neutral lipids, phospholipids and a molecule capable of reducing particle aggregation (e.g., polyethylene glycol (PEG), PEG-modified lipid), such as those described above.

In some embodiments, an LNP described herein may include one or more ionizable molecules (e.g., amino lipids or ionizable lipids). The ionizable molecule may comprise a charged group and may have a certain pKa. In certain embodiments, the pKa of the ionizable molecule may be greater than or equal to about 6, greater than or equal to about 6.2, greater than or equal to about 6.5, greater than or equal to about 6.8, greater than or equal to about 7, greater than or equal to about 7.2, greater than or equal to about 7.5, greater than or equal to about 7.8, greater than or equal to about 8. In some embodiments, the pKa of the ionizable molecule may be less than or equal to about 10, less than or equal to about 9.8, less than or equal to about 9.5, less than or equal to about 9.2, less than or equal to about 9.0, less than or equal to about 8.8, or less than or equal to about 8.5. Combinations of the above referenced ranges are also possible (e.g., greater than or equal to 6 and less than or equal to about 8.5). Other ranges are also possible. In embodiments in which more than one type of ionizable molecule are present in a particle, each type of ionizable molecule may independently have a pKa in one or more of the ranges described above.

In general, an ionizable molecule comprises one or more charged groups. In some embodiments, an ionizable molecule may be positively charged or negatively charged. For instance, an ionizable molecule may be positively charged. For example, an ionizable molecule may comprise an amine group. As used herein, the term “ionizable molecule” has its ordinary meaning in the art and may refer to a molecule or matrix comprising one or more charged moiety. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidazolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule and/or matrix may be selected as desired.

In some cases, an ionizable molecule (e.g., an amino lipid or ionizable lipid) may include one or more precursor moieties that can be converted to charged moieties. For instance, the ionizable molecule may include a neutral moiety that can be hydrolyzed to form a charged moiety, such as those described above. As a non-limiting specific example, the molecule or matrix may include an amide, which can be hydrolyzed to form an amine, respectively. Those of ordinary skill in the art will be able to determine whether a given chemical moiety carries a formal electronic charge (for example, by inspection, pH titration, ionic conductivity measurements, etc.), and/or whether a given chemical moiety can be reacted (e.g., hydrolyzed) to form a chemical moiety that carries a formal electronic charge.

The ionizable molecule (e.g., amino lipid or ionizable lipid) may have any suitable molecular weight. In certain embodiments, the molecular weight of an ionizable molecule is less than or equal to about 2,500 g/mol, less than or equal to about 2,000 g/mol, less than or equal to about 1,500 g/mol, less than or equal to about 1,250 g/mol, less than or equal to about 1,000 g/mol, less than or equal to about 900 g/mol, less than or equal to about 800 g/mol, less than or equal to about 700 g/mol, less than or equal to about 600 g/mol, less than or equal to about 500 g/mol, less than or equal to about 400 g/mol, less than or equal to about 300 g/mol, less than or equal to about 200 g/mol, or less than or equal to about 100 g/mol. In some instances, the molecular weight of an ionizable molecule is greater than or equal to about 100 g/mol, greater than or equal to about 200 g/mol, greater than or equal to about 300 g/mol, greater than or equal to about 400 g/mol, greater than or equal to about 500 g/mol, greater than or equal to about 600 g/mol, greater than or equal to about 700 g/mol, greater than or equal to about 1000 g/mol, greater than or equal to about 1,250 g/mol, greater than or equal to about 1,500 g/mol, greater than or equal to about 1,750 g/mol, greater than or equal to about 2,000 g/mol, or greater than or equal to about 2,250 g/mol. Combinations of the above ranges (e.g., at least about 200 g/mol and less than or equal to about 2,500 g/mol) are also possible. In embodiments in which more than one type of ionizable molecules are present in a particle, each type of ionizable molecule may independently have a molecular weight in one or more of the ranges described above.

In some embodiments, the percentage (e.g., by weight, or by mole) of a single type of ionizable molecule (e.g., amino lipid or ionizable lipid) and/or of all the ionizable molecules within a particle may be greater than or equal to about 15%, greater than or equal to about 16%, greater than or equal to about 17%, greater than or equal to about 18%, greater than or equal to about 19%, greater than or equal to about 20%, greater than or equal to about 21%, greater than or equal to about 22%, greater than or equal to about 23%, greater than or equal to about 24%, greater than or equal to about 25%, greater than or equal to about 30%, greater than or equal to about 35%, greater than or equal to about 40%, greater than or equal to about 42%, greater than or equal to about 45%, greater than or equal to about 48%, greater than or equal to about 50%, greater than or equal to about 52%, greater than or equal to about 55%, greater than or equal to about 58%, greater than or equal to about 60%, greater than or equal to about 62%, greater than or equal to about 65%, or greater than or equal to about 68%. In some instances, the percentage (e.g., by weight, or by mole) may be less than or equal to about 70%, less than or equal to about 68%, less than or equal to about 65%, less than or equal to about 62%, less than or equal to about 60%, less than or equal to about 58%, less than or equal to about 55%, less than or equal to about 52%, less than or equal to about 50%, or less than or equal to about 48%. Combinations of the above referenced ranges are also possible (e.g., greater than or equal to 20% and less than or equal to about 60%, greater than or equal to 40% and less than or equal to about 55%, etc.). In embodiments in which more than one type of ionizable molecule is present in a particle, each type of ionizable molecule may independently have a percentage (e.g., by weight, or by mole) in one or more of the ranges described above. The percentage (e.g., by weight, or by mole) may be determined by extracting the ionizable molecule(s) from the dried particles using, e.g., organic solvents, and measuring the quantity of the agent using high pressure liquid chromatography (i.e., HPLC), liquid chromatography-mass spectrometry (LC-MS), nuclear magnetic resonance (NMR), or mass spectrometry (MS). Those of ordinary skill in the art would be knowledgeable of techniques to determine the quantity of a component using the above-referenced techniques. For example, HPLC may be used to quantify the amount of a component, by, e.g., comparing the area under the curve of a HPLC chromatogram to a standard curve.

It should be understood that the terms “charged” or “charged moiety” does not refer to a “partial negative charge” or “partial positive charge” on a molecule. The terms “partial negative charge” and “partial positive charge” are given their ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

According to the disclosures herein, a lipid composition may comprise one or more lipids as described herein. Such lipids may include those useful in the preparation of lipid nanoparticle formulations as described above or as known in the art.

### Multivalent Vaccines

As described herein, the term “monovalent” refers to an mRNA polynucleotide having an ORF encoding one Borrelia proteins. As described herein, the term “multivalent” refers to an mRNA polynucleotide (or an mRNA vaccine) encoding multiple Borrelia proteins (on one or more mRNAs). As described herein, the term “hexavalent” refers to six mRNA polynucleotides, each mRNA comprising an ORF encoding one or more Borrelia proteins. As described herein, the term “heptavalent” refers to seven mRNA polynucleotides, each mRNA comprising an ORF encoding one or more Borrelia proteins.

The compositions, as provided herein, may include one mRNA polynucleotide or multiple RNA polynucleotides (e.g., mRNAs) collectively encoding two or more antigens of the same or different species. In some embodiments, composition includes an mRNA polynucleotide or multiple mRNA polynucleotides collectively encoding two or more Borrelia proteins.

In some embodiments, a single mRNA polynucleotide encodes 2, 3, 4, 5, 6, 7, 8, 9, or 10, or more Borrelia proteins. In some embodiments, the mRNA polynucleotide comprises an ORF that encodes 2 or more Borrelia proteins (e.g., 2 or more of OspA S1-S7). In some embodiments, the mRNA polynucleotide comprises an ORF that encodes 3 or more Borrelia proteins (e.g., 3 or more of OspA S1-S7). In some embodiments, the mRNA polynucleotide comprises an ORF that encodes 4 or more Borrelia proteins (e.g., 4 or more of OspA S1-S7). In some embodiments, the mRNA polynucleotide comprises an ORF that encodes 5 or more Borrelia proteins (e.g., 5 or more of OspA S1-S7). In some embodiments, the mRNA polynucleotide comprises an ORF that encodes 6 or more Borrelia proteins (e.g., 6 or more of OspA S1-S7). In some embodiments, the mRNA polynucleotide comprises an ORF that encodes 7 or more Borrelia proteins (e.g., each of OspA S1-S7).

In preferred embodiments, a composition comprises multiple mRNA polynucleotides each encoding a distinct Borrelia protein. In some embodiments, a composition comprises a first mRNA polynucleotide comprising an ORF encoding a first Borrelia protein (e.g., one of OspA S1-S7) and a second mRNA polynucleotide comprising an ORFencoding a second Borrelia protein (e.g., one of OspA S1-S7), wherein the first Borrelia protein and the second Borrelia protein are distinct proteins. In some embodiments, a composition comprises a first mRNA polynucleotide encoding a first Borrelia protein (e.g., one of OspA S1-S7), a second RNA polynucleotide encoding a second Borrelia protein (e.g., one of OspA S1-S7), and a third RNA polynucleotide encoding a third Borrelia protein (e.g., one of OspA S1-S7), wherein the first Borrelia protein, the second Borrelia protein, and the third Borrelia protein are distinct proteins. In some embodiments, a composition comprises a first mRNA polynucleotide encoding a first Borrelia protein (e.g., one of OspA S1-S7), a second mRNA polynucleotide encoding a second Borrelia protein (e.g., one of OspA S1-S7), a third mRNA polynucleotide encoding a third Borrelia protein (e.g., one of OspA S1-S7), and a fourth mRNA polynucleotide encoding a fourth Borrelia protein (e.g., one of OspA S1-S7), wherein the first Borrelia protein, the second Borrelia protein, the third Borrelia protein, and the fourth Borrelia protein are distinct proteins. In some embodiments, a composition comprises a first mRNA polynucleotide encoding a first Borrelia protein (e.g., one of OspA S1-S7), a second mRNA polynucleotide encoding a second Borrelia protein (e.g., one of OspA S1-S7), a third mRNA polynucleotide encoding a third Borrelia protein (e.g., one of OspA S1-S7), a fourth mRNA polynucleotide encoding a fourth Borrelia protein (e.g., one of OspA S1-S7), and a fifth mRNA polynucleotide encoding a fifth Borrelia protein (e.g., one of OspA S1-S7), wherein the first Borrelia protein, the second Borrelia protein, the third Borrelia protein, the fourth Borrelia protein, and the fifth Borrelia protein are distinct proteins. In some embodiments, a composition comprises a first mRNA polynucleotide encoding a first Borrelia protein (e.g., one of OspA S1-S7), a second mRNA polynucleotide encoding a second Borrelia protein (e.g., one of OspA S1-S7), a third mRNA polynucleotide encoding a third Borrelia protein (e.g., one of OspA S1-S7), a fourth RNA encoding a fourth Borrelia protein (e.g., one of OspA S1-S7), a fifth mRNA polynucleotide encoding a fifth Borrelia proteins (e.g., one of OspA S1-S7), and a sixth mRNA polynucleotide encoding a sixth Borrelia protein (e.g., one of OspA S1-S7), wherein the first Borrelia protein, the second Borrelia protein, the third Borrelia protein, the fourth Borrelia protein, the fifth Borrelia protein, and the sixth Borrelia protein are distinct proteins. In some embodiments, a composition comprises a first mRNA polynucleotide encoding a first Borrelia protein (e.g., OspA S1), a second mRNA polynucleotide encoding a second Borrelia protein (e.g., OspA S2), a third mRNA polynucleotide encoding a third Borrelia protein (e.g., OspA S3), a fourth mRNA polynucleotide encoding a fourth Borrelia protein (e.g., OspA S4), a fifth mRNA polynucleotide encoding a fifth Borrelia proteins (e.g., OspA S4), a sixth mRNA polynucleotide encoding a sixth Borrelia protein (e.g., OspA S6), and a seventh mRNA polynucleotide encoding a seventh Borrelia protein (e.g., OspA S7)

wherein the first Borrelia protein, the second Borrelia protein, the third Borrelia protein, the fourth Borrelia protein, the fifth Borrelia protein, and the sixth Borrelia protein are distinct proteins.

In some embodiments, two or more different mRNA polynucleotides encoding antigens may be formulated in the same lipid nanoparticle. In other embodiments, two or more different mRNA encoding antigens may be formulated in separate lipid nanoparticles (each mRNA formulated in a single lipid nanoparticle). Lipid nanoparticles may then be combined and administered as a single vaccine composition (e.g., comprising multiple RNAs (e.g., mRNAs) encoding multiple antigens) or may be administered separately.

### Pharmaceutical Formulations

Provided herein are compositions (e.g., pharmaceutical compositions, such as vaccines), methods, kits and reagents for prevention of Lyme disease and other conditions directly or indirectly cause by Borrelia infection in humans and other mammals, for example. The compositions provided herein can be used as a prophylactic agent to prevent a Borrelia infection, and thus Lyme disease, cause by a Borrelia infection.

In some embodiments, the compositions containing mRNA polynucleotide(s) as described herein can be administered to a subject (e.g., a mammalian subject, such as a human subject), and the mRNA polynucleotides are translated in vivo to produce an antigenic polypeptide (antigen).

An “effective amount” of a composition (e.g., an mRNA vaccine of the instant invention) is based, at least in part, on the target tissue, target cell type, means of administration, physical characteristics of the mRNA (e.g., length, nucleotide composition, and/or extent of modified nucleosides), other components of the vaccine, and other determinants, such as age, body weight, height, sex and general health of the subject. Typically, an effective amount of a composition induces or boosts an immune response as a function of antigen production in the cells of the subject. In some embodiments, an effective amount of the composition containing mRNA polynucleotide(s) having at least one chemical modification are more efficient than a composition containing a corresponding unmodified polynucleotide encoding the same antigen or a peptide antigen. Increased antigen production may be demonstrated by increased cell transfection (the percentage of cells transfected with the mRNA vaccine), increased protein translation and/or expression from the polynucleotide, decreased nucleic acid degradation (as demonstrated, for example, by increased duration of protein translation from a modified polynucleotide), or altered antigen specific immune response of the host cell.

The term “pharmaceutical composition” refers to the combination of an active agent (e.g., mRNA polynucleotide) with a carrier (e.g., lipid composition, e.g., LNP)), inert or active, making the composition especially suitable for prophylactic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carriers include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences.

In some embodiments, the compositions (comprising polynucleotides and their encoded polypeptides) in accordance with the present disclosure may be used for the prevention of a Borrelia infection. A composition may be administered prophylactically as part of an active immunization scheme to healthy individuals or early in infection during the incubation phase. In some embodiments, the amount of mRNA provided to a cell, a tissue or a subject may be an amount effective for immune prophylaxis.

In some embodiments, the compositions (comprising polynucleotides and their encoded polypeptides) in accordance with the present disclosure may be used in a method of vaccinating a subject. As used herein, the term “vaccinating” refers to a method of inducing an immune response in a subject to a particular antigen or pathogen.

As used herein, when referring to a prophylactic composition, such as a vaccine, the term “booster” refers to administration of the prophylactic (vaccine) composition to a subject that has had a previous infection or has had a previous vaccine. A booster (or booster vaccine) may be given after an earlier administration of the prophylactic composition. The time of administration between the initial administration of the prophylactic composition and the booster may be, but is not limited to, 12 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, or more. In exemplary embodiments, the time of administration between the initial administration of the prophylactic composition and the booster may be, but is not limited to, 1 week, 2 weeks, 3 weeks, 1 month, 2 months, 3 months, or 6 months. As is described herein, the booster may comprise the same or different mRNA as compared to the earlier administration of the prophylactic composition. The booster, in some embodiments is monovalent (e.g., the mRNA polynucleotide comprises an ORF that encodes a single antigen). In some embodiments, the booster is multivalent (e.g., the mRNA polynucleotide comprises an ORF that encodes more than one antigen and/or the booster comprises two or more mRNApolynucleotides, collectively encoding more than one antigen).

In some embodiments, “administering” or “administration” means providing a material to a subject in a manner that is pharmacologically useful. In some embodiments, a composition disclosed herein is administered to the subject parenterally. In some embodiments, a composition disclosed herein is administered to a subject subcutaneously or, intramuscularly.

A composition may be utilized in various settings depending on the prevalence of the infection or the degree or level of unmet medical need. As a non-limiting example, the mRNA vaccines may be utilized to prevent Lyme disease. mRNA vaccines have superior properties in that they produce much larger antibody titers, better neutralizing immunity, produce more durable immune responses, and/or produce responses earlier than commercially available vaccines.

Provided herein are pharmaceutical compositions including mRNA and/or complexes optionally in combination with one or more pharmaceutically acceptable excipients.

The mRNA may be formulated or administered alone or in conjunction with one or more other components. For example, a vaccine may comprise other components including, but not limited to, adjuvants.

In a preferred embodiment, a vaccine does not include an adjuvant (they are adjuvant free).

An mRNA may be formulated or administered in combination with one or more pharmaceutically-acceptable excipients. In some embodiments, vaccines comprise at least one additional active substance, such as, for example, a therapeutically-active substance, a prophylactically-active substance, or a combination of both. Vaccine compositions may be sterile, pyrogen-free or both sterile and pyrogen-free. General considerations in the formulation and/or manufacture of pharmaceutical agents, such as vaccines, may be found, for example, in Remington: The Science and Practice of Pharmacy 21st ed., Lippincott Williams & Wilkins, 2005 (incorporated herein by reference in its entirety).

In some embodiments, a vaccine is administered to humans, human patients or subjects. For the purposes of the present disclosure, the phrase “active ingredient” generally refers to the mRNA contained therein, for example, mRNA encoding Borrelia protein antigens.

Formulations of the vaccines described herein may be prepared by any method known or hereafter developed in the art of pharmacology. In general, such preparatory methods include the step of bringing the active ingredient (e.g., mRNA) into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered.

### Methods of Prophylaxis

Provided herein are compositions (e.g., vaccines), methods, kits and reagents for the prevention of a Borrelia infection in humans and other mammals. The compositions can be used as prophylactic agents, for example. In some embodiments, the compositions are used to provide prophylactic protection from a Borrelia infection.

A subject may be any mammal, including non-human primate and human subjects. Typically, a subject is a human subject.

In some embodiments, a composition is administered to a subject (e.g., a mammalian subject, such as a human subject) in an effective amount to induce an antigen-specific immune response. The RNA encoding the Borrelia protein is expressed and translated in vivo to produce the antigen, which then stimulates an immune response in the subject.

Prophylactic protection from Lyme disease or other condition cause directly or as a result of Borrelia infection can be achieved following administration of a composition of the present disclosure. The compositions can be administered once, twice, three times, four times or more but it is likely sufficient to administer the composition once (optionally followed by repeated administration). Dosing may need to be adjusted accordingly.

A method of eliciting an immune response in a subject against a Borrelia protein (or multiple Borrelia antigens) is provided in aspects of the present disclosure. In some embodiments, a method involves administering to the subject a vaccine comprising a mRNA polynucleotide having an open reading frame encoding a Borrelia protein (or multiple Borrelia antigens), thereby inducing in the subject an immune response specific to the Borrelia protein (or multiple Borrelia antigens), wherein anti-Borrelia antibody titer in the subject is increased following vaccination relative to a subject that has not been vaccinated.

Some aspects of the disclosure relate to methods and compositions of eliciting an immune response in a subject against a Borrelia protein (or multiple Borrelia antigens) that is not encoded by an mRNA of a vaccine. As described herein, the term “cross-reactivity” refers to an immune response against an antigen that is not encoded by an mRNA of the subject vaccines. As described herein the terms “cross-protect” and “cross-protection” refer to clinical protection against related antigens not encoded by the mRNA polynucleotide(s) of the vaccine (e.g., cross-protection as measured according to methods described in Example 11).

In some embodiments, the immune response in the subject is induced 2 days, 3 days, 1 week, 2 weeks, 3 weeks, 5 weeks, or 10 weeks earlier relative to an immune response induced in a subject vaccinated with a prophylactically effective dose of a traditional vaccine.

Also provided herein are methods of eliciting an immune response in a subject against Borrelia by administering to the subject an mRNA having an open reading frame encoding at least one Borrelia protein, wherein the mRNA does not include a stabilization element, and wherein an adjuvant is not co-formulated or co-administered with the vaccine.

A composition may be administered by any route that results in a prophylactically effective outcome. These include, but are not limited, to intradermal, intramuscular, and/or subcutaneous administration. The present disclosure provides methods comprising administering mRNA vaccines to a subject in need thereof. The exact amount required will vary from subject to subject, depending on the species, age, and general condition of the subject, the severity of the disease, the particular composition, its mode of administration, its mode of activity, and the like. The mRNA is typically formulated in dosage unit form for ease of administration and uniformity of dosage. It will be understood, however, that the total daily usage of the mRNA may be decided by the attending physician within the scope of sound medical judgment. The specific prophylactically effective dose level for any particular patient will depend upon a variety of factors including the disorder being addressed and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The effective amount of the mRNA (e.g., an effective dose), as provided herein, may be as low as 20 μg, administered for example as a single dose or as two 10 μg doses (e.g., a first effective vaccine dose and a second effective vaccine dose). In some embodiments, the first effective vaccine dose and the second effective vaccine dose are the same amount. In some embodiments, the first effective vaccine dose and the second effective vaccine dose are different amounts. In some embodiments, the effective amount is a total dose of 5 μg-30 μg, 5 μg-25 μg, μg-20 μg, 5 μg-15 μg, 5 μg-10 μg, 10 μg-30 μg, 10 μg-25 μg, 10 μg-20 μg, 10 μg-15 μg, μg-30 μg, 15 μg-25 μg, 15 μg-20 μg, 20 μg-30 μg, 25 μg-30 μg, or 25 μg-300 μg. In some embodiments, the effective dose (e.g., effective amount) is at least 10 μg and less than 25 μg of the composition. In some embodiments, the effective dose (e.g., effective amount) is at least 5 μg and less than 25 μg of the composition. For example, the effective amount may be a total dose of 5 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 110 μg, 120 μg, 130 μg, 140 μg, 150 μg, 160 μg, 170 μg, 180 μg, 190 μg, 200 μg, 250 μg, or 300 μg. In some embodiments, the effective amount (e.g., effective dose) is a total dose of 10 μg. In some embodiments, the effective amount is a total dose of 20 g (e.g., two 10 μg doses). In some embodiments, the effective amount is a total dose of 25 μg. In some embodiments, the effective amount is a total dose of 30 μg. In some embodiments, the effective amount is a total dose of 50 μg. In some embodiments, the effective amount is a total dose of 60 μg (e.g., two 30 μg doses). In some embodiments, the effective amount is a total dose of 75 μg. In some embodiments, the effective amount is a total dose of 100 μg. In some embodiments, the effective amount is a total dose of 150 μg. In some embodiments, the effective amount is a total dose of 200 μg. In some embodiments, the effective amount is a total dose of 250 g. In some embodiments, the effective amount is a total dose of 300 μg.

In some embodiments, an mRNA vaccine is a monovalent mRNA vaccine comprising a single mRNA polynucleotide comprising an ORF that encodes one OspA serotype (i.e., one of OspA S1-S7), such as a construct comprising any one of SEQ ID NOs: 8-14, including the constructs of Table 1). For example, in some embodiments, a monovalent mRNA vaccine comprises an mRNA having an ORF that encodes for OspA S1 (e.g., comprises the sequence of SEQ ID NO: 8), but lacks a mRNA(s) comprising one or more ORFs encoding for any of OspA S2-S7. In some embodiments, the effective dose (e.g., effective amount) of monovalent mRNA vaccine (e.g., a construct comprising the sequence of SEQ ID NO: 8) is between about 10 μg and about 150 μg (e.g., between about 12.5 μg and about 50 μg). In some embodiments, the effective dose of monovalent mRNA vaccine (e.g., a construct comprising the sequence of SEQ ID NO: 8) is about 10 μg, about 12.5 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 110 μg, about 120 μg, about 130 μg, about 140 μg, or about 150 μg. In some embodiments, the effective dose of monovalent mRNA vaccine (e.g., a construct comprising the sequence of SEQ ID NO: 8), is 10 μg, 12.5 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 110 μg, 120 μg, 130 μg, 140 μg, or 150 μg. In some embodiments, the effective dose of monovalent mRNA vaccine (e.g., a construct comprising the sequence of SEQ ID NO: 8) is about 12.5 μg. In some embodiments, the effective dose of monovalent mRNA vaccine (e.g., a construct comprising the sequence of SEQ ID NO: 8) is about 25 μg. In some embodiments, the effective dose of monovalent mRNA vaccine (e.g., a construct comprising the sequence of SEQ ID NO: 8) is about 50 μg.

The effective dose (e.g., effective amount) of monovalent mRNA vaccine (e.g., a construct comprising the sequence of SEQ ID NO: 8) may be administered for example as a single dose. The effective does of monovalent vaccine (e.g., a construct comprising the sequence of SEQ ID NO: 8) may be administered as multiple doses (e.g., a first effective vaccine dose, a second effective vaccine dose, a third effective vaccine dose). In some embodiments, the first effective vaccine dose, the second effective vaccine dose, and/or the third effective vaccine dose are the same amount (e.g., each 25 μg, 50 μg, 100 μg, or 150 μg). In some embodiments, first effective vaccine dose, the second effective vaccine dose, and/or the third effective vaccine dose are different amounts. In some embodiments, the effective vaccine doses are administered at least about 2 months (e.g., 2 weeks, 3 weeks, 4 weeks, 1 month, 1.5 months, 2 months, 3 months, 4 months, 5 months, 6 months) apart. In some embodiments, the effective vaccine doses are administered at least about 2 weeks, 3 weeks, 4 weeks, 1 month, 1.5 months, 2 months, 3 months, 4 months, 5 months, 6 months apart. In some embodiments, the effective vaccine doses are administered at least about 2 months apart (e.g., a second does at 2 months and a third does at 3 months).

In some embodiments, an mRNA vaccine is a multivalent mRNA vaccine comprising an mRNA polynucleotide comprising at least two ORF (e.g., encoding for two or more of OspA S1-S7, such as two or more of an ORF comprising the sequence of SEQ ID NO: 8, an ORF comprising the sequence of SEQ ID NO: 9, an ORF comprising the sequence of SEQ ID NO: 10, an ORF comprising the sequence of SEQ ID NO: 11, an ORF comprising the sequence of SEQ ID NO: 12, an ORF comprising the sequence of SEQ ID NO: 13, and an ORF comprising the sequence of SEQ ID NO: 14). In some embodiments, the effective dose (e.g., effective amount) of multivalent mRNA vaccine is between about 10 μg and about 150 μg (e.g., between about 12.5 μg and about 50 μg). In some embodiments, the effective dose of multivalent mRNA vaccine is about 10 μg, about 12.5 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 110 μg, about 120 μg, about 130 μg, about 140 μg, or about 150 μg. In some embodiments, the effective dose of multivalent mRNA vaccine is 10 μg, 12.5 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 110 μg, 120 μg, 130 μg, 140 μg, or 150 μg. In some embodiments, the effective dose of multivalent mRNA vaccine is about 12.5 μg. In some embodiments, the effective dose of multivalent mRNA vaccine is about 25 μg. In some embodiments, the effective dose of multivalent mRNA vaccine is about 50 μg.

The effective dose (e.g., effective amount) of multivalent mRNA vaccine may be administered for example as a single dose. The effective does of multivalent vaccine may be administered as multiple doses (e.g., a first effective vaccine dose, a second effective vaccine dose, a third effective vaccine dose). In some embodiments, the first effective vaccine dose, the second effective vaccine dose, and/or the third effective vaccine dose are the same amount (e.g., each 25 μg, 50 μg, 100 μg, or 150 μg). In some embodiments, first effective vaccine dose, the second effective vaccine dose, and/or the third effective vaccine dose are different amounts. In some embodiments, the effective vaccine doses are administered at least about 2 months (e.g., 2 weeks, 3 weeks, 4 weeks, 1 month, 1.5 months, 2 months, 3 months, 4 months, 5 months, 6 months) apart. In some embodiments, the effective vaccine doses are administered at least about 2 weeks, 3 weeks, 4 weeks, 1 month, 1.5 months, 2 months, 3 months, 4 months, 5 months, 6 months apart. In some embodiments, the effective vaccine doses are administered at least about 2 months apart (e.g., a second does at 2 months and a third does at 3 months).

In some embodiments, an mRNA vaccine is a multivalent mRNA vaccine comprising two or more mRNA constructs, each comprising one or more ORF encoding for a distinct OspA serotype (e.g., a mRNA vaccine comprising two, three, four, five, six, seven, or more of the constructs provided in Table 1).

In some embodiments, an mRNA vaccine is a multivalent mRNA vaccine comprising at least one mRNA construct(s), each comprising an ORF, collectively encoding for OspA S1-S7 (e.g., an ORF comprising the sequence of SEQ ID NO: 8, an ORF comprising the sequence of SEQ ID NO: 9, an ORF comprising the sequence of SEQ ID NO: 10, an ORF comprising the sequence of SEQ ID NO: 11, an ORF comprising the sequence of SEQ ID NO: 12, an ORF comprising the sequence of SEQ ID NO: 13, and an ORF comprising the sequence of SEQ ID NO: 14). In some embodiments, the effective dose of multivalent mRNA vaccine (e.g., comprising an ORF comprising the sequence of SEQ ID NO: 8, an ORF comprising the sequence of SEQ ID NO: 9, an ORF comprising the sequence of SEQ ID NO: 10, an ORF comprising the sequence of SEQ ID NO: 11, an ORF comprising the sequence of SEQ ID NO: 12, an ORF comprising the sequence of SEQ ID NO: 13, and an ORF comprising the sequence of SEQ ID NO: 14) is about 20 μg, about 25 μg, about 30 μg, about 35 μg, about 40 μg, about μg, about 50 μg, about 55 μg, about 60 μg, about 65 μg, about 70 μg, about 75 μg, about 80 μg, about 85 μg, about 90 μg, about 95 μg, about 100 μg, about 110 μg, about 120 μg, about 130 μg, about 140 μg, about 150 μg, about 160 μg, about 170 μg, about 180 μg, about 190 μg, about 200 μg, about 250 μg, about 300 μg, or about 450 g. In some embodiments, the effective dose of multivalent mRNA vaccine (e.g., comprising an ORF comprising the sequence of SEQ ID NO: 8, an ORF comprising the sequence of SEQ ID NO: 9, an ORF comprising the sequence of SEQ ID NO: 10, an ORF comprising the sequence of SEQ ID NO: 11, an ORF comprising the sequence of SEQ ID NO: 12, an ORF comprising the sequence of SEQ ID NO: 13, and an ORF comprising the sequence of SEQ ID NO: 14), is 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 110 μg, 120 μg, 130 μg, 140 μg, 150 μg, 160 μg, 170 μg, 180 μg, 190 μg, 200 μg, 250 μg, 300 μg, or 450 μg. In some embodiments, the effective dose of multivalent mRNA vaccine (e.g., comprising an ORF comprising the sequence of SEQ ID NO: 8, an ORF comprising the sequence of SEQ ID NO: 9, an ORF comprising the sequence of SEQ ID NO: 10, an ORF comprising the sequence of SEQ ID NO: 11, an ORF comprising the sequence of SEQ ID NO: 12, an ORF comprising the sequence of SEQ ID NO: 13, and an ORF comprising the sequence of SEQ ID NO: 14) is about μg. In some embodiments, the effective dose of multivalent mRNA vaccine (e.g., comprising an ORF comprising the sequence of SEQ ID NO: 8, an ORF comprising the sequence of SEQ ID NO: 9, an ORF comprising the sequence of SEQ ID NO: 10, an ORF comprising the sequence of SEQ ID NO: 11, an ORF comprising the sequence of SEQ ID NO: 12, an ORF comprising the sequence of SEQ ID NO: 13, and an ORF comprising the sequence of SEQ ID NO: 14) is about 50 μg. In some embodiments, the effective dose of multivalent mRNA vaccine (e.g., comprising an ORF comprising the sequence of SEQ ID NO: 8, an ORF comprising the sequence of SEQ ID NO: 9, an ORF comprising the sequence of SEQ ID NO: 10, an ORF comprising the sequence of SEQ ID NO: 11, an ORF comprising the sequence of SEQ ID NO: 12, an ORF comprising the sequence of SEQ ID NO: 13, and an ORF comprising the sequence of SEQ ID NO: 14) is about 100 μg. In some embodiments, the effective dose of multivalent mRNA vaccine (e.g., comprising an ORF comprising the sequence of SEQ ID NO: 8, an ORF comprising the sequence of SEQ ID NO: 9, an ORF comprising the sequence of SEQ ID NO: 10, an ORF comprising the sequence of SEQ ID NO: 11, an ORF comprising the sequence of SEQ ID NO: 12, an ORF comprising the sequence of SEQ ID No: 13, and an ORF comprising the sequence of SEQ ID NO: 14) is about 150 μg.

The effective dose (e.g., effective amount) of multivalent mRNA vaccine (e.g., comprising an ORF comprising the sequence of SEQ ID NO: 8, an ORF comprising the sequence of SEQ ID NO: 9, an ORF comprising the sequence of SEQ ID NO: 10, an ORF comprising the sequence of SEQ ID NO: 11, an ORF comprising the sequence of SEQ ID NO: 12, an ORF comprising the sequence of SEQ ID No: 13, and an ORF comprising the sequence of SEQ ID NO: 14) may be administered for example as a single dose. The effective does of multivalent mRNA vaccine (e.g., comprising an ORF comprising the sequence of SEQ ID NO: 8, an ORF comprising the sequence of SEQ ID NO: 9, an ORF comprising the sequence of SEQ ID NO: 10, an ORF comprising the sequence of SEQ ID NO: 11, an ORF comprising the sequence of SEQ ID NO: 12, an ORF comprising the sequence of SEQ ID NO: 13, and an ORF comprising the sequence of SEQ ID NO: 14) may be administered as multiple doses (e.g., a first effective vaccine dose, a second effective vaccine dose, a third effective vaccine dose). In some embodiments, the first effective vaccine dose, the second effective vaccine dose, and/or the third effective vaccine dose are the same amount (e.g., each 12.5 μg, 25, or 50 μg). In some embodiments, first effective vaccine dose, the second effective vaccine dose, and/or the third effective vaccine dose are different amounts. In some embodiments, the effective vaccine doses are administered at least about 2 months (e.g., 2 weeks, 3 weeks, 4 weeks, 1 month, 1.5 months, 2 months, 3 months, 4 months, 5 months, 6 months) apart. In some embodiments, the effective vaccine doses are administered at least about 2 weeks, 3 weeks, 4 weeks, 1 month, 1.5 months, 2 months, 3 months, 4 months, 5 months, 6 months apart. In some embodiments, the effective vaccine doses are administered at least about 2 months apart (e.g., a second does at 2 months and a third does at 3 months).

In some embodiments, an mRNA vaccine is a heptavalent mRNA vaccine comprising seven mRNA constructs, each comprising an ORF, collectively encoding for OspA S1-S7 (e.g., a first construct comprising the sequence of SEQ ID NO: 8, a second construct comprising the sequence of SEQ ID NO: 9, a third construct comprising the sequence of SEQ ID NO: 10, a fourth construct comprising the sequence of SEQ ID NO: 11, a fifth construct comprising the sequence of SEQ ID NO: 12, a sixth construct comprising the sequence of SEQ ID NO: 13, and a seventh construct comprising the sequence of SEQ ID NO: 14). In some embodiments, the effective dose (e.g., effective amount) of heptavalent mRNA vaccine (e.g., comprising a first construct comprising the sequence of SEQ ID NO: 8, a second construct comprising the sequence of SEQ ID NO: 9, a third construct comprising the sequence of SEQ ID NO: 10, a fourth construct comprising the sequence of SEQ ID NO: 11, a fifth construct comprising the sequence of SEQ ID NO: 12, a sixth construct comprising the sequence of SEQ ID NO: 13, and a seventh construct comprising the sequence of SEQ ID NO: 14). In some embodiments, the effective dose of heptavalent mRNA vaccine (e.g., comprising a first construct comprising the sequence of SEQ ID NO: 8, a second construct comprising the sequence of SEQ ID NO: 9, a third construct comprising the sequence of SEQ ID NO: 10, a fourth construct comprising the sequence of SEQ ID NO: 11, a fifth construct comprising the sequence of SEQ ID NO: 12, a sixth construct comprising the sequence of SEQ ID NO: 13, and a seventh construct comprising the sequence of SEQ ID NO: 14) is about 20 μg, about 25 μg, about 30 μg, about 35 μg, about μg, about 45 μg, about 50 μg, about 55 μg, about 60 μg, about 65 μg, about 70 g, about 75 μg, about 80 μg, about 85 μg, about 90 g, about 95 μg, about 100 μg, about 110 μg, about 120 μg, about 130 μg, about 140 μg, about 150 μg, about 160 μg, about 170 μg, about 180 μg, about 190 μg, about 200 μg, about 250 μg, about 300 μg, or about 450 μg. In some embodiments, the effective dose of heptavalent mRNA vaccine (e.g., comprising a first construct comprising the sequence of SEQ ID NO: 8, a second construct comprising the sequence of SEQ ID NO: 9, a third construct comprising the sequence of SEQ ID NO: 10, a fourth construct comprising the sequence of SEQ ID NO: 11, a fifth construct comprising the sequence of SEQ ID NO: 12, a sixth construct comprising the sequence of SEQ ID NO: 13, and a seventh construct comprising the sequence of SEQ ID NO: 14), is 20 g, 25 g, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 110 μg, 120 μg, 130 μg, 140 μg, 150 μg, 160 g, 170 μg, 180 μg, 190 μg, 200 μg, 250 μg, 300 μg, or 450 μg. In some embodiments, the effective dose of heptavalent mRNA vaccine (e.g., comprising a first construct comprising the sequence of SEQ ID NO: 8, a second construct comprising the sequence of SEQ ID NO: 9, a third construct comprising the sequence of SEQ ID NO: 10, a fourth construct comprising the sequence of SEQ ID NO: 11, a fifth construct comprising the sequence of SEQ ID NO: 12, a sixth construct comprising the sequence of SEQ ID NO: 13, and a seventh construct comprising the sequence of SEQ ID NO: 14) is about 25 μg. In some embodiments, the effective dose of heptavalent mRNA vaccine (e.g., comprising a first construct comprising the sequence of SEQ ID NO: 8, a second construct comprising the sequence of SEQ ID NO: 9, a third construct comprising the sequence of SEQ ID NO: 10, a fourth construct comprising the sequence of SEQ ID NO: 11, a fifth construct comprising the sequence of SEQ ID NO: 12, a sixth construct comprising the sequence of SEQ ID NO: 13, and a seventh construct comprising the sequence of SEQ ID NO: 14) is about 50 μg. In some embodiments, the effective dose of heptavalent mRNA vaccine (e.g., comprising a first construct comprising the sequence of SEQ ID NO: 8, a second construct comprising the sequence of SEQ ID NO: 9, a third construct comprising the sequence of SEQ ID NO: 10, a fourth construct comprising the sequence of SEQ ID NO: 11, a fifth construct comprising the sequence of SEQ ID NO: 12, a sixth construct comprising the sequence of SEQ ID NO: 13, and a seventh construct comprising the sequence of SEQ ID NO: 14) is about 100 μg. In some embodiments, the effective dose of heptavalent mRNA vaccine (e.g., comprising a first construct comprising the sequence of SEQ ID NO: 8, a second construct comprising the sequence of SEQ ID NO: 9, a third construct comprising the sequence of SEQ ID NO: 10, a fourth construct comprising the sequence of SEQ ID NO: 11, a fifth construct comprising the sequence of SEQ ID NO: 12, a sixth construct comprising the sequence of SEQ ID No: 13, and a seventh construct comprising the sequence of SEQ ID NO: 14) is about 150 μg.

The effective dose (e.g., effective amount) of heptavalent mRNA vaccine (e.g., comprising a first construct comprising the sequence of SEQ ID NO: 8, a second construct comprising the sequence of SEQ ID NO: 9, a third construct comprising the sequence of SEQ ID NO: 10, a fourth construct comprising the sequence of SEQ ID NO: 11, a fifth construct comprising the sequence of SEQ ID NO: 12, a sixth construct comprising the sequence of SEQ ID No: 13, and a seventh construct comprising the sequence of SEQ ID NO: 14) may be administered for example as a single dose. The effective does of heptavalent mRNA vaccine (e.g., comprising a first construct comprising the sequence of SEQ ID NO: 8, a second construct comprising the sequence of SEQ ID NO: 9, a third construct comprising the sequence of SEQ ID NO: 10, a fourth construct comprising the sequence of SEQ ID NO: 11, a fifth construct comprising the sequence of SEQ ID NO: 12, a sixth construct comprising the sequence of SEQ ID NO: 13, and a seventh construct comprising the sequence of SEQ ID NO: 14) may be administered as multiple doses (e.g., a first effective vaccine dose, a second effective vaccine dose, a third effective vaccine dose). In some embodiments, the first effective vaccine dose, the second effective vaccine dose, and/or the third effective vaccine dose are the same amount (e.g., each 12.5 μg, 25 μg, or 50 μg). In some embodiments, first effective vaccine dose, the second effective vaccine dose, and/or the third effective vaccine dose are different amounts. In some embodiments, the effective vaccine doses are administered at least about 2 months (e.g., 2 weeks, 3 weeks, 4 weeks, 1 month, 1.5 months, 2 months, 3 months, 4 months, 5 months, 6 months) apart. In some embodiments, the effective vaccine doses are administered at least about 2 weeks, 3 weeks, 4 weeks, 1 month, 1.5 months, 2 months, 3 months, 4 months, 5 months, 6 months apart. In some embodiments, the effective vaccine doses are administered at least about 2 months apart (e.g., a second does at 2 months and a third does at 3 months).

In some embodiments, the ratio of mRNA comprising an ORF encoding OspA S1: OspA S2: OspA S3: OspA S4: OspA S5: OspA S6: OspA S7 is 1:1:1:1:1:1:1. In some embodiments, the ratio of mRNA comprising an ORF encoding OspA S1: OspA S2: OspA S3: OspA S4: OspA S5: OspA S6: OspA S7 is 1:2:1:1:1:1:1. In some embodiments, the ratio of mRNA comprising an ORF encoding OspA S1: OspA S2: OspA S3: OspA S4: OspA S5: OspA S6: OspA S7 is 1:1:2:1:1:1:1. In some embodiments, the ratio of mRNA comprising an ORF encoding OspA S1: OspA S2: OspA S3: OspA S4: OspA S5: OspA S6: OspA S7 is 1:1:1:2:1:1:1. In some embodiments, the ratio of mRNA comprising an ORF encoding OspA S1: OspA S2: OspA S3: OspA S4: OspA S5: OspA S6: OspA S7 is 1:1:1:1:2:1:1. In some embodiments, the ratio of mRNA comprising an ORF encoding OspA S1: OspA S2: OspA S3: OspA S4: OspA S5: OspA S6: OspA S7 is 1:1:1:1:1:2:1. In some embodiments, the ratio of mRNA comprising an ORF encoding OspA S1: OspA S2: OspA S3: OspA S4: OspA S5: OspA S6: OspA S7 is 1:1:1:1:1:1:2.

### Vaccine Efficacy

Some aspects of the present disclosure provide formulations of the compositions (e.g., RNA vaccines), wherein the mRNA is formulated in an effective amount to produce an antigen specific immune response in a subject (e.g., production of antibodies specific to a Borrelia antigen). “An effective amount” is a dose of the mRNA effective to produce a protective antigen-specific immune response in a subject.

As used herein, an immune response to a vaccine of the present disclosure is the development in a subject of a protective humoral and/or a cellular immune response to a (one or more) Borrelia protein(s) encoded by the mRNA present in the vaccine. For purposes of the present disclosure, a “humoral” immune response refers to an immune response mediated by antibody molecules, including, e.g., secretory (IgA) or TgG molecules, while a “cellular” immune response is one mediated by T-lymphocytes (e.g., CD4+ helper and/or CD8+ T cells (e.g., CTLs) and/or other white blood cells. One important aspect of cellular immunity involves an antigen-specific response by cytolytic T-cells (CTLs). CTLs have specificity for peptide antigens that are presented in association with proteins encoded by the major histocompatibility complex (MHC) and expressed on the surfaces of cells. CTLs help induce and promote the destruction of intracellular microbes or the lysis of cells infected with such microbes. Another aspect of cellular immunity involves and antigen-specific response by helper T-cells. Helper T-cells act to help stimulate the function and focus the activity nonspecific effector cells against cells displaying peptide antigens in association with MHC molecules on their surface. A cellular immune response also leads to the production of cytokines, chemokines, and other such molecules produced by activated T-cells and/or other white blood cells including those derived from CD4+ and CD8+ T-cells. Immune responses may be further divided into Th1 and Th2 responses, resulting the production of Th1-type cytokines and Th2-type cytokines, respectively. Th1-type cytokines tend to produce the proinflammatory responses responsible for killing intracellular parasites and for perpetuating autoimmune responses. The main Th1 cytokine is interferon gamma. Excessive proinflammatory responses (e.g., Th1-based responses), in some embodiments, can lead to uncontrolled tissue damage, and are counteracted by the Th2-type cytokines. The Th2-type cytokines include interleukins 4, 5, and 13, which are associated with the promotion of IgE and eosinophilic responses in atopy, and also interleukin-10, which is anti-inflammatory. In excess, Th2 responses will counteract the Th1 mediated microbicidal action. Accordingly, in some embodiments, the vaccines provided herein elicit a balanced Th1 and Th2 response. In some embodiments, administration of the vaccines provided herein may result in a Th17 response. T helper 17 cells (Th17) are a subset of pro-inflammatory T helper cells defined by their production of interleukin 17. Th17 cells maintain mucosal barriers and contribute to pathogen clearance at the mucosal surfaces. The Th17-type cytokines target innate immune cells and epithelial cells to produce G-CSF and 11-8, leading to neutrophil production and recruitment. In some embodiments, the compositions (e.g., vaccines) of the present disclosure produce a Th1 response. In some embodiments, the compositions (e.g., vaccines) of the present disclosure produce a Th2 response. In some embodiments, the compositions (e.g., vaccines) of the present disclosure produce a Th17 response. In some embodiments, the compositions (e.g., vaccines) of the present disclosure produce Th1 and Th2 responses, Th1 and Th17 responses, Th2 and Th17 responses, or Th1, Th2, and Th17 responses.

A variety of serological tests can be used to measure antibody against encoded antigen of interest, for example, a Borrelia antigen. These tests include the hemagglutination-inhibition test, complement fixation test, fluorescent antibody test, enzyme-linked immunosorbent assay (ELISA), and plaque reduction neutralization test (PRNT). Each of these tests measures different antibody activities. In exemplary embodiments, A plaque reduction neutralization test, or PRNT (e.g., PRNT50 or PRNT90) is used as a serological correlate of protection. PRNT measures the biological parameter of in vitro virus neutralization and is the most serologically virus-specific test among certain classes of viruses, correlating well to serum levels of protection from virus infection. The basic design of the PRNT allows for virus-antibody interaction to occur in a test tube or microtiter plate, and then measuring antibody effects on viral infectivity by plating the mixture on virus-susceptible cells, preferably cells of mammalian origin. The cells are overlaid with a semi-solid media that restricts spread of progeny virus. Each virus that initiates a productive infection produces a localized area of infection (a plaque), that can be detected in a variety of ways. Plaques are counted and compared back to the starting concentration of virus to determine the percent reduction in total virus infectivity. In PRNT, the serum sample being tested is usually subjected to serial dilutions prior to mixing with a standardized amount of virus. The concentration of virus is held constant such that, when added to susceptible cells and overlaid with semi-solid media, individual plaques can be discerned and counted. In this way, PRNT end-point titers can be calculated for each serum sample at any selected percent reduction of virus activity.

In functional assays intended to assess vaccine immunogenicity, the serum sample dilution series for antibody titration should ideally start below the “seroprotective” threshold titer. Regarding Borrelia neutralizing antibodies, a seropositivity threshold of 1:10 can be considered a seroprotection threshold in certain embodiments.

PRNT end-point titers are expressed as the reciprocal of the last serum dilution showing the desired percent reduction in plaque counts. The PRNT titer can be calculated based on a 50% or greater reduction in plaque counts (PRNT50). A PRNT50 titer is preferred over titers using higher cut-offs (e.g., PRNT90) for vaccine sera, providing more accurate results from the linear portion of the titration curve.

There are several ways to calculate PRNT titers. The simplest and most widely used way to calculate titers is to count plaques and report the titer as the reciprocal of the last serum dilution to show >50% reduction of the input plaque count as based on the back-titration of input plaques. Use of curve fitting methods from several serum dilutions may permit calculation of a more precise result. There are a variety of computer analysis programs available for this (e.g., SPSS or GraphPad Prism).

In some embodiments, an antibody titer is used to assess whether a subject has had an infection or to determine whether an immunizations are required. In some embodiments, an antibody titer is used to determine the strength of an autoimmune response, to determine whether a booster immunization is needed, to determine whether a previous vaccine was effective, and to identify any recent or prior infections. In accordance with the present disclosure, an antibody titer may be used to determine the strength of an immune response induced in a subject by an RNA vaccine.

In some embodiments, the anti-Borrelia antigen antibody titer, produced in a subject is increased by at least 1 log relative to a control that has not been vaccinated. For example, the anti-Borrelia antigen antibody titer produced in a subject may be increased by at least 1.5, at least 2, at least 2.5, or at least 3 log relative to a control. In some embodiments, the anti-Borrelia antigen antibody titer produced in the subject is increased by 1, 1.5, 2, 2.5 or 3 log relative to a control. In some embodiments, the anti-Borrelia antigen antibody titer produced in the subject is increased by 1-3 log relative to a control. For example, the anti-Borrelia antigen antibody titer produced in a subject may be increased by 1-1.5, 1-2, 1-2.5, 1-3, 1.5-2, 1.5-2.5, 1.5-3, 2-2.5, 2-3, or 2.5-3 log relative to a control.

In some embodiments, the anti-Borrelia antigen antibody titer produced in a subject is increased at least 2 times relative to a control that has not been vaccinated. For example, the anti-Borrelia antigen antibody titer produced in a subject may be increased at least 3 times, at least 4 times, at least 5 times, at least 6 times, at least 7 times, at least 8 times, at least 9 times, or at least 10 times relative to a control. In some embodiments, the anti-Borrelia antigen antibody titer produced in the subject is increased 2, 3, 4, 5, 6, 7, 8, 9, or 10 times relative to a control. In some embodiments, the anti-Borrelia antigen antibody titer produced in a subject is increased 2-times relative to a control. For example, the anti-Borrelia antigen antibody titer produced in a subject may be increased 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 times relative to a control.

In some embodiments, an antigen-specific immune response is measured as a ratio of geometric mean titer (GMT), referred to as a geometric mean ratio (GMR), of serum neutralizing antibody titers to LD. A geometric mean titer (GMT) is the average antibody titer for a group of subjects calculated by multiplying all values and taking the nth root of the number, where n is the number of subjects with available data.

A control, in some embodiments, is an anti-Borrelia antigen antibody titer produced in a subject who has not been administered an mRNA vaccine. In some embodiments, a control is an anti-Borrelia antigen antibody titer produced in a subject administered a recombinant or purified protein vaccine. Recombinant protein vaccines typically include protein antigens that either have been produced in a heterologous expression system (e.g., bacteria or yeast) or purified from large amounts of the pathogenic organism.

Vaccine efficacy may be assessed using standard analyses (see, e.g., Weinberg et al., J Infect Dis. 2010 Jun. 1; 201(11):1607-10). For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas:

Efficacy=(ARU−ARV)/ARU×100; and

Efficacy=(1−RR)×100.

Likewise, vaccine effectiveness may be assessed using standard analyses (see, e.g., Weinberg et al., J Infect Dis. 2010 Jun. 1; 201(11):1607-10). Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination:

Effectiveness=(1−OR)×100.

In some embodiments, efficacy of an mRNA vaccine is at least 60% relative to unvaccinated control subjects. For example, efficacy of the composition may be at least 65%, at least 70%, at least 75%, at least 80%, at least 85%, at least 95%, at least 98%, or 100% relative to unvaccinated control subjects.

Titer. An antibody titer is a measurement of the number of antibodies within a subject, for example, antibodies that are specific to a particular antigen (e.g., an anti-Borrelia antigen). Antibody titer is typically expressed as the inverse of the greatest dilution that provides a positive result. Enzyme-linked immunosorbent assay (ELISA) is a common assay for determining antibody titers, for example.

In some embodiments, the effective amount of a composition of the present disclosure is sufficient to produce a 1:1,000-10,000 neutralizing antibody titer produced by neutralizing antibody against the specific antigen as measured in serum of the subject at 1-72 hours post administration. In some embodiments, the effective amount is sufficient to produce a 1:1,000-5,000 neutralizing antibody titer produced by neutralizing antibody against the specific antigen as measured in serum of the subject at 1-72 hours post administration. In some embodiments, the effective amount is sufficient to produce a 1:5,000-10,000 neutralizing antibody titer produced by neutralizing antibody against the specific antigen as measured in serum of the subject at 1-72 hours post administration.

In some embodiments, the neutralizing antibody titer is at least 100 NT₅₀. For example, the neutralizing antibody titer may be at least 200, 300, 400, 500, 600, 700, 800, 900 or 1000 NT₅₀. In some embodiments, the neutralizing antibody titer is at least 10,000 NT₅₀.

In some embodiments, the neutralizing antibody titer is at least 100 neutralizing units per milliliter (NU/mL). For example, the neutralizing antibody titer may be at least 200, 300, 400, 500, 600, 700, 800, 900 or 1000 NU/mL. In some embodiments, the neutralizing antibody titer is at least 10,000 NU/mL.

In some embodiments, an anti-Borrelia antigen antibody titer produced in the subject is increased by at least 1 log relative to an unvaccinated control subject. For example, an anti-Borrelia antigen antibody titer produced in the subject may be increased by at least 2, 3, 4, 5, 6, 7, 8, 9 or 10 log relative to an unvaccinated control subject.

In some embodiments, an anti-Borrelia antigen antibody titer produced in the subject is increased at least 2 times relative to an unvaccinated control subject. For example, an anti-Borrelia antigen antibody titer produced in the subject is increased by at least 3, 4, 5, 6, 7, 8, 9 or 10 times relative to an unvaccinated control subject.

In some embodiments, the anti-Borrelia antigen antibody titer produced in the subject is a population of neutralizing antibodies. In some embodiments, the population of neutralizing antibodies comprises antibodies that bind to OspA S1, antibodies that bind to OspA S2, antibodies that bind to OspA S3, antibodies that bind to OspA S4, antibodies that bind to OspA S5, antibodies that bind to OspA S6, and/or antibodies that bind to OspA S7.

### Methods of Inducing an Immune Response

In some aspects, the disclosure relates to methods of inducing an immune response in a subject (e.g., a human subject) an immune response to a Borrelia bacterial infection. In some embodiments, the method comprises administering to the subject a mRNA vaccine described herein in an amount effective at inducing in the subject a population of neutralizing antibodies.

In some embodiments, the population of neutralizing antibodies comprises IgG antibodies.

In some embodiments, the population of neutralizing antibodies comprises antibodies that bind to OspA S1, antibodies that bind to OspA S2, antibodies that bind to OspA S3, antibodies that bind to OspA S4, antibodies that bind to OspA S5, antibodies that bind to OspA S6, antibodies that bind to OspA S7, or any combination thereof.

### EXAMPLES

According to the present disclosure, the manufacture of polynucleotides and/or parts or regions thereof may be accomplished utilizing the methods taught in International Publication WO2014/152027, entitled “Manufacturing Methods for Production of RNA Transcripts,” the content of which is incorporated herein by reference in its entirety.

Purification methods may include those taught in International Publication WO2014/152030 and International Publication WO2014/152031, each of which is incorporated herein by reference in its entirety.

Detection and characterization methods of the polynucleotides may be performed as taught in International Publication WO2014/144039, which is incorporated herein by reference in its entirety.

Characterization of the polynucleotides of the disclosure may be accomplished using polynucleotide mapping, reverse transcriptase sequencing, charge distribution analysis, detection of RNA impurities, or any combination of two or more of the foregoing. “Characterizing” comprises determining the RNA transcript sequence, determining the purity of the RNA transcript, or determining the charge heterogeneity of the RNA transcript, for example. Such methods are taught in, for example, International Publication WO2014/144711 and International Publication WO2014/144767, the contents of each of which are incorporated herein by reference in their entirety.

Production, purification, and evaluation of mRNA vaccines encoding Lyme Disease (LD) antigens may be accomplished using methods described in International Publication WO 2017/070623 and International Publication WO 2018/170256, the contents of each of which are incorporated herein by reference in their entirety.

In experiments where a lipid nanoparticle (LNP) formulation was used, the formulation includes 0.5-15% PEG-modified lipid, 5-25% non-cationic lipid, 25-55% sterol, and 20-60% ionizable amino lipid. The PEG-modified lipid is 1,2 dimyristoyl-sn-glycerol, methoxypolyethyleneglycol (PEG2000 DMG), the non-cationic lipid is 1,2 distearoyl-sn-glycero-3-phosphocholine (DSPC), the sterol is cholesterol, and the ionizable amino lipid is

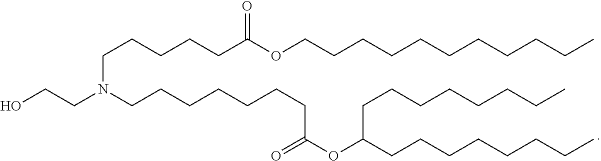 [SMILES: CCCCCCCCCCCOC(=O)CCCCCN(CCO)CCCCCCCC(=O)OC(CCCCCCCC)CCCCCCCC]

### Example 1. Expression of mRNA Encoding Borrelia OspA S1-S7 Proteins

Seven different serotypes (S) of OspA (S1, S2, S3, S4, S5, S6, and S7) were tested for their ability to be expressed in vitro. mRNA having an ORF encoding one of the OspA S1-S7 (seven mRNAs, each comprising an ORF encoding one of the OspA serotypes) were introduced to cells. The mRNA having ORF(s) encoding each serotype (S) of OspA (S1, S2, S3, S4, S5, S6 and S7) was modified to include an N-terminal transmembrane (TM) domain (SEQ ID NOs: 8-12 and 14). The transmembrane constructs were analyzed for protein expression using flow cytometry. The results indicate that OspA S6 had markedly less expression than the other OspA serotypes (S1-S5, and S7) (data not shown).

To determine why there were relatively low levels of OspA S6 expression compared to other OspA serotypes, the amino acid sequence of OspA S6 was aligned with amino acid sequences of the other OspA serotypes (S1-S5, and S7). Based on these alignments it was determined that there were two amino acids in OspA S6 that were different from other OspA serotypes (S1-S5, and S7) and may cause destabilization. At amino acid position 88 of OspA S6 there was a serine, whereas other OspA serotypes (S1-S5, and S7) had a leucine at this position. At amino acid position 200 of OspA S6 there was an alanine, whereas other OspA serotypes (S1-S5, and S7) had a valine at this position.

To test whether modifications of the mRNA having an ORF encoding OspA S6 would result in detectable levels of protein expression, mRNA constructs expressing a variant of OspA S6 was expressed in vitro (SEQ ID NO: 13). In the OspA S6 variant a serine to leucine mutation was engineered at amino acid position 88 (S88L) and an alanine to valine mutation was engineered at amino acid position 200 (A200V).

Further, an OspA S1 variant was engineered and expressed in vitro. The modifications to OspA S1 include removal of the leukocyte function-associated antigen (LFA) epitope (amino acid residues 165-173) and the N-terminus (Nterm) was modified to include a transmembrane (TM) domain from neuraminidase. The modified OspA S1 and S6 variant constructs (SEQ ID NO: 8 and SEQ ID NO: 13) were expressed in cells at a concentration of 500 ng/μL and 125 ng/μL.

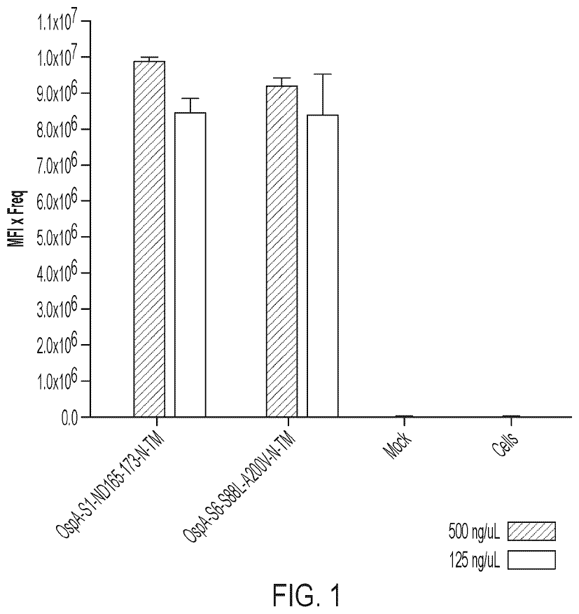

The results indicate that at both concentrations (500 ng/μL and 125 ng/μL), the modification made to OspA S1 and S6 variant constructs (SEQ ID NO: 8 and SEQ ID NO: 13) resulted in detectable levels of protein expression in vitro compared to mock-transfected cells and non-transfected cells (FIG. 1). These expression levels in vitro were comparable to OspA S2-S5 and S7 with N-terminal TM domains (SEQ ID NOs: 9-12 and SEQ ID NO: 14).

The OspA S1-S7 constructs having open reading frames encoding the nucleotide sequence of SEQ ID NOs: 8-14 encode for proteins having the amino acid sequence of SEQ ID NOs: 1-7, respectively. Alignments of the full-length of OspA proteins encoded by SEQ ID NOs: 1-7 were performed to compare their percent identity to their wildtype counterparts (SEQ ID NOs: 24-30). The results were as follows:
Borrelia burgdorferi OspA S1 (SEQ ID NO: 24) is 89.59% identical to OspA S1 (SEQ ID NO: 1);
Borrelia afzelii OspA S2 (SEQ ID NO: 25) is 90.71% identical to OspA S2 (SEQ ID NO: 2);
Borrelia garinii OspA S3 (SEQ ID NO: 26) is 90.37% identical to OspA S3 (SEQ ID NO: 3);
Borrelia bavariensis OspA S4 (SEQ ID NO: 27) is 90.71% identical to OspA S4 (SEQ ID NO: 4);
Borrelia garinii OspA S5 (SEQ ID NO: 28) is 91.85% identical to OspA S5 (SEQ ID NO: 5);
Borrelia garinii OspA S6 (SEQ ID NO: 29) is 91.11% identical to OspA S6 (SEQ ID NO: 6);
Borrelia garinii OspA S7 (SEQ ID NO: 30) is 91.48% identical to OspA S7 (SEQ ID NO: 7).

### Example 2. Total IgG, LA-2 Competition ELISAs and In Vivo Challenge Study

A screen was conducted using vaccines comprising lipid nanoparticles encapsulating an mRNA having an ORF encoding OspA S1 wild-type or OspA S1 variants to test the immunogenicity of the vaccines. Additionally, the ability of the mRNA vaccine compositions to elicit antibodies that recognize the same epitope as a known bactericidal antibody (LA-2) was assessed. Construct designs of immunogens encoded by the mRNA are indicated in Table 3.

The groups tested were mice administered either a non-translating mRNA (NTFIX), or an mRNA having an ORF encoding either a wild-type (WT) full-length, soluble, OspA Serotype 1 (S1) (WT-Soluble) protein, or an OspA S1 with the LFA epitope removed (Soluble), or an OspA S1 with the LFA epitope removed and an N-terminal transmembrane domain (Nterm-TM) (SEQ ID NO: 8), or an OspA S1 with the LFA epitope removed and a C-terminal transmembrane domain (Cterm-TM).

TABLE 3
OspA Serotype 1 construct design

| | Dose level | | | | |
|---|---|---|---|---|---|
| | (μg/ | | Dosing | | |
| N= | animal) | Immunogen | regimen | Bleeds | Readout |
| 10 | 2 | NTFIX | D 1, D 22, | D 1, D 21, D 43, | S1 OspA |
| | | | D 45 | D 56 (terminal) | binding ELISA |
| 10 | 2 | OspA-S1-ND165-173 | | | LA2 |
| | | (Soluble) | | | Competition |
| 10 | 2 | OspA-S1-ND165-173- | | | ELISA |
| | | Nterm-TM (Nterm-TM) | | | |
| | | (SEQ ID NO: 8) | | | |
| 10 | 2 | OspA-S1-ND165-173- | | | |
| | | Cterm-TM(Cterm-TM) | | | |
| 10 | 2 | OspA-S1 (WT Soluble) | | | |
| | | protein | | | |

Mice were immunized with three doses (2 μg/animal) of one of the vaccines listed in Table 3. The first dose was administered on day 1 (d1), the second dose on day 22 (d22), and the third dose on day 45 (d45). Blood samples were collected on d1, day 21 (d21), day 43 (d43), and day 56 (d56). Two readout enzyme-linked immunosorbent assay (ELISA) were performed to determine antibody titers. An S1 OspA binding ELISA and LA2 competition ELISA were performed. Additionally, a serum bactericidal assay was performed to evaluate the bactericidal activity of OspA S1 constructs.

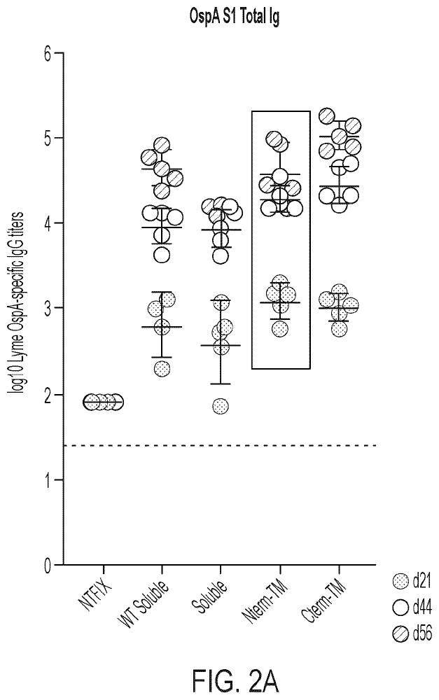

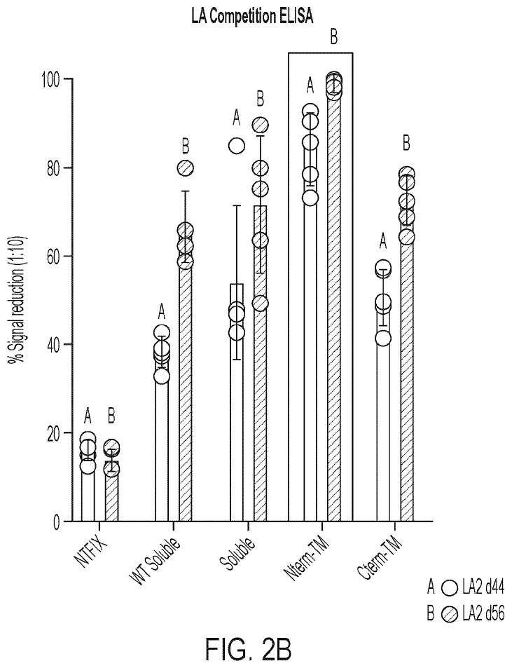

The total OspA IgG levels were measured on d21, d44 and d56. Compared to NTFIX treated mice, all groups immunized with mRNA having an ORF encoding an OspA S1 antigen or OspA S1 variant showed enhanced production of OspA IgG at d21, d44, and d56. Among the mice that were immunized with mRNA having an ORF encoding an OspA S1 antigen or OspA S1 variant, the third dose administered on d56 did not enhance OspA IgG beyond levels measured on d44 (FIG. 2A). Therefore, several OspA S1 constructs demonstrate high induction of total IgG.

LA2 competition ELISA assays were performed on serum samples from d44 and d56. Compared to NTFIX treated mice, mice administered vaccines comprising mRNA having an ORF encoding an OspA S1 antigen or OspA S1 variant showed significant LA2 signal reduction at d44 and a further decrease at d56 (FIG. 2B). Results from the two ELISA assays indicate that treatment with OspA-S1-A results in high total IgG and LA2 signal reduction. Therefore, the mRNA OspA S1 constructs result in high levels of functional antibodies.

### Example 3. Serum Bactericidal Activity of Borrelia OspA S1 Immunized Mouse Sera

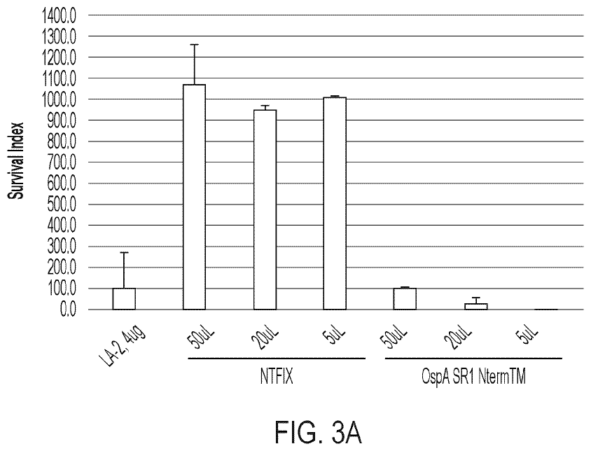

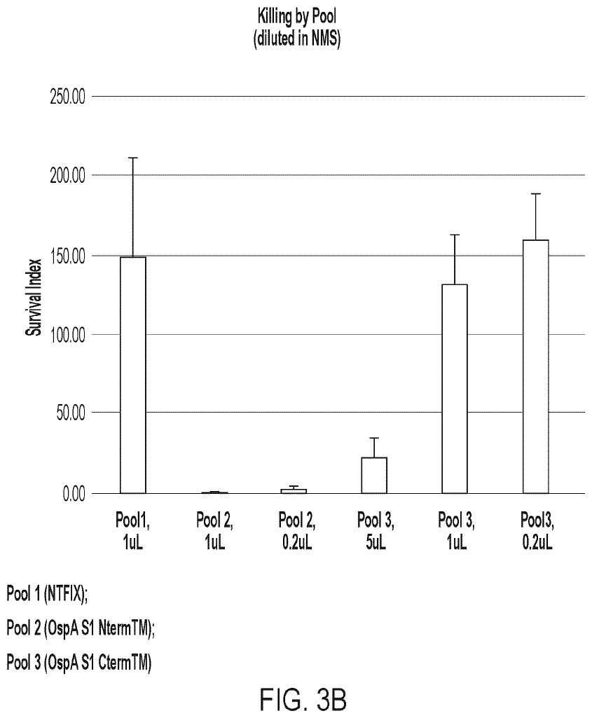

To determine the bactericidal activity of OspA-S1-ND165-173-Nterm-TM (Nterm-TM) (SEQ ID NO: 8), a serum bactericidal assay was performed. Survival indexes were measured in serum collected from OspA-S1-ND165-173-Nterm-TM (Nterm-TM) (SEQ ID NO: 8) treated and positive (NTFIX) and negative controls (LA-2). The serum from OspA-S1-ND165-173-Nterm-TM (Nterm-TM) (SEQ ID NO: 8) treated samples resulted in significantly less bacterial survival compared to the NTFIX condition (FIG. 3A).

The bactericidal activity of the OspA-S1-ND165-173-Nterm-TM (Nterm-TM) (SEQ ID NO: 8) (Pool 2) was compared to the OspA-S1-ND165-173-Cterm-TM (Pool 3) and non-translating mRNA (NTFIX) controls (Pool 1) using a serum bactericidal assay. The results indicate that the survival index of N-terminal transmembrane constructs (Pool 2) was much less than the C-terminal transmembrane constructs (Pool 3). For example, at comparable volumes (1 μL) the survival index of Pool 2 is about 0, whereas the survival index of Pool 3 is greater than 100 (FIG. 3B).

These results indicate that the sera from the OspA-S1-ND165-173-Nterm-TM (Nterm-TM) (SEQ ID NO: 8) immunized mice can kill bacteria.

### Example 4. In Vivo Challenge Study

The immunogenicity of OspA S1 vaccines comprising lipid nanoparticles encapsulating an mRNA having an ORF encoding OspA S1 or OspA S1 variant was tested using a tick bite model of infection. Constructs were tested for their ability to prevent infection and analyze the functional abilities of the antibodies generated by vaccination to kill Borrelia. Study design and construct designs of immunogens encoded by the mRNA are indicated in Table 4.

The groups tested were mice administered a non-translating mRNA (NTFIX), an mRNA having an ORF encoding an OspA S1 with the LFA epitope removed (Soluble), an mRNA having an ORF encoding OspA S1 with the LFA epitope removed and an N-terminal transmembrane domain (Nterm-TM) (SEQ ID NO: 8), or an mRNA having an ORF encoding OspA S1 with the LFA epitope removed and a C-terminal transmembrane domain (Cterm-TM).

TABLE 4
Immunogenicity and challenge study design and OspA Serotype 1 construct design

| | | Dose level | | | | |
|---|---|---|---|---|---|---|
| | | (μg/ | | Dosing | | |
| GR# | N= | animal) | Immunogen | regimen | Bleeding | Readout |
| 1 | 20 | 2 | NTFIX | D 1, 22, 45 | D 1, 22, 45 | S1 OspA Binding ELISA |
| 2 | 5 | 2 | OspA-S1- | | (pre-vax), | Tick bite challenge |
| | | | ND165-173 | | 55 | |
| | | | (Soluble) | | | |
| 3 | 5 | 2 | OspA-S1- | | | |
| | | | ND165-173- | | | |
| | | | Nterm-TM | | | |
| | | | (Nterm-TM) | | | |
| | | | (SEQ ID NO: 8) | | | |
| 4 | 5 | 2 | OspA-S1- | | | |
| | | | ND165-173- | | | |
| | | | Cterm- | | | |
| | | | TM(Cterm-TM) | | | |

Mice were immunized with three doses (2 μg/animal) of one of the vaccines listed in Table 4. The dosing regimen was as follows: the first dose was administered on day 1 (d1), the second dose on day 22 (d22), and the third dose on day 45 (d45). Blood samples were collected for all groups on d1, d22, d45 (pre-vaccination), and day 55 (d55).

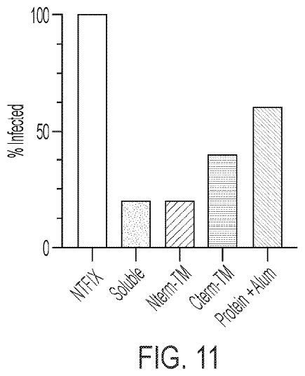

The readouts were a S1 OspA binding enzyme-linked immunosorbent assay (ELISA) and LA2 ELISA competition assay and a tick bite challenge. The tick bite challenge was performed using Borrelia burgdorferi strain B31. Mice were challenged with five ticks, resulting in an unknown bacterial load administered, and 2 weeks post tick feed mice were sacrificed, and skin, hearts and bladders were collected. These tissues were then cultured and monitored for the presence of spirochetes for 3 weeks. The tick bite challenge readout was performed 2 weeks post tick feed and the presence of spirochetes on skin, heart and bladder was observed for 3 weeks (no quantification). The results, as measured by the percentage of infected mice, demonstrates that each of the OspA S1 mRNA constructs prevented infection, and they did so to a greater extent relative to administration of OspA S1 protein (FIG. 11—the sample labeled “soluble” corresponds to OspA-S1-ND165-173 (Soluble)).

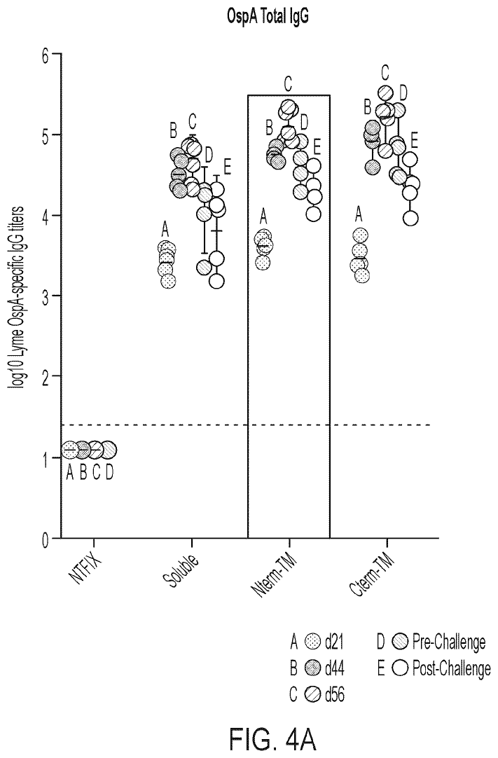

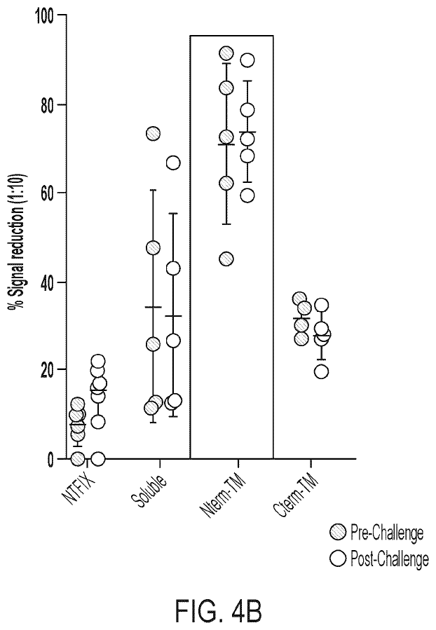

The total OspA IgG levels were measured on d22, d45 and d55. Compared to NTFIX treated mice, all groups immunized with mRNA having an ORF encoding an OspA S1 antigen or OspA S1 variant showed enhanced production of OspA IgG at d22, d45 and d55 (FIG. 4A).

LA2 ELISA competition assays were performed on blood samples pre-tick bite and post-tick bite challenge. Groups of mice with low LA2 competition titers were considered protected in the challenge. Therefore, in this model protection is also provided by an LA-2 independent mechanism. Compared to NTFIX treated mice, mice administered vaccine compositions comprising mRNA having an ORF encoding an OspA-S1-ND165-173-Nterm-TM (Nterm-TM) (SEQ ID NO: 8) showed significant LA2 signal reduction (FIG. 4B). Results from the two ELISA assays indicate that OspA-S1-ND165-173-Nterm-TM (Nterm-TM) (SEQ ID NO: 8) treatment results in high total IgG and LA2 signal reduction.

Results of the tick bite challenge indicate that several constructs, including OspA-S1-ND165-173-Nterm-TM (Nterm-TM) (SEQ ID NO: 8), resulted in high induction of total IgG. Also, the results of the LA-2 competition ELISAs suggest high levels of functional antibodies produced in response to several OspA S1 constructs, notably, the OspA-S1-ND165-173-Nterm-TM (Nterm-TM) (SEQ ID NO: 8). These results are reproducible and demonstrate variation in ability of constructs to protect mice from challenge.

### Example 5. In Vivo Challenge Study

To test if mice immunized twice with a lower dose of OspA S1 NtermTM mRNA, or with six additional mRNAs, OspA S1-S7 NtermTM, are still protected in a tick bite challenge model the following experiments were performed, or if the six additional mRNAs would interfere with the ability to elicit a protective response with the serotype 1 mRNA. Additionally, the total and functional antibody titers to recombinant antibodies were compared using LA-2 (only bactericidal against S1) and a positive control antibody (bactericidal against all 7 serotypes).

Mice were administered vaccines comprising lipid nanoparticles encapsulating an mRNA having one ORF encoding OspA S1 (e.g., monovalent) (SEQ ID NO: 8) or seven mRNAs collectively encoding OspA S1-S7 (e.g., heptavalent; seven mRNAs, each mRNA having an ORF encoding one of the seven OspA serotypes) (SEQ ID NOs: 8-14). Study design and construct designs of immunogens encoded by the mRNA are indicated in TABLE 5.

Briefly, mRNA having an ORF encoding OspA S1 (e.g., monovalent) (SEQ ID NO: 8) or OspA S1-S7 (e.g., heptavalent) (SEQ ID NOs: 8-14) were designed as previously described in Example 1. The OspA S1 was engineered to include removal of the leukocyte function-associated antigen (LFA) epitope (e.g., amino acid residues 165-173) and the N-terminus (Nterm) was modified to include a transmembrane (TM) domain (SEQ ID NO: 8). The S6 was engineered to include a serine to leucine mutation at amino acid position 88 (S88L) and an alanine to valine mutation at amino acid position 200 (A200V) and the N-terminus (Nterm) was modified to include a transmembrane (TM) domain (SEQ ID NO: 13). Some mice were immunized with non-translating mRNA (NTFTX) as a negative control. Other mice were immunized with OspA S1 Cterm ferritin stabilized or Recombitek® (Santa Cruz Biotechnology) with alum.

TABLE 5
Study design to compare monovalent and heptavalent constructs.

| GR | | Dose | | | | | |
|---|---|---|---|---|---|---|---|
| # | N= | (μg/animal) | Immunogen | Doses | Bleeding | Challenge | Readout |
| 1 | 5 | 1 | NTFIX | D 1, 21 | D −1, 20, | Tick bite | 3-4 week |
| 2 | 5 | 1 | OspA-S1-ND165- | | pre- | challenge | monitoring |
| | | | 173-Nterm-TM | | challenge, | (5 ticks | of combined |
| | | | (Nterm-TM) (SEQ | | and post- | per | cultures |
| | | | ID NO: 8) | | challenge | mouse) | from |
| 3 | 5 | 3.5 | OspA S1-S7 | | | | multiple |
| | | | NtermTM (SEQ ID | | | | tissues |
| | | | NOs: 8-14) | | | | |
| 4 | 5 | 1 | OspA S1 Cterm | | | | |
| | | | ferritin stabilized | | | | |
| 5 | 5 | 1 | Recombitek ® + alum | | | | |

Mice (n=5 animals/group) were immunized with two doses of one of the vaccines listed in Table 5. The dosing regimen was as follows: the first dose was administered on day 1 (d1), and the second dose on day 21 (d21). Blood samples were collected for all groups on d1, day 20 (d20), and pre-challenge and post-challenge. The readouts were measuring OspA antibody response and a tick bite challenge. The tick bite challenge was performed using Borrelia burgdorferi. Mice were challenged with five ticks. Mice were monitored for 3-4 weeks comprising of combined cultures from multiple tissues.

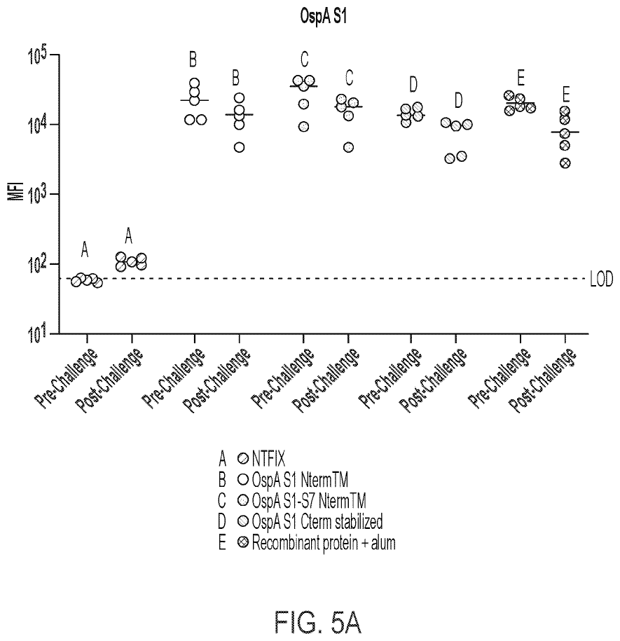

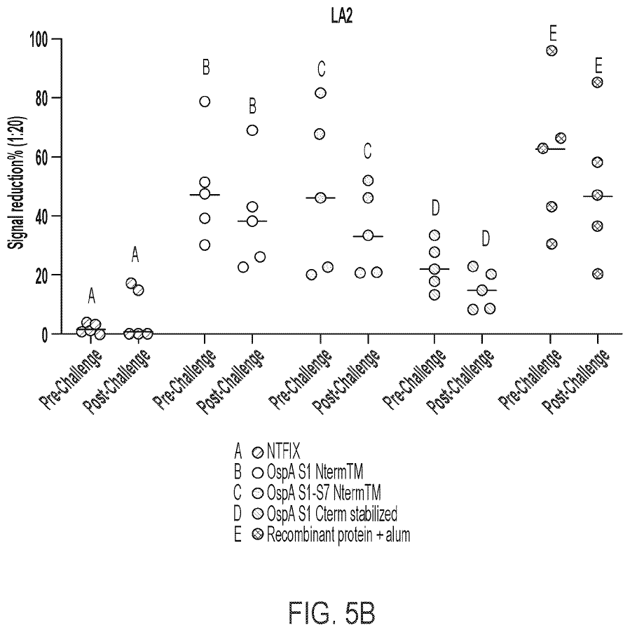

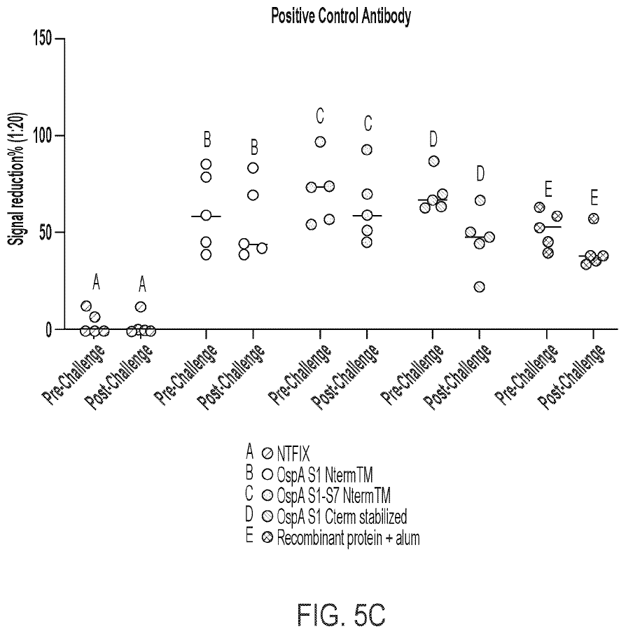

The total OspA S1 IgG levels were measured. Compared to NTFIX treated mice, all groups immunized with mRNA having an ORF(s) encoding OspA S1 (e.g., monovalent) (SEQ ID NO: 8) or OspA S1-S7 (e.g., heptavalent) (SEQ ID NOs: 8-14) showed enhanced production of OspA S1 IgG (FIG. 5A).

LA2 ELISA competition assays were performed. Groups of mice with low LA2 competition titers were considered protected in the challenge. Compared to NTFIX treated mice, mice administered vaccines comprising mRNA having an ORF(s) encoding OspA S1 (e.g., monovalent) (SEQ ID NO: 8) or OspA S1-S7 (e.g., heptavalent) (SEQ ID NOs: 8-14) showed significant LA2 signal reduction (FIG. 5B).

A positive control antibody was also used in an ELISA competition assay that was performed. The positive control antibody is an additional monoclonal antibody to OspA that is bactericidal. In contrast to LA-2, which only recognizes an epitope on OspA S1, this positive control antibody recognizes all seven OspA serotypes. Compared to NTFIX treated mice, mice administered vaccines comprising mRNA having an ORF(s) encoding OspA S1 (e.g., monovalent) (SEQ ID NO: 8) or OspA S1-S7 (e.g., heptavalent) (SEQ ID NOs: 8-14) showed significant signal reduction compared to positive control antibody (FIG. 5C).

These results indicate that mice immunized with 2 doses of OspA S1 NtermTM at 1 μg each are protected from infection. Also, mice immunized with 2 doses OspA S1-S7 NtermTM at 0.5 μg per serotype (3.5 μg total dose) are protected from infection. Finally, the total and functional antibody titers were not significantly different between mRNA groups and protein groups.

### Example 6. Characterization of OspA Antibodies and OspA Antigens

To determine the immunogenicity of OspA-S1-ND165-173-Nterm-TM (Nterm-TM) (SEQ ID NO: 8) and OspA S1-S7 (SEQ ID NOs: 8-14) mice were administered vaccines comprising lipid nanoparticles encapsulating an mRNA having an ORF(s) encoding OspA-S1-ND165-173-Nterm-TM (OspA S1 NtermTM; mRNA-1982) (SEQ ID NO: 8) and OspA S1-S7(OspA S1-S7 NtermTM; mRNA-1975; seven mRNAs, each mRNA having an ORF encoding one of the seven OspA serotypes) (SEQ ID NOs: 8-14) constructs. Other mice were immunized with S1-S7 soluble protein with alum or Recombitek® (Santa Cruz Biotechnology) with Alum or PBS control. OspA S1 NtermTM was administered at either 0.5 μg or 1 μg and OspA S1-S7 NtermTM was administered at either 3.5 μg or 7 μg.

TABLE 6
Study design to test immunogenicity of modified OspA S1 and S6

| | | Dose | | | | |
|---|---|---|---|---|---|---|
| GR# | N= | (μg/animal) | Immunogen | Doses | Bleeding | Readout |
| 1 | 10 | 0.5 | OspA NtermTM S1 | D 1, 21 | D −1, 20, | Luminex |
| | | | (SEQ ID NO: 8) | | D 36 | |
| 2 | 10 | 1 | OspA NtermTM | | (Terminal) | |
| | | | S1(SEQ ID NO: 8) | | | |
| 3 | 10 | 3.5 | OspA NtermTM S1-S7 | | | |
| | | | (SEQ ID NOs: 8-14) | | | |
| 4 | 10 | 7 | OspA NtermTM S1-S7 | | | |
| | | | (SEQ ID NOs: 8-14) | | | |
| 5 | 10 | 1 | Recombitek ® + Alum | | | |
| 6 | 10 | 2 | Recombitek ® + Alum | | | |
| 7 | 10 | 5 | Recombitek ® + Alum | | | |
| 8 | 10 | 7 | OspA S1-S7 soluble | | | |
| | | | protein + Alum | | | |
| 9 | 10 | 14 | OspA S1-S7 soluble | | | |
| | | | protein + Alum | | | |
| 10 | 10 | 0 | PBS | | | |

Mice (n=10 animals) were immunized with one of the vaccines listed in Table 6. The dosing regimen was that administrations occurred on day 1 (d1) and day 21 (d21). Blood samples were collected on d1, day 20 (d20) and day 36 (d36). The readout was Luminex binding assay.

### Total IgG

The immunogenicity of OspA NtermTM S1 (SEQ ID NO: 8) and OspA NtermTM S1-S7 (SEQ ID NOs: 8-14) in vivo was determined by administering vaccines comprising lipid nanoparticles encapsulating an mRNA having an ORF(s) encoding OspA NtermTM S1 (SEQ ID NO: 8) and OspA NtermTM S1-S7 (SEQ ID NOs: 8-14) constructs to mice and assessing total IgG.

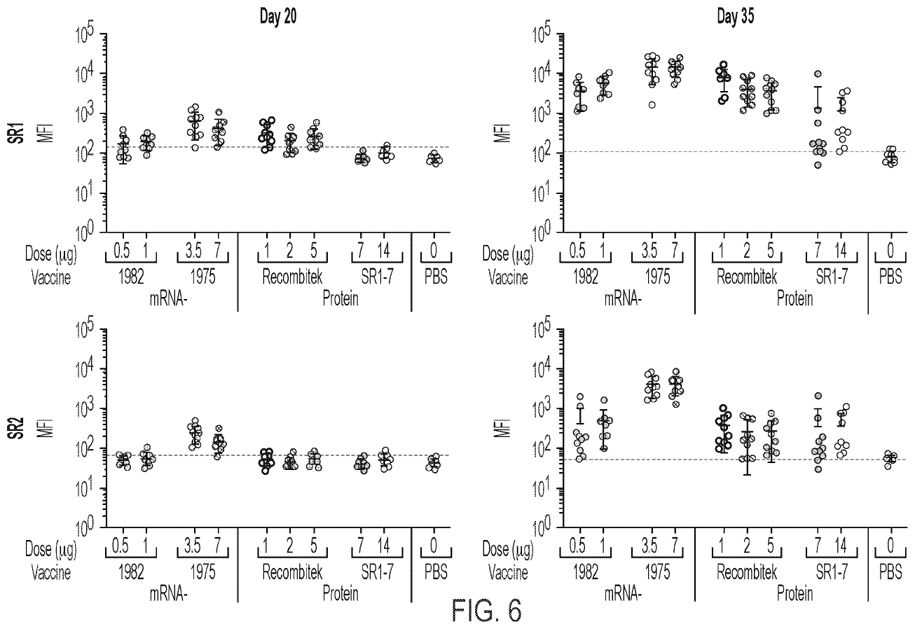

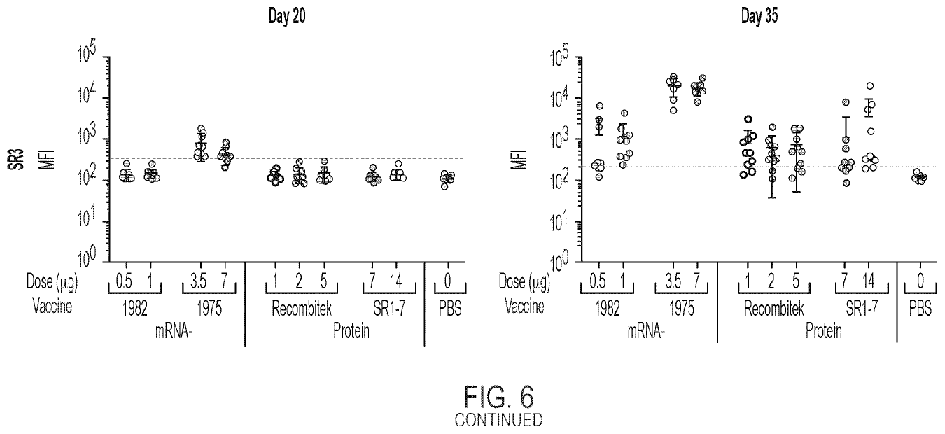

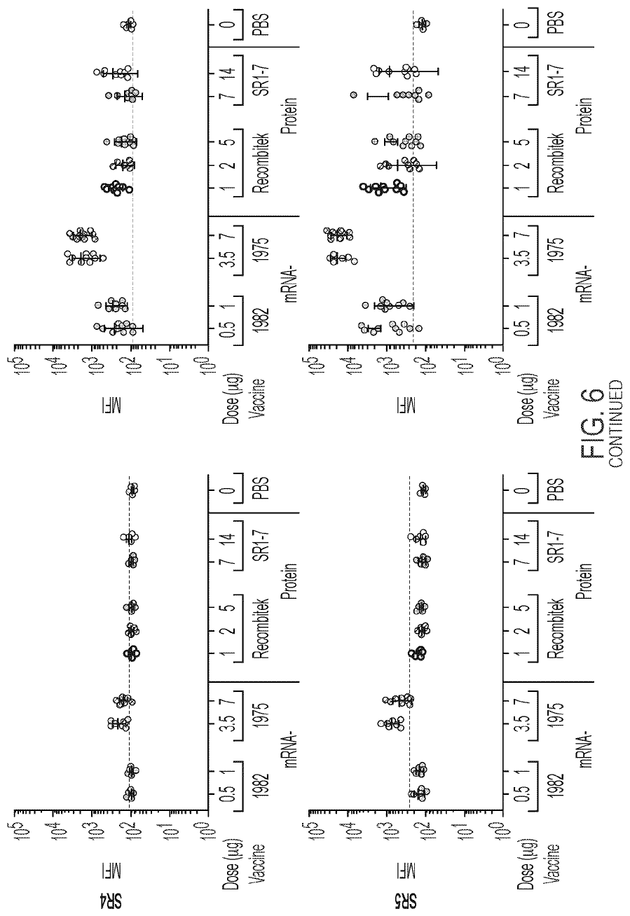

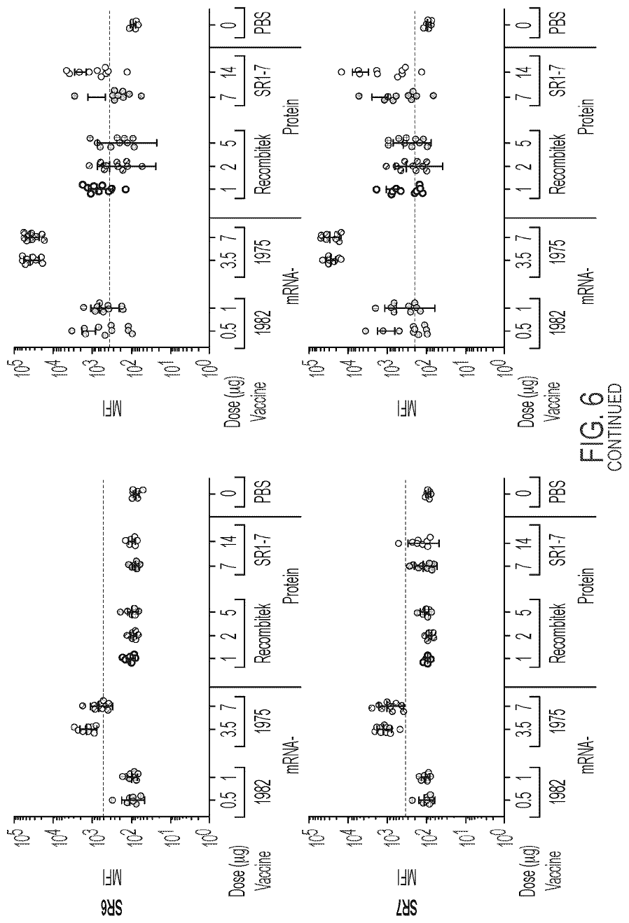

The total OspA IgG levels were measured on d20 and d36 using Luminex. The mean fluorescence intensity (MFI) measured across all serotypes indicate that higher titers were observed in OspA NtermTM S1 and OspA NtermTM S1-S7 compared to mice that were administered protein or PBS. These results indicate that the modified OspA S1 and S6 or monovalent OspA S1 induce potent antibody responses (FIG. 6).

### Surface Binding of Borrelia by OspA Antibodies

The ability of antibodies generated in response to OspA NtermTM S1 (SEQ ID NO: 8) and OspA NtermTM S1-S7 (SEQ ID NOs: 8-14) to bind to Borrelia bacteria were tested using a surface binding assay. The mRNA vaccines were compared to mice administered PBS or Recombitek® or S1-S7 protein, LA-2 or S1 Hyper+(hyperimmune sera generated by immunizing mice with 2 doses of 25 μg of the OspA S1 mRNA).

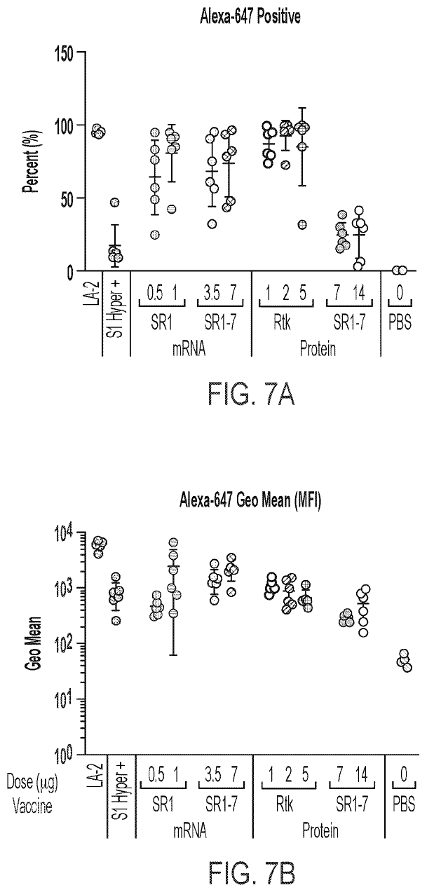

The results of the surface binding assay demonstrate that antibodies generated by OspA NtermTM S1 (SEQ ID NO: 8) and OspA NtermTM S1-S7 (SEQ ID NOs: 8-14) mRNA immunization bind to the surface of Borrelia bacteria (FIGS. 7A-7B).

### Agglutination/Membrane Integrity Study

The ability of antibodies generated in response to OspA NtermTM S1 (SEQ ID NO: 8) and OspA NtermTM S1-S7 (SEQ ID NOs: 8-14) to agglutinate Borrelia bacteria was tested using an Agglutination assay. Additionally, the ability of antibodies generated in response to OspA NtermTM S1 (SEQ ID NO: 8) and OspA NtermTM S1-S7 (SEQ ID NOs: 8-14) to compromise the membranes of Borrelia bacteria was tested using a Membrane Integrity assay.

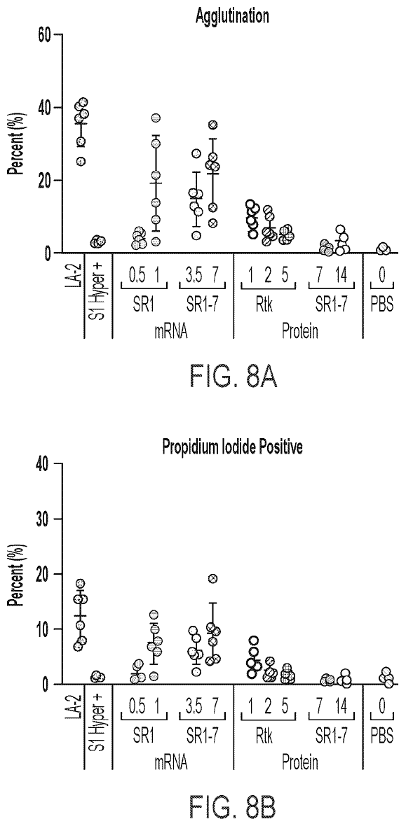

The results of the agglutination and membrane integrity studies demonstrate that antibodies generated by OspA NtermTM S1 (SEQ ID NO: 8) and OspA NtermTM S1-S7 (SEQ ID NOs: 8-14) mRNA promote agglutination of Borrelia bacteria. Additionally, antibodies generated by OspA NtermTM S1 (SEQ ID NO: 8) and OspA NtermTM S1-S7 (SEQ ID NOs: 8-14) mRNA compromise the membrane integrity of Borrelia bacteria (FIGS. 8A-8B).

### Serum Bactericidal Assay (SBA)

Serum bactericidal activity of antibodies generated in response OspA NtermTM S1 (SEQ ID NO: 8) and OspA NtermTM S1-S7 (SEQ ID NOs: 8-14) to kill an OspA S1 Borrelia burgdorferi bacteria was tested using a Serum Bactericidal Assay (SBA). In the SBA, fluorescence is induced in bacteria that are alive after incubation with serum and complement. The OspA S1-7 recombinant protein used as a control is not lipidated, however Recombitek is lipidated. Lipidated proteins are highly reactogenic.

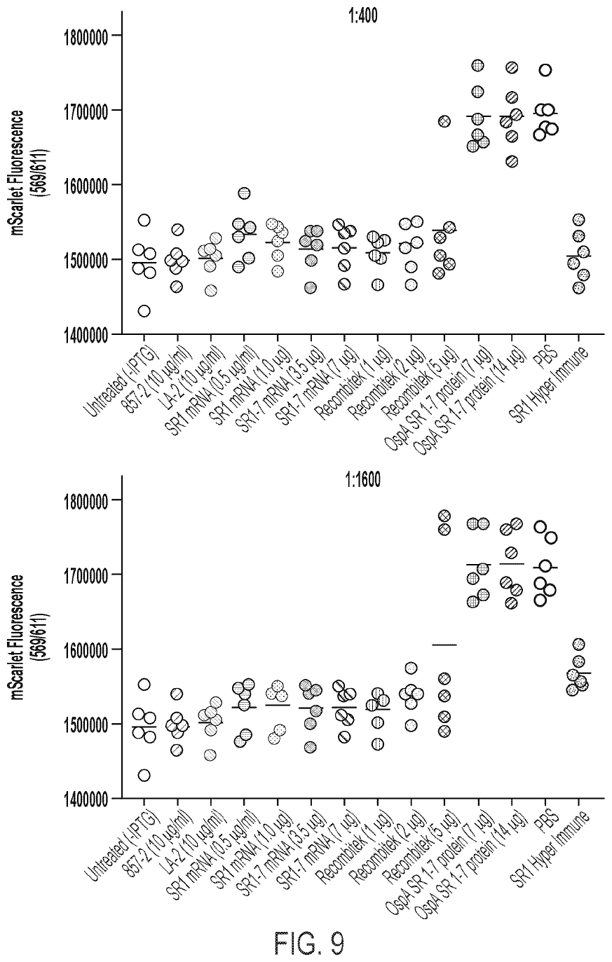

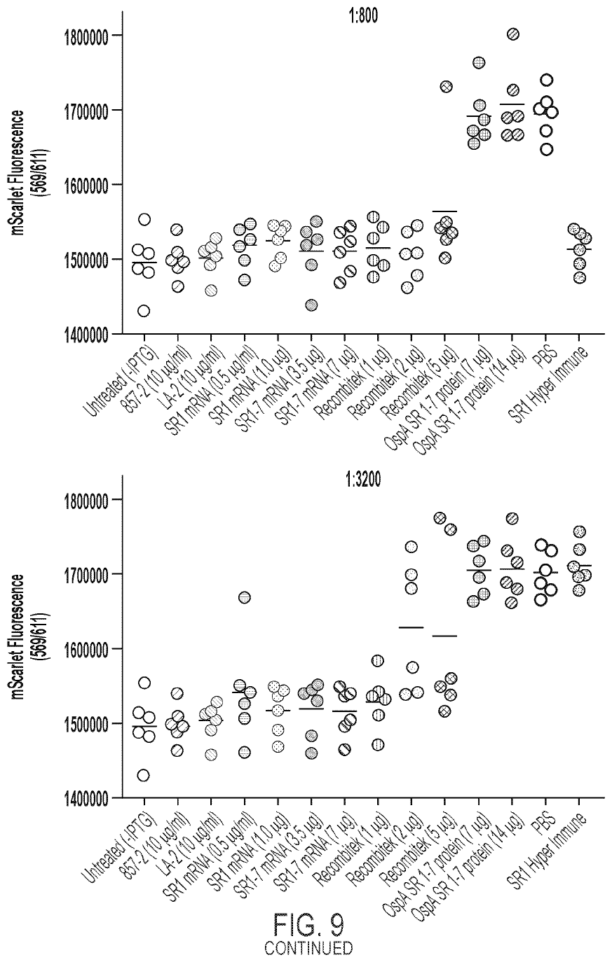

The results of the SBA demonstrate that antibodies generated by OspA NtermTM S1 (SEQ ID NO: 8) and OspA NtermTM S1-S7 (SEQ ID NOs: 8-14) mRNA immunization can kill Borrelia bacteria. Therefore, even though the mRNA encoded proteins are not lipidated, they are still functional and bactericidal. (FIG. 9).

Characterization of Mammalian-expressed OspA-S1 with Monoclonal Antibodies Binding studies with bacterial-specific antibodies Fab LA-2, Fab 184.1 and positive control antibodies show that binding to bacterial or mammalian-expressed OspA-S1 is similar to that of OspA S1 produced from bacterial cells (Table 7).

TABLE 7
Measured K_{d} (nM)

| | Bacterial-expressed | Mammalian-expressed |
|---|---|---|
| Fab | OspA-SR1 | OspA-S1 |
| LA-2 | 27.7 ± 1.0 | 62.0 ± 1.0 |
| 184.1 | 84.9 ± 8.1 | 27.0 ± 2.3 |
| Positive | 4.4 ± 0.1 | 7.1 ± 0.1 |
| control antibody | | |

### Example 7. Dosing Study

The following dosing study was conducted for OspA S1 NtermTM (SEQ ID NO: 8) to determine the lower limit of dosing amount to result in breakthrough infection in a tick bite model and whether 2 doses is as effective at protecting as 3 doses in a tick bite model. The experimental design of the dosing study is indicated in Table 8.

TABLE 8
Dosing Study Experimental Design

| | | Dose | | | | | |
|---|---|---|---|---|---|---|---|
| GR# | N= | (μg/animal) | Immunogen | Doses | Bleeding | Challenge | Readout |
| 1 | 5 | 1 | NTFIX | 1, 21, 42 | D −1, 20, | Tick bite | Total OspA |
| 2 | 5 | 1 | OspA S1 NtermTM | | 41, pre- | challenge | ELISA, LA-2 |
| | | | (SEQ ID NO: 8) | | challenge | (5 ticks | competition, |
| 3 | 5 | 0.5 | OspA S1 NtermTM | | (56), | per | positive |
| | | | (SEQ ID NO: 8) | | post- | mouse) | control |
| 4 | 5 | 0.2 | OspA S1 NtermTM | | challenge | | antibody |
| | | | (SEQ ID NO: 8) | | (74) | | competition, |
| 5 | 5 | 1 | Recombitek ® + Alum | | | | 4 week |
| | | | | | | | monitoring |
| | | | | | | | of cultured |
| | | | | | | | tissues |
| 6 | 5 | 1 | NTFIX | 1, 21 | D −1, 20, | | |
| 7 | 5 | 1 | OspA S1 NtermTM | | pre- | | |
| | | | (SEQ ID NO: 8) | | challenge | | |
| 8 | 5 | 0.5 | OspA S1 NtermTM | | (35), | | |
| | | | (SEQ ID NO: 8) | | post- | | |
| 9 | 5 | 0.2 | OspA S1 NtermTM | | challenge | | |
| | | | (SEQ ID NO: 8) | | (54) | | |
| 10 | 5 | 1 | Recombitek ® + Alum | | | | |

Mice (n=5) were administered either 2 or 3 doses of OspA S1 NtermTM (SEQ ID NO: 8) at 0.2 μg/animal, 0.5 μg/animal, or 1 μg/animal. Control animals were administered 1 lag/animal of either NTFIX or Recombitek® (Santa Cruz Biotechnology)+Alum. The first dose was administered on day 1 (d1), the second dose was administered on day 21 (d21) and the third dose was administered on day 42 (d42). For the 3 dose regime, blood samples were collected on d1, day 20 (d20), day 41 (d41), pre-challenge on day 56 (d56) and post-challenge day 74 (d74). For the 2 dose regime, blood samples were collected on d1, day 20 (d20), pre-challenge on day 35 (d35) and post-challenge day 54 (d54). For the tick bite challenge, 5 ticks were administered per mouse. The readout assays performed were Total OspA ELISA, LA-2 competition, positive control antibody competition, 4 week monitoring of cultured tissues.

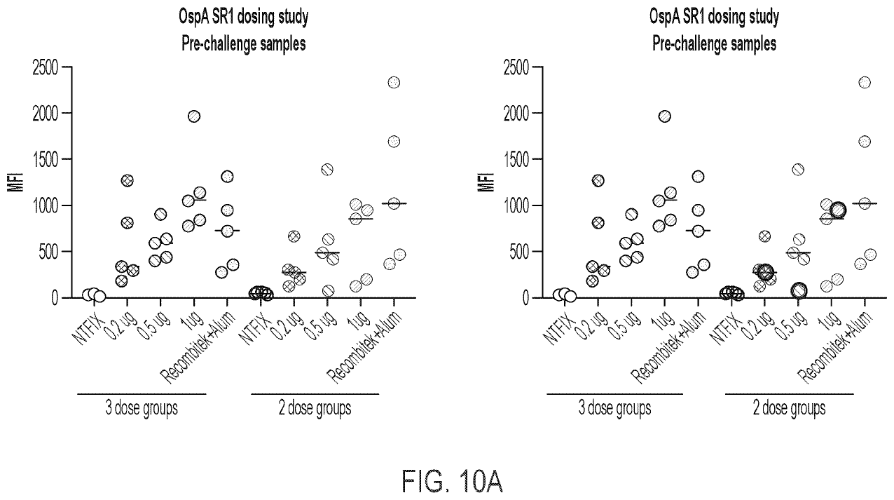

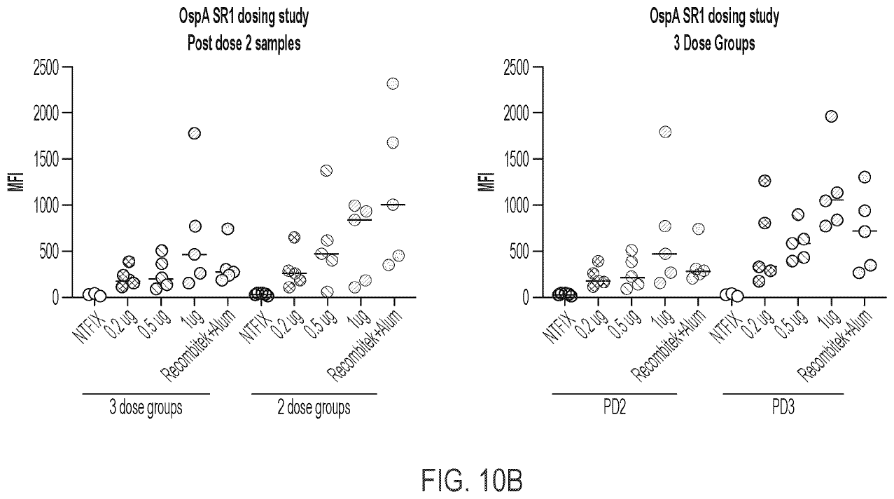

The results, as measured by total IgG levels demonstrate that 2 doses of OspA S11 NtermTM (SEQ ID NO: 8) resulted in 1 mouse per group getting infected and that antibody titers were not predictive of infection. Also, 3 doses of OspA S1 NtermTM (SEQ ID NO: 8) provided full protection to all mice, even at 0.2 μg dose. Results also showed that antibody titers were similar when comparing 2 doses of OspA S1 NtermTM (SEQ ID NO: 8) to 3 doses of OspA S1 NtermTM (SEQ ID NO: 8) and a boost was seen after the third dose in the 3 dose group (FIGS. 10A-10B).

### Example 8. Phase I/II Study

This is a Phase 1/2, randomized, observer-blind, placebo-controlled, dose-ranging study to evaluate the safety and immunogenicity of heptavalent mRNA vaccine (seven mRNAs, each mRNA having an ORF encoding one of the seven OspA serotypes) (SEQ ID NOs: 8-14) and monovalent mRNA vaccine (SEQ ID NO: 8).

This study will evaluate heptavalent mRNA vaccine (SEQ ID NOs: 8-14) and monovalent mRNA vaccine (SEQ ID NO: 8) as 3-dose prophylactic vaccines against Borrelia infection by eliciting anti-OspA antibody responses, which had been shown to block the transmission of Borrelia spirochetes from the bite of an infected tick and prevent Lyme disease. Heptavalent mRNA vaccine (SEQ ID NOs: 8-14) encodes for 7 different serotype OspA for 4 major Borrelia species causing disease in the US and Europe: Borrelia burgdorferi (SR1), afzelii (SR2), bavariensis (SR4), and garinii (SR3, 5-7). In comparison, monovalent mRNA vaccine (SEQ ID NO: 8) encodes for OspA from SR1 that is specific for B. burgdorferi, the dominant species circulating in the US that causes Lyme disease.

The primary objective of the study is to evaluate the safety and reactogenicity of heptavalent mRNA vaccine (SEQ ID NOs: 8-14) and monovalent mRNA vaccine (SEQ ID NO: 8). The primary endpoints are: solicited local and systemic adverse reactions (ARs) through 7 days after each study injection; unsolicited AEs through 28 days after each study injection; medically attended adverse events (MAAEs) from Day 1 to 6 months after last study injection; adverse event of special interests (AESIs) from Day 1 to end-of-study (EoS); serious adverse events (SAEs) from Day 1 to EoS; AEs leading to discontinuation of study injection or study participation from Day 1 to EoS; and safety laboratory abnormalities through 7 days after each study injection.

The secondary objective of the study is to evaluate the humoral immunogenicity of heptavalent mRNA vaccine (SEQ ID NOs: 8-14) and monovalent mRNA vaccine (SEQ ID NO: 8) at Days 1, 29, 85, and 197. Secondary endpoints included: geometric mean titer (GMT) of anti-OspA binding IgG antibodies measured by ELISA or a similar method at Days 1, 29, 85, and 197; and geometric mean fold rise (GMFR) of anti-OspA binding IgG Ab titer at Days 29, 85, and 197 compared to Day 1 (baseline).

The exploratory objectives of the study are to evaluate the humoral immunogenicity of heptavalent mRNA vaccine (SEQ ID NOs: 8-14) and monovalent mRNA vaccine (SEQ ID NO: 8); to further characterize antibody responses of heptavalent mRNA vaccine (SEQ ID NOs: 8-14) and monovalent mRNA vaccine (SEQ ID NO: 8); and to evaluate procedures in determining cases of Borrelia infection using a combination of clinical assessments and Lyme infection serology test results during the study. Exploratory endpoints include: GMT, GMFR, and seroresponse of anti-OspA-binding IgG antibodies at all evaluable timepoints; GMT and GMFR of serum bactericidal activity (SBA) elicited against Borrelia species expressing each of the SR1-7 OspA for a subset of participants; inhibition of LA-2 or other bactericidal epitope-specific antibodies when competing with elicited anti-OspA antibodies post study injection; and frequency of Borrelia infection assessed by combination of serological tests and clinical assessments.

In this study, safety and immunogenicity against Lyme disease in parallel of heptavalent mRNA vaccine (SEQ ID NOs: 8-14) and monovalent mRNA vaccine (SEQ ID NO: 8) will be studied in healthy adult participants aged 18 through 70 years. A group of participants receiving placebo will also be enrolled. A total of 800 healthy adult participants will be enrolled, comprised of 100 participants randomized equally into each dose group, including a placebo group (Table 9). All participants will receive a 3-dose series of the assigned study injection or placebo. Doses will be administered on Day 1, Day 57, and Day 169. Safety and immunogenicity data will be collected on Day 85 and Day 197.

Selected dose group(s) based on interim safety and immunogenicity data will be invited to enroll in an unblinded extension period for continued follow-up of up to 24 months post dose 3 (up to Month 30).

TABLE 9
Randomized Dose Groups

| Product | Dose | | Schedule | Sample Size |
|---|---|---|---|---|
| heptavalent mRNA | 25 | μg | 3 doses (0, 2, | 100 |
| vaccine (SEQ ID NOs: | | | and 6 months) | |
| 8-14) | | | | |
| heptavalent mRNA | 50 | μg | 3 doses (0, 2, | 100 |
| vaccine (SEQ ID NOs: | | | and 6 months) | |
| 8-14) | | | | |
| heptavalent mRNA | 100 | μg | 3 doses (0, 2, | 100 |
| vaccine (SEQ ID NOs: | | | and 6 months) | |
| 8-14) | | | | |
| heptavalent mRNA | 150 | μg | 3 doses (0, 2, | 100 |
| vaccine (SEQ ID NOs: | | | and 6 months) | |
| 8-14) | | | | |
| monovalent mRNA | 12.5 | μg | 3 doses (0, 2, | 100 |
| vaccine (SEQ ID NO: | | | and 6 months) | |
| 8) | | | | |
| monovalent mRNA | 25 | μg | 3 doses (0, 2, | 100 |
| vaccine (SEQ ID NO: | | | and 6 months) | |
| 8) | | | | |
| monovalent mRNA | 50 | μg | 3 doses (0, 2, | 100 |

| | | | |
|---|---|---|---|
| vaccine (SEQ ID NO: | | and 6 months) | |
| 8) | | | |
| Placebo (saline) | N/A (not | 3 doses (0, 2, | 100 |
| | applicable) | and 6 months) | |

### Example 9. Dose Response In Vivo Challenge Study

To test if mice immunized with a lower dose of OspA S1-S7 NtermTM mRNA are still protected in a tick bite challenge model the following experiments were performed.

Mice were administered vaccines comprising lipid nanoparticles encapsulating an mRNA having an ORF(s) encoding OspA S1-S7 (e.g., heptavalent; seven mRNAs, each mRNA having an ORF encoding one of the seven OspA serotypes) (SEQ ID NOs: 8-14). Study design and construct designs of immunogens encoded by the mRNA are indicated in TABLE 10.

Briefly, mRNA having an ORE(s) encoding OspA T1-M7 (e.g., heptavalent) (SEQ TD NOs: 8-14) were designed as previously described in Example 5. Some mice were immunized with non-translating mRNA (NTFIX) as a negative control. Other mice were immunized with Recombitek® (Santa Cruz Biotechnology) with alum.

TABLE 10
Study design to assess dose response to heptavalent constructs.

| | | Dose | | | | | |
|---|---|---|---|---|---|---|---|
| GR# | N= | (μg/animal) | Immunogen | Doses | Bleeding | Challenge | Readout |
| 1 | 5 | 3.5 | NTFIX | D 1, 21 | D −1, 20, | Tick bite | 3-4 week |
| 2 | 5 | 3.5 | OspA S1-S7 | | pre- | challenge | monitoring |
| | | | NtermTM (SEQ ID | | challenge, | (5 ticks per | of combined |
| | | | NOs: 8-14) | | and post- | mouse) | cultures |
| 3 | 5 | 1.4 | OspA S1-S7 | | challenge | | from |
| | | | NtermTM (SEQ ID | | | | multiple |
| | | | NOs: 8-14) | | | | tissues |
| 4 | 5 | 0.7 | OspA S1-S7 | | | | |
| | | | NtermTM (SEQ ID | | | | |
| | | | NOs: 8-14) | | | | |
| 5 | 5 | 1 | Recombitek ® + alum | | | | |
| 6 | 5 | 3.5 | NTFIX | D 1, | D −1, 20, | Tick bite | 3-4 week |
| 7 | 5 | 3.5 | OspA S1-S7 | 21, 42 | 41, pre- | challenge | monitoring |
| | | | NtermTM (SEQ ID | | challenge, | (5 ticks per | of combined |
| | | | NOs: 8-14) | | and post- | mouse) | cultures |
| 8 | 5 | 1.4 | OspA S1-S7 | | challenge | | from |
| | | | NtermTM (SEQ ID | | | | multiple |
| | | | NOs: 8-14) | | | | tissues |
| 9 | 5 | 0.7 | OspA S1-S7 | | | | |
| | | | NtermTM (SEQ ID | | | | |
| | | | NOs: 8-14) | | | | |
| 10 | 5 | 1 | Recombitek ® + alum | | | | |

Mice (n=5 animals/group) were immunized with two or three doses of one of the vaccines listed in Table 10. The two dose regimen was as follows: the first dose was administered on day 1 (d1), and the second dose on day 21 (d21). Blood samples were collected for all groups on d−1, day 20 (d20), and pre-challenge and post-challenge. The three dose regimen was as follows: the first dose was administered on day 1 (d1), the second dose on day 21 (d21), and the third does on day 42 (d42). Blood samples were collected for all groups on d-1, day 20 (d20), day 41 (d41), and pre-challenge and post-challenge. The readouts were measuring OspA antibody response and a tick bite challenge. The tick bite challenge was performed using Borrelia burgdorferi. Mice were challenged with five ticks. Two weeks after tick feeding, mice were sacrificed and skin samples, ankles, bladders, and hearts were collected. These tissues were cultures and monitored for 3-4 weeks to monitor for the presence of spirochetes.

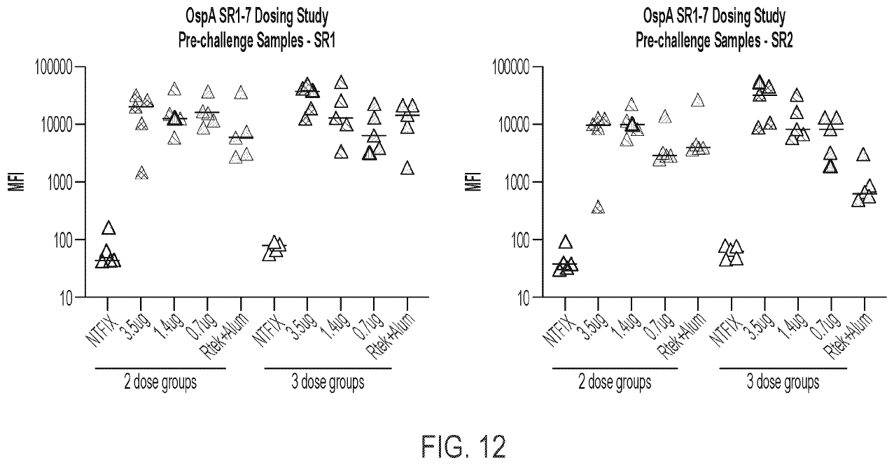

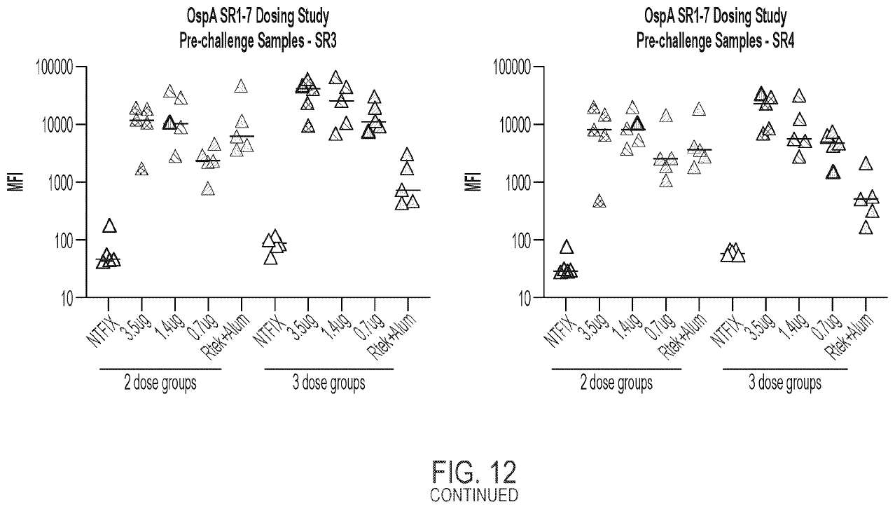

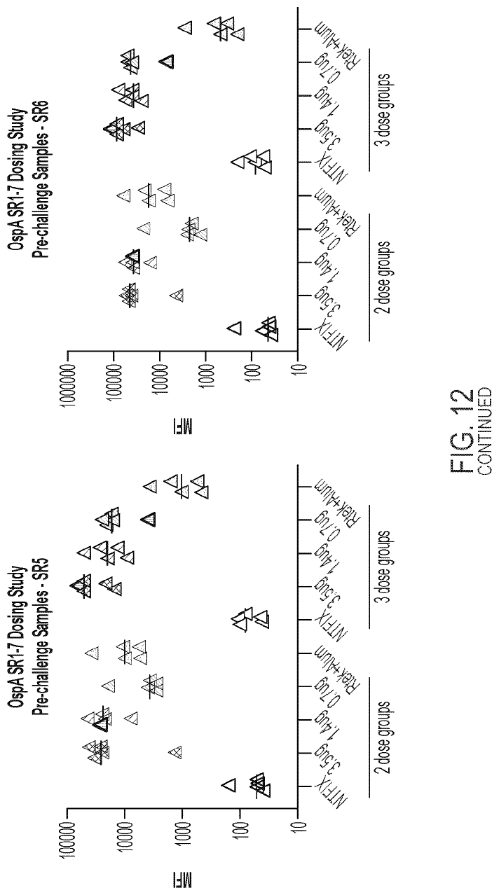

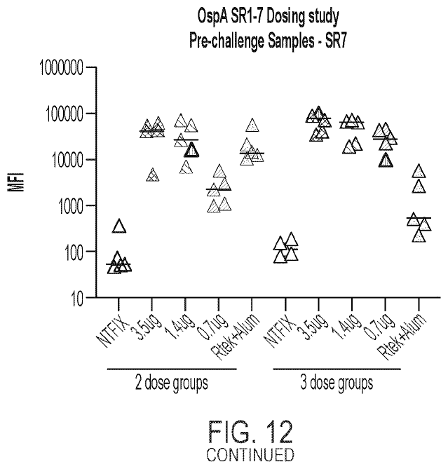

The total OspA S1-S7 IgG levels were measured pre-challenge. Compared to NTFIX treated mice, all groups immunized with mRNA having an ORFs encoding OspA S1-S7 (e.g., heptavalent) (SEQ ID NOs: 8-14) showed enhanced production of OspA S1-S7 IgGs (FIG. 12).

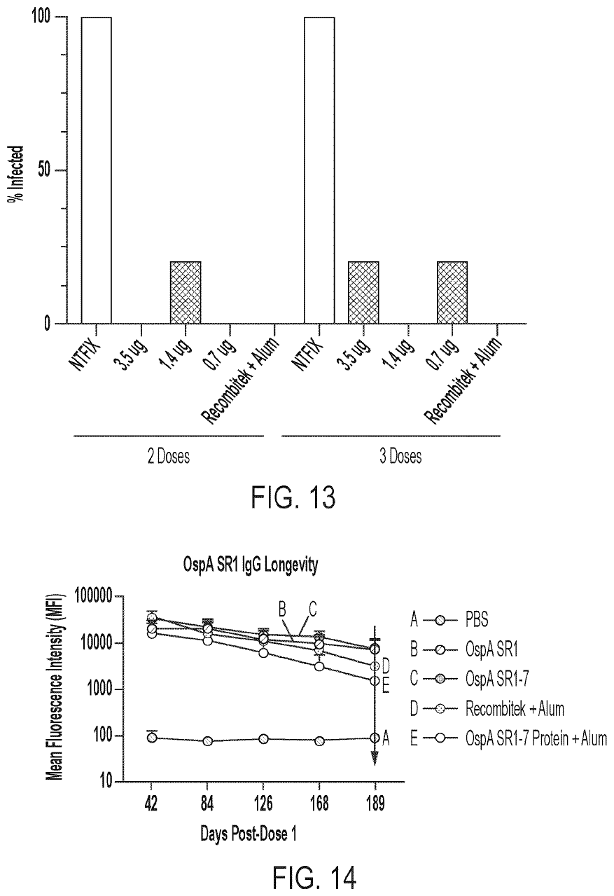

Mice immunized with 2 and 3 doses of mRNA having an ORFs encoding OspA S1-S7 (e.g., heptavalent) (SEQ ID NOs: 8-14) at 0.5 μg per serotype (3.5 μg total dose), 0.2 μg per serotype (1.4 μg total dose), and 0.1 μg per serotype (0.7 μg total dose) were protected from infection (FIG. 13).

### Example 10. OspA S1-S7 IgG Longevity Study

Experiments were performed to test OspA SR1-S7 IgG longevity in mice immunized twice with a OspA S1 NtermTM mRNA or a OspA S1-S7 NtermTM mRNA or protein.

Mice were administered vaccines comprising lipid nanoparticles encapsulating an mRNA having an ORF(s) encoding OspA S1 (e.g., monovalent) (SEQ ID NO: 8), OspA S1-S7 (e.g., heptavalent; seven mRNAs, each mRNA having an ORF encoding one of the seven OspA serotypes) (SEQ ID NOs: 8-14), lipidated purified OspA with alum, or purified OspA proteins of serotypes 1-7 with alum. Study design and construct designs of immunogens encoded by the mRNA are indicated in TABLE 11.

Briefly, mRNA having an ORF(s) encoding OspA S1 (e.g., monovalent) (SEQ ID NO: 8) or OspA S1-S7 (e.g., heptavalent) (SEQ ID NOs: 8-14) were designed as previously described in Example 5. Some mice were immunized with PBS as a negative control. Other mice were immunized with OspA S1-S7 protein or Recombitek® (Santa Cruz Biotechnology) with alum.

TABLE 11
Study design to compare OspA S1 IgG longevity.

| | | Dose | | | |
|---|---|---|---|---|---|
| GR# | N= | (μg/animal) | Immunogen | Doses | Bleeding |
| 1 | 8 | 0.5 | OspA-S1-ND165- | D 1, 29 | D 29, 42, |
| | | | 173-Nterm-TM | | 84, 126, |
| | | | (Nterm-TM) (SEQ | | 168, 189 |
| | | | ID NO: 8) | | |
| 2 | 8 | 3.5 | OspA S1-S7 | | |
| | | | NtermTM (SEQ ID | | |
| | | | NOs: 8-14) | | |
| 3 | 8 | 1 | Recombitek ® + alum | | |
| 4 | 8 | 7 | OspA S1-S7 | | |
| | | | protein + Alum | | |

Mice (n=8 animals/group) were immunized with two doses of one of the vaccines listed in Table 11. The dosage regimen was as follows: the first dose was administered on day 1 (d1), and the second dose on day 29 (d29). Blood samples were collected for all groups on day 29 (d29), day 42 (d42), day 84 (d84), day 126 (d126), day 168 (d168), and day 189 (d189).

The total OspA S1 IgG levels were measured in the serum samples using Luminex (readout in mean fluorescence intensity (MFI)) (FIG. 14). The half-lives (T50) for the IgGs were determined to be: T50 OspA SR1 mRNA=153 Days; T50 OspA SR1-7 mRNA=114 Days; T50 Recombitek+Alum=68 Days; and T50 OspASR1-7 Protein+Alum=83 Days.

Total OspA S1-S7 IgG levels were also measured in serum samples collected from the OspA S1-S7 NtermTM (SEQ ID NOs: 8-14) injected mice and the OspA S1-S7 protein+Alum injected mice (data not shown). T50 for these IgGs are provided in Table 12.

TABLE 12
Comparison of OspA S1-S7 IgG T50s.

T50 (Days)

| | OspA-S1-ND165-173- | |
|---|---|---|
| | Nterm-TM (Nterm-TM) | OspA S1-S7 |
| IgG | (SEQ ID NO: 8) | protein + Alum |
| S1 | 114 | 83 |
| S2 | 104 | 89 |
| S3 | 134 | 96 |
| S4 | 101 | 88 |
| S5 | 177 | 87 |
| S6 | 230 | 99 |
| S7 | 173 | 100 |

Thus, OspA S1-S7 IgGs produced following administration of the mRNA constructs have increased half-lives relative to OspA S1-S7 IgGs produced following administration of the protein constructs.

### Example 11. Cross-Protection Study

Experiments were performed to test for potential cross-protection activity among OspA antigens.

C₃H/HeN mice were administered vaccines comprising lipid nanoparticles encapsulating an mRNA having an ORF(s) encoding OspA S1 (monovalent) (SEQ ID NO: 8), OspA S1-S7 (heptavalent; seven mRNAs, each mRNA having an ORF encoding one of the seven OspA serotypes) (SEQ ID NOs: 8-14), OspA S2-S7 (hexavalent; six mRNAs, each mRNA having an ORF encoding one of the six OspA serotypes) (SEQ ID NOs: 9-14), non-translating mRNA (NTFIX), or Recombitek® (Santa Cruz Biotechnology) with alum. Study design and designs of immunogens encoded by the mRNA are indicated in TABLE 13.

Briefly, mRNA having an ORF(s) encoding OspA S1 (monovalent) (SEQ ID NO: 8), OspA S1-S7 (heptavalent) (SEQ ID NOs: 8-14), or OspA S2-S7 (hexavalent) (SEQ ID NOs: 9-14) were designed as previously described in Example 5. Some mice were immunized with NTFIX as a negative control. Other mice were immunized with Recombitek® with alum.

TABLE 13
Study design to assess cross-protection.

| | | Dose | | | | | |
|---|---|---|---|---|---|---|---|
| GR # | N= | (μg/animal) | Immunogen | Doses | Bleeding | Challenge | Readout |
| 1 | 5 | 3.5 | Non- | Day 1 | Day 1 | Tick bite | Total OspA |
| | | | translating | (D 1), | (D 1), Day | challenge | ELISA, LA-2 |
| | | | mRNA | and | 20 (D 20), | (5 ticks per | competition, |
| | | | (NTFIX) | Day 21 | Pre- | mouse) | positive |
| 2 | 5 | 3.5 | OspA S1-S7 | (D 21) | challenge, | | control |
| | | | (heptavalent) | | and post | | antibody |
| | | | (SEQ ID NOS: | | challenge | | competition, |
| | | | 8-14) | | | | 4 week |
| 3 | 5 | 3.0 | OspA S2-S7 | | | | monitoring |
| | | | (hexavalent) | | | | of cultured |
| | | | (SEQ ID NOS: | | | | tissues |
| | | | 9-14) | | | | |
| 4 | 5 | 0.5 | OspA S1 | | | | |
| | | | (monovalent) | | | | |
| | | | (SEQ ID NO: 8) | | | | |
| 5 | 5 | 1 | Recombitek ® | | | | |
| | | | with alum | | | | |

Mice (n=5 animals/group) were immunized with two doses of one of the vaccines listed in Table 13 at 0.5 μg/serotype. Control animals were administered 3.5 μg/animal of non-translating mRNA (NTFIX), or 1 μg/animal of Recombitek®+Alum. The dosage regimen was as follows: the first dose was administered on Day 1 (D1), and the second dose on day 21 (D21). Blood samples were collected for all groups on D1, and Day 20 (D20), pre-challenge on day 35 (D35) and post-challenge day 53 (D53). For the tick bite challenge, 5 ticks (S1 Borrelia burgdorferi) were administered per mouse. The readout assays performed were Total OspA ELISA, LA-2 competition, positive control antibody competition, and 4 week monitoring of cultured tissues.

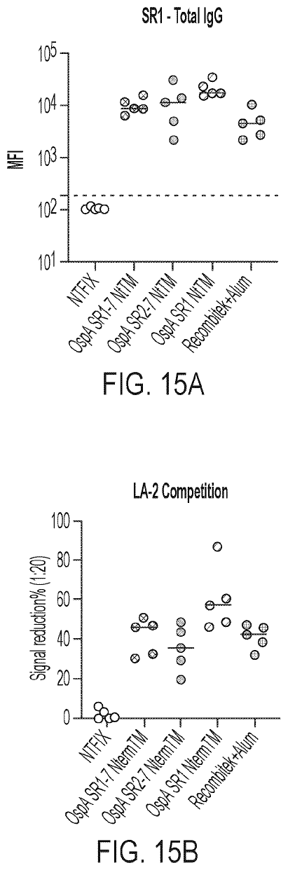

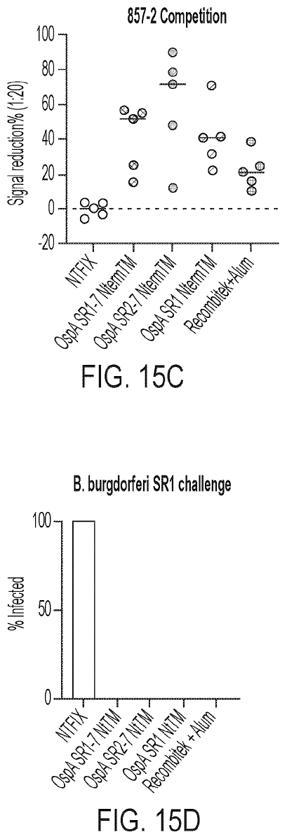

The total OspA S1 IgG levels were measured. Compared to NTFIX treated mice, all groups immunized with mRNA having an ORF(s) encoding OspA S1 (monovalent) (SEQ ID NO: 8), OspA S1-S7 (heptavalent) (SEQ ID NOs: 8-14), or OspA S2-S7 (hexavalent) (SEQ ID NOs: 9-14) showed enhanced production of OspA S1 IgG relative to NTFIX treated controls (FIG. 15A).

LA2 ELISA competition assays were performed. Groups of mice with high LA2 competition titers were considered protected in the challenge. Compared to NTFIX treated mice, mice administered vaccines comprising mRNA having an ORF(s) encoding OspA S1 (monovalent) (SEQ ID NO: 8), OspA S1-S7 (heptavalent) (SEQ ID NOs: 8-14), or OspA S2-S7 (hexavalent) (SEQ ID NOs: 9-14) showed significant LA2 signal reduction (FIG. 15B).

A positive control antibody, which is a known bactericidal monoclonal antibody that recognizes all 7 OspA serotypes, was also used in an ELISA competition assay. Compared to NTFIX treated mice, mice administered vaccines comprising mRNA having an ORF(s) encoding OspA S1 (monovalent) (SEQ ID NO: 8), OspA S1-S7 (heptavalent) (SEQ ID NOs: 8-14), or OspA S2-S7 (hexavalent) (SEQ ID NOs: 9-14) showed significant positive control antibody signal reduction (FIG. 15C).

The percent of infected mice was also measured. There was 100% infection in mice that received NTFIX treatment, and 0% infection in mice administered vaccines comprising mRNA having an ORF encoding OspA S1 (monovalent) (SEQ ID NO: 8), OspA S1-S7 (heptavalent) (SEQ ID NOs: 8-14), or OspA S2-S7 (hexavalent) (SEQ ID NOs: 9-14) (FIG. 15D).

These results show that even without being administered the OspA S1 mRNA (SEQ ID NO: 8), cross-reactive antibodies were elicited by OspA S2-S7 (hexavalent) (SEQ ID NOs: 9-14) as demonstrated by looking at total IgG. LA-2 and positive control antibody competition ELISAs show that functional antibodies were elicited by OspA S2-S7 (hexavalent) (SEQ ID NOs: 9-14) against OspA S1. This was further validated as none of the OspA S2-S7 (hexavalent) (SEQ ID NOs: 9-14) immunized mice were infected when challenged with an SR1 strain of Borrelia. These results demonstrate cross-protective ability of OspA antigens.

### Additional Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims. For example, the Additional Embodiments below are expressly contemplated:

### Additional Embodiments

1. A messenger ribonucleic acid (mRNA) vaccine, comprising:
an mRNA polynucleotide comprising an open reading frame encoding a protein comprising a Borrelia outer surface protein A (OspA) extracellular domain and optionally a heterologous amino terminal transmembrane domain; and
a lipid nanoparticle.

2. A messenger ribonucleic acid (mRNA) vaccine, comprising:
an mRNA polynucleotide comprising an open reading frame encoding a nonlipidated Borrelia outer surface protein A (OspA) protein; and
a lipid nanoparticle.

3. The mRNA vaccine of embodiment 1 or 2, wherein the Borrelia OspA protein comprises a heterologous amino terminal transmembrane domain.

4. The mRNA vaccine of any one of the preceding embodiments, wherein the heterologous amino terminal transmembrane domain is from an influenza virus neuraminidase protein.

5. The mRNA vaccine of any one of the preceding embodiments, wherein the Borrelia OspA protein comprises one or more modified N glycosylation sites, relative to a corresponding naturally occurring Borrelia OspA protein.

6. The mRNA vaccine of any one of the preceding embodiments, wherein the Borrelia OspA protein comprises a Borrelia burgdorferi OspA serotype 1 (S1) extracellular domain.

7. The mRNA vaccine of embodiment 6, wherein the Borrelia OspA S1 protein comprises a human leukocyte function-associated antigen (hLFA-1) epitope that is modified relative to a corresponding naturally occurring Borrelia OspA S1 protein.

8. The mRNA vaccine of embodiment 7, wherein the hLFA-1 epitope of the Borrelia OspA S1 protein comprises the following mutations Y165F, V166I, T170R, and L171F, relative to a naturally occurring Borrelia OspA S1 protein comprising the amino acid sequence of SEQ ID NO: 24.

9. The mRNA vaccine of any one of embodiments 6-8, further comprising at least one additional mRNA polynucleotide comprising an open reading frame encoding a nonlipidated Borrelia OspA protein comprising a Borrelia OspA extracellular domain selected from a Borrelia afzelii OspA serotype 2 (S2) extracellular domain, a Borrelia garinii OspA serotype 3 (S3) extracellular domain, a Borrelia bavariensis OspA serotype 4 (S4) extracellular domain, a Borrelia garinii OspA serotype 5 (S5) extracellular domain, a Borrelia garinii OspA serotype 6 (S6) extracellular domain, and a Borrelia garinii OspA serotype 7 (S7) extracellular domain, optionally wherein the nonlipidated Borrelia OspA protein encoded by the additional mRNA polynucleotide comprises a heterologous amino terminal transmembrane domain and/or N-linked glycan site mutations.

10. The mRNA vaccine of embodiment 9, further comprising two, three, four, five or six additional mRNAs, each comprising an open reading frame encoding a nonlipidated Borrelia OspA protein comprising a different Borrelia OspA extracellular domain selected from a Borrelia afzelii OspA S2 extracellular domain, a Borrelia garinii OspA S3 extracellular domain, a Borrelia bavariensis OspA S4 extracellular domain, a Borrelia garinii OspA S5 extracellular domain, a Borrelia garinii S6 protein, and a Borrelia garinii OspA S7 extracellular domain, optionally wherein each of the nonlipidated Borrelia OspA proteins encoded by the additional mRNAs comprises a heterologous amino terminal transmembrane domain and/or N-linked glycan site mutations.

11. The mRNA vaccine of any one of embodiments 6-8, further comprising a second mRNA polynucleotide comprising an open reading frame encoding a nonlipidated Borrelia OspA protein comprising a Borrelia afzelii OspA S2 extracellular domain, optionally wherein the nonlipidated Borrelia OspA protein encoded by the second mRNA polynucleotide comprises a heterologous amino terminal transmembrane domain and/or N-linked glycan site mutations.

12. The mRNA vaccine of embodiment 11, further comprising a third mRNA polynucleotide comprising an open reading frame encoding a nonlipidated Borrelia OspA protein comprising a Borrelia garinii OspA S3 extracellular domain, optionally wherein the nonlipidated Borrelia OspA protein encoded by the third mRNA polynucleotide comprises a heterologous amino terminal transmembrane domain and/or N-linked glycan site mutations.

13. The mRNA vaccine of embodiment 12, further comprising a fourth mRNA polynucleotide comprising an open reading frame encoding a nonlipidated Borrelia OspA protein comprising a Borrelia bavariensis OspA S4 extracellular domain, optionally wherein the nonlipidated Borrelia OspA protein encoded by the fourth mRNA polynucleotide comprises a heterologous amino terminal transmembrane domain and/or N-linked glycan site mutations.

14. The mRNA vaccine of embodiment 13, further comprising a fifth mRNA polynucleotide comprising an open reading frame encoding a nonlipidated Borrelia OspA protein comprising a Borrelia garinii OspA S5 extracellular domain, optionally wherein the nonlipidated Borrelia OspA protein encoded by the fifth mRNA polynucleotide comprises a heterologous amino terminal transmembrane domain and/or N-linked glycan site mutations.

15. The mRNA vaccine of embodiment 14, further comprising a six mRNA polynucleotide comprising an open reading frame encoding a nonlipidated Borrelia OspA protein comprising a Borrelia garinii OspA S6 extracellular domain, optionally wherein the nonlipidated Borrelia OspA protein encoded by the sixth mRNA polynucleotide comprises a heterologous amino terminal transmembrane domain and/or N-linked glycan site mutations.

16. The mRNA vaccine of embodiment 15, wherein the nonlipidated Borrelia OspA protein comprising a Borrelia garinii OspA S6 extracellular domain further comprises one or more amino acid modification(s), relative to a corresponding naturally occurring Borrelia garinii OspA S6 protein, to stabilize expression in mammalian cells.

17. The mRNA vaccine of embodiment 16, wherein the one or more amino acid modification(s) is a mutation corresponding to a S88L mutation or an A200V mutation of the naturally occurring Borrelia garinii OspA S6 protein.

18. The mRNA vaccine of embodiment 17, wherein the one or more amino acid modification(s) is a mutation corresponding to a S88L mutation and an A200V mutation of the naturally occurring Borrelia garinii OspA S6 protein.

19. The mRNA vaccine of any one of embodiments 16-18, further comprising a seventh mRNA polynucleotide comprising an open reading frame encoding a nonlipidated Borrelia OspA protein comprising a Borrelia garinii OspA S7 extracellular domain, optionally wherein the nonlipidated Borrelia OspA protein encoded by the seventh mRNA polynucleotide comprises a heterologous amino terminal transmembrane domain and/or N-linked glycan site mutations.

20. A messenger ribonucleic acid (mRNA) vaccine, comprising:
(a) an mRNA polynucleotide comprising an open reading frame encoding an outer surface protein A (OspA) protein comprising an extracellular domain of a first serotype of Borrelia;
(b) an mRNA polynucleotide comprising an open reading frame encoding an OspA protein comprising an extracellular domain of a second serotype of Borrelia, wherein the second serotype of Borrelia is of a species that is different from that of the first serotype of Borrelia; and
a lipid nanoparticle.

21. A messenger ribonucleic acid (mRNA) vaccine, comprising:
at least two, three, four, five, six, or seven mRNAs, each comprising an open reading frame encoding a Borrelia outer surface protein A (OspA) protein comprising an extracellular domain, wherein the extracellular domain of each of the Borrelia OspA proteins is of a different Borrelia serotype; and
a lipid nanoparticle.

22. A messenger ribonucleic acid (mRNA) vaccine, comprising at least four mRNAs, optionally seven mRNAs, each encoding a Borrelia OspA protein comprising an extracellular domain of one of four different serotypes of Borrelia, optionally selected from Borrelia burgdorferi, Borrelia afzelii, Borrelia garinii, and Borrelia bavariensis; and
a lipid nanoparticle.

23. The mRNA vaccine of any one of embodiments 20-22, wherein each of the Borrelia OspA proteins comprises a heterologous amino terminal transmembrane domain, optionally an influenza virus neuraminidase transmembrane domain.

24. A messenger ribonucleic acid (mRNA) vaccine, comprising:
an mRNA polynucleotide comprising an open reading frame encoding a protein comprising an extracellular domain from a Borrelia garinii OspA serotype 6 protein, wherein the extracellular domain comprises one or more amino acid modification(s), optionally selected from a S88L mutation and an A200V mutation, relative to a corresponding naturally occurring Borrelia garinii OspA S6 extracellular domain, to stabilize expression in mammalian cells; and
a lipid nanoparticle.

25. The mRNA of embodiment 24, wherein the protein further comprises a heterologous amino terminal transmembrane domain and/or one or more N-linked glycan site mutation(s).

26. The mRNA vaccine of any one of the preceding embodiments, wherein the one or more mRNA(s) of the vaccine comprises a chemical modification.

27. The mRNA vaccine of embodiment 26, wherein 100% of the uracil nucleotides of the one or more mRNA(s) comprise a chemical modification.

28. The mRNA vaccine of embodiment 26 or 27, wherein the chemical modification is 1-methylpseudouracil.

29. The mRNA vaccine of any one of the preceding embodiments, wherein the lipid nanoparticle comprises an ionizable lipid, a neutral lipid, a sterol, and a PEG-modified lipid.

30. The mRNA vaccine of embodiment 29, wherein the lipid nanoparticle comprises 40-50 mol % ionizable lipid, 5-15 mol % neutral lipid, 30-50 mol % sterol, and 0.5-3 mol % PEG-modified lipid.

31. The mRNA vaccine of embodiment 29 or 30, wherein the ionizable lipid comprises a structure of Compound (I):

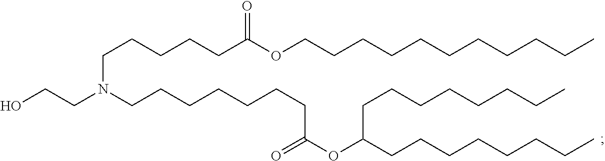 [SMILES: CCCCCCCCCCCOC(=O)CCCCCN(CCO)CCCCCCCC(=O)OC(CCCCCCCC)CCCCCCCC]

the neutral lipid is distearoylphosphatidylcholine (DSPC);
the sterol is cholesterol; and/or
the PEG-modified lipid is 1,2 dimyristoyl-sn-glycerol, methoxypolyethyleneglycol (PEG-DMG).

32. The mRNA vaccine of any one of the preceding embodiments, wherein the Borrelia burgdorferi OspA S1 protein comprises an amino acid sequence having at least 80%, at least 85%, at least 90%, at least 95%, or 100% identity to the amino acid sequence of SEQ ID NO: 1.

33. The mRNA vaccine of any one of the preceding embodiments, wherein the Borrelia afzelii OspA S2 protein comprises an amino acid sequence having at least 80%, at least 85%, at least 90%, at least 95%, or 100% identity to the amino acid sequence of SEQ ID NO: 2.

34. The mRNA vaccine of any one of the preceding embodiments, wherein the Borrelia garinii OspA S3 protein comprises an amino acid sequence having at least 80%, at least 85%, at least 90%, at least 95%, or 100% identity to the amino acid sequence of SEQ ID NO: 3.

35. The mRNA vaccine of any one of the preceding embodiments, wherein the Borrelia bavariensis OspA S4 protein comprises an amino acid sequence having at least 80%, at least 85%, at least 90%, at least 95%, or 100% identity to the amino acid sequence of SEQ ID NO: 4.

36. The mRNA vaccine of any one of the preceding embodiments, wherein the Borrelia garinii OspA S5 protein comprises an amino acid sequence having at least 80%, at least 85%, at least 90%, at least 95%, or 100% identity to the amino acid sequence of SEQ ID NO: 5.

37. The mRNA vaccine of any one of the preceding embodiments, wherein the Borrelia garinii OspA S6 protein comprises an amino acid sequence having at least 80%, at least 85%, at least 90%, at least 95%, or 100% identity to the amino acid sequence of SEQ ID NO: 6.

38. The mRNA vaccine of any one of the preceding embodiments, wherein the Borrelia garinii OspA S7 protein comprises an amino acid sequence having at least 80%, at least 85%, at least 90%, at least 95%, or 100% identity to the amino acid sequence of SEQ ID NO: 7.

39. The mRNA vaccine of any one of the preceding embodiments, wherein:
(a) the Borrelia burgdorferi OspA S1 protein comprises an amino acid sequence having at least 90% identity to the amino acid sequence of SEQ ID NO: 1;
(b) the Borrelia afzelii OspA S2 protein comprises an amino acid sequence having at least 90% identity to the amino acid sequence of SEQ ID NO: 2;
(c) the Borrelia garinii OspA S3 protein comprises an amino acid sequence having at least 90% identity to the amino acid sequence of SEQ ID NO: 3;
(d) the Borrelia bavariensis OspA S4 protein comprises an amino acid sequence having at least 90% identity to the amino acid sequence of SEQ ID NO: 4;
(e) the Borrelia garinii OspA S5 protein comprises an amino acid sequence having at least 90% identity to the amino acid sequence of SEQ ID NO: 5; (f) the Borrelia garinii OspA S6 protein comprises an amino acid sequence having at least 90% identity to the amino acid sequence of SEQ ID NO: 6; and
(g) the Borrelia garinii OspA S7 protein comprises an amino acid sequence having at least 90% identity to the amino acid sequence of SEQ ID NO: 7.

40. The mRNA vaccine of any one of the preceding embodiments, wherein:
(a) the Borrelia burgdorferi OspA S1 protein comprises an amino acid sequence having at least 95% identity to the amino acid sequence of SEQ ID NO: 1;
(b) the Borrelia afzelii OspA S2 protein comprises an amino acid sequence having at least 95% identity to the amino acid sequence of SEQ ID NO: 2; (c) the Borrelia garinii OspA S3 protein comprises an amino acid sequence having at least 95% identity to the amino acid sequence of SEQ ID NO: 3;
(d) the Borrelia bavariensis OspA S4 protein comprises an amino acid sequence having at least 95% identity to the amino acid sequence of SEQ ID NO: 4;
(e) the Borrelia garinii OspA S5 protein comprises an amino acid sequence having at least 95% identity to the amino acid sequence of SEQ ID NO: 5;
(f) the Borrelia garinii OspA S6 protein comprises an amino acid sequence having at least 95% identity to the amino acid sequence of SEQ ID NO: 6; and
(g) the Borrelia garinii OspA S7 protein comprises an amino acid sequence having at least 95% identity to the amino acid sequence of SEQ ID NO: 7.

41. The mRNA vaccine of any one of the preceding embodiments, wherein:
(a) the Borrelia burgdorferi OspA S1 protein comprises an amino acid sequence having at least 98% identity to the amino acid sequence of SEQ ID NO: 1;
(b) the Borrelia afzelii OspA S2 protein comprises an amino acid sequence having at least 98% identity to the amino acid sequence of SEQ ID NO: 2;
(c) the Borrelia garinii OspA S3 protein comprises an amino acid sequence having at least 98% identity to the amino acid sequence of SEQ ID NO: 3;
(d) the Borrelia bavariensis OspA S4 protein comprises an amino acid sequence having at least 98% identity to the amino acid sequence of SEQ ID NO: 4;
(e) the Borrelia garinii OspA S5 protein comprises an amino acid sequence having at least 98% identity to the amino acid sequence of SEQ ID NO: 5;
(f) the Borrelia garinii OspA S6 protein comprises an amino acid sequence having at least 98% identity to the amino acid sequence of SEQ ID NO: 6; and
(g) the Borrelia garinii OspA S7 protein comprises an amino acid sequence having at least 98% identity to the amino acid sequence of SEQ ID NO: 7.

42. The mRNA vaccine of any one of the preceding embodiments, wherein:
(a) the Borrelia burgdorferi OspA S1 protein comprises an amino acid sequence having 100% identity to the amino acid sequence of SEQ ID NO: 1;
(b) the Borrelia afzelii OspA S2 protein comprises an amino acid sequence having 100% identity to the amino acid sequence of SEQ ID NO: 2;
(c) the Borrelia garinii OspA S3 protein comprises an amino acid sequence having 100% identity to the amino acid sequence of SEQ ID NO: 3;
(d) the Borrelia bavariensis OspA S4 protein comprises an amino acid sequence having 100% identity to the amino acid sequence of SEQ ID NO: 4;
(e) the Borrelia garinii OspA S5 protein comprises an amino acid sequence having 100% identity to the amino acid sequence of SEQ ID NO: 5;
(f) the Borrelia garinii OspA S6 protein comprises an amino acid sequence having 100% identity to the amino acid sequence of SEQ ID NO: 6; and
(g) the Borrelia garinii OspA S7 protein comprises an amino acid sequence having 100% identity to the amino acid sequence of SEQ ID NO: 7.

43. A method of preventing Lyme disease in a subject in need thereof, the method comprising administering to the subject one or more doses of the vaccine of any one of the preceding embodiments in an effective amount to produce an immune response to a Borrelia bacterial infection.

44. The method of embodiment 43, wherein the vaccine is administered intramuscularly.

45. The method of embodiment 43 or embodiment 44, comprising administering a dose of 12.5-150 μg of the mRNA.

46. The method of any one of embodiments 43-45, comprising administering a single dose of the vaccine to the subject.

47. The method of any one of embodiments 43-45, comprising administering a prime dose and a booster dose of the vaccine to the subject.

48. The method of any one of embodiments 43-45, comprising administering a first dose of the vaccine, a second dose of the vaccine, and a third dose of the vaccine.

49. The method embodiment 48, wherein in second dose is administered two months following the first dose.

50. The method of embodiment 48 or embodiment 49, wherein the third dose is administered six months following the first dose.

51. The method of any one of embodiments 48-50, wherein the first dose, the second dose, and the third dose each comprise 12.5-150 μg of the RNA.

52. The method of embodiment 51, wherein the first dose, the second dose, and the third dose each comprise 12.5, 25, 50, 100, 150 μg of the RNA.

53. The method of embodiment 51 or embodiment 52, wherein the mRNA vaccine comprises a first mRNA polynucleotide comprising an open reading frame encoding a protein comprising a Borrelia burgdorferi outer surface protein A serotype 1 (OspA S1 protein), a second mRNA polynucleotide comprising an open reading frame encoding a protein comprising a Borrelia afzelii outer surface protein A serotype 2 (OspA S2 protein), a third mRNA polynucleotide comprising an open reading frame encoding a protein comprising a Borrelia garinii outer surface protein A serotype 3 (OspA S3 protein), a fourth mRNA polynucleotide comprising an open reading frame encoding a protein comprising a Borrelia bavariensis outer surface protein A serotype 4 (OspA S4 protein), a fifth mRNA polynucleotide comprising an open reading frame encoding a protein comprising a Borrelia garinii outer surface protein A serotype 5 (OspA S5 protein), a sixth mRNA polynucleotide comprising an open reading frame encoding a protein comprising a Borrelia garinii outer surface protein A serotype 6 (OspA S6 protein), and a seventh mRNA polynucleotide comprising an open reading frame encoding a protein comprising a Borrelia garinii outer surface protein A serotype 7 (OspA S7 protein), wherein:
(a) the Borrelia burgdorferi OspA S1 protein comprises an amino acid sequence having 100% identity to the amino acid sequence of SEQ ID NO: 1;
(b) the Borrelia afzelii OspA S2 protein comprises an amino acid sequence having 100% identity to the amino acid sequence of SEQ ID NO: 2;
(c) the Borrelia garinii OspA S3 protein comprises an amino acid sequence having 100% identity to the amino acid sequence of SEQ ID NO: 3;
(d) the Borrelia bavariensis OspA S4 protein comprises an amino acid sequence having 100% identity to the amino acid sequence of SEQ ID NO: 4;
(e) the Borrelia garinii OspA S5 protein comprises an amino acid sequence having 100% identity to the amino acid sequence of SEQ ID NO: 5;
(f) the Borrelia garinii OspA S6 protein comprises an amino acid sequence having 100% identity to the amino acid sequence of SEQ ID NO: 6; and
(g) the Borrelia garinii OspA S7 protein comprises an amino acid sequence having 100% identity to the amino acid sequence of SEQ ID NO: 7.

54. The method of embodiment 51, wherein the first dose, the second dose, and the third dose each comprise 12.5, 25, or 50 μg of the RNA.

55. The method of embodiment 51 or embodiment 54, wherein the mRNA vaccine comprises a mRNA polynucleotide comprising an open reading frame encoding a protein comprising a Borrelia burgdorferi outer surface protein A serotype 1 (OspA S1 protein), wherein the Borrelia burgdorferi OspA S1 protein comprises an amino acid sequence having 100% identity to the amino acid sequence of SEQ ID NO: 1.

56. The method of anyone of embodiments 43-55, wherein the lipid nanoparticle comprises a mixture of lipids that comprises 20-60 mol % ionizable lipid heptadecan-9-yl 8 ((2 hydroxyethyl)(6 oxo 6-(undecyloxy)hexyl)amino)octanoate (Compound 1); 5-25 mol % 1,2 distearoyl sn glycero-3 phosphocholine (DSPC); 25-55 mol % cholesterol; and 0.5-15 mol % 1-monomethoxypolyethyleneglycol-2,3-dimyristylglycerol with polyethylene glycol of average molecular weight 2000 (PEG2000 DMG).

57. The method of embodiment 56, wherein the lipid nanoparticle comprises:
47 mol % ionizable cationic lipid; 11.5 mol % neutral lipid; 38.5 mol % sterol; and 3.0 mol % PEG-modified lipid;
48 mol % ionizable cationic lipid; 11 mol % neutral lipid; 38.5 mol % sterol; and 2.5 mol % PEG-modified lipid;
49 mol % ionizable cationic lipid; 10.5 mol % neutral lipid; 38.5 mol % sterol; and 2.0 mol % PEG-modified lipid;
50 mol % ionizable cationic lipid; 10 mol % neutral lipid; 38.5 mol % sterol; and 1.5 mol % PEG-modified lipid; or
51 mol % ionizable cationic lipid; 9.5 mol % neutral lipid; 38.5 mol % sterol; and 1.0 mol % PEG-modified lipid.

58. The method of any one of embodiments 43-57, wherein the mRNA vaccine further comprises Tris buffer, sucrose, and sodium acetate, or any combination thereof.

59. The method of embodiment 58, wherein the mRNA vaccine further comprises 30-40 mM Tris buffer, 80-95 mg/mL sucrose, and 5-15 mM sodium acetate.

60. The method of any one of embodiments 43-59, wherein the mRNA vaccine has a pH value of 6-8, optionally 7.5.

61. The method of any one of embodiments 54-60, wherein the first dose, the second dose, and the third dose is 12.5 g of the mRNA, and wherein the second dose is administered 2 months after the first dose, and the third dose is administered 4 months after the second dose.

62. The method of any one of embodiments 54-60, wherein the first dose, the second dose, and the third dose is 50 μg of the mRNA, and wherein the second dose is administered 2 months after the first dose, and the third dose is administered 4 months after the second dose.

63. The method of any one of embodiments 54-60, wherein the first dose, the second dose, and the third dose is 100 μg of the mRNA, and wherein the second dose is administered 2 months after the first dose, and the third dose is administered 4 months after the second dose.

64. The method of any one of embodiments 54-60, wherein the first dose, the second dose, and the third dose is 150 μg of the mRNA, and wherein the second dose is administered 2 months after the first dose, and the third dose is administered 4 months after the second dose.

65. A method of inducing in a subject an immune response to a Borrelia bacterial infection, the method comprising:
administering to the subject the mRNA vaccine of any one of embodiments 1-42 in an amount effective at inducing in the subject a population of neutralizing antibodies.

66. The method of embodiment 65, wherein the population of neutralizing antibodies comprises IgG antibodies.

67. The method of embodiment 65 or embodiment 66, wherein the population of neutralizing antibodies comprises antibodies that bind to OspA S1, antibodies that bind to OspA S2, antibodies that bind to OspA S3, antibodies that bind to OspA S4, antibodies that bind to OspA S5, antibodies that bind to OspA S6, antibodies that bind to OspA S7, or any combination thereof.

68. A polynucleotide comprising a sequence having at least 80%, 85%, 90%, 95%, or 98% identity to the nucleic acid sequence of any one of SEQ ID NOs: 8-14.

69. A polynucleotide comprising the nucleic acid sequence of any one of SEQ ID NOs: 8-14.

70. A composition comprising a plurality of polynucleotides, wherein the plurality comprises two or more of:
a first polynucleotide comprising a sequence having at least 80%, 85%, 90%, 95%, or 98% identity to the nucleic acid sequence of SEQ ID NO: 8;
a second polynucleotide comprising a sequence having at least 80%, 85%, 90%, 95%, or 98% identity to the nucleic acid sequence of SEQ ID NO: 9;
a third polynucleotide comprising a sequence having at least 80%, 85%, 90%, 95%, or 98% identity to the nucleic acid sequence of SEQ ID NO: 10;
a fourth polynucleotide comprising a sequence having at least 80%, 85%, 90%, 95%, or 98% identity to the nucleic acid sequence of SEQ ID NO: 11;
a fifth polynucleotide comprising a sequence having at least 80%, 85%, 90%, 95%, or 98% identity to the nucleic acid sequence of SEQ ID NO: 12;
a sixth polynucleotide comprising a sequence having at least 80%, 85%, 90%, 95%, or 98% identity to the nucleic acid sequence of SEQ ID NO: 13; and
a seventh polynucleotide comprising a sequence having at least 80%, 85%, 90%, 95%, or 98% identity to the nucleic acid sequence of SEQ ID NO: 14.

71. The composition of embodiment 70, wherein:
the first polynucleotide comprises the nucleic acid sequence of SEQ ID NO: 8;
the second polynucleotide comprises the nucleic acid sequence of SEQ ID NO: 9;
the third polynucleotide comprises the nucleic acid sequence of SEQ ID NO: 10;
the fourth polynucleotide comprises the nucleic acid sequence of SEQ ID NO: 11;
the fifth polynucleotide comprises the nucleic acid sequence of SEQ ID NO: 12;
the sixth polynucleotide comprises the nucleic acid sequence of SEQ ID NO: 13; and
the seventh polynucleotide comprises the nucleic acid sequence of SEQ ID NO: 14.

72. The composition of embodiment 70 or embodiment 71, wherein the plurality comprises the second, third, fourth, fifth, sixth, and seventh polynucleotides.

73. The composition of any one of embodiments 70-72, wherein the plurality comprises the first, second, third, fourth, fifth, sixth, and seventh polynucleotides.

74. The mRNA vaccine of any one of embodiments 1-42 or the method of any one of embodiments 43-67, wherein the mRNA vaccine, when administered to a subject, cross-protects against a Borrelia outer surface protein A (OspA) protein that is not encoded by the mRNA vaccine, optionally wherein the cross-protection comprises generation of cross-reactive antibodies against the Borrelia outer surface protein A (OspA) protein that is not encoded by the mRNA vaccine.

75. A messenger ribonucleic acid (mRNA) vaccine, comprising:
an mRNA polynucleotide comprising an open reading frame encoding a Borrelia outer surface protein A (OspA) extracellular domain and optionally a heterologous amino terminal transmembrane domain, optionally wherein the heterologous amino terminal transmembrane domain is from an influenza virus neuraminidase protein; and
a lipid nanoparticle.

76. The mRNA vaccine of embodiment 75, wherein the mRNA polynucleotide comprises an open reading frame encoding a nonlipidated Borrelia OspA protein comprising a Borrelia burgdorferi OspA serotype 1 (S1) extracellular domain, optionally wherein the nonlipidated Borrelia OspA mRNA polynucleotide comprises a heterologous amino terminal transmembrane domain and/or N-linked glycan site mutations.

77. The mRNA vaccine of embodiment 76, comprising:
a first mRNA polynucleotide comprising an open reading frame encoding a nonlipidated Borrelia OspA protein comprising a Borrelia burgdorferi OspA serotype 1 (S1) extracellular domain, optionally wherein the nonlipidated Borrelia OspA protein encoded by the first mRNA polynucleotide comprises a heterologous amino terminal transmembrane domain and/or N-linked glycan site mutations;
a second mRNA polynucleotide comprising an open reading frame encoding a nonlipidated Borrelia OspA protein comprising a Borrelia afzelii OspA S2 extracellular domain, optionally wherein the nonlipidated Borrelia OspA protein encoded by the second mRNA polynucleotide comprises a heterologous amino terminal transmembrane domain and/or N-linked glycan site mutations;
a third mRNA polynucleotide comprising an open reading frame encoding a nonlipidated Borrelia OspA protein comprising a Borrelia garinii OspA S3 extracellular domain, optionally wherein the nonlipidated Borrelia OspA protein encoded by the third mRNA polynucleotide comprises a heterologous amino terminal transmembrane domain and/or N-linked glycan site mutations;
a fourth mRNA polynucleotide comprising an open reading frame encoding a nonlipidated Borrelia OspA protein comprising a Borrelia bavariensis OspA S4 extracellular domain, optionally wherein the nonlipidated Borrelia OspA protein encoded by the fourth mRNA polynucleotide comprises a heterologous amino terminal transmembrane domain and/or N-linked glycan site mutations;
a fifth mRNA polynucleotide comprising an open reading frame encoding a nonlipidated Borrelia OspA protein comprising a Borrelia garinii OspA S5 extracellular domain, optionally wherein the nonlipidated Borrelia OspA protein encoded by the fifth mRNA polynucleotide comprises a heterologous amino terminal transmembrane domain and/or N-linked glycan site mutations;
a six mRNA polynucleotide comprising an open reading frame encoding a nonlipidated Borrelia OspA protein comprising a Borrelia garinii OspA S6 extracellular domain, optionally wherein the nonlipidated Borrelia OspA protein encoded by the sixth mRNA polynucleotide comprises a heterologous amino terminal transmembrane domain and/or N-linked glycan site mutations; and
a seventh mRNA polynucleotide comprising an open reading frame encoding a nonlipidated Borrelia OspA protein comprising a Borrelia garinii OspA S7 extracellular domain, optionally wherein the nonlipidated Borrelia OspA protein encoded by the seventh mRNA polynucleotide comprises a heterologous amino terminal transmembrane domain and/or N-linked glycan site mutations.

78. The mRNA vaccine of embodiment 75 or embodiment 76, wherein the mRNA polynucleotide comprises at least two open reading frames, each encoding a Borrelia OspA extracellular domain and optionally a heterologous amino terminal transmembrane domain.

79. A messenger ribonucleic acid (mRNA) vaccine, comprising:
at least two, three, four, five, six, or seven mRNAs, each comprising an open reading frame encoding a Borrelia outer surface protein A (OspA) protein comprising an extracellular domain, wherein the extracellular domain of each of the Borrelia OspA proteins is of a different Borrelia serotype; and
a lipid nanoparticle.

80. The mRNA vaccine of embodiment 79, wherein the mRNA vaccine, when administered to a subject, cross-protects against a Borrelia outer surface protein A (OspA) protein that is not encoded by the mRNA vaccine, optionally wherein the cross-protection comprises generation of cross-reactive antibodies against the Borrelia outer surface protein A (OspA) protein that is not encoded by the mRNA vaccine.

81. A messenger ribonucleic acid (mRNA) vaccine, comprising an mRNA polynucleotide comprising an open reading frame encoding a protein comprising an extracellular domain from a Borrelia garinii OspA serotype 6 protein, wherein the extracellular domain comprises one or more amino acid modification(s), optionally selected from a S88L mutation and an A200V mutation, relative to a corresponding naturally occurring Borrelia garinii OspA S6 extracellular domain, to stabilize expression in mammalian cells.

82. The mRNA vaccine of any one of the preceding embodiments, wherein the one or more mRNA(s) of the vaccine comprises a chemical modification.

83. The mRNA vaccine of embodiment 82, wherein 100% of the uracil nucleotides of the one or more mRNA(s) comprise a chemical modification, wherein the chemical modification is 1-methylpseudouracil.

84. The mRNA vaccine of any one of the preceding embodiments, comprising a lipid nanoparticle, wherein the lipid nanoparticle comprises an ionizable lipid, a neutral lipid, a sterol, and a PEG-modified lipid, optionally wherein the lipid nanoparticle comprises 40-50 mol % ionizable lipid, 5-15 mol % neutral lipid, 30-50 mol % sterol, and 0.5-3 mol % PEG-modified lipid.

85. The mRNA vaccine of embodiment 84, wherein the ionizable lipid comprises a structure of Compound (I):

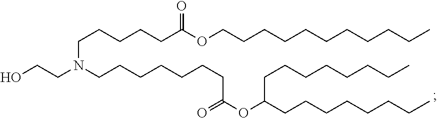 [SMILES: CCCCCCCCCCCOC(=O)CCCCCN(CCO)CCCCCCCC(=O)OC(CCCCCCCC)CCCCCCCC]

the neutral lipid is distearoylphosphatidylcholine (DSPC);
the sterol is cholesterol; and/or
the PEG-modified lipid is 1,2 dimyristoyl-sn-glycerol, methoxypolyethyleneglycol (PEG-DMG).

86. The mRNA vaccine of any one of embodiments 75-85, comprising:
(a) an mRNA polynucleotide comprising an open reading frame encoding a protein comprising a Borrelia burgdorferi OspA S1 protein comprising an amino acid sequence having at least 80%, at least 85%, at least 90%, at least 95%, or 98% identity to the amino acid sequence of SEQ ID NO: 1;
(b) an mRNA polynucleotide comprising an open reading frame encoding a protein comprising a Borrelia afzelii OspA S2 protein comprises an amino acid sequence having at least 80%, at least 85%, at least 90%, at least 95%, or 98% identity to the amino acid sequence of SEQ ID NO: 2;
(c) an mRNA polynucleotide comprising an open reading frame encoding a protein comprising a Borrelia garinii OspA S3 protein comprises an amino acid sequence having at least 80%, at least 85%, at least 90%, at least 95%, or 98% identity to the amino acid sequence of SEQ ID NO: 3;
(d) an mRNA polynucleotide comprising an open reading frame encoding a protein comprising a Borrelia bavariensis OspA S4 protein comprises an amino acid sequence having at least 80%, at least 85%, at least 90%, at least 95%, or 98% identity to the amino acid sequence of SEQ ID NO: 4;
(e) an mRNA polynucleotide comprising an open reading frame encoding a protein comprising a Borrelia garinii OspA S5 protein comprises an amino acid sequence having at least 80%, at least 85%, at least 90%, at least 95%, or 98% identity to the amino acid sequence of SEQ ID NO: 5;
(f) an mRNA polynucleotide comprising an open reading frame encoding a protein comprising a Borrelia garinii OspA S6 protein comprises an amino acid sequence having at least 80%, at least 85%, at least 90%, at least 95%, or 98% identity to the amino acid sequence of SEQ ID NO: 6;
(g) an mRNA polynucleotide comprising an open reading frame encoding a protein comprising a Borrelia garinii OspA S7 protein comprises an amino acid sequence having at least 80%, at least 85%, at least 90%, at least 95%, or 98% identity to the amino acid sequence of SEQ ID NO: 7; or
(h) any combination thereof.

87. The mRNA vaccine of embodiment 86, wherein:
the Borrelia burgdorferi OspA S1 protein comprises the amino acid sequence of SEQ ID NO: 1;
the Borrelia afzelii OspA S2 protein comprises the amino acid sequence of SEQ ID NO: 2;
the Borrelia garinii OspA S3 protein comprises the amino acid sequence of SEQ ID NO: 3;
the Borrelia bavariensis OspA S4 protein comprises the amino acid sequence of SEQ ID NO: 4;
the Borrelia garinii OspA S5 protein comprises the amino acid sequence of SEQ ID NO: 5;
the Borrelia garinii OspA S6 protein comprises the amino acid sequence of SEQ ID NO: 6; and
the Borrelia garinii OspA S7 protein comprises the amino acid sequence of SEQ ID NO: 7.

88. A method of inducing an immune response to a Borrelia antigen in a subject, the method comprising administering to the subject one or more doses of the mRNA vaccine of any one of the preceding embodiments in an effective amount to produce an immune response to a Borrelia antigen in the subject.

89. The method of embodiment 88, wherein the vaccine is administered intramuscularly.

90. The method of embodiment 88 or embodiment 89, comprising administering a dose of 12.5-150 μg of the mRNA comprised in the mRNA vaccine.

91. The method of any one of embodiments 88-90, comprising administering a single dose of the vaccine to the subject.

92. The method of any one of embodiments 88-90, comprising administering a first dose of the vaccine, a second dose of the vaccine, and a third dose of the vaccine.

93. The method of embodiment 92, wherein the second dose is administered two months following the first dose, the third dose is administered six months following the first dose, or a combination thereof.

94. The method of embodiment 92 or embodiment 93, wherein the first dose, the second dose, and the third dose is 50 g of the mRNA, and wherein the second dose is administered 2 months after the first dose, and the third dose is administered 4 months after the second dose.

95. The method of embodiment 92 or embodiment 93, wherein the first dose, the second dose, and the third dose is 100 μg of the mRNA, and wherein the second dose is administered 2 months after the first dose, and the third dose is administered 4 months after the second dose.

96. The method of embodiment 92 or embodiment 93, wherein the first dose, the second dose, and the third dose is 150 μg of the mRNA, and wherein the second dose is administered 2 months after the first dose, and the third dose is administered 4 months after the second dose.

97. The method of anyone of embodiments 88-96, wherein the lipid nanoparticle comprises a mixture of lipids that comprises 20-60 mol % ionizable lipid heptadecan-9-yl 8 ((2 hydroxyethyl)(6 oxo 6-(undecyloxy)hexyl)amino)octanoate (Compound 1); 5-25 mol % 1,2 distearoyl sn glycero-3 phosphocholine (DSPC); 25-55 mol % cholesterol; and 0.5-15 mol % 1-monomethoxypolyethyleneglycol-2,3-dimyristylglycerol with polyethylene glycol of average molecular weight 2000 (PEG2000 DMG).

98. The method of embodiment 97, wherein the lipid nanoparticle comprises:
47 mol % ionizable cationic lipid; 11.5 mol % neutral lipid; 38.5 mol % sterol; and 3.0 mol % PEG-modified lipid;
48 mol % ionizable cationic lipid; 11 mol % neutral lipid; 38.5 mol % sterol; and 2.5 mol % PEG-modified lipid;
49 mol % ionizable cationic lipid; 10.5 mol % neutral lipid; 38.5 mol % sterol; and 2.0 mol % PEG-modified lipid;
50 mol % ionizable cationic lipid; 10 mol % neutral lipid; 38.5 mol % sterol; and 1.5 mol % PEG-modified lipid; or
51 mol % ionizable cationic lipid; 9.5 mol % neutral lipid; 38.5 mol % sterol; and 1.0 mol % PEG-modified lipid.

99. The method of any one of embodiments 88-98, wherein the mRNA vaccine further comprises Tris buffer, sucrose, and sodium acetate, or any combination thereof.

100. A method of inducing in a subject an immune response to a Borrelia bacterial infection, the method comprising:
administering to the subject the mRNA vaccine of any one of embodiments 77-89 in an amount effective at inducing in the subject a population of neutralizing antibodies.

101. A polynucleotide comprising a sequence having at least 80%, 85%, 90%, 95%, or 98% identity to the nucleic acid sequence of any one of SEQ ID NOs: 8-14.

102. A composition comprising a plurality of polynucleotides, wherein the plurality comprises two or more of:
a first polynucleotide comprising a sequence having at least 80%, 85%, 90%, 95%, or 98% identity to the nucleic acid sequence of SEQ ID NO: 8;
a second polynucleotide comprising a sequence having at least 80%, 85%, 90%, 95%, or 98% identity to the nucleic acid sequence of SEQ ID NO: 9;
a third polynucleotide comprising a sequence having at least 80%, 85%, 90%, 95%, or 98% identity to the nucleic acid sequence of SEQ ID NO: 10;
a fourth polynucleotide comprising a sequence having at least 80%, 85%, 90%, 95%, or 98% identity to the nucleic acid sequence of SEQ ID NO: 11;
a fifth polynucleotide comprising a sequence having at least 80%, 85%, 90%, 95%, or 98% identity to the nucleic acid sequence of SEQ ID NO: 12;
a sixth polynucleotide comprising a sequence having at least 80%, 85%, 90%, 95%, or 98% identity to the nucleic acid sequence of SEQ ID NO: 13; and
a seventh polynucleotide comprising a sequence having at least 80%, 85%, 90%, 95%, or 98% identity to the nucleic acid sequence of SEQ ID NO: 14.

103. The composition of embodiment 102, wherein:
the first polynucleotide comprises the nucleic acid sequence of SEQ ID NO: 8;
the second polynucleotide comprises the nucleic acid sequence of SEQ ID NO: 9;
the third polynucleotide comprises the nucleic acid sequence of SEQ ID NO: 10;
the fourth polynucleotide comprises the nucleic acid sequence of SEQ ID NO: 11;
the fifth polynucleotide comprises the nucleic acid sequence of SEQ ID NO: 12;
the sixth polynucleotide comprises the nucleic acid sequence of SEQ ID NO: 13; and
the seventh polynucleotide comprises the nucleic acid sequence of SEQ ID NO: 14.

What is claimed is:

1. A messenger ribonucleic acid (mRNA) vaccine, comprising:
an mRNA polynucleotide comprising an open reading frame encoding a Borrelia outer surface protein A (OspA) extracellular domain and optionally a heterologous amino terminal transmembrane domain, optionally wherein the heterologous amino terminal transmembrane domain is from an influenza virus neuraminidase protein; and
a lipid nanoparticle.

2. The mRNA vaccine of claim 1, wherein the mRNA polynucleotide comprises an open reading frame encoding a nonlipidated Borrelia OspA protein comprising a Borrelia burgdorferi OspA serotype 1 (S1) extracellular domain, optionally wherein the nonlipidated Borrelia OspA mRNA polynucleotide comprises a heterologous amino terminal transmembrane domain and/or N-linked glycan site mutations.

3. The mRNA vaccine of claim 2, comprising:
a first mRNA polynucleotide comprising an open reading frame encoding a nonlipidated Borrelia OspA protein comprising a Borrelia burgdorferi OspA serotype 1 (S1) extracellular domain, optionally wherein the nonlipidated Borrelia OspA protein encoded by the first mRNA polynucleotide comprises a heterologous amino terminal transmembrane domain and/or N-linked glycan site mutations;
a second mRNA polynucleotide comprising an open reading frame encoding a nonlipidated Borrelia OspA protein comprising a Borrelia afzelii OspA S2 extracellular domain, optionally wherein the nonlipidated Borrelia OspA protein encoded by the second mRNA polynucleotide comprises a heterologous amino terminal transmembrane domain and/or N-linked glycan site mutations;
a third mRNA polynucleotide comprising an open reading frame encoding a nonlipidated Borrelia OspA protein comprising a Borrelia garinii OspA S3 extracellular domain, optionally wherein the nonlipidated Borrelia OspA protein encoded by the third mRNA polynucleotide comprises a heterologous amino terminal transmembrane domain and/or N-linked glycan site mutations;
a fourth mRNA polynucleotide comprising an open reading frame encoding a nonlipidated Borrelia OspA protein comprising a Borrelia bavariensis OspA S4 extracellular domain, optionally wherein the nonlipidated Borrelia OspA protein encoded by the fourth mRNA polynucleotide comprises a heterologous amino terminal transmembrane domain and/or N-linked glycan site mutations;
a fifth mRNA polynucleotide comprising an open reading frame encoding a nonlipidated Borrelia OspA protein comprising a Borrelia garinii OspA S5 extracellular domain, optionally wherein the nonlipidated Borrelia OspA protein encoded by the fifth mRNA polynucleotide comprises a heterologous amino terminal transmembrane domain and/or N-linked glycan site mutations;
a six mRNA polynucleotide comprising an open reading frame encoding a nonlipidated Borrelia OspA protein comprising a Borrelia garinii OspA S6 extracellular domain, optionally wherein the nonlipidated Borrelia OspA protein encoded by the sixth mRNA polynucleotide comprises a heterologous amino terminal transmembrane domain and/or N-linked glycan site mutations; and
a seventh mRNA polynucleotide comprising an open reading frame encoding a nonlipidated Borrelia OspA protein comprising a Borrelia garinii OspA S7 extracellular domain, optionally wherein the nonlipidated Borrelia OspA protein encoded by the seventh mRNA polynucleotide comprises a heterologous amino terminal transmembrane domain and/or N-linked glycan site mutations.

4. The mRNA vaccine of claim 1 or claim 2, wherein the mRNA polynucleotide comprises at least two open reading frames, each encoding a Borrelia OspA extracellular domain and optionally a heterologous amino terminal transmembrane domain.

5. A messenger ribonucleic acid (mRNA) vaccine, comprising:
at least two, three, four, five, six, or seven mRNAs, each comprising an open reading frame encoding a Borrelia outer surface protein A (OspA) protein comprising an extracellular domain, wherein the extracellular domain of each of the Borrelia OspA proteins is of a different Borrelia serotype; and
a lipid nanoparticle.

6. The mRNA vaccine of claim 5, wherein the mRNA vaccine, when administered to a subject, cross-protects against a Borrelia outer surface protein A (OspA) protein that is not encoded by the mRNA vaccine, optionally wherein the cross-protection comprises generation of cross-reactive antibodies against the Borrelia outer surface protein A (OspA) protein that is not encoded by the mRNA vaccine.

7. A messenger ribonucleic acid (mRNA) vaccine, comprising an mRNA polynucleotide comprising an open reading frame encoding a protein comprising an extracellular domain from a Borrelia garinii OspA serotype 6 protein, wherein the extracellular domain comprises one or more amino acid modification(s), optionally selected from a S88L mutation and an A200V mutation, relative to a corresponding naturally occurring Borrelia garinii OspA S6 extracellular domain, to stabilize expression in mammalian cells.

8. The mRNA vaccine of any one of the preceding claims, wherein the one or more mRNA(s) of the vaccine comprises a chemical modification.

9. The mRNA vaccine of claim 8, wherein 100% of the uracil nucleotides of the one or more mRNA(s) comprise a chemical modification, wherein the chemical modification is 1-methylpseudouracil.

10. The mRNA vaccine of any one of the preceding claims, comprising a lipid nanoparticle, wherein the lipid nanoparticle comprises an ionizable lipid, a neutral lipid, a sterol, and a PEG-modified lipid, optionally wherein the lipid nanoparticle comprises 40-50 mol % ionizable lipid, 5-15 mol % neutral lipid, 30-50 mol % sterol, and 0.5-3 mol % PEG-modified lipid.

11. The mRNA vaccine of claim 10, wherein the ionizable lipid comprises a structure of Compound (I): 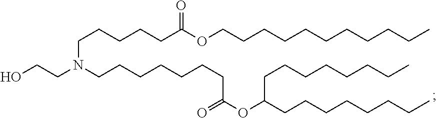 [SMILES: CCCCCCCCCCCOC(=O)CCCCCN(CCO)CCCCCCCC(=O)OC(CCCCCCCC)CCCCCCCC]
the neutral lipid is distearoylphosphatidylcholine (DSPC);
the sterol is cholesterol; and/or
the PEG-modified lipid is 1,2 dimyristoyl-sn-glycerol, methoxypolyethyleneglycol (PEG-DMG).

12. The mRNA vaccine of any one of claims 1-11, comprising:
(a) an mRNA polynucleotide comprising an open reading frame encoding a protein comprising a Borrelia burgdorferi OspA S1 protein comprising an amino acid sequence having at least 80%, at least 85%, at least 90%, at least 95%, or 98% identity to the amino acid sequence of SEQ ID NO: 1;
(b) an mRNA polynucleotide comprising an open reading frame encoding a protein comprising a Borrelia afzelii OspA S2 protein comprises an amino acid sequence having at least 80%, at least 85%, at least 90%, at least 95%, or 98% identity to the amino acid sequence of SEQ ID NO: 2;
(c) an mRNA polynucleotide comprising an open reading frame encoding a protein comprising a Borrelia garinii OspA S3 protein comprises an amino acid sequence having at least 80%, at least 85%, at least 90%, at least 95%, or 98% identity to the amino acid sequence of SEQ ID NO: 3;
(d) an mRNA polynucleotide comprising an open reading frame encoding a protein comprising a Borrelia bavariensis OspA S4 protein comprises an amino acid sequence having at least 80%, at least 85%, at least 90%, at least 95%, or 98% identity to the amino acid sequence of SEQ ID NO: 4;
(e) an mRNA polynucleotide comprising an open reading frame encoding a protein comprising a Borrelia garinii OspA S5 protein comprises an amino acid sequence having at least 80%, at least 85%, at least 90%, at least 95%, or 98% identity to the amino acid sequence of SEQ ID NO: 5;
(f) an mRNA polynucleotide comprising an open reading frame encoding a protein comprising a Borrelia garinii OspA S6 protein comprises an amino acid sequence having at least 80%, at least 85%, at least 90%, at least 95%, or 98% identity to the amino acid sequence of SEQ ID NO: 6;
(g) an mRNA polynucleotide comprising an open reading frame encoding a protein comprising a Borrelia garinii OspA S7 protein comprises an amino acid sequence having at least 80%, at least 85%, at least 90%, at least 95%, or 98% identity to the amino acid sequence of SEQ ID NO: 7; or
(h) any combination thereof.

13. The mRNA vaccine of claim 12, wherein:
the Borrelia burgdorferi OspA S1 protein comprises the amino acid sequence of SEQ ID NO: 1;
the Borrelia afzelii OspA S2 protein comprises the amino acid sequence of SEQ ID NO: 2;
the Borrelia garinii OspA S3 protein comprises the amino acid sequence of SEQ ID NO: 3;
the Borrelia bavariensis OspA S4 protein comprises the amino acid sequence of SEQ ID NO: 4;
the Borrelia garinii OspA S5 protein comprises the amino acid sequence of SEQ ID NO: 5;
the Borrelia garinii OspA S6 protein comprises the amino acid sequence of SEQ ID NO: 6; and
the Borrelia garinii OspA S7 protein comprises the amino acid sequence of SEQ ID NO: 7.

14. A method of inducing an immune response to a Borrelia antigen in a subject, the method comprising administering to the subject one or more doses of the mRNA vaccine of any one of the preceding claims in an effective amount to produce an immune response to a Borrelia antigen in the subject.

15. The method of claim 14, wherein the vaccine is administered intramuscularly.

16. The method of claim 14 or claim 15, comprising administering a dose of 12.5-150 μg of the mRNA comprised in the mRNA vaccine.

17. The method of any one of claims 14-16, comprising administering a single dose of the vaccine to the subject.

18. The method of any one of claims 14-16, comprising administering a first dose of the vaccine, a second dose of the vaccine, and a third dose of the vaccine.

19. The method of claim 18, wherein the second dose is administered two months following the first dose, the third dose is administered six months following the first dose, or a combination thereof.

20. The method of claim 18 or claim 19, wherein the first dose, the second dose, and the third dose is 50 μg of the mRNA, and wherein the second dose is administered 2 months after the first dose, and the third dose is administered 4 months after the second dose.

21. The method of claim 18 or claim 19, wherein the first dose, the second dose, and the third dose is 100 μg of the mRNA, and wherein the second dose is administered 2 months after the first dose, and the third dose is administered 4 months after the second dose.

22. The method of claim 18 or claim 19, wherein the first dose, the second dose, and the third dose is 150 μg of the mRNA, and wherein the second dose is administered 2 months after the first dose, and the third dose is administered 4 months after the second dose.

23. The method of anyone of claims 14-22, wherein the lipid nanoparticle comprises a mixture of lipids that comprises 20-60 mol % ionizable lipid heptadecan-9-yl 8 ((2 hydroxyethyl)(6 oxo 6-(undecyloxy)hexyl)amino)octanoate (Compound 1); 5-25 mol % 1,2 distearoyl sn glycero-3 phosphocholine (DSPC); 25-55 mol % cholesterol; and 0.5-15 mol % 1-monomethoxypolyethyleneglycol-2,3-dimyristylglycerol with polyethylene glycol of average molecular weight 2000 (PEG2000 DMG).

24. The method of claim 23, wherein the lipid nanoparticle comprises:
47 mol % ionizable cationic lipid; 11.5 mol % neutral lipid; 38.5 mol % sterol; and 3.0 mol % PEG-modified lipid;
48 mol % ionizable cationic lipid; 11 mol % neutral lipid; 38.5 mol % sterol; and 2.5 mol % PEG-modified lipid;
49 mol % ionizable cationic lipid; 10.5 mol % neutral lipid; 38.5 mol % sterol; and 2.0 mol % PEG-modified lipid;
50 mol % ionizable cationic lipid; 10 mol % neutral lipid; 38.5 mol % sterol; and 1.5 mol % PEG-modified lipid; or
51 mol % ionizable cationic lipid; 9.5 mol % neutral lipid; 38.5 mol % sterol; and 1.0 mol % PEG-modified lipid.

25. The method of any one of claims 14-24, wherein the mRNA vaccine further comprises Tris buffer, sucrose, and sodium acetate, or any combination thereof.

26. A method of inducing in a subject an immune response to a Borrelia bacterial infection, the method comprising:
administering to the subject the mRNA vaccine of any one of claims 1-13 in an amount effective at inducing in the subject a population of neutralizing antibodies.

27. A polynucleotide comprising a sequence having at least 80%, 85%, 90%, 95%, or 98% identity to the nucleic acid sequence of any one of SEQ ID NOs: 8-14.

28. A composition comprising a plurality of polynucleotides, wherein the plurality comprises two or more of:
a first polynucleotide comprising a sequence having at least 80%, 85%, 90%, 95%, or 98% identity to the nucleic acid sequence of SEQ ID NO: 8;
a second polynucleotide comprising a sequence having at least 80%, 85%, 90%, 95%, or 98% identity to the nucleic acid sequence of SEQ ID NO: 9;
a third polynucleotide comprising a sequence having at least 80%, 85%, 90%, 95%, or 98% identity to the nucleic acid sequence of SEQ ID NO: 10;
a fourth polynucleotide comprising a sequence having at least 80%, 85%, 90%, 95%, or 98% identity to the nucleic acid sequence of SEQ ID NO: 11;
a fifth polynucleotide comprising a sequence having at least 80%, 85%, 90%, 95%, or 98% identity to the nucleic acid sequence of SEQ ID NO: 12;
a sixth polynucleotide comprising a sequence having at least 80%, 85%, 90%, 95%, or 98% identity to the nucleic acid sequence of SEQ ID NO: 13; and
a seventh polynucleotide comprising a sequence having at least 80%, 85%, 90%, 95%, or 98% identity to the nucleic acid sequence of SEQ ID NO: 14.

29. The composition of claim 28, wherein:
the first polynucleotide comprises the nucleic acid sequence of SEQ ID NO: 8;
the second polynucleotide comprises the nucleic acid sequence of SEQ ID NO: 9;
the third polynucleotide comprises the nucleic acid sequence of SEQ ID NO: 10;
the fourth polynucleotide comprises the nucleic acid sequence of SEQ ID NO: 11;
the fifth polynucleotide comprises the nucleic acid sequence of SEQ ID NO: 12;
the sixth polynucleotide comprises the nucleic acid sequence of SEQ ID NO: 13; and
the seventh polynucleotide comprises the nucleic acid sequence of SEQ ID NO: 14.


SEQUENCE LISTING (68 sequences)

SEQ ID NO: 1 - protein, 287 aa, synthetic construct
MNPNQKIITIGSVSLTISTICFFMQIAILITTVTLHFKQEKNSVSVDLPGEMKVLVSKEKNKDGKYDLIATVDKLELKGTSDKNNGAGVLEGVKADKSKVKLTISDDLGQTTLEVFKEDGKTLVSKKVTSKDKSSTEEKFNEKGEVSEKIITRADGTRLEYTGIKSDGSGKAKEVLKGFILEGRFTAEKTTLVVKEGTVTLSKDISKSGEVSVELQDTDSSAATKKTAAWNSGTSTLTITVNSKKTKDLVFTKENTITVQQYDSNGVKLEGSAVEITKLDEIKNALK

SEQ ID NO: 2 - protein, 287 aa, synthetic construct
MNPNQKIITIGSVSLTISTICFFMQIAILITTVTLHFKQEKNSASVDLPGEMKVLVSKEKDKDGKYSLKATVDKIELKGTSDKDNGAGVLEGTKDDKSKAKLTIADDLSKTTFELFKEDGKTLVSRKVSSKDKTSTDEMFNEKGELSAKTMTREDGTKLEYTEMKSDGTGKAKEVLKGFTLEGKVANDKVTLEVKEGTVTLSKEIAKSGEVTVALQDTDTTQATKKTGAWDSKTSTLTISVNSKKTTQLVFTKQDTITVQKYDSAGTNLEGTAVEIKTLDELKNALK

SEQ ID NO: 3 - protein, 288 aa, synthetic construct
MNPNQKIITIGSVSLTISTICFFMQIAILITTVTLHFKQEKNSVSVDLPGGMKVLVSKEKDKDGKYSLMATVEKLELKGTSDKSNGAGVLEGEKADKSKAKLTISQDLSQTTFEIFKEDGKTLVSRKVNSKDKSSTEEKFNDKGKLSEKVVTRADGTRLEYTEIKNDGSGKAKEVLKGFALEGTLTDGGETKLTVTEGTVTLSKDISKSGEITVALQDTETTPADKKTGEWKSDTSTLTISKNSQKTKQLVFTKENTITVQNYNRAGNALEGSPAEIKDLAELKAALK

SEQ ID NO: 4 - protein, 287 aa, synthetic construct
MNPNQKIITIGSVSLTISTICFFMQIAILITTVTLHFKQEKNSVSVDLPGEMKVLVSKEKDKDGKYSLMATVDKLELKGTSDKSNGAGTLEGEKSDKSKAKLTISEDLSKTTFEIFKEDGKTLVSKKVNSKDKSSIEEKFNAKGELSEKTILRADGTRLEYTEIKSDGTGKAKEVLKDFALEGTLAADKTTLKVTEGTVVLSKHIPNSGEITVELQDSDSTQATKKTGKWDSQTSTLTISVNSKKTKNIVFTKEDTITVQKYDSAGTNLEGNAVEIKTLDELKNALK

SEQ ID NO: 5 - protein, 288 aa, synthetic construct
MNPNQKIITIGSVSLTISTICFFMQIAILITTVTLHFKQEKNSVSVDLPGGMKVLVSKEKDKDGKYSLMATVEKLELKGTSDKNNGAGTLEGEKTDKSKVKLTIAEDLSKTTFEIFKEDGKTLVSKKVTLKDKSSTEEKFNEKGEISEKTIVRADGTRLEYTDIKSDGSGKAKEVLKDFTLEGTLAADGKTTLKVTEGTVVLSKNILKSGEITVALDDSDTTQATKKTGKWDSKTSTLTISVNSQKTKNLVFTKEDTITVQKYDSAGTNLEGKAVEITTLEKLKDALK

SEQ ID NO: 6 - protein, 288 aa, synthetic construct
MNPNQKIITIGSVSLTISTICFFMQIAILITTVTLHFKQEKNSVSVDLPGGMTVLVSKEKDKDGKYSLEATVDKLELKGTSDKNNGAGTLEGEKTDKSKVKLTIADDLSQTKFEIFKEDGKTLVSKKVTLKDKSSTEEKFNGKGETSEKTIVRADGTRLEYTDIKSDGSGKAKEVLKDFTLEGTLAADGKTTLKVTEGTVVLSKNILKSGEITVALDDSDTTRATKKTGKWDSKTSTLTISVNSQKTKNLVFTKEDTITVQRYDSAGTNLEGKAVEITTLKELKNALK

SEQ ID NO: 7 - protein, 288 aa, synthetic construct
MNPNQKIITIGSVSLTISTICFFMQIAILITTVTLHFKQEKNSVSVDLPGEMKVLVSKEKDKDGKYSLEATVDKLELKGTSDKNNGAGVLEGVKAAKSKAKLTIADDLSQTKFEIFKEDGKTLVSKKVTLKDKSSTEEKFNDKGKLSEKVVTRADGTRLEYTEIQNDGSGKAKEVLKSLTLEGTLTADGETKLTVEAGTVTLSKDISESGEITVELKDTETTPADKKSGTWDSKTSTLTISKNSQKTKQLVFTKENTITVQKYNTAGTKLEGSPAEIKDLEALKAALK

SEQ ID NO: 8 - RNA, 861 nt, synthetic construct
  source 1..861: mol_type = other RNA
atgaaccccaaccagaagatcatcaccatcggcagcgtgagcctgacaatcagcaccatctgcttcttcatgcagatcgccatcctgatcaccaccgtcaccctgcacttcaagcaggagaagaacagcgtgagcgtggatctgcccggcgagatgaaggtgctggtcagcaaagagaagaacaaggacggtaagtacgacctgatcgccaccgtggacaagctggagctgaagggcaccagcgacaagaacaacggagctggcgtgctggagggcgtgaaggccgacaagagcaaggtgaagctgaccatcagcgacgacctgggccagacaaccctggaggtgttcaaggaggacggcaagaccctggtgagcaagaaggtgaccagcaaggacaagagcagcaccgaggagaagttcaacgagaagggcgaggtgagcgagaagatcatcacaagggctgacggcaccagactggagtacaccggcatcaagagcgacggcagtggcaaggccaaggaggtgctgaagggcttcatcctggagggccggtttaccgccgagaagaccaccctggtggtgaaggagggcaccgtgaccctgagcaaggacatcagcaagagcggcgaggtgtctgtggagctgcaggacaccgacagcagcgccgccacaaagaagaccgccgcctggaatagcggcaccagcaccctgaccatcaccgtgaacagcaagaagaccaaggacctggtgttcaccaaggagaacaccatcactgtgcagcagtacgacagcaacggcgtgaaactggagggcagcgccgtggagatcaccaagctggacgagatcaagaacgccctgaag

SEQ ID NO: 9 - RNA, 861 nt, synthetic construct
  source 1..861: mol_type = other RNA
atgaaccccaaccagaagatcatcaccatcggcagcgtgtcgttgaccatcagcaccatctgcttcttcatgcagatcgccatcctgatcaccaccgtgaccctgcacttcaagcaggagaagaacagcgccagcgtggacctgcccggagagatgaaggtgctggtgagtaaggagaaggacaaagacggcaagtacagcctgaaggccaccgtggataagatcgagctgaagggcaccagcgacaaggacaacggagccggcgtgctggagggaaccaaggacgacaagagcaaggcaaagctgaccatcgccgacgacctgagtaagaccaccttcgagctgttcaaggaggacggcaagaccctggtgagccggaaggtgagcagcaaggataagaccagcaccgacgagatgttcaacgagaagggcgaactgagcgccaagaccatgaccagagaggacggcaccaagctggagtacaccgagatgaagagcgacggcaccggcaaggccaaggaggtgctgaagggcttcaccctggaaggcaaggtggccaacgacaaggtgaccctggaggtgaaggagggcaccgtgaccctgagcaaggagatcgctaagagcggcgaggtgaccgtggccctgcaggacaccgacaccacccaggccaccaagaagaccggcgcctgggacagcaagaccagcaccctgaccatctccgtgaacagcaagaagaccacccagctggtgttcacaaagcaggacaccatcaccgtgcagaagtacgacagcgccggcaccaacctggagggcaccgccgtggagatcaagacactggacgagctgaagaacgccctgaag

SEQ ID NO: 10 - RNA, 864 nt, synthetic construct
  source 1..864: mol_type = other RNA
atgaaccccaaccagaagatcatcaccatcggcagcgtgtcgttgaccatcagcaccatctgcttcttcatgcagatcgccatcctgatcaccaccgtgaccctgcacttcaagcaggagaagaacagcgtgagcgtggacctgcccggaggcatgaaggtgctggtgagtaaggagaaggacaaagacggcaagtacagcctgatggccaccgtggagaagctggagctgaagggcaccagcgacaagtccaacggagccggcgtgctggagggagagaaggccgacaagagcaaggcaaagctgaccatcagccaggacctgagtcagaccaccttcgagatcttcaaggaggacggcaagaccctggtgagccggaaggtgaacagcaaggataagagcagcaccgaggagaagttcaacgacaagggcaagctgagcgagaaggtggtgaccagagcagacggcacccggctggagtacaccgagatcaagaacgacggcagcggcaaggccaaggaggtgctgaagggcttcgccctggaaggcaccctgaccgacggcggcgagaccaagctgaccgtgaccgagggcaccgtgaccctgagcaaggacatcagcaagagcggcgagatcaccgtggccctgcaggacaccgagaccaccccagccgacaagaagaccggcgagtggaagagcgacaccagcaccctgaccatctccaagaacagccagaagaccaagcagctggtgttcacaaaggagaacaccatcaccgtgcagaactacaaccgggccggcaacgcactggagggcagccccgcagagatcaaggacctggccgagctgaaggccgccctgaag

SEQ ID NO: 11 - RNA, 861 nt, synthetic construct
  source 1..861: mol_type = other RNA
atgaaccccaaccagaagatcatcaccatcggcagcgtgtcgttgaccatcagcaccatctgcttcttcatgcagatcgccatcctgatcaccaccgtgaccctgcacttcaagcaggagaagaacagcgtgagcgtggacctgcccggagagatgaaggtgctggtgagtaaggagaaggacaaagacggcaagtacagcctgatggccaccgtggataagctggagctgaagggcaccagcgacaagtccaacggagccggcactctggagggagagaagagcgacaagagcaaggcaaagctgaccatcagcgaggacctgagtaagaccaccttcgagatcttcaaggaggacggcaagaccctggtgagcaagaaggtgaacagcaaggataagagcagcatcgaggagaagttcaacgccaagggcgaactgagcgagaagaccatcctgagagcagacggcacccggctggagtacaccgagatcaagagcgacggcaccggcaaggccaaggaggtgctgaaggacttcgccctggaaggcaccctggccgccgacaagaccaccctgaaggtgaccgagggcaccgtggtgctgagcaagcacatccccaacagcggcgagatcaccgtggagctgcaggacagcgacagcacccaggccaccaagaagaccggcaagtgggacagccagaccagcaccctgaccatctccgtgaacagcaagaagaccaagaacatcgtgttcacaaaggaggacaccatcaccgtgcagaagtacgacagcgccggcaccaacctggagggcaacgccgtggagatcaagacactggacgagctgaagaacgccctgaag

SEQ ID NO: 12 - RNA, 864 nt, synthetic construct
  source 1..864: mol_type = other RNA
atgaaccccaaccagaagatcatcaccatcggcagcgtgtcgttgaccatcagcaccatctgcttcttcatgcagatcgccatcctgatcaccaccgtgaccctgcacttcaagcaggagaagaacagcgtgagcgtggacctgcccggaggcatgaaggtgctggtgagtaaggagaaggacaaagacggcaagtacagcctgatggccaccgtggagaagctggagctgaagggcaccagcgacaagaacaacggagccggcactctggagggagagaagacagacaagagcaaggtgaagctgaccatcgccgaggacctgagtaagaccaccttcgagatcttcaaggaggacggcaagaccctggtgagcaagaaggtgacactgaaggataagagcagcaccgaggagaagttcaacgagaagggcgaaatcagcgagaagaccatcgtgagagcagacggcacccggctggagtacaccgacatcaagagcgacggcagcggcaaggccaaggaggtgctgaaggacttcaccctggaaggcaccctggccgccgacggcaagaccaccctgaaggtgaccgagggcaccgtggtgctgagcaagaacatcctgaagagcggcgagatcaccgtggccctggacgacagcgacaccacccaggccaccaagaagaccggcaagtgggacagcaagaccagcaccctgaccatctccgtgaacagccagaagaccaagaacctggtgttcacaaaggaggacaccatcaccgtgcagaagtacgacagcgccggcaccaacctggagggcaaggccgtggagatcaccacactggagaaactgaaggacgccctgaag

SEQ ID NO: 13 - RNA, 864 nt, synthetic construct
  source 1..864: mol_type = other RNA
atgaaccccaaccagaagatcatcaccatcggcagcgtgtcgttgaccatcagcaccatctgcttcttcatgcagatcgccatcctgatcaccaccgtgaccctgcacttcaagcaggagaagaacagcgtgagcgtggacctgcccggaggcatgaccgtgctggtgagtaaggagaaggacaaagacggcaagtacagcctggaggccaccgtggataagctggagctgaagggcaccagcgacaagaacaacggagccggcactctggagggagagaagacagacaagagcaaggtgaagctgaccatcgccgacgacctgagtcagaccaagttcgagatcttcaaggaggacggcaagaccctggtgagcaagaaggtgacactgaaggataagagcagcaccgaggagaagttcaacggcaagggcgaaaccagcgagaagaccatcgtgagagcagacggcacccggctggagtacaccgacatcaagagcgacggcagcggcaaggccaaggaggtgctgaaggacttcaccctggaaggcaccctggccgccgacggcaagaccaccctgaaggtgaccgagggcaccgtggtgctgagcaagaacatcctgaagagcggcgagatcaccgtggccctggacgacagcgacaccacccgggccaccaagaagaccggcaagtgggacagcaagaccagcaccctgaccatctccgtgaacagccagaagaccaagaacctggtgttcacaaaggaggacaccatcaccgtgcagcggtacgacagcgccggcaccaacctggagggcaaggccgtggagatcaccacactgaaagagctgaagaacgccctgaag

SEQ ID NO: 14 - RNA, 864 nt, synthetic construct
  source 1..864: mol_type = other RNA
atgaaccccaaccagaagatcatcaccatcggcagcgtgtcgttgaccatcagcaccatctgcttcttcatgcagatcgccatcctgatcaccaccgtgaccctgcacttcaagcaggagaagaacagcgtgagcgtggacctgcccggagagatgaaggtgctggtgagtaaggagaaggacaaagacggcaagtacagcctggaggccaccgtggataagctggagctgaagggcaccagcgacaagaacaacggagccggcgtgctggagggagtgaaggccgccaagagcaaggcaaagctgaccatcgccgacgacctgagtcagaccaagttcgagatcttcaaggaggacggcaagaccctggtgagcaagaaggtgacactgaaggataagagcagcaccgaggagaagttcaacgacaagggcaagctgagcgagaaggtggtgaccagagcagacggcacccggctggagtacaccgagatccagaacgacggcagcggcaaggccaaggaggtgctgaagagcctgaccctggaaggcaccctgaccgccgacggcgagaccaagctgaccgtggaggccggcaccgtgaccctgagcaaggacatcagcgagagcggcgagatcaccgtggagctgaaagacaccgagaccaccccagccgacaagaagagcggcacctgggacagcaagaccagcaccctgaccatctccaagaacagccagaagaccaagcagctggtgttcacaaaggagaacaccatcaccgtgcagaagtacaacaccgccggcaccaagctggagggcagccccgcagagatcaaggacctggaggccctgaaggccgccctgaag

SEQ ID NO: 15 - RNA, 1057 nt, synthetic construct
  source 1..1057: mol_type = other RNA
gaggaaatcgcaaaatttgctcttcgcgttagatttcttttagttttctcgcaactagcaagctttttgttctcgccatgaaccccaaccagaagatcatcaccatcggcagcgtgagcctgacaatcagcaccatctgcttcttcatgcagatcgccatcctgatcaccaccgtcaccctgcacttcaagcaggagaagaacagcgtgagcgtggatctgcccggcgagatgaaggtgctggtcagcaaagagaagaacaaggacggtaagtacgacctgatcgccaccgtggacaagctggagctgaagggcaccagcgacaagaacaacggagctggcgtgctggagggcgtgaaggccgacaagagcaaggtgaagctgaccatcagcgacgacctgggccagacaaccctggaggtgttcaaggaggacggcaagaccctggtgagcaagaaggtgaccagcaaggacaagagcagcaccgaggagaagttcaacgagaagggcgaggtgagcgagaagatcatcacaagggctgacggcaccagactggagtacaccggcatcaagagcgacggcagtggcaaggccaaggaggtgctgaagggcttcatcctggagggccggtttaccgccgagaagaccaccctggtggtgaaggagggcaccgtgaccctgagcaaggacatcagcaagagcggcgaggtgtctgtggagctgcaggacaccgacagcagcgccgccacaaagaagaccgccgcctggaatagcggcaccagcaccctgaccatcaccgtgaacagcaagaagaccaaggacctggtgttcaccaaggagaacaccatcactgtgcagcagtacgacagcaacggcgtgaaactggagggcagcgccgtggagatcaccaagctggacgagatcaagaacgccctgaagtaaagctccccgggggcctcggtggcctagcttcttgccccttgggcctccccccagcccctcctccccttcctgcacccgtacccccgtggtctttgaataaagtctgagtgggcggc

SEQ ID NO: 16 - RNA, 1057 nt, synthetic construct
  source 1..1057: mol_type = other RNA
gaggaaatcgcaaaatttgctcttcgcgttagatttcttttagttttctcgcaactagcaagctttttgttctcgccatgaaccccaaccagaagatcatcaccatcggcagcgtgtcgttgaccatcagcaccatctgcttcttcatgcagatcgccatcctgatcaccaccgtgaccctgcacttcaagcaggagaagaacagcgccagcgtggacctgcccggagagatgaaggtgctggtgagtaaggagaaggacaaagacggcaagtacagcctgaaggccaccgtggataagatcgagctgaagggcaccagcgacaaggacaacggagccggcgtgctggagggaaccaaggacgacaagagcaaggcaaagctgaccatcgccgacgacctgagtaagaccaccttcgagctgttcaaggaggacggcaagaccctggtgagccggaaggtgagcagcaaggataagaccagcaccgacgagatgttcaacgagaagggcgaactgagcgccaagaccatgaccagagaggacggcaccaagctggagtacaccgagatgaagagcgacggcaccggcaaggccaaggaggtgctgaagggcttcaccctggaaggcaaggtggccaacgacaaggtgaccctggaggtgaaggagggcaccgtgaccctgagcaaggagatcgctaagagcggcgaggtgaccgtggccctgcaggacaccgacaccacccaggccaccaagaagaccggcgcctgggacagcaagaccagcaccctgaccatctccgtgaacagcaagaagaccacccagctggtgttcacaaagcaggacaccatcaccgtgcagaagtacgacagcgccggcaccaacctggagggcaccgccgtggagatcaagacactggacgagctgaagaacgccctgaagtaaagctccccgggggcctcggtggcctagcttcttgccccttgggcctccccccagcccctcctccccttcctgcacccgtacccccgtggtctttgaataaagtctgagtgggcggc

SEQ ID NO: 17 - RNA, 1060 nt, synthetic construct
  source 1..1060: mol_type = other RNA
gaggaaatcgcaaaatttgctcttcgcgttagatttcttttagttttctcgcaactagcaagctttttgttctcgccatgaaccccaaccagaagatcatcaccatcggcagcgtgtcgttgaccatcagcaccatctgcttcttcatgcagatcgccatcctgatcaccaccgtgaccctgcacttcaagcaggagaagaacagcgtgagcgtggacctgcccggaggcatgaaggtgctggtgagtaaggagaaggacaaagacggcaagtacagcctgatggccaccgtggagaagctggagctgaagggcaccagcgacaagtccaacggagccggcgtgctggagggagagaaggccgacaagagcaaggcaaagctgaccatcagccaggacctgagtcagaccaccttcgagatcttcaaggaggacggcaagaccctggtgagccggaaggtgaacagcaaggataagagcagcaccgaggagaagttcaacgacaagggcaagctgagcgagaaggtggtgaccagagcagacggcacccggctggagtacaccgagatcaagaacgacggcagcggcaaggccaaggaggtgctgaagggcttcgccctggaaggcaccctgaccgacggcggcgagaccaagctgaccgtgaccgagggcaccgtgaccctgagcaaggacatcagcaagagcggcgagatcaccgtggccctgcaggacaccgagaccaccccagccgacaagaagaccggcgagtggaagagcgacaccagcaccctgaccatctccaagaacagccagaagaccaagcagctggtgttcacaaaggagaacaccatcaccgtgcagaactacaaccgggccggcaacgcactggagggcagccccgcagagatcaaggacctggccgagctgaaggccgccctgaagtaaagctccccgggggcctcggtggcctagcttcttgccccttgggcctccccccagcccctcctccccttcctgcacccgtacccccgtggtctttgaataaagtctgagtgggcggc

SEQ ID NO: 18 - RNA, 1057 nt, synthetic construct
  source 1..1057: mol_type = other RNA
gaggaaatcgcaaaatttgctcttcgcgttagatttcttttagttttctcgcaactagcaagctttttgttctcgccatgaaccccaaccagaagatcatcaccatcggcagcgtgtcgttgaccatcagcaccatctgcttcttcatgcagatcgccatcctgatcaccaccgtgaccctgcacttcaagcaggagaagaacagcgtgagcgtggacctgcccggagagatgaaggtgctggtgagtaaggagaaggacaaagacggcaagtacagcctgatggccaccgtggataagctggagctgaagggcaccagcgacaagtccaacggagccggcactctggagggagagaagagcgacaagagcaaggcaaagctgaccatcagcgaggacctgagtaagaccaccttcgagatcttcaaggaggacggcaagaccctggtgagcaagaaggtgaacagcaaggataagagcagcatcgaggagaagttcaacgccaagggcgaactgagcgagaagaccatcctgagagcagacggcacccggctggagtacaccgagatcaagagcgacggcaccggcaaggccaaggaggtgctgaaggacttcgccctggaaggcaccctggccgccgacaagaccaccctgaaggtgaccgagggcaccgtggtgctgagcaagcacatccccaacagcggcgagatcaccgtggagctgcaggacagcgacagcacccaggccaccaagaagaccggcaagtgggacagccagaccagcaccctgaccatctccgtgaacagcaagaagaccaagaacatcgtgttcacaaaggaggacaccatcaccgtgcagaagtacgacagcgccggcaccaacctggagggcaacgccgtggagatcaagacactggacgagctgaagaacgccctgaagtaaagctccccgggggcctcggtggcctagcttcttgccccttgggcctccccccagcccctcctccccttcctgcacccgtacccccgtggtctttgaataaagtctgagtgggcggc

SEQ ID NO: 19 - RNA, 1060 nt, synthetic construct
  source 1..1060: mol_type = other RNA
gaggaaatcgcaaaatttgctcttcgcgttagatttcttttagttttctcgcaactagcaagctttttgttctcgccatgaaccccaaccagaagatcatcaccatcggcagcgtgtcgttgaccatcagcaccatctgcttcttcatgcagatcgccatcctgatcaccaccgtgaccctgcacttcaagcaggagaagaacagcgtgagcgtggacctgcccggaggcatgaaggtgctggtgagtaaggagaaggacaaagacggcaagtacagcctgatggccaccgtggagaagctggagctgaagggcaccagcgacaagaacaacggagccggcactctggagggagagaagacagacaagagcaaggtgaagctgaccatcgccgaggacctgagtaagaccaccttcgagatcttcaaggaggacggcaagaccctggtgagcaagaaggtgacactgaaggataagagcagcaccgaggagaagttcaacgagaagggcgaaatcagcgagaagaccatcgtgagagcagacggcacccggctggagtacaccgacatcaagagcgacggcagcggcaaggccaaggaggtgctgaaggacttcaccctggaaggcaccctggccgccgacggcaagaccaccctgaaggtgaccgagggcaccgtggtgctgagcaagaacatcctgaagagcggcgagatcaccgtggccctggacgacagcgacaccacccaggccaccaagaagaccggcaagtgggacagcaagaccagcaccctgaccatctccgtgaacagccagaagaccaagaacctggtgttcacaaaggaggacaccatcaccgtgcagaagtacgacagcgccggcaccaacctggagggcaaggccgtggagatcaccacactggagaaactgaaggacgccctgaagtaaagctccccgggggcctcggtggcctagcttcttgccccttgggcctccccccagcccctcctccccttcctgcacccgtacccccgtggtctttgaataaagtctgagtgggcggc

SEQ ID NO: 20 - RNA, 1060 nt, synthetic construct
  source 1..1060: mol_type = other RNA
gaggaaatcgcaaaatttgctcttcgcgttagatttcttttagttttctcgcaactagcaagctttttgttctcgccatgaaccccaaccagaagatcatcaccatcggcagcgtgtcgttgaccatcagcaccatctgcttcttcatgcagatcgccatcctgatcaccaccgtgaccctgcacttcaagcaggagaagaacagcgtgagcgtggacctgcccggaggcatgaccgtgctggtgagtaaggagaaggacaaagacggcaagtacagcctggaggccaccgtggataagctggagctgaagggcaccagcgacaagaacaacggagccggcactctggagggagagaagacagacaagagcaaggtgaagctgaccatcgccgacgacctgagtcagaccaagttcgagatcttcaaggaggacggcaagaccctggtgagcaagaaggtgacactgaaggataagagcagcaccgaggagaagttcaacggcaagggcgaaaccagcgagaagaccatcgtgagagcagacggcacccggctggagtacaccgacatcaagagcgacggcagcggcaaggccaaggaggtgctgaaggacttcaccctggaaggcaccctggccgccgacggcaagaccaccctgaaggtgaccgagggcaccgtggtgctgagcaagaacatcctgaagagcggcgagatcaccgtggccctggacgacagcgacaccacccgggccaccaagaagaccggcaagtgggacagcaagaccagcaccctgaccatctccgtgaacagccagaagaccaagaacctggtgttcacaaaggaggacaccatcaccgtgcagcggtacgacagcgccggcaccaacctggagggcaaggccgtggagatcaccacactgaaagagctgaagaacgccctgaagtaaagctccccgggggcctcggtggcctagcttcttgccccttgggcctccccccagcccctcctccccttcctgcacccgtacccccgtggtctttgaataaagtctgagtgggcggc

SEQ ID NO: 21 - RNA, 1060 nt, synthetic construct
  source 1..1060: mol_type = other RNA
gaggaaatcgcaaaatttgctcttcgcgttagatttcttttagttttctcgcaactagcaagctttttgttctcgccatgaaccccaaccagaagatcatcaccatcggcagcgtgtcgttgaccatcagcaccatctgcttcttcatgcagatcgccatcctgatcaccaccgtgaccctgcacttcaagcaggagaagaacagcgtgagcgtggacctgcccggagagatgaaggtgctggtgagtaaggagaaggacaaagacggcaagtacagcctggaggccaccgtggataagctggagctgaagggcaccagcgacaagaacaacggagccggcgtgctggagggagtgaaggccgccaagagcaaggcaaagctgaccatcgccgacgacctgagtcagaccaagttcgagatcttcaaggaggacggcaagaccctggtgagcaagaaggtgacactgaaggataagagcagcaccgaggagaagttcaacgacaagggcaagctgagcgagaaggtggtgaccagagcagacggcacccggctggagtacaccgagatccagaacgacggcagcggcaaggccaaggaggtgctgaagagcctgaccctggaaggcaccctgaccgccgacggcgagaccaagctgaccgtggaggccggcaccgtgaccctgagcaaggacatcagcgagagcggcgagatcaccgtggagctgaaagacaccgagaccaccccagccgacaagaagagcggcacctgggacagcaagaccagcaccctgaccatctccaagaacagccagaagaccaagcagctggtgttcacaaaggagaacaccatcaccgtgcagaagtacaacaccgccggcaccaagctggagggcagccccgcagagatcaaggacctggaggccctgaaggccgccctgaagtaaagctccccgggggcctcggtggcctagcttcttgccccttgggcctccccccagcccctcctccccttcctgcacccgtacccccgtggtctttgaataaagtctgagtgggcggc

SEQ ID NO: 22 - RNA, 77 nt, synthetic construct
  source 1..77: mol_type = other RNA
gaggaaatcgcaaaatttgctcttcgcgttagatttcttttagttttctcgcaactagcaagctttttgttctcgcc

SEQ ID NO: 23 - RNA, 119 nt, synthetic construct
  source 1..119: mol_type = other RNA
taaagctccccgggggcctcggtggcctagcttcttgccccttgggcctccccccagcccctcctccccttcctgcacccgtacccccgtggtctttgaataaagtctgagtgggcggc

SEQ ID NO: 24 - protein, 273 aa, Borrelia burgdorferi
  source 1..273: note = serotype 1
MKKYLLGIGLILALIACKQNVSSLDEKNSVSVDLPGEMKVLVSKEKNKDGKYDLIATVDKLELKGTSDKNNGSGVLEGVKADKSKVKLTISDDLGQTTLEVFKEDGKTLVSKKVTSKDKSSTEEKFNEKGEVSEKIITRADGTRLEYTGIKSDGSGKAKEVLKGYVLEGTLTAEKTTLVVKEGTVTLSKNISKSGEVSVELNDTDSSAATKKTAAWNSGTSTLTITVNSKKTKDLVFTKENTITVQQYDSNGTKLEGSAVEITKLDEIKNALK

SEQ ID NO: 25 - protein, 273 aa, Borrelia afzelii
  source 1..273: note = serotype 2
MKKYLLGIGLILALIACKQNVSSLDEKNSASVDLPGEMKVLVSKEKDKDGKYSLKATVDKIELKGTSDKDNGSGVLEGTKDDKSKAKLTIADDLSKTTFELFKEDGKTLVSRKVSSKDKTSTDEMFNEKGELSAKTMTRENGTKLEYTEMKSDGTGKAKEVLKNFTLEGKVANDKVTLEVKEGTVTLSKEIAKSGEVTVALNDTNTTQATKKTGAWDSKTSTLTISVNSKKTTQLVFTKQDTITVQKYDSAGTNLEGTAVEIKTLDELKNALK

SEQ ID NO: 26 - protein, 274 aa, Borrelia garinii
  source 1..274: note = serotype 3
MKKYLLGIGLILALIACKQNVSSLDEKNSVSVDLPGGMKVLVSKEKDKDGKYSLMATVEKLELKGTSDKSNGSGVLEGEKADKSKAKLTISQDLNQTTFEIFKEDGKTLVSRKVNSKDKSSTEEKFNDKGKLSEKVVTRANGTRLEYTEIKNDGSGKAKEVLKGFALEGTLTDGGETKLTVTEGTVTLSKNISKSGEITVALNDTETTPADKKTGEWKSDTSTLTISKNSQKPKQLVFTKENTITVQNYNRAGNALEGSPAEIKDLAELKAALK

SEQ ID NO: 27 - protein, 273 aa, unidentified
  source 1..273: note = Borrelia bavariensis; note = serotype 4
MKKYLLGIGLILALIACKQNVSSLDEKNSVSVDLPGEMKVLVSKEKDKDGKYSLMATVDKLELKGTSDKSNGSGTLEGEKSDKSKAKLTISEDLSKTTFEIFKEDGKTLVSKKVNSKDKSSIEEKFNAKGELSEKTILRANGTRLEYTEIKSDGTGKAKEVLKDFALEGTLAADKTTLKVTEGTVVLSKHIPNSGEITVELNDSNSTQATKKTGKWDSNTSTLTISVNSKKTKNIVFTKEDTITVQKYDSAGTNLEGNAVEIKTLDELKNALK

SEQ ID NO: 28 - protein, 274 aa, Borrelia garinii
  source 1..274: note = serotype 5
MKKYLLGIGLILALIACKQNVSSLDEKNSVSVDLPGGMKVLVSKEKDKDGKYSLMATVEKLELKGTSDKNNGSGTLEGEKTDKSKVKLTIAEDLSKTTFEIFKEDGKTLVSKKVTLKDKSSTEEKFNEKGEISEKTIVRANGTRLEYTDIKSDGSGKAKEVLKDFTLEGTLAADGKTTLKVTEGTVVLSKNILKSGEITVALDDSDTTQATKKTGKWDSKTSTLTISVNSQKTKNLVFTKEDTITVQKYDSAGTNLEGKAVEITTLEKLKDALK

SEQ ID NO: 29 - protein, 274 aa, Borrelia garinii
  source 1..274: note = serotype 6
MKKYLLGIGLILALIACKQNVSSLDEKNSVSVDLPGGMTVLVSKEKDKDGKYSLEATVDKLELKGTSDKNNGSGTLEGEKTDKSKVKSTIADDLSQTKFEIFKEDGKTLVSKKVTLKDKSSTEEKFNGKGETSEKTIVRANGTRLEYTDIKSDGSGKAKEVLKDFTLEGTLAADGKTTLKVTEGTVVLSKNILKSGEITAALDDSDTTRATKKTGKWDSKTSTLTISVNSQKTKNLVFTKEDTITVQRYDSAGTNLEGKAVEITTLKELKNALK

SEQ ID NO: 30 - protein, 274 aa, Borrelia garinii
  source 1..274: note = serotype 7
MKKYLLGIGLILALIACKQNVSSLDEKNSVSVDLPGEMKVLVSKEKDKDGKYSLEATVDKLELKGTSDKNNGSGVLEGVKAAKSKAKLTIADDLSQTKFEIFKEDGKTLVSKKVTLKDKSSTEEKFNDKGKLSEKVVTRANGTRLEYTEIQNDGSGKAKEVLKSLTLEGTLTADGETKLTVEAGTVTLSKNISESGEITVELKDTETTPADKKSGTWDSKTSTLTISKNSQKTKQLVFTKENTITVQKYNTAGTKLEGSPAEIKDLEALKAALK

SEQ ID NO: 31 - protein, 15 aa, synthetic construct
GGSGGSGGSGGSGGG

SEQ ID NO: 32 - protein, 8 aa, synthetic construct
GGGSGGGS

SEQ ID NO: 33 - protein, 7 aa, synthetic construct
GGGSGGG

SEQ ID NO: 34 - protein, 4 aa, synthetic construct
GGGS

SEQ ID NO: 35 - protein, 20 aa, synthetic construct
  VARIANT 5..20: note = Up to 4 copies of GGGS may be absent
GGGSGGGSGGGSGGGSGGGS

SEQ ID NO: 36 - protein, 4 aa, synthetic construct
GSGG

SEQ ID NO: 37 - protein, 20 aa, synthetic construct
  VARIANT 5..20: note = Up to 4 copies of GSGG may be absent
GSGGGSGGGSGGGSGGGSGG

SEQ ID NO: 38 - RNA, 47 nt, synthetic construct
  source 1..47: mol_type = other RNA
gggaaataagagagaaaagaagagtaagaagaaatataagagccacc

SEQ ID NO: 39 - RNA, 57 nt, synthetic construct
  source 1..57: mol_type = other RNA
gggaaataagagagaaaagaagagtaagaagaaatataagaccccggcgccgccacc

SEQ ID NO: 40 - RNA, 1055 nt, synthetic construct
  source 1..1055: mol_type = other RNA
aggaaatcgcaaaatttgctcttcgcgttagatttcttttagttttctcgcaactagcaagctttttgttctcgccatgaaccccaaccagaagatcatcaccatcggcagcgtgagcctgacaatcagcaccatctgcttcttcatgcagatcgccatcctgatcaccaccgtcaccctgcacttcaagcaggagaagaacagcgtgagcgtggatctgcccggcgagatgaaggtgctggtcagcaaagagaagaacaaggacggtaagtacgacctgatcgccaccgtggacaagctggagctgaagggcaccagcgacaagaacaacggagctggcgtgctggagggcgtgaaggccgacaagagcaaggtgaagctgaccatcagcgacgacctgggccagacaaccctggaggtgttcaaggaggacggcaagaccctggtgagcaagaaggtgaccagcaaggacaagagcagcaccgaggagaagttcaacgagaagggcgaggtgagcgagaagatcatcacaagggctgacggcaccagactggagtacaccggcatcaagagcgacggcagtggcaaggccaaggaggtgctgaagggcttcatcctggagggccggtttaccgccgagaagaccaccctggtggtgaaggagggcaccgtgaccctgagcaaggacatcagcaagagcggcgaggtgtctgtggagctgcaggacaccgacagcagcgccgccacaaagaagaccgccgcctggaatagcggcaccagcaccctgaccatcaccgtgaacagcaagaagaccaaggacctggtgttcaccaaggagaacaccatcactgtgcagcagtacgacagcaacggcgtgaaactggagggcagcgccgtggagatcaccaagctggacgagatcaagaacgccctgaagtaaagctccccgggggcctcggtggcctagcttcttgccccttgggcctccccccagcccctcctccccttcctgcaggttaaggaagtggtctttgaataaagtctgagtgggcggc

SEQ ID NO: 41 - RNA, 75 nt, synthetic construct
  source 1..75: mol_type = other RNA
ggaaatcgcaaaatttgctcttcgcgttagatttcttttagttttctcgcaactagcaagctttttgttctcgcc

SEQ ID NO: 42 - RNA, 119 nt, synthetic construct
  source 1..119: mol_type = other RNA
tgataataggctggagcctcggtggccatgcttcttgccccttgggcctccccccagcccctcctccccttcctgcacccgtacccccgtggtctttgaataaagtctgagtgggcggc

SEQ ID NO: 43 - RNA, 119 nt, synthetic construct
  source 1..119: mol_type = other RNA
tgataataggctggagcctcggtggcctagcttcttgccccttgggcctccccccagcccctcctccccttcctgcacccgtacccccgtggtctttgaataaagtctgagtgggcggc

SEQ ID NO: 44 - RNA, 129 nt, synthetic construct
  source 1..129: mol_type = other RNA
taaagctccccgggggcctcggtggcctagcttcttgccccttgggcctccccccagcccctcctccccttcctgcaggagattgagtgtagtgactagtggtctttgaataaagtctgagtgggcggc

SEQ ID NO: 45 - RNA, 123 nt, synthetic construct
  source 1..123: mol_type = other RNA
taaagctccccgggggcctcggtggcctagcttcttgccccttgggcctccccccagcccctcctccccttcctgcaggattgagactacgggtggtctttgaataaagtctgagtgggcggc

SEQ ID NO: 46 - RNA, 121 nt, synthetic construct
  source 1..121: mol_type = other RNA
taaagctccccgggggcctcggtggcctagcttcttgccccttgggcctccccccagcccctcctccccttcctgcagcatagacactacgtggtctttgaataaagtctgagtgggcggc

SEQ ID NO: 47 - RNA, 125 nt, synthetic construct
  source 1..125: mol_type = other RNA
taaagctccccgggggcctcggtggcctagcttcttgccccttgggcctccccccagcccctcctccccttcctgcaggagattgagtgtagtggtggtctttgaataaagtctgagtgggcggc

SEQ ID NO: 48 - RNA, 127 nt, synthetic construct
  source 1..127: mol_type = other RNA
taaagctccccgggggcctcggtggcctagcttcttgccccttgggcctccccccagcccctcctccccttcctgcaggagattgagtgtagtgacgtggtctttgaataaagtctgagtgggcggc

SEQ ID NO: 49 - RNA, 108 nt, synthetic construct
  source 1..108: mol_type = other RNA
taaagctccccgggggcctcggtggcctagcttcttgccccttgggcctccccccagcccctcctccccttcctgcagtggtctttgaataaagtctgagtgggcggc

SEQ ID NO: 50 - RNA, 76 nt, synthetic construct
  source 1..76: mol_type = other RNA
aggaaatcgcaaaatttgctcttcgcgttagatttcttttagttttctcgcaactagcaagctttttgttctcgcc

SEQ ID NO: 51 - RNA, 78 nt, synthetic construct
  source 1..78: mol_type = other RNA
taaagctccccgggggcctcggtggcctagcttcttgccccttgggcctccccccagcccctcctccccttcctgcag

SEQ ID NO: 52 - RNA, 30 nt, synthetic construct
  source 1..30: mol_type = other RNA
tggtctttgaataaagtctgagtgggcggc

SEQ ID NO: 53 - RNA, 118 nt, synthetic construct
  source 1..118: mol_type = other RNA
taaagctccccgggggcctcggtggcctagcttcttgccccttgggcctccccccagcccctcctccccttcctgcaggttaaggaagtggtctttgaataaagtctgagtgggcggc

SEQ ID NO: 54 - RNA, 11 nt, synthetic construct
  source 1..11: mol_type = other RNA
gggatcctacc

SEQ ID NO: 55 - RNA, 1057 nt, synthetic construct
  source 1..1057: mol_type = other RNA
aggaaatcgcaaaatttgctcttcgcgttagatttcttttagttttctcgcaactagcaagctttttgttctcgccatgaaccccaaccagaagatcatcaccatcggcagcgtgtcgttgaccatcagcaccatctgcttcttcatgcagatcgccatcctgatcaccaccgtgaccctgcacttcaagcaggagaagaacagcgccagcgtggacctgcccggagagatgaaggtgctggtgagtaaggagaaggacaaagacggcaagtacagcctgaaggccaccgtggataagatcgagctgaagggcaccagcgacaaggacaacggagccggcgtgctggagggaaccaaggacgacaagagcaaggcaaagctgaccatcgccgacgacctgagtaagaccaccttcgagctgttcaaggaggacggcaagaccctggtgagccggaaggtgagcagcaaggataagaccagcaccgacgagatgttcaacgagaagggcgaactgagcgccaagaccatgaccagagaggacggcaccaagctggagtacaccgagatgaagagcgacggcaccggcaaggccaaggaggtgctgaagggcttcaccctggaaggcaaggtggccaacgacaaggtgaccctggaggtgaaggagggcaccgtgaccctgagcaaggagatcgctaagagcggcgaggtgaccgtggccctgcaggacaccgacaccacccaggccaccaagaagaccggcgcctgggacagcaagaccagcaccctgaccatctccgtgaacagcaagaagaccacccagctggtgttcacaaagcaggacaccatcaccgtgcagaagtacgacagcgccggcaccaacctggagggcaccgccgtggagatcaagacactggacgagctgaagaacgccctgaagtaaagctccccgggggcctcggtggcctagcttcttgccccttgggcctccccccagcccctcctccccttcctgcaggtgaccttagagtggtctttgaataaagtctgagtgggcggc

SEQ ID NO: 56 - RNA, 120 nt, synthetic construct
  source 1..120: mol_type = other RNA
taaagctccccgggggcctcggtggcctagcttcttgccccttgggcctccccccagcccctcctccccttcctgcaggtgaccttagagtggtctttgaataaagtctgagtgggcggc

SEQ ID NO: 57 - RNA, 1062 nt, synthetic construct
  source 1..1062: mol_type = other RNA
aggaaatcgcaaaatttgctcttcgcgttagatttcttttagttttctcgcaactagcaagctttttgttctcgccatgaaccccaaccagaagatcatcaccatcggcagcgtgtcgttgaccatcagcaccatctgcttcttcatgcagatcgccatcctgatcaccaccgtgaccctgcacttcaagcaggagaagaacagcgtgagcgtggacctgcccggaggcatgaaggtgctggtgagtaaggagaaggacaaagacggcaagtacagcctgatggccaccgtggagaagctggagctgaagggcaccagcgacaagtccaacggagccggcgtgctggagggagagaaggccgacaagagcaaggcaaagctgaccatcagccaggacctgagtcagaccaccttcgagatcttcaaggaggacggcaagaccctggtgagccggaaggtgaacagcaaggataagagcagcaccgaggagaagttcaacgacaagggcaagctgagcgagaaggtggtgaccagagcagacggcacccggctggagtacaccgagatcaagaacgacggcagcggcaaggccaaggaggtgctgaagggcttcgccctggaaggcaccctgaccgacggcggcgagaccaagctgaccgtgaccgagggcaccgtgaccctgagcaaggacatcagcaagagcggcgagatcaccgtggccctgcaggacaccgagaccaccccagccgacaagaagaccggcgagtggaagagcgacaccagcaccctgaccatctccaagaacagccagaagaccaagcagctggtgttcacaaaggagaacaccatcaccgtgcagaactacaaccgggccggcaacgcactggagggcagccccgcagagatcaaggacctggccgagctgaaggccgccctgaagtaaagctccccgggggcctcggtggcctagcttcttgccccttgggcctccccccagcccctcctccccttcctgcagtccacatatcgaagtggtctttgaataaagtctgagtgggcggc

SEQ ID NO: 58 - RNA, 122 nt, synthetic construct
  source 1..122: mol_type = other RNA
taaagctccccgggggcctcggtggcctagcttcttgccccttgggcctccccccagcccctcctccccttcctgcagtccacatatcgaagtggtctttgaataaagtctgagtgggcggc

SEQ ID NO: 59 - RNA, 1061 nt, synthetic construct
  source 1..1061: mol_type = other RNA
aggaaatcgcaaaatttgctcttcgcgttagatttcttttagttttctcgcaactagcaagctttttgttctcgccatgaaccccaaccagaagatcatcaccatcggcagcgtgtcgttgaccatcagcaccatctgcttcttcatgcagatcgccatcctgatcaccaccgtgaccctgcacttcaagcaggagaagaacagcgtgagcgtggacctgcccggagagatgaaggtgctggtgagtaaggagaaggacaaagacggcaagtacagcctgatggccaccgtggataagctggagctgaagggcaccagcgacaagtccaacggagccggcactctggagggagagaagagcgacaagagcaaggcaaagctgaccatcagcgaggacctgagtaagaccaccttcgagatcttcaaggaggacggcaagaccctggtgagcaagaaggtgaacagcaaggataagagcagcatcgaggagaagttcaacgccaagggcgaactgagcgagaagaccatcctgagagcagacggcacccggctggagtacaccgagatcaagagcgacggcaccggcaaggccaaggaggtgctgaaggacttcgccctggaaggcaccctggccgccgacaagaccaccctgaaggtgaccgagggcaccgtggtgctgagcaagcacatccccaacagcggcgagatcaccgtggagctgcaggacagcgacagcacccaggccaccaagaagaccggcaagtgggacagccagaccagcaccctgaccatctccgtgaacagcaagaagaccaagaacatcgtgttcacaaaggaggacaccatcaccgtgcagaagtacgacagcgccggcaccaacctggagggcaacgccgtggagatcaagacactggacgagctgaagaacgccctgaagtaaagctccccgggggcctcggtggcctagcttcttgccccttgggcctccccccagcccctcctccccttcctgcagataactgaaggaagagtggtctttgaataaagtctgagtgggcggc

SEQ ID NO: 60 - RNA, 124 nt, synthetic construct
  source 1..124: mol_type = other RNA
taaagctccccgggggcctcggtggcctagcttcttgccccttgggcctccccccagcccctcctccccttcctgcagataactgaaggaagagtggtctttgaataaagtctgagtgggcggc

SEQ ID NO: 61 - RNA, 1067 nt, synthetic construct
  source 1..1067: mol_type = other RNA
aggaaatcgcaaaatttgctcttcgcgttagatttcttttagttttctcgcaactagcaagctttttgttctcgccatgaaccccaaccagaagatcatcaccatcggcagcgtgtcgttgaccatcagcaccatctgcttcttcatgcagatcgccatcctgatcaccaccgtgaccctgcacttcaagcaggagaagaacagcgtgagcgtggacctgcccggaggcatgaaggtgctggtgagtaaggagaaggacaaagacggcaagtacagcctgatggccaccgtggagaagctggagctgaagggcaccagcgacaagaacaacggagccggcactctggagggagagaagacagacaagagcaaggtgaagctgaccatcgccgaggacctgagtaagaccaccttcgagatcttcaaggaggacggcaagaccctggtgagcaagaaggtgacactgaaggataagagcagcaccgaggagaagttcaacgagaagggcgaaatcagcgagaagaccatcgtgagagcagacggcacccggctggagtacaccgacatcaagagcgacggcagcggcaaggccaaggaggtgctgaaggacttcaccctggaaggcaccctggccgccgacggcaagaccaccctgaaggtgaccgagggcaccgtggtgctgagcaagaacatcctgaagagcggcgagatcaccgtggccctggacgacagcgacaccacccaggccaccaagaagaccggcaagtgggacagcaagaccagcaccctgaccatctccgtgaacagccagaagaccaagaacctggtgttcacaaaggaggacaccatcaccgtgcagaagtacgacagcgccggcaccaacctggagggcaaggccgtggagatcaccacactggagaaactgaaggacgccctgaagtaaagctccccgggggcctcggtggcctagcttcttgccccttgggcctccccccagcccctcctccccttcctgcaggagagtgagtgtagtgaggtggtctttgaataaagtctgagtgggcggc

SEQ ID NO: 62 - RNA, 127 nt, synthetic construct
  source 1..127: mol_type = other RNA
taaagctccccgggggcctcggtggcctagcttcttgccccttgggcctccccccagcccctcctccccttcctgcaggagagtgagtgtagtgaggtggtctttgaataaagtctgagtgggcggc

SEQ ID NO: 63 - RNA, 1070 nt, synthetic construct
  source 1..1070: mol_type = other RNA
aggaaatcgcaaaatttgctcttcgcgttagatttcttttagttttctcgcaactagcaagctttttgttctcgccatgaaccccaaccagaagatcatcaccatcggcagcgtgtcgttgaccatcagcaccatctgcttcttcatgcagatcgccatcctgatcaccaccgtgaccctgcacttcaagcaggagaagaacagcgtgagcgtggacctgcccggaggcatgaccgtgctggtgagtaaggagaaggacaaagacggcaagtacagcctggaggccaccgtggataagctggagctgaagggcaccagcgacaagaacaacggagccggcactctggagggagagaagacagacaagagcaaggtgaagctgaccatcgccgacgacctgagtcagaccaagttcgagatcttcaaggaggacggcaagaccctggtgagcaagaaggtgacactgaaggataagagcagcaccgaggagaagttcaacggcaagggcgaaaccagcgagaagaccatcgtgagagcagacggcacccggctggagtacaccgacatcaagagcgacggcagcggcaaggccaaggaggtgctgaaggacttcaccctggaaggcaccctggccgccgacggcaagaccaccctgaaggtgaccgagggcaccgtggtgctgagcaagaacatcctgaagagcggcgagatcaccgtggccctggacgacagcgacaccacccgggccaccaagaagaccggcaagtgggacagcaagaccagcaccctgaccatctccgtgaacagccagaagaccaagaacctggtgttcacaaaggaggacaccatcaccgtgcagcggtacgacagcgccggcaccaacctggagggcaaggccgtggagatcaccacactgaaagagctgaagaacgccctgaagtaaagctccccgggggcctcggtggcctagcttcttgccccttgggcctccccccagcccctcctccccttcctgcaggagagtgagtgtagtgagtaagtggtctttgaataaagtctgagtgggcggc

SEQ ID NO: 64 - RNA, 130 nt, synthetic construct
  source 1..130: mol_type = other RNA
taaagctccccgggggcctcggtggcctagcttcttgccccttgggcctccccccagcccctcctccccttcctgcaggagagtgagtgtagtgagtaagtggtctttgaataaagtctgagtgggcggc

SEQ ID NO: 65 - RNA, 1073 nt, synthetic construct
  source 1..1073: mol_type = other RNA
aggaaatcgcaaaatttgctcttcgcgttagatttcttttagttttctcgcaactagcaagctttttgttctcgccatgaaccccaaccagaagatcatcaccatcggcagcgtgtcgttgaccatcagcaccatctgcttcttcatgcagatcgccatcctgatcaccaccgtgaccctgcacttcaagcaggagaagaacagcgtgagcgtggacctgcccggagagatgaaggtgctggtgagtaaggagaaggacaaagacggcaagtacagcctggaggccaccgtggataagctggagctgaagggcaccagcgacaagaacaacggagccggcgtgctggagggagtgaaggccgccaagagcaaggcaaagctgaccatcgccgacgacctgagtcagaccaagttcgagatcttcaaggaggacggcaagaccctggtgagcaagaaggtgacactgaaggataagagcagcaccgaggagaagttcaacgacaagggcaagctgagcgagaaggtggtgaccagagcagacggcacccggctggagtacaccgagatccagaacgacggcagcggcaaggccaaggaggtgctgaagagcctgaccctggaaggcaccctgaccgccgacggcgagaccaagctgaccgtggaggccggcaccgtgaccctgagcaaggacatcagcgagagcggcgagatcaccgtggagctgaaagacaccgagaccaccccagccgacaagaagagcggcacctgggacagcaagaccagcaccctgaccatctccaagaacagccagaagaccaagcagctggtgttcacaaaggagaacaccatcaccgtgcagaagtacaacaccgccggcaccaagctggagggcagccccgcagagatcaaggacctggaggccctgaaggccgccctgaagtaaagctccccgggggcctcggtggcctagcttcttgccccttgggcctccccccagcccctcctccccttcctgcagttgaggcacgcgtcggcaggcgcagtggtctttgaataaagtctgagtgggcggc

SEQ ID NO: 66 - RNA, 133 nt, synthetic construct
  source 1..133: mol_type = other RNA
taaagctccccgggggcctcggtggcctagcttcttgccccttgggcctccccccagcccctcctccccttcctgcagttgaggcacgcgtcggcaggcgcagtggtctttgaataaagtctgagtgggcggc

SEQ ID NO: 67 - protein, 144 aa, synthetic construct
EKNSXSVDLPGXMXVLVSKEKXKDGKYXLXATVXKXELKGTSDKXNGXGXLEGXKXXKSKXKXTIXXDLXXTXXEXFKEDGKTLVSXKVXXKDKXSXXEXFNXKGXXSXKXXXRXXGTXLEYTXXXXDGXGKAKEVLKXXXLEG

SEQ ID NO: 68 - protein, 96 aa, synthetic construct
LXVXXGTVXLSKXIXXSGEXXXXLXDXXXXXAXKKXXXWXSXTSTLTIXXNSXKXXXXVFTKXXTITVQXYXXXGXXLEGXXXEIXXLXXXKXALK